(19) United States
(12) Patent Application Publication
(10) Pub. No.: US 2016/0362408 A1
(43) Pub. Date: Dec. 15, 2016

(54) ARYL- AND HETARYL-SUBSTITUTED IMIDAZO[1,2-A]PYRIDINE-3-CARBOXAMIDES AND USE THEREOF

(71) Applicant: Bayer Pharma Aktiengesellschaft, Berlin (DE)

(72) Inventors: Alexandros VAKALOPOULOS, Hilden (DE); Ingo HARTUNG, Berlin (DE); Niels LINDNER, Wuppertal (DE); Rolf JAUTELAT, Haan (DE); Jorma HASSFELD, Dusseldorf (DE); Dirk SCHNEIDER, Wuppertal (DE); Frank WUNDER, Wuppertal (DE); Johannes-Peter STASCH, Grottaferrata (RM) (IT); Gorden REDLICH, Bochum (DE); Volkhart Min-Jian LI, Velbert (DE); Eva Maria BECKER-PELSTER, Wuppertal (DE); Andreas KNORR, Erkrath (DE)

(73) Assignee: Bayer Pharma Aktiengesellschaft, Berlin (DE)

(21) Appl. No.: 15/038,914

(22) PCT Filed: Dec. 1, 2014
(86) PCT No.: PCT/EP2014/076124

§ 371 Date: May 24, 2016

(30) Foreign Application Priority Data
Dec. 5, 2013 (EP) 13195887.8

(51) Int. Cl. C07D 471/04; A61K 31/437; A61K 31/444; A61K 31/538; A61K 31/4545; A61K 31/4709; A61K 31/502; A61K 31/496; A61K 31/506; A61K 31/541; A61K 31/5377

(52) U.S. Cl. CPC C07D 471/04; A61K 31/541; A61K 31/437; A61K 31/444; A61K 31/538; A61K 31/5377; A61K 31/4709; A61K 31/502; A61K 31/496; A61K 31/506; A61K 31/4545

(57) ABSTRACT

The present application relates to novel aryl- and hetaryl-substituted imidazo[1,2-a]pyridine-3-carboxamides, to processes for preparation thereof, to the use thereof, alone or in combinations, for treatment and/or prophylaxis of diseases, and to the use thereof for production of medicaments for treatment and/or prophylaxis of diseases, especially for treatment and/or prophylaxis of cardiovascular disorders.

The present application relates to novel aryl- and hetaryl-substituted imidazo[1,2-a]pyridine-3-carboxamides, to processes for preparation thereof, to the use thereof, alone or in combinations, for treatment and/or prophylaxis of diseases, and to the use thereof for production of medicaments for treatment and/or prophylaxis of diseases, especially for treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family known to date can be classified into two groups either by structural features or by the type of ligands: the particulate guanylate cyclases which can be stimulated by natriuretic peptides, and the soluble guanylate cyclases which can be stimulated by NO. The soluble guanylate cyclases consist of two subunits and very probably contain one haem per heterodimer, which is part of the regulatory centre. This is of central importance for the activation mechanism. NO is able to bind to the iron atom of haem and thus markedly increase the activity of the enzyme. Haem-free preparations cannot, by contrast, be stimulated by NO. Carbon monoxide (CO) is also able to bind to the central iron atom of haem, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the aforementioned processes. Under pathophysiological conditions, the NO/cGMP system can be suppressed, which can lead, for example, to hypertension, platelet activation, increased cell proliferation, endothelial dysfunction, atherosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Hitherto, for the therapeutic stimulation of the soluble guanylate cyclase, use has exclusively been made of compounds such as organic nitrates whose effect is based on NO. The latter is formed by bioconversion and activates soluble guanylate cyclase by attack at the central iron atom of haem. In addition to the side effects, the development of tolerance is one of the crucial disadvantages of this mode of treatment.

In the last few years, there have been descriptions of some compounds which stimulate soluble guanylate cyclase directly, i.e. without prior release of NO, for example 3-(5′-hydroxymethyl-2′-furyl)-1-benzylindazole [YC-1; Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

Various imidazo[1,2-a]pyridine derivatives which can be used for treating disorders are described, inter alia, in EP 0 266 890-A1, WO 89/03833-A1, JP 01258674-A [cf. Chem. Abstr. 112:178986], WO 96/34866-A1, EP 1 277 754-A1, WO 2006/015737-A1, WO 2008/008539-A2, WO 2008/082490-A2, WO 2008/134553-A1, WO 2010/030538-A2, WO 2011/113606-A1 and WO 2012/165399-A1.

It was an object of the present invention to provide novel substances which act as stimulators of soluble guanylate cyclase and are suitable as such for treatment and/or prophylaxis of diseases.

The present invention provides compounds of the general formula (I)

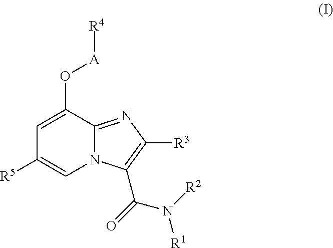

in which
A represents CH₂, CD₂ or CH(CH₃),
R¹ represents phenyl, naphthyl or 5- to 10-membered heteroaryl,
   where phenyl, naphthyl and 5- to 10-membered heteroaryl may be substituted by 1 to 4 substituents independently of one another selected from the group consisting of halogen, cyano, difluoromethyl, trifluoromethyl, (C₁-C₆)-alkyl, (C₃-C₇)-cycloalkyl, (C₁-C₄)-alkylsulphonyl, (C₃-C₆)-cycloalkylsulphonyl, (C₁-C₄)-alkylsulphonylamino, (C₃-C₆)-cycloalkylsulphonylamino, hydroxy, difluoromethoxy, trifluoromethoxy, (C₁-C₄)-alkoxy, (C₁-C₄)-alkylcarbonylamino, amino, mono-(C₁-C₄)-alkylamino, di-(C₁-C₄)-alkylamino, mono-(C₁-C₄)-alkylaminocarbonyl, di-(C₁-C₄)-alkylaminocarbonyl, phenyl, benzyl, 4- to 7-membered heterocyclyl and 5-membered heteroaryl,
      in which (C₁-C₆)-alkyl, mono-(C₁-C₄)-alkylamino and di-(C₁-C₄)-alkylamino may be substituted by 1 to 3 substituents independently of one another selected from the group consisting of fluorine, trifluoromethyl, (C₃-C₇)-cycloalkyl, hydroxy, (C₁-C₄)-alkoxy, trifluoromethoxy, 2,2,2-trifluoroethoxy, hydroxycarbonyl, (C₁-C₄)-alkoxycarbonyl, (C₁-C₄)-alkylcarbonylamino, aminocarbonyl, mono-(C₁-C₄)-alkylaminocarbonyl, di-(C₁-C₄)-alkylaminocarbonyl, (C₁-C₄)-alkylsulphonyl, (C₁-C₄)-alkylsulphonylamino, aminocarbonyloxy, phenyl, 4- to 7-membered heterocyclyl, 5-membered heteroaryl and a —NR⁶R⁷ group,
         in which
         R⁶ represents hydrogen, (C₁-C₄)-alkyl or (C₃-C₇)-cycloalkyl,
            in which (C₁-C₄)-alkyl for its part may be substituted by 1 or 2 substituents independently of one another selected from the group consisting of fluorine, trifluoromethyl, (C₃-C₇)-cycloalkyl, hydroxy, (C₁-C₄)-alkoxy, amino, mono-(C₁-C₄)-alkylamino and di-(C₁-C₄)-alkylamino,
         R⁷ represents hydrogen or (C₁-C₄)-alkyl,
         or
         in which R⁶ and R⁷ together with the nitrogen atom to which they are attached form a 4- to 7-membered heterocycle,
            in which the 4- to 7-membered heterocycle for its part may be substituted by 1 to 3 substituents independently of one another selected from the group consisting of fluorine, (C₁-C₄)-alkyl, (C₃-C₇)-cycloalkyl, hydroxy, hydroxymethyl, oxo, (C₁-C₄)-alkoxy, amino, mono-(C₁-C₄)-alkylamino and di-(C₁-C₄)-alkylamino,
      and
      in which phenyl, benzyl, 4- to 7-membered heterocyclyl and 5-membered heteroaryl may be substituted by 1 to 3 substituents independently of one another selected from the group consisting of halogen, difluoromethyl, trifluoromethyl, (C₁-C₄)-alkyl, hydroxy, difluoromethoxy, trifluoromethoxy and (C₁-C₄)-alkoxy,
   or
   where two adjacent radicals at the phenyl together with the carbon atoms to which they are attached form a 5- or 6-membered heterocycle,
      in which the 5- or 6-membered heterocycle for its part may be substituted by 1 to 3 substituents independently of one another selected from the group consisting of fluorine, trifluoromethyl, (C₁-C₄)-alkyl, hydroxy, hydroxymethyl, oxo and (C₁-C₄)-alkoxy,
R² represents hydrogen,
R³ represents hydrogen, (C₁-C₄)-alkyl, cyclopropyl, monofluoromethyl, difluoromethyl or trifluoromethyl,
R⁴ represents (C₄-C₆)-alkyl, (C₃-C₇)-cycloalkyl or phenyl,
   where (C₄-C₆)-alkyl may be substituted by 1 or 2 substituents independently of one another selected from the group consisting of fluorine and trifluoromethyl,
   where (C₃-C₇)-cycloalkyl may be substituted by 1 to 4 substituents selected independently from the group of fluorine, trifluoromethyl and (C₁-C₄)-alkyl,
   and
   where phenyl may be substituted by 1 to 4 substituents independently selected from the group consisting of halogen, cyano, monofluoromethyl, difluoromethyl, trifluoromethyl, (C₁-C₄)-alkyl, (C₁-C₄)-alkoxy, difluoromethoxy and trifluoromethoxy,
R⁵ represents hydrogen, halogen, cyano, difluoromethyl, trifluoromethyl, (C₁-C₄)-alkyl, ethynyl, (C₃-C₇)-cycloalkyl, (C₁-C₄)-alkoxy or 4- to 7-membered heterocyclyl,
   and the N-oxides, salts, solvates, salts of the N-oxides and solvates of the N-oxides or salts thereof.

The present invention provides compounds of the general formula (I)

in which
A represents CH₂, CD₂ or CH(CH₃),
R₁ represents phenyl, naphthyl or 5- to 10-membered heteroaryl,
   where phenyl, naphthyl and 5- to 10-membered heteroaryl may be substituted by 1 to 4 substituents independently of one another selected from the group consisting of halogen, cyano, difluoromethyl, trifluoromethyl, (C₁-C₆)-alkyl, (C₃-C₇)-cycloalkyl, hydroxy, difluoromethoxy, trifluoromethoxy, (C₁-C₄)-alkoxy, (C₁-C₄)-alkylcarbonylamino, amino, mono-(C₁-C₄)-alkylamino, di-(C₁-C₄)-alkylamino, mono-(C₁-C₄)-alkylaminocarbonyl, di-(C₁-C₄)-alkylaminocarbonyl, phenyl, benzyl, 4- to 7-membered heterocyclyl and 5-membered heteroaryl,
      in which (C₁-C₆)-alkyl, mono-(C₁-C₄)-alkylamino and di-(C₁-C₄)-alkylamino may be substituted by 1 to 3 substituents independently of one another selected from the group consisting of fluorine, trifluoromethyl, (C₃-C₇)-cycloalkyl, hydroxy, (C₁-C₄)-alkoxy, trifluoromethoxy, 2,2,2-trifluoroethoxy, hydroxycarbonyl, (C₁-C₄)-alkoxycarbonyl, (C₁-C₄)-alkylcarbonylamino, aminocarbonyl, mono-(C₁-C₄)-alkylaminocarbonyl, di-(C₁-C₄)-alkylaminocarbonyl, (C₁-C₄)-alkylsulphonyl, (C₁-C₄)-alkylsulphonylamino, aminocarbonyloxy, phenyl, 4- to 7-membered heterocyclyl, 5-membered heteroaryl and a —NR⁶R⁷ group,
         in which
         R⁶ represents hydrogen, (C₁-C₄)-alkyl or (C₃-C₇)-cycloalkyl,
            in which (C₁-C₄)-alkyl for its part may be substituted by 1 or 2 substituents independently of one another selected from the group consisting of fluorine, trifluoromethyl, (C₃-C₇)-cycloalkyl, hydroxy, (C₁-C₄)-alkoxy, amino, mono-(C₁-C₄)-alkylamino and di-(C₁-C₄)-alkylamino,
         R⁷ represents hydrogen or (C₁-C₄)-alkyl,
         or
         in which R⁶ and R⁷ together with the nitrogen atom to which they are attached form a 4- to 7-membered heterocycle,
            in which the 4- to 7-membered heterocycle for its part may be substituted by 1 to 3 substituents independently of one another selected from the group consisting of fluorine, (C₁-C₄)-alkyl, (C₃-C₇)-cycloalkyl, hydroxy, hydroxymethyl, oxo, (C₁-C₄)-alkoxy, amino, mono-(C₁-C₄)-alkylamino and di-(C₁-C₄)-alkylamino,
      and
      in which phenyl, benzyl, 4- to 7-membered heterocyclyl and 5-membered heteroaryl may be substituted by 1 to 3 substituents independently of one another selected from the group consisting of halogen, difluoromethyl, trifluoromethyl, (C₁-C₄)-alkyl, hydroxy, difluoromethoxy, trifluoromethoxy and (C₁-C₄)-alkoxy,
   or
   where two adjacent radicals at the phenyl together with the carbon atoms to which they are attached form a 5- or 6-membered heterocycle,
      in which the 5- or 6-membered heterocycle for its part may be substituted by 1 to 3 substituents independently of one another selected from the group consisting of fluorine, trifluoromethyl, (C₁-C₄)-alkyl, hydroxy, hydroxymethyl, oxo and (C₁-C₄)-alkoxy,
R² represents hydrogen,
R³ represents hydrogen, (C₁-C₄)-alkyl, cyclopropyl, monofluoromethyl, difluoromethyl or trifluoromethyl,
R⁴ represents (C₄-C₆)-alkyl, (C₃-C₇)-cycloalkyl or phenyl,
   where (C₄-C₆)-alkyl may be substituted by 1 or 2 substituents independently of one another selected from the group consisting of fluorine and trifluoromethyl,
   where (C₃-C₇)-cycloalkyl may be substituted by 1 to 4 substituents selected independently from the group of fluorine, trifluoromethyl and (C₁-C₄)-alkyl,
   and
   where phenyl may be substituted by 1 to 4 substituents independently selected from the group consisting of halogen, cyano, monofluoromethyl, difluoromethyl, trifluoromethyl, (C₁-C₄)-alkyl, (C₁-C₄)-alkoxy, difluoromethoxy and trifluoromethoxy,
R⁵ represents hydrogen, halogen, cyano, difluoromethyl, trifluoromethyl, (C₁-C₄)-alkyl, ethynyl, (C₃-C₇)-cycloalkyl, (C₁-C₄)-alkoxy or 4- to 7-membered heterocyclyl,
   and the N-oxides, salts, solvates, salts of the N-oxides and solvates of the N-oxides or salts thereof.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by the formula (I) and are mentioned below as embodiments and the salts, solvates and solvates of the salts thereof if the compounds that are encompassed by the formula (I) and are mentioned below are not already salts, solvates and solvates of the salts.

Preferred salts in the context of the present invention are physiologically acceptable salts of the compounds of the invention. Also encompassed are salts which are not themselves suitable for pharmaceutical applications but can be used, for example, for isolation or purification of the compounds of the invention.

Physiologically acceptable salts of the compounds of the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatography processes are preferably used for this purpose, especially HPLC chromatography on an achiral or chiral phase.

If the compounds according to the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting compounds.

The present invention additionally also encompasses prodrugs of the compounds of the invention. The term “prodrugs” in this context refers to compounds which may themselves be biologically active or inactive but are converted (for example metabolically or hydrolytically) to compounds of the invention during their residence time in the body.

In the context of the present invention, unless specified otherwise, the substituents are defined as follows:

Alkyl in the context of the invention is a straight-chain or branched alkyl radical having 1 to 6 carbon atoms. Preferred examples include: methyl, ethyl, n-propyl, isopropyl, n-butyl, isobutyl, 1-methylpropyl, tert-butyl, n-pentyl, 1-methylbutyl, 2-methylbutyl, 3-methylbutyl, isopentyl, n-hexyl, 1-methylpentyl, 1-ethylbutyl, 2-methylpentyl, 2-ethylbutyl, 3-methylpentyl, 4-methylpentyl.
Cycloalkyl in the context of the invention is a monocyclic saturated alkyl radical having 3 to 7 carbon atoms. Preferred examples include: cyclopropyl, cyclobutyl, cyclopentyl, cyclohexyl and cycloheptyl.
Alkylcarbonyl in the context of the invention is a straight-chain or branched alkyl radical having 1 to 4 carbon atoms and a carbonyl group attached in the 1 position. Preferred examples include: methylcarbonyl, ethylcarbonyl, n-propylcarbonyl, isopropylcarbonyl, n-butylcarbonyl, isobutylcarbonyl and tert-butylcarbonyl.
Alkylcarbonylamino in the context of the invention is an amino group having a straight-chain or branched alkylcarbonyl substituent which has 1 to 4 carbon atoms in the alkyl chain and is attached to the nitrogen atom via the carbonyl group. Preferred examples include: methylcarbonylamino, ethylcarbonylamino, propylcarbonylamino, n-butylcarbonylamino, isobutylcarbonylamino and tert-butylcarbonylamino.
Alkoxy in the context of the invention is a straight-chain or branched alkoxy radical having 1 to 4 carbon atoms. Preferred examples include: methoxy, ethoxy, n-propoxy, isopropoxy, 1-methylpropoxy, n-butoxy, isobutoxy and tert-butoxy.
Monoalkylamino in the context of the invention is an amino group having a straight-chain or branched alkyl substituent having 1 to 4 carbon atoms. Preferred examples include: methylamino, ethylamino, n-propylamino, isopropylamino and tert-butylamino.
Dialkylamino in the context of the invention is an amino group having two identical or different, straight-chain or branched alkyl substituents each having 1 to 4 carbon atoms. Preferred examples include: N,N-dimethylamino, N,N-diethylamino, N-ethyl-N-methylamino, N-methyl-N-n-propylamino, N-isopropyl-N-n-propylamino and N-tert-butyl-N-methylamino.
Monoalkylaminocarbonyl in the context of the invention is an amino group which is attached via a carbonyl group and has a straight-chain or branched alkyl substituent having 1 to 4 carbon atoms. Preferred examples include: methylaminocarbonyl, ethylaminocarbonyl, n-propylaminocarbonyl, isopropylaminocarbonyl, n-butylaminocarbonyl, tert-butylaminocarbonyl, n-pentylaminocarbonyl and n-hexylaminocarbonyl.
Dialkylaminocarbonyl in the context of the invention is an amino group which is attached via a carbonyl group and has two identical or different, straight-chain or branched alkyl substituents each having 1 to 4 carbon atoms. Preferred examples include: N,N-dimethylaminocarbonyl, N,N-diethylaminocarbonyl, N-ethyl-N-methylaminocarbonyl, N-methyl-N-n-propylaminocarbonyl, N-n-butyl-N-methylaminocarbonyl, N-tert-butyl-N-methylaminocarbonyl, N-n-pentyl-N-methylaminocarbonyl and N-n-hexyl-N-methylaminocarbonyl.
Alkylsulphonyl in the context of the invention is a straight-chain or branched alkyl radical which has 1 to 4 carbon atoms and is attached via a sulphonyl group. Preferred examples include: methylsulphonyl, ethylsulphonyl, n-propylsulphonyl, isopropylsulphonyl, n-butylsulphonyl and tert-butylsulphonyl.
(C₁-C₄)-Alkylsulphonylamino in the context of the invention is an amino group having a straight-chain or branched alkylsulphonyl substituent which has 1 to 4 carbon atoms in the alkyl radical and is attached to the nitrogen atom via the sulphonyl group. Preferred examples include: methylsulphonylamino, ethylsulphonylamino, propylsulphonylamino, n-butylsulphonylamino, isobutylsulphonylamino and tert-butylsulphonylamino.
Heterocyclyl or heterocycle in the context of the invention is a monocyclic saturated or partially unsaturated heterocycle having a total of 4 to 7 ring atoms which contains one to three ring heteroatoms from the group consisting of N, O and S and is attached via a ring carbon atom or optionally a ring nitrogen atom. Examples include: azetidinyl, oxetanyl, pyrrolidinyl, pyrazolidinyl, tetrahydrofuranyl, thiolanyl, piperidinyl, piperazinyl, tetrahydropyranyl, morpholinyl, thiomorpholinyl, azepanyl, diazepanyl, dihydropyrrolyl, tetrahydropyridinyl, dihydrooxazinyl or dihydropyrazinyl. Preference is given to a saturated 5- or 6-membered heterocycle having one or two ring heteroatoms from the group consisting of N, O and S. Examples include: azetidinyl, oxetanyl, pyrrolidinyl, pyrazolidinyl, tetrahydrofuranyl, piperidinyl, piperazinyl, tetrahydropyranyl, morpholinyl and thiomorpholinyl.
Heteroaryl in the context of the invention is a monocyclic or optionally bicyclic aromatic heterocycle (heteroaromatic) which has a total of 5 to 10 ring atoms, contains up to three identical or different ring heteroatoms from the group consisting of N, O and S and is attached via a ring carbon atom or optionally via a ring nitrogen atom. Examples include: furyl, pyrrolyl, thienyl, pyrazolyl, imidazolyl, thiazolyl, oxazolyl, isoxazolyl, isothiazolyl, triazolyl, oxadiazolyl, thiadiazolyl, tetrazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, quinolinyl, isoquinolinyl, naphthyridinyl, quinazolinyl, quinoxalinyl, phthalazinyl, pyrrolo[2,3-b]pyridine, pyrazolo[1,5-a]pyridine, pyrazolo[3,4-b]pyridinyl. Preferred examples include: pyrazolyl, imidazolyl, isoxazolyl, pyridyl, indolyl, indazolyl, quinolinyl, isoquinolinyl, naphthyridinyl, quinazolinyl, quinoxalinyl, phthalazinyl, pyrrolo[2,3-b]pyridine, pyrazolo[1,5-a]pyridine, pyrazolo[3,4-b]pyridinyl.
Halogen in the context of the invention includes fluorine, chlorine, bromine and iodine. Preference is given to chlorine or fluorine.

An oxo group in the context of the invention is an oxygen atom bonded via a double bond to a carbon or sulphur atom.

When radicals in the compounds of the invention are substituted, the radicals may be mono- or polysubstituted, unless specified otherwise. In the context of the present invention, all radicals which occur more than once are defined independently of one another. Substitution by one, two or three identical or different substituents is preferred.

Preference is given in the context of the present invention to compounds of the formula (I) in which
A represents CH₂,
R¹ represents phenyl, naphthyl, pyrazolyl, imidazolyl, isoxazolyl, 1,3,4-thiadiazol-2-yl, 1,3-thiazol-2-yl, 1,3-oxazol-2-yl, pyridyl, pyrimidin-2-yl, indolyl, pyrrolo[2,3-b]pyridine, indazolyl, pyrazolo[1,5-a]pyridine, quinolinyl, isoquinolinyl or cinnolinyl,
   where phenyl, naphthyl, pyrazolyl, isoxazolyl, 1,3,4-thiadiazol-2-yl, 1,3-thiazol-2-yl, 1,3-oxazol-2-yl, pyridyl, pyrimidin-2-yl, indolyl, pyrrolo[2,3-b]pyridine, indazolyl, pyrazolo[1,5-a]pyridine, quinolinyl, isoquinolinyl and cinnolinyl may be substituted by 1 to 4 substituents independently of one another selected from the group consisting of fluorine, chlorine, trifluoromethyl, (C₁-C₆)-alkyl, cyclopropyl, cyclobutyl, cyclopentyl, (C₁-C₄)-alkylsulphonyl, (C₁-C₄)-alkylsulphonylamino, trifluoromethoxy, (C₁-C₄)-alkoxy, methylcarbonylamino, ethylcarbonylamino, methylamino, ethylamino, dimethylamino, diethylamino, methylaminocarbonyl, ethylaminocarbonyl, dimethylaminocarbonyl, diethylaminocarbonyl, phenyl, benzyl, azetidinyl, pyrrolidinyl, piperidinyl, tetrahydrofuranyl, tetrahydropyranyl, piperazinyl, morpholinyl and tetrazolyl,
      in which (C₁-C₆)-alkyl, ethylamino and diethylamino may be substituted by 1 to 3 substituents independently of one another selected from the group consisting of fluorine, trifluoromethyl, cyclopropyl, cyclobutyl, hydroxy, methoxy, ethoxy, 2,2,2-trifluoroethoxy, methylcarbonylamino, ethylcarbonylamino, methylaminocarbonyl, ethylaminocarbonyl, dimethylaminocarbonyl, diethylaminocarbonyl, methylsulphonyl, ethylsulphonyl, aminocarbonyloxy, azetidin-3-yl, pyrrolidin-2-yl, pyrrolidin-3-yl, piperidin-2-yl, piperidin-3-yl, piperidin-4-yl, tetrahydrofuranyl, tetrahydropyranyl, piperazin-2-yl, piperazin-3-yl, morpholin-2-yl, morpholin-3-yl and tetrazolyl and a —NR⁶R⁷ group,
         in which
         R⁶ represents hydrogen, (C₁-C₄)-alkyl, cyclopropyl or cyclobutyl,
            in which (C₁-C₄)-alkyl may itself be substituted by 1 or 2 substituents each independently selected from the group of fluorine, trifluoromethyl, cyclopropyl, cyclobutyl, hydroxyl, methoxy and ethoxy,
         R⁷ represents hydrogen or (C₁-C₄)-alkyl,
         or
         in which R⁶ and R⁷ together with the nitrogen atom to which they are attached form an azetidinyl, pyrrolidinyl, piperidinyl, piperazinyl or morpholinyl ring,
            in which the azetidinyl, pyrrolidinyl, piperidinyl, piperazinyl and morpholinyl ring for its part may be substituted by 1 to 3 substituents independently of one another selected from the group consisting of fluorine, methyl, ethyl, cyclopropyl, cyclobutyl, hydroxy, hydroxymethyl, oxo, methoxy and ethoxy,
   or
   where two adjacent radicals at the phenyl together with the carbon atoms to which they are attached form a dihydropyrrolyl, tetrahydropyridinyl, dihydrooxazinyl or dihydropyrazinyl ring,
      in which the dihydropyrrolyl, tetrahydropyridinyl, dihydrooxazinyl and dihydropyrazinyl ring for its part may be substituted by 1 to 3 substituents independently of one another selected from the group consisting of fluorine, methyl, ethyl, hydroxy, hydroxymethyl and oxo,
R² represents hydrogen,
R³ represents methyl,
R⁴ represents phenyl,
   where phenyl is substituted by 1 to 4 substituents independently of one another selected from the group consisting of fluorine and chlorine,
R⁵ represents hydrogen, fluorine, chlorine or methyl,
   and the salts, solvates and solvates of the salts thereof.

Preference is given in the context of the present invention to compounds of the formula (I) in which
A represents CH₂,
R¹ represents phenyl, naphthyl, pyrazolyl, imidazolyl, isoxazolyl, 1,3,4-thiadiazol-2-yl, 1,3-thiazol-2-yl, 1,3-oxazol-2-yl, pyridyl, pyrimidin-2-yl, indolyl, pyrrolo[2,3-b]pyridine, indazolyl, pyrazolo[1,5-a]pyridine, quinolinyl, isoquinolinyl or cinnolinyl,
   where phenyl, naphthyl, pyrazolyl, isoxazolyl, 1,3,4-thiadiazol-2-yl, 1,3-thiazol-2-yl, 1,3-oxazol-2-yl, pyridyl, pyrimidin-2-yl, indolyl, pyrrolo[2,3-b]pyridine, indazolyl, pyrazolo[1,5-a]pyridine, quinolinyl, isoquinolinyl and cinnolinyl may be substituted by 1 to 4 substituents independently of one another selected from the group consisting of fluorine, chlorine, trifluoromethyl, (C₁-C₆)-alkyl, cyclopropyl, cyclobutyl, cyclopentyl, trifluoromethoxy, (C₁-C₄)-alkoxy, methylcarbonylamino, ethylcarbonylamino, methylamino, ethylamino, dimethylamino, diethylamino, methylaminocarbonyl, ethylaminocarbonyl, dimethylaminocarbonyl, diethylaminocarbonyl, phenyl, benzyl, azetidinyl, pyrrolidinyl, piperidinyl, tetrahydrofuranyl, tetrahydropyranyl, piperazinyl, morpholinyl and tetrazolyl,
      in which (C₁-C₆)-alkyl, ethylamino and diethylamino may be substituted by 1 to 3 substituents independently of one another selected from the group consisting of fluorine, trifluoromethyl, cyclopropyl, cyclobutyl, hydroxy, methoxy, ethoxy, 2,2,2-trifluoroethoxy, methylcarbonylamino, ethylcarbonylamino, methylaminocarbonyl, ethylaminocarbonyl, dimethylaminocarbonyl, diethylaminocarbonyl, methylsulphonyl, ethylsulphonyl, aminocarbonyloxy, azetidin-3-yl, pyrrolidin-2-yl, pyrrolidin-3-yl, piperidin-2-yl, piperidin-3-yl, piperidin-4-yl, tetrahydrofuranyl, tetrahydropyranyl, piperazin-2-yl, piperazin-3-yl, morpholin-2-yl, morpholin-3-yl and tetrazolyl and a —NR⁶R⁷ group,
         in which
         R⁶ represents hydrogen, (C₁-C₄)-alkyl, cyclopropyl or cyclobutyl,
            in which (C₁-C₄)-alkyl may itself be substituted by 1 or 2 substituents each independently selected from the group of fluorine, trifluoromethyl, cyclopropyl, cyclobutyl, hydroxyl, methoxy and ethoxy,
         R⁷ represents hydrogen or (C₁-C₄)-alkyl,
         or
         in which R⁶ and R⁷ together with the nitrogen atom to which they are attached form an azetidinyl, pyrrolidinyl, piperidinyl, piperazinyl or morpholinyl ring,
            in which the azetidinyl, pyrrolidinyl, piperidinyl, piperazinyl and morpholinyl ring for its part may be substituted by 1 to 3 substituents independently of one another selected from the group consisting of fluorine, methyl, ethyl, cyclopropyl, cyclobutyl, hydroxy, hydroxymethyl, oxo, methoxy and ethoxy,
   or
   where two adjacent radicals at the phenyl together with the carbon atoms to which they are attached form a dihydropyrrolyl, tetrahydropyridinyl, dihydrooxazinyl or dihydropyrazinyl ring,
      in which the dihydropyrrolyl, tetrahydropyridinyl, dihydrooxazinyl and dihydropyrazinyl ring for its part may be substituted by 1 to 3 substituents independently of one another selected from the group consisting of fluorine, methyl, ethyl, hydroxy, hydroxymethyl and oxo,
R² represents hydrogen,
R³ represents methyl,
R⁴ represents phenyl,
   where phenyl is substituted by 1 to 4 substituents independently of one another selected from the group consisting of fluorine and chlorine,
R⁵ represents hydrogen, fluorine, chlorine or methyl,
   and the salts, solvates and solvates of the salts thereof.

In the context of the present invention, particular preference is given to compounds of the formula (I) in which
A represents CH₂,
R¹ represents indolyl, pyrrolo[2,3-b]pyridine, indazolyl, pyrazolo[1,5-a]pyridine, quinolinyl or isoquinolinyl,
   where pyrrolo[2,3-b]pyridine, indolyl, indazolyl, pyrazolo[1,5-a]pyridine, quinolinyl and isoquinolinyl may be substituted by 1 to 3 substituents independently of one another selected from the group consisting of fluorine, chlorine, trifluoromethyl, (C₁-C₄)-alkyl, methoxy and ethoxy,
      in which (C₁-C₄)-alkyl may be substituted by 1 to 3 substituents independently of one another selected from the group consisting of fluorine, trifluoromethyl, cyclopropyl, hydroxy, methoxy, ethoxy and methylsulphonyl,
R² represents hydrogen,
R³ represents methyl,
R⁴ represents phenyl,
   where phenyl is substituted by 1 to 3 substituents independently of one another selected from the group consisting of fluorine and chlorine,
R⁵ represents hydrogen, fluorine, chlorine or methyl,
   and the salts, solvates and solvates of the salts thereof.

In the context of the present invention, particular preference is also given to compounds of the formula (I) in which
A represents CH₂,
R¹ represents pyrazol-4-yl,
   where pyrazol-4-yl may be substituted by 1 to 3 substituents independently of one another selected from the group consisting of trifluoromethyl, (C₁-C₄)-alkyl and cyclopropyl,
      in which (C₁-C₄)-alkyl may be substituted by 1 to 3 substituents independently of one another selected from the group consisting of fluorine, trifluoromethyl, cyclopropyl, hydroxy, methoxy, ethoxy, 2,2,2-trifluoroethoxy, methylsulphonyl and a —NR⁶R⁷ group,
         in which
         R⁶ represents hydrogen or (C₁-C₄)-alkyl,
            in which (C₁-C₄)-alkyl may itself be substituted by 1 or 2 substituents each independently selected from the group consisting of fluorine, trifluoromethyl, cyclopropyl, hydroxy, methoxy and ethoxy,
         R⁷ represents hydrogen or (C₁-C₄)-alkyl,
         or
         in which R⁶ and R⁷ together with the nitrogen atom to which they are attached form an azetidinyl, pyrrolidinyl, piperidinyl, piperazinyl or morpholinyl ring,
            in which the azetidinyl, pyrrolidinyl, piperidinyl, piperazinyl and morpholinyl ring for its part may be substituted by 1 to 3 substituents independently of one another selected from the group consisting of fluorine, methyl, ethyl, hydroxy, oxo, methoxy and ethoxy,
R² represents hydrogen,
R³ represents methyl,
R⁴ represents phenyl,
   where phenyl is substituted by 1 to 3 substituents independently of one another selected from the group consisting of fluorine and chlorine,
R⁵ represents hydrogen, fluorine, chlorine or methyl,
   and the salts, solvates and solvates of the salts thereof.

In the context of the present invention, preference is also given to compounds of the formula (I) in which
A is CH₂,
   and the N-oxides, salts, solvates, salts of the N-oxides and solvates of the N-oxides or salts thereof.

In the context of the present invention, preference is also given to compounds of the formula (I) in which
R¹ represents indolyl, pyrrolo[2,3-b]pyridine, indazolyl, pyrazolo[1,5-a]pyridine, quinolinyl or isoquinolinyl,
   where pyrrolo[2,3-b]pyridine, indolyl, indazolyl, pyrazolo[1,5-a]pyridine, quinolinyl and isoquinolinyl may be substituted by 1 to 3 substituents independently of one another selected from the group consisting of fluorine, chlorine, trifluoromethyl, (C₁-C₄)-alkyl, methoxy and ethoxy,
      in which (C₁-C₄)-alkyl may be substituted by 1 to 3 substituents independently of one another selected from the group consisting of fluorine, trifluoromethyl, cyclopropyl, hydroxy, methoxy, ethoxy and methylsulphonyl,
         and the N-oxides, salts, solvates, salts of the N-oxides and solvates of the N-oxides or salts thereof.

In the context of the present invention, preference is also given to compounds of the formula (I) in which
R¹ represents pyrazol-4-yl,
   where pyrazol-4-yl may be substituted by 1 to 3 substituents independently of one another selected from the group consisting of trifluoromethyl, (C₁-C₄)-alkyl and cyclopropyl,
      in which (C₁-C₄)-alkyl may be substituted by 1 to 3 substituents independently of one another selected from the group consisting of fluorine, trifluoromethyl, cyclopropyl, hydroxy, methoxy, ethoxy, 2,2,2-trifluoroethoxy, methylsulphonyl and a —NR⁶R⁷ group,
         in which
         R⁶ represents hydrogen or (C₁-C₄)-alkyl,
            in which (C₁-C₄)-alkyl may itself be substituted by 1 or 2 substituents each independently selected from the group consisting of fluorine, trifluoromethyl, cyclopropyl, hydroxy, methoxy and ethoxy,
         R⁷ represents hydrogen or (C₁-C₄)-alkyl,
         or
         in which R⁶ and R⁷ together with the nitrogen atom to which they are attached form an azetidinyl, pyrrolidinyl, piperidinyl, piperazinyl or morpholinyl ring,
            in which the azetidinyl, pyrrolidinyl, piperidinyl, piperazinyl and morpholinyl ring for its part may be substituted by 1 to 3 substituents independently of one another selected from the group consisting of fluorine, methyl, ethyl, hydroxy, oxo, methoxy and ethoxy,
               and the N-oxides, salts, solvates, salts of the N-oxides and solvates of the N-oxides or salts thereof.

In the context of the present invention, preference is also given to compounds of the formula (I) in which
R² represents hydrogen,
   and the N-oxides, salts, solvates, salts of the N-oxides and solvates of the N-oxides or salts thereof.

In the context of the present invention, preference is also given to compounds of the formula (I) in which
R³ represents methyl,
   and the N-oxides, salts, solvates, salts of the N-oxides and solvates of the N-oxides or salts thereof.

In the context of the present invention, preference is also given to compounds of the formula (I) in which
R⁴ represents phenyl,
   where phenyl is substituted by 1 to 3 substituents independently of one another selected from the group consisting of fluorine and chlorine,
      and the N-oxides, salts, solvates, salts of the N-oxides and solvates of the N-oxides or salts thereof.

In the context of the present invention, preference is also given to compounds of the formula (I) in which
R⁴ represents phenyl,
   where phenyl is substituted by 1 to 3 fluorine substituents,
      and the N-oxides, salts, solvates, salts of the N-oxides and solvates of the N-oxides or salts thereof.

In the context of the present invention, preference is also given to compounds of the formula (I) in which
R⁵ represents hydrogen, chlorine or methyl,
   and the N-oxides, salts, solvates, salts of the N-oxides and solvates of the N-oxides or salts thereof.

In the context of the present invention, preference is also given to compounds of the formula (I) in which
R⁵ represents hydrogen,
   and the N-oxides, salts, solvates, salts of the N-oxides and solvates of the N-oxides or salts thereof.

In the context of the present invention, preference is also given to compounds of the formula (I) in which
R⁵ represents chlorine,
   and the N-oxides, salts, solvates, salts of the N-oxides and solvates of the N-oxides or salts thereof.

In the context of the present invention, preference is also given to compounds of the formula (I) in which
R⁵ represents methyl,
   and the N-oxides, salts, solvates, salts of the N-oxides and solvates of the N-oxides or salts thereof.

Irrespective of the particular combinations of the radicals specified, the individual radical definitions specified in the particular combinations or preferred combinations of radicals are also replaced as desired by radical definitions of other combinations.

Particular preference is given to combinations of two or more of the preferred ranges mentioned above.

The invention further provides a process for preparing the compounds of the formula (I) according to the invention, characterized in that

[A] a compound of the formula (II)

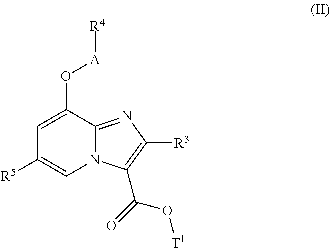

in which A, R³, R⁴ and R⁵ each have the meanings given above and
T¹ represents (C₁-C₄)-alkyl or benzyl,
is converted in an inert solvent in the presence of a suitable base or acid into a carboxylic acid of the formula (III)

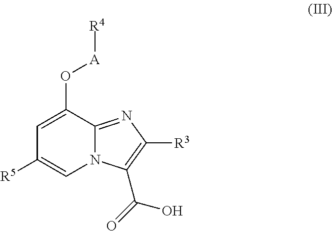

in which A, R³, R⁴ and R⁵ are each as defined above,
and this is subsequently reacted in an inert solvent under amide coupling conditions with an amine of the formula (IV)

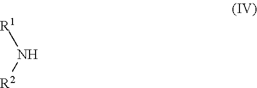

in which R¹ and R² each have the meanings given above,
   or
   [B] a compound of the formula (III-B)

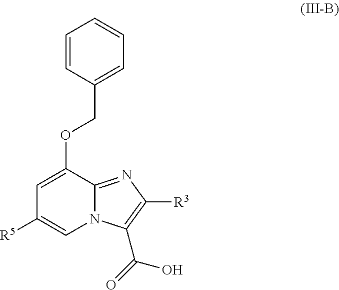

in which R³ and R⁵ each have the meanings given above,
is converted in an inert solvent under amide coupling conditions with an amine of the formula (IV) into a compound of the formula (I-B)

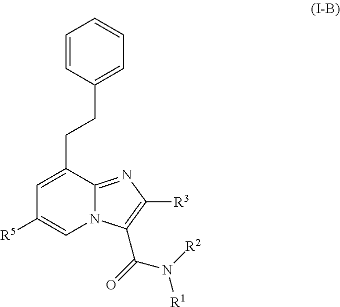

in which R¹, R², R³ and R⁵ each have the meanings given above,
and the benzyl group is subsequently detached therefrom by the methods known to the person skilled in the art and the resulting compound of the formula (V)

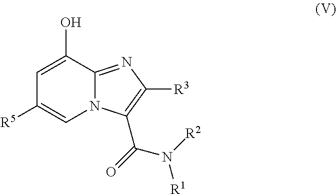

in which R¹, R², R³ and R⁵ each have the meanings given above,
is reacted in an inert solvent in the presence of a suitable base with a compound of the formula (VI)

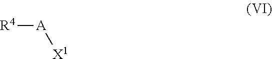

in which A and R⁴ have the meaning given above and
X¹ represents a suitable leaving group, in particular chlorine, bromine, iodine, mesylate or tosylate,
   the resulting compounds of the formulae (I) are, where appropriate, converted with the appropriate (i) solvents and/or (ii) acids or bases into their solvates, salts and/or solvates of the salts.

The compounds of the formula (I-B) form a subgroup of compounds of the formula (I) according to the invention.

The preparation processes described can be illustrated by way of example by the following synthesis schemes (Schemes 1 and 2):

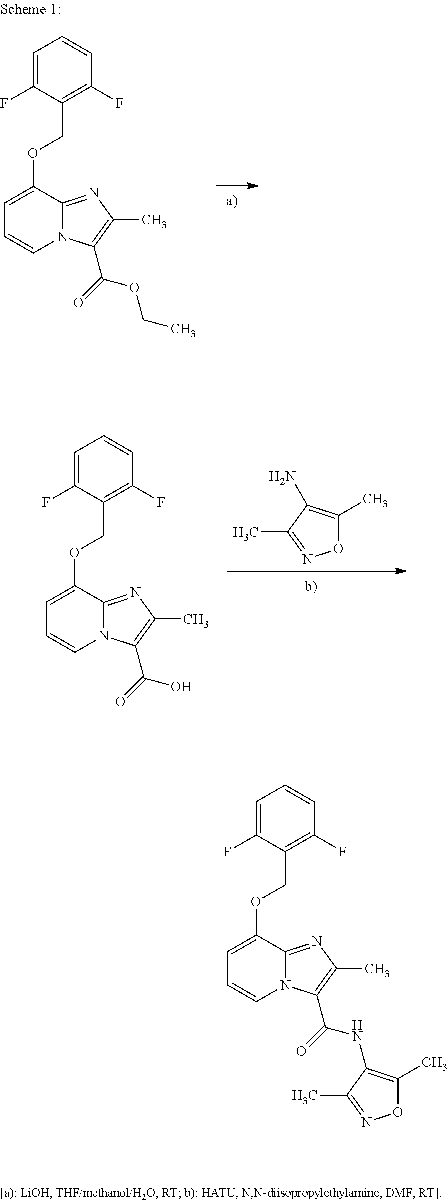

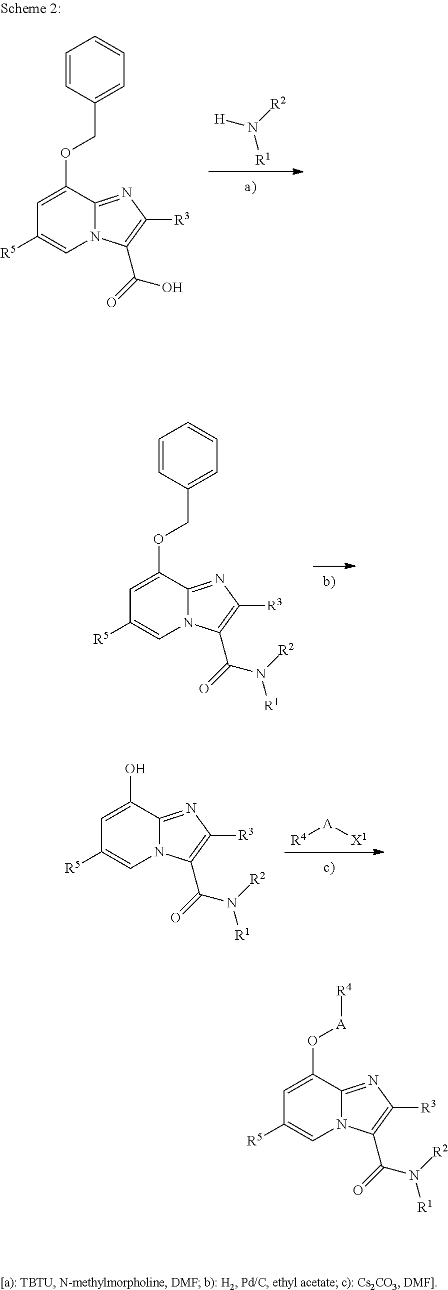

The compounds of the formulae (IV) and (VI) are commercially available, known from the literature or can be prepared in analogy to literature processes.

Inert solvents for the process step (III)+(IV)→(I) and (III-B)+(IV)→(I-B) are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, halohydrocarbons such as dichloromethane, trichloromethane, tetrachloromethane, 1,2-dichloroethane, trichloroethylene or chlorobenzene, or other solvents such as acetone, ethyl acetate, acetonitrile, pyridine, dimethyl sulphoxide, N,N-dimethylformamide, N,N′-dimethylpropyleneurea (DMPU) or N-methylpyrrolidone (NMP). It is likewise possible to use mixtures of the solvents mentioned. Preference is given to dichloromethane, tetrahydrofuran, dimethylformamide or mixtures of these solvents.

Suitable condensing agents for the amide formation in process steps (III)+(IV)→(I) and (III-B)+(IV)→(I-B) are, for example, carbodiimides such as N,N′-diethyl-, N,N′-dipropyl-, N,N′-diisopropyl- and N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulphate or 2-tert-butyl-5-methylisoxazolium perchlorate, acylamino compounds such as 2-ethoxy-1-ethoxycarbonyl-1,2-dihydroquinoline, or isobutyl chloroformate, propanephosphonic anhydride (T3P), 1-chloro-N,N,2-trimethylprop-1-en-1-amine, diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), optionally in combination with further auxiliaries such as 1-hydroxybenzotriazole (HOBt) or N-hydroxysuccinimide (HOSu), and also as bases alkali metal carbonates, for example sodium carbonate or potassium carbonate or sodium hydrogencarbonate or potassium hydrogencarbonate, or organic bases such as trialkylamines, e.g. triethylamine, N-methylmorpholine, N-methylpiperidine or N,N-diisopropylethylamine. Preference is given to using TBTU in combination with N-methylmorpholine, HATU in combination with N,N-diisopropylethylamine or 1-chloro-N,N,2-trimethylprop-1-en-1-amine.

The condensation (III)+(IV)→(I) and (III-B)+(IV)→(I-B) is generally conducted within a temperature range from −20° C. to +100° C., preferably at 0° C. to +60° C. The conversion can be carried out under atmospheric, elevated or reduced pressure (for example from 0.5 to 5 bar). In general, the reactions are carried out at atmospheric pressure.

Alternatively, the carboxylic acid of the formula (III) can also first be converted to the corresponding carbonyl chloride and the latter can then be converted directly or in a separate reaction with an amine of the formula (IV) to the compounds of the invention. The formation of carbonyl chlorides from carboxylic acids is effected by the methods known to those skilled in the art, for example by treatment with thionyl chloride, sulphuryl chloride or oxalyl chloride, in the presence of a suitable base, for example in the presence of pyridine, and optionally with addition of dimethylformamide, optionally in a suitable inert solvent.

The hydrolysis of the ester group T¹ in the compounds of the formula (II) is effected by customary methods, by treating the esters in inert solvents with acids or bases, in which latter case the salts formed at first are converted to the free carboxylic acids by treating with acid. In the case of the tert-butyl esters, the ester hydrolysis is preferably effected with acids. In the case of the benzyl esters, the ester hydrolysis is preferably effected by hydrolysis with palladium on activated carbon or Raney nickel.

Suitable inert solvents for this reaction are water or the organic solvents customary for ester hydrolysis. These preferably include alcohols such as methanol, ethanol, n-propanol, isopropanol, n-butanol or tert-butanol, or ethers such as diethyl ether, tetrahydrofuran, dioxane or glycol dimethyl ether, or other solvents such as acetone, dichloromethane, dimethylformamide or dimethyl sulphoxide. It is also possible to use mixtures of the solvents mentioned. In the case of a basic ester hydrolysis, preference is given to using mixtures of water with dioxane, tetrahydrofuran, methanol and/or ethanol.

Suitable bases for the ester hydrolysis are the customary inorganic bases. These preferably include alkali metal or alkaline earth metal hydroxides, for example sodium hydroxide, lithium hydroxide, potassium hydroxide or barium hydroxide, or alkali metal or alkaline earth metal carbonates, such as sodium carbonate, potassium carbonate or calcium carbonate. Particular preference is given to sodium hydroxide or lithium hydroxide.

Suitable acids for the ester cleavage are generally sulphuric acid, hydrogen chloride/hydrochloric acid, hydrogen bromide/hydrobromic acid, phosphoric acid, acetic acid, trifluoroacetic acid, toluenesulphonic acid, methanesulphonic acid or trifluoromethanesulphonic acid, or mixtures thereof, optionally with addition of water. Preference is given to hydrogen chloride or trifluoroacetic acid in the case of the tert-butyl esters and to hydrochloric acid in the case of the methyl esters.

The ester hydrolysis is generally carried out within a temperature range from 0° C. to +100° C., preferably at +0° C. to +50° C.

These conversions can be performed at atmospheric, elevated or reduced pressure (for example from 0.5 to 5 bar). In general, the reactions are in each case carried out at atmospheric pressure.

Inert solvents for the process step (V)+(VI)→(I) are, for example, halohydrocarbons such as dichloromethane, trichloromethane, tetrachloromethane, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as acetone, methyl ethyl ketone, ethyl acetate, acetonitrile, N,N-dimethylformamide, dimethyl sulphoxide, N,N′-dimethylpropyleneurea (DMPU), N-methylpyrrolidone (NMP) or pyridine. It is also possible to use mixtures of the solvents mentioned. Preference is given to using dimethylformamide or dimethyl sulphoxide.

Suitable bases for the process step (V)+(VI)→(I) are the customary inorganic or organic bases. These preferably include alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or caesium carbonate, optionally with addition of an alkali metal iodide, for example sodium iodide or potassium iodide, alkali metal alkoxides such as sodium methoxide or potassium methoxide, sodium ethoxide or potassium ethoxide or sodium or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo[4.3.0]non-5-ene (DBN), 1,8-diazabicyclo[5.4.0]undec-7-ene (DBU) or 1,4-diazabicyclo[2.2.2]octane (DABCO®). Preference is given to using potassium carbonate, caesium carbonate or sodium methoxide.

The reaction is generally effected within a temperature range from 0° C. to +120° C., preferably at +20° C. to +80° C., optionally in a microwave. The reaction can be carried out under atmospheric, elevated or reduced pressure (for example from 0.5 to 5 bar).

The removal of the benzyl group in the reaction step (I-B)→(V) is carried out here by customary methods known from protecting group chemistry, preferably by hydrogenolysis in the presence of a palladium catalyst, for example palladium on activated carbon, in an inert solvent, for example ethanol or ethyl acetate [see also, for example, T. W. Greene and P. G. M. Wuts, Protective Groups in Organic Synthesis, Wiley, New York, 1999].

The compounds of the formula (II) are known from the literature or can be prepared by reacting a compound of the formula (VII)

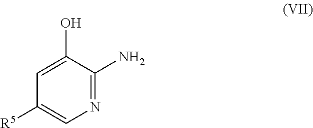

in which R⁵ has the meaning given above
in an inert solvent in the presence of a suitable base with a compound of the formula (VI) to give a compound of the formula (VIII)

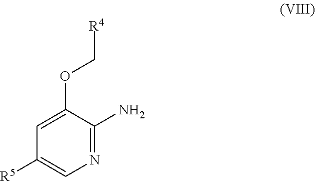

in which R⁴ and R⁵ each have the meanings given above,
and then reacting the latter in an inert solvent with a compound of the formula (IX)

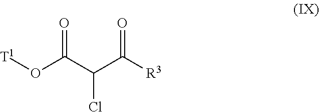

in which R³ and T¹ each have the meanings given above.

The process described is illustrated in an exemplary manner by the scheme below (Scheme 3):

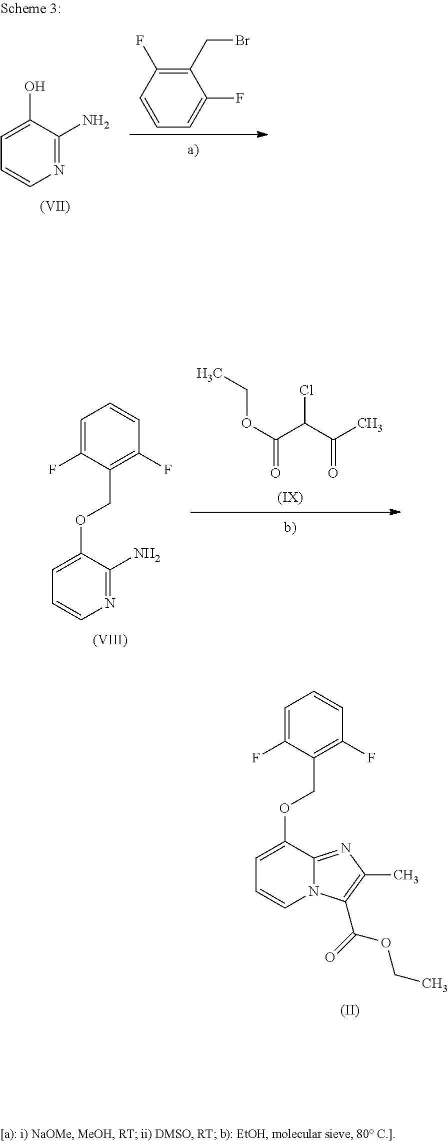

The synthesis sequence shown can be modified such that the respective reaction steps are carried out in a different order. An example of such a modified synthesis sequence is shown in Scheme 4.

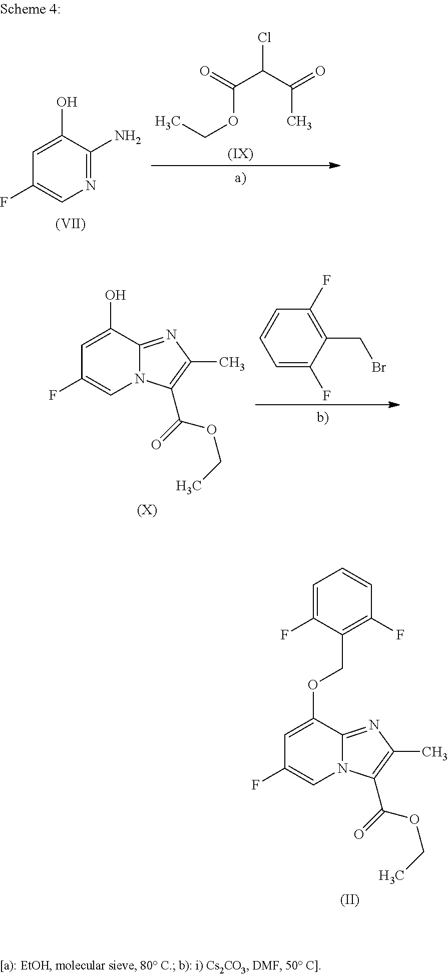

Inert solvents for the ring closure to give the imidazo[1,2-a]pyridine base skeleton (VIII)+(IX)→(II) or (VII)+(IX)→(X) are the customary organic solvents. These preferably include alcohols such as methanol, ethanol, n-propanol, isopropanol, n-butanol or tert-butanol, or ethers such as diethyl ether, tetrahydrofuran, dioxane or glycol dimethyl ether, or other solvents such as acetone, dichloromethane, dimethylformamide or dimethyl sulphoxide. It is also possible to use mixtures of the solvents mentioned. Preference is given to using ethanol.

The ring closure is generally effected within a temperature range from +50° C. to +150° C., preferably at +50° C. to +100° C., optionally in a microwave.

The ring closure (VIII)+(IX)→(II) or (VII)+(IX)→(X) is optionally effected in the presence of dehydrating reaction additives, for example in the presence of molecular sieve (pore size 4 Å). The reaction (VIII)+(IX)→(II) or (VII)+(IX)→(X) is effected using an excess of the reagent of the formula (IX), for example with 1 to 20 equivalents of the reagent (IX), where the addition of this reagent can be carried out all at once or in several portions.

As an alternative to the introduction of R⁴ by reaction of the compounds (V), (VII) or (X) with compounds of the formula (VI), as shown in Schemes 1 to 4, it is likewise possible—as shown in Scheme 5—to react these intermediates with alcohols of the formula under conditions of the Mitsunobu reaction.

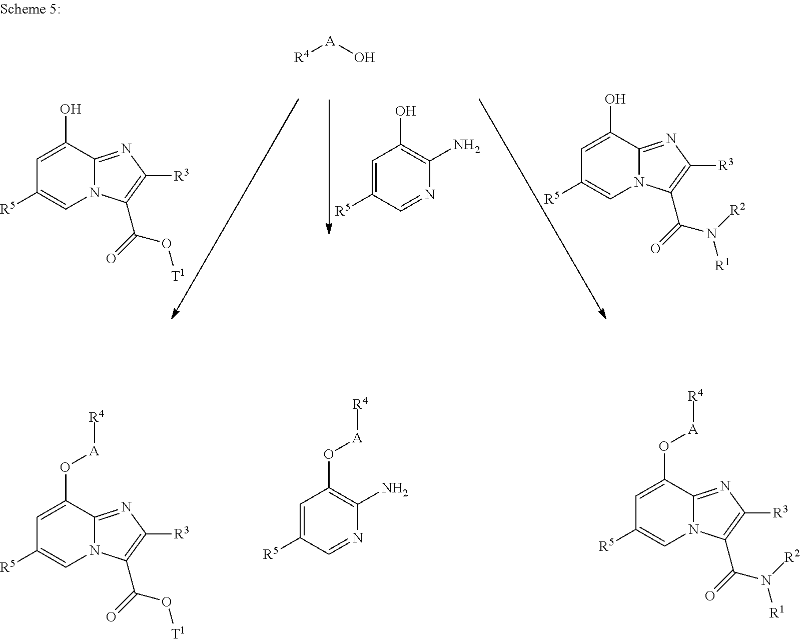

Typical reaction conditions for such Mitsunobu condensations of phenols with alcohols can be found in the relevant literature, e.g. Hughes, D. L. Org. React. 1992, 42, 335; Dembinski, R. Eur. J. Org. Chem. 2004, 2763. Typically, the reaction is carried out using an activating agent, e.h. diethyl azodicarboxylate (DEAD) or diisopropyl azodicarboxylate (DIAD), and a phosphine reagent, e.g. triphenylphosphine or tributylphosphine, in an inert solvent, e.g. THF, DCM, toluene or DMF, at a temperature between 0° C. and the boiling point of the solvent employed.

Further compounds of the invention can optionally also be prepared by conversions of functional groups of individual substituents, especially those listed for le, proceeding from the compounds of the formula (I) obtained by above processes. These conversions are performed by customary methods known to those skilled in the art and include, for example, reactions such as nucleophilic and electrophilic substitutions, oxidations, reductions, hydrogenations, transition metal-catalysed coupling reactions, eliminations, alkylation, amination, esterification, ester cleavage, etherification, ether cleavage, formation of carbonamides, and introduction and removal of temporary protective groups.

The compounds of the invention have valuable pharmacological properties and can be used for prevention and treatment of diseases in humans and animals. The compounds of the invention offer a further treatment alternative and thus enlarge the field of pharmacy.

The compounds of the invention bring about vasorelaxation and inhibition of platelet aggregation, and lead to a decrease in blood pressure and to a rise in coronary blood flow. These effects are mediated by a direct stimulation of soluble guanylate cyclase and an intracellular rise in cGMP. In addition, the compounds of the invention enhance the action of substances which increase the cGMP level, for example EDRF (endothelium-derived relaxing factor), NO donors, protoporphyrin IX, arachidonic acid or phenylhydrazine derivatives.

The compounds of the invention are suitable for treatment and/or prophylaxis of cardiovascular, pulmonary, thromboembolic and fibrotic disorders.

The compounds of the invention can therefore be used in medicaments for treatment and/or prophylaxis of cardiovascular disorders, for example hypertension, resistant hypertension, acute and chronic heart failure, coronary heart disease, stable and unstable angina pectoris, peripheral and cardiac vascular disorders, arrhythmias, atrial and ventricular arrhythmias and impaired conduction, for example atrioventricular blocks degrees I-III (AB block supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation, for example pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for treatment and/or prophylaxis of erectile dysfunction and female sexual dysfunction.

In the context of the present invention, the term “heart failure” encompasses both acute and chronic forms of heart failure, and also more specific or related types of disease, such as acute decompensated heart failure, right heart failure, left heart failure, global failure, ischaemic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure, and acute phases of worsening of existing chronic heart failure (worsening heart failure).

In addition, the compounds of the invention can also be used for treatment and/or prophylaxis of arteriosclerosis, impaired lipid metabolism, hypolipoproteinaemias, dyslipidaemias, hypertriglyceridaemias, hyperlipidaemias, hypercholesterolaemias, abetalipoproteinaemia, sitosterolaemia, xanthomatosis, Tangier disease, adiposity, obesity and of combined hyperlipidaemias and metabolic syndrome.

The compounds of the invention can also be used for treatment and/or prophylaxis of primary and secondary Raynaud's phenomenon, microcirculation impairments, claudication, peripheral and autonomic neuropathies, diabetic microangiopathies, diabetic retinopathy, diabetic ulcers on the extremities, gangrene, CREST syndrome, erythematosis, onychomycosis, rheumatic disorders and for promoting wound healing.

The compounds of the invention are furthermore suitable for treating urological disorders, for example benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic over-active bladder (OAB) and (IC), incontinence (UI), for example mixed urinary incontinence, urge urinary incontinence, stress urinary incontinence or overflow urinary incontinence (MUI, UUI, SUI, OUI), pelvic pain, benign and malignant disorders of the organs of the male and female urogenital system.

The compounds of the invention are also suitable for treatment and/or prophylaxis of kidney disorders, in particular of acute and chronic renal insufficiency and acute and chronic renal failure. In the context of the present invention, the term “renal insufficiency” encompasses both acute and chronic manifestations of renal insufficiency, and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or need for dialysis. The present invention also encompasses the use of the compounds of the invention for the treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

In addition, the compounds of the invention are also suitable for treatment and/or prophylaxis of asthmatic disorders, pulmonary arterial hypertension (PAH) and other forms of pulmonary hypertension (PH) including left-heart disease, HIV, sickle cell anaemia, thromboembolisms (CTEPH), sarcoidosis, COPD or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelinization, multiple sclerosis, thalamic degeneration, Creutzfeld-Jacob dementia, HIV dementia, schizophrenia with dementia or Korsakoff's psychosis. They are also suitable for treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

In addition, the compounds of the invention are also suitable for controlling cerebral blood flow and are thus effective agents for controlling migraines. They are also suitable for the prophylaxis and control of sequelae of cerebral infarction (cerebral apoplexy) such as stroke, cerebral ischaemia and craniocerebral trauma. The compounds of the invention can likewise be used for controlling states of pain and tinnitus.

In addition, the compounds of the invention have anti-inflammatory action and can therefore be used as anti-inflammatory agents for treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds according to the invention can also be used for treatment and/or prophylaxis of autoimmune diseases.

The compounds of the invention are also suitable for treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds of the invention are also suitable for controlling postoperative scarring, for example as a result of glaucoma operations.

The compounds of the invention can also be used cosmetically for ageing and keratinized skin.

Moreover, the compounds of the invention are suitable for treatment and/or prophylaxis of hepatitis, neoplasms, osteoporosis, glaucoma and gastroparesis.

The present invention further provides for the use of the compounds according to the invention for treatment and/or prophylaxis of disorders, especially the disorders mentioned above.

The present invention further provides for the use of the compounds according to the invention for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides the compounds of the invention for use in a method for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides for the use of the compounds according to the invention for preparing a medicament for treatment and/or prophylaxis of disorders, especially the disorders mentioned above.

The present invention further provides for the use of the compounds according to the invention for preparing a medicament for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides a method for treatment and/or prophylaxis of disorders, in particular the disorders mentioned above, using an effective amount of at least one of the compounds of the invention.

The present invention further provides a method for treatment and/or prophylaxis of heart failure, angina pectoris, hypertension, pulmonary hypertension, ischaemias, vascular disorders, renal insufficiency, thromboembolic disorders, fibrotic disorders and arteriosclerosis using an effective amount of at least one of the compounds of the invention.

The compounds of the invention can be used alone or, if required, in combination with other active ingredients. The present invention further provides medicaments comprising at least one of the compounds of the invention and one or more further active ingredients, especially for treatment and/or prophylaxis of the aforementioned disorders. Preferred examples of active compounds suitable for combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil and tadalafil;
antithrombotic agents, by way of example and with preference from the group of the platelet aggregation inhibitors, the anticoagulants or the profibrinolytic substances;
hypotensive active compounds, by way of example and with preference from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics; and/or
active compounds altering lipid metabolism, for example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

Antithrombotic agents are preferably understood to mean compounds from the group of the platelet aggregation inhibitors, the anticoagulants or the profibrinolytic substances.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, dabigatran, melagatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds of the invention are administered in combination with an alpha-1-receptor blocker, by way of example and with preference prazosin.

In a preferred embodiment of the invention, the compounds of the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin AII antagonist, by way of example and with preference losartan, candesartan, valsartan, telmisartan or embursatan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, ambrisentan or sitaxsentan.

In a preferred embodiment of the invention, the compounds of the invention are administered in combination with a renin inhibitor, by way of example and with preference aliskiren, SPP-600 or SPP-800.

In a preferred embodiment of the invention, the compounds of the invention are administered in combination with a mineralocorticoid receptor antagonist, by way of example and with preference spironolactone or eplerenone.

In a preferred embodiment of the invention, the compounds of the invention are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds of the invention are administered in combination with a CETP inhibitor, by way of example and with preference dalcetrapib, BAY 60-5521, anacetrapib or CETP vaccine (CETi-1).

In a preferred embodiment of the invention, the compounds of the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a PPAR-gamma agonist, by way of example and with preference pioglitazone or rosiglitazone.

In a preferred embodiment of the invention, the compounds of the invention are administered in combination with a PPAR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorbent, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

The present invention further provides medicaments which comprise at least one compound of the invention, typically together with one or more inert, nontoxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds of the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

It may nevertheless be necessary in some cases to deviate from the stated amounts, specifically as a function of body weight, route of administration, individual response to the active ingredient, nature of the preparation and time or interval over which administration takes place. Thus, in some cases less than the abovementioned minimum amount may be sufficient, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for the liquid/liquid solutions are based in each case on volume.

### A. EXAMPLES

### Abbreviations and Acronyms

abs. absolute (=dried)
aq. aqueous solution
br broad signal (NMR coupling pattern)
δ shift in the NMR spectrum (stated in ppm)
d doublet (NMR coupling pattern)
DCI direct chemical ionization (in MS)

### DMAP 4-N,N-dimethylaminopyridine

DMF dimethylformamide
DMSO dimethyl sulphoxide
of th. of theory (in yield)
eq. equivalent(s)
ESI electrospray ionization (in MS)
Et ethyl
h hour(s)
HPLC high-pressure, high-performance liquid chromatography
HRMS high-resolution mass spectrometry
conc. concentrated
LC/MS liquid chromatography-coupled mass spectrometry
LiHMDS lithium hexamethyldisilazide
m multiplet
Me methyl
min minute(s)
MS mass spectrometry
NMR nuclear magnetic resonance spectrometry
Ph phenyl
q quartet (NMR coupling pattern)
quint. quintet (NMR coupling pattern)
RT room temperature
Rₜ retention time (in HPLC)
s singlet (NMR coupling pattern)
t triplet (NMR coupling pattern)
tert tertiary
THF tetrahydrofuran
TBTU (benzotriazol-1-yloxy)bisdimethylaminomethylium fluoroborate
UV ultraviolet spectrometry
v/v volume to volume ratio (of a solution)
XPHOS dicyclohexyl(2′,4′,6′-triisopropylbiphenyl-2-yl)phosphine

### LC/MS and HPLC Methods:

### Method 1 (LC-MS):

Instrument: Micromass QuattroPremier with Waters UPLC Acquity; column: Thermo Hypersil GOLD 1.9μ 50 mm×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A; flow rate: 0.33 ml/min; oven: 50° C.; UV detection: 210 nm.

### Method 2 (LC-MS):

Instrument: Waters ACQUITY SQD UPLC system; column: Waters Acquity UPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

### Method 3 (LC-MS):

MS instrument type: Waters Micromass Quattro Micro; HPLC instrument type: Agilent 1100 series; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.01 min 100% A (flow rate 2.5 ml/min)→5.00 min 100% A; oven: 50° C.; flow rate: 2 ml/min; UV detection: 210 nm.

### Method 4 (DCI-MS):

Instrument: DSQ II; Thermo Fisher-Scientific; DCI with ammonia, flow rate: 1.1 ml/min; source temperature: 200° C.; ionization energy 70 eV; DCI filament heated to 800° C.; mass range 80-900.

### Method 5 (LCMS):

MS instrument: Waters SQD; HPLC instrument: Waters UPLC; column: Zorbax SB-Aq (Agilent), 50 mm×2.1 mm, 1.8 μm; mobile phase A: water+0.025% formic acid, mobile phase B: acetonitrile (ULC)+0.025% formic acid; gradient: 0.0 min 98% A-0.9 min 25% A-1.0 min 5% A-1.4 min 5% A-1.41 min 98% A-1.5 min 98% A; oven: 40° C.; flow rate: 0,600 ml/min; UV detection: DAD; 210 nm.

### Method 6 (Preparative LCMS):

MS instrument: Waters; HPLC instrument: Waters (column Waters X-Bridge C18, 18 mm×50 mm, 5 μm, mobile phase A: water+0.05% triethylamine, mobile phase B: acetonitrile (ULC)+0.05% triethylamine; gradient: 0.0 min 95% A-0.15 min 95% A-8.0 min 5% A-9.0 min 5% A; flow rate: 40 ml/min; UV detection: DAD; 210-400 nm).

or:

MS instrument: Waters; HPLC instrument: Waters (column Phenomenex Luna 5μ C18(2) 100A, AXIA Tech. 50×21.2 mm, mobile phase A: water+0.05% formic acid, mobile phase B: acetonitrile (ULC)+0.05% formic acid; gradient: 0.0 min 95% A-0.15 min 95% A-8.0 min 5% A-9.0 min 5% A; flow rate: 40 ml/min; UV detection: DAD; 210-400 nm).

### Method 7 (Preparative HPLC):

Variant a) Column: Macherey-Nagel VP 50/21 Nucleosil 100-5 C18 Nautilus. Flow rate: 25 ml/min Gradient: A=water+0.1% formic acid, B=methanol, 0 min=30% B, 2 min=30% B, 6 min=100% B, 7 min=100% B, 7.1 min=30% B, 8 min=30% B, flow rate 25 ml/min, UV detection 220 nm.

Variant b) Column: Macherey-Nagel VP 50/21 Nucleosil 100-5 C18 Nautilus. Flow rate: 25 ml/min Gradient: A=water+0.1% conc. aq ammonia, B=methanol, 0 min=30% B, 2 min=30% B, 6 min=100% B, 7 min=100% B, 7.1 min=30% B, 8 min=30% B, flow rate 25 ml/min, UV detection 220 nm.

### Method 8 (Preparative HPLC):

Column: Phenomenex Gemini C18; 110A, AXIA, 5 μm, 21.2×50 mm 5 micron; gradient: A=water+0.1% conc. ammonia, B=acetonitrile, 0 min=10% B, 2 min=10% B, 6 min=90% B, 7 min=90% B, 7.1 min=10% B, 8 min=10% B, flow rate 25 ml/min, UV detection 220 nm.

### Method 9 (Preparative HPLC):

Column: Axia Gemini 5μ C18; 110 A, 50×21.5 mm, P/NO: 00B-4435-PO-AX, S/NO: 35997-2, gradient: A=water+0.1% conc. ammonia, B=acetonitrile, 0 min=30% B, 2 min=30% B, 6 min=100% B, 7 min=100% B, 7.1 min=30% B, 8 min=30% B, flow rate 25 ml/min, UV detection 220 nm.

### Method 10:

Instrument: Waters ACQUITY SQD UPLC system; column: Waters Acquity UPLC HSS T3 1.8μ 30×2 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A oven: 50° C.; flow rate: 0.60 ml/min; UV detection: 208-400 nm.

### Method 11:

MS instrument type: Waters (Micromass) Quattro Micro; HPLC instrument type: Agilent 1100 series; column: Thermo Hypersil GOLD 3μ 20×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A; oven: 50° C.; flow rate: 2 ml/min; UV detection: 210 nm.

### Method 12:

Instrument: Thermo DFS, Trace GC Ultra; column: Restek RTX-35, 15 m×200 μm×0.33 μm; constant helium flow rate: 1.20 ml/min; oven: 60° C.; inlet: 220° C.; gradient: 60° C., 30° C./min→300° C. (maintain for 3.33 min).

Unless stated otherwise, the percentages in the tests and examples which follow are percentages by weight; parts are parts by weight. Solvent ratios, dilution ratios and concentration data for the liquid/liquid solutions are based in each case on volume.

The multiplicities of proton signals in ¹H NMR spectra reported in the paragraphs which follow represent the signal form observed in each case and do not take account of any higher-order signal phenomena. In all ¹H NMR spectra data, the chemical shifts 6 are stated in ppm.

When compounds of the invention are purified by preparative HPLC by the above-described methods in which the eluents contain additives, for example trifluoroacetic acid, formic acid or ammonia, the compounds of the invention may be obtained in salt form, for example as trifluoroacetate, formate or ammonium salt, if the compounds of the invention contain a sufficiently basic or acidic functionality. Such a salt can be converted to the corresponding free base or acid by various methods known to the person skilled in the art.

Salts may be present in sub- or superstoichiometric form, especially in the presence of an amine or a carboxylic acid. In addition, in the case of the present imidazopyridines, under acidic conditions salts may always be present, even in substoichiometric amounts, without this being apparent in the ¹H NMR and without any particular specification and notification thereof in the respective IUPAC names and structural formulae.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### General Procedures

### General Procedure 1: Amide Formation Using TBTU as Coupling Agent

1 equivalent of the carboxylic acid to be coupled (e.g. Example 3A), 1.2-1.3 equivalents of (benzotriazol-1-yloxy)bisdimethylaminomethylium fluoroborate (TBTU) and 6 equivalents of 4-methylmorpholine were initially charged in DMF (about 0.1-0.2 M based on the carboxylic acid to be coupled), and 1.2 to 1.5 equivalents of the amine to be coupled were then added and the mixture was stirred at RT overnight.

Illustrative workup of the reaction mixture: Water was added to the reaction solution, the resulting precipitate was stirred for another 30 min, filtered off with suction and washed thoroughly with water and dried under high vacuum overnight. Alternatively, the crude reaction mixture was concentrated directly and purified further by preparative HPLC.

### General Procedure 2: Amide Formation Using HATU as Coupling Agent

1 equivalent of the carboxylic acid to be coupled (e.g. Example 3A, 6A, 11A, 16A, 19A, 21A, 23A, 25A or 26A), 1.2 to 1.3 equivalents of O-(7-azabenzotriazol-1-yl)-N,N,N′N′-tetramethyluronium hexafluorophosphate (HATU) and 3 to 4 equivalents of N,N-diisopropylethylamine were initially charged in DMF (about 0.2 M based on the carboxylic acid to be coupled), and 1.2 to 1.5 equivalents of the amine to be coupled were added and the mixture was stirred at RT overnight.

Illustrative workup of the reaction mixture: Water was added to the reaction solution, the resulting precipitate was stirred for another 30 min, filtered off with suction and washed thoroughly with water and dried under high vacuum overnight. Alternatively, the crude reaction mixture was, either directly after concentration under reduced pressure or after extractive work-up, purified further by preparative HPLC.

### General Procedure 3: Amide Formation Using the Ghosez Reagent for Carboxylic Acid Activation

1 equivalent of the carboxylic acid to be coupled (e.g. Example 3A, 6A, 11A, 16A, 19A, 21A, 23A, 25A or 26A) was initially charged in THF (about 0.1 to 0.2 M based on the carboxylic acid to be coupled), and 1.5 equivalents of 1-chloro-N,N,2-trimethylprop-1-en-1-amine (Ghosez reagent) were added and the mixture was stirred at RT for 30 min 1.2 equivalents of the amine component were then added, and the suspension was stirred at RT overnight. Optionally (e.g. in the case of incomplete conversion), another 1.5 equivalents of 1-chloro-N,N,2-trimethylprop-1-en-1-amine and then further amine to be coupled were added, and the suspension was once more stirred at RT overnight. The reaction mixture was concentrated and the crude product was purified, for example by preparative HPLC.

### Representative Procedure 4: Amide Formation Using the Carbonyl Chloride

1 equivalent of the carbonyl chloride to be coupled (e.g. Example 27A) was initially charged in THF (about 0.02 to 0.03 M), and 1.2 equivalents of the amine to be coupled and 4 equivalents of N,N-diisopropylethylamine were added and the mixture was stirred at RT overnight. The reaction solution was concentrated by rotary evaporation and re-dissolved in a little acetonitrile, and water was added. The precipitated solid was stirred for about 30 min, filtered off and washed thoroughly with water. Alternatively, the crude reaction product was purified further by preparative HPLC.

### Starting Materials and Intermediates

### Example 1A

### 3-[(2,6-Difluorobenzyl)oxy]pyridine-2-amine

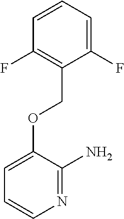 [SMILES: Nc1ncccc1OCc1c(F)cccc1F]

At RT, 51 g of sodium methoxide (953 mmol, 1.05 equivalents) were initially charged in 1000 ml of methanol, 100 g of 2-amino-3-hydroxypyridine (908 mmol, 1 equivalent) were added and the mixture was stirred at RT for another 15 min. The reaction mixture was substantially concentrated under reduced pressure, the residue was taken up in 2500 ml of DMSO, and 197 g of 2,6-difluorobenzyl bromide (953 mmol, 1.05 equivalents) were added. After 4 h at RT, the reaction mixture was added to 20 l of water, the mixture was stirred for 15 min and the solid was filtered off with suction. The filter cake was washed with 1 l of water and 100 ml of isopropanol and 500 ml of petroleum ether and dried under high vacuum. This gave 171 g of the title compound (78% of theory).

¹H-NMR (400 MHz, DMSO-d₆): δ=5.10 (s, 2H); 5.52 (br. s, 2H), 6.52 (dd, 1H); 7.16-7.21 (m, 3H); 7.49-7.56 (m, 2H).

### Example 2A

### Ethyl 8-[(2,6-difluorobenzyl)oxy]-2-methylimidazo[1,2-a]pyridine-3-carboxylate

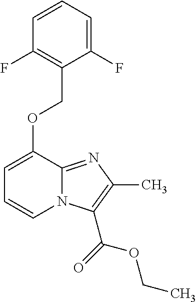 [SMILES: CCOC(=O)c1c(C)nc2c(OCc3c(F)cccc3F)cccn12]

170 g of 3-[(2,6-difluorobenzyl)oxy]pyridine-2-amine (Example 1A; 719 mmol, 1 equivalent) were initially charged in 3800 ml of ethanol, and 151 g of powdered molecular sieve 3 Å and 623 g of ethyl 2-chloroacetoacetate (3.6 mol, 5 equivalents) were added. The resulting reaction mixture was heated at reflux for 24 h and then filtered off with suction through kieselguhr and concentrated under reduced pressure. The residue crystallized on prolonged standing (48 h) at RT. The crystal slurry was filtered, three times stirred with a little isopropanol and in each case filtered off with suction, and finally washed with diethyl ether. This gave 60.8 g (23.4% of theory) of the title compound. The combined mother liquor of the filtration steps was chromatographed on silica gel using the mobile phase cyclohexane/diethyl ether, giving a further 46.5 g (18.2% of theory; total yield: 41.6% of theory) of the title compound.

LC-MS (Method 2): Rₜ=1.01 min

MS (ESpos): m/z=347 (M+H)⁺

¹H-NMR (400 MHz, DMSO-d₆): δ=1.36 (t, 3H); 2.54 (s, 3H; obscured by DMSO signal); 4.36 (q, 2H); 5.33 (s, 2H); 7.11 (t, 1H); 7.18-7.27 (m, 3H); 7.59 (quint, 1H); 8.88 (d, 1H).

### Example 3A

### 8-[(2,6-Difluorobenzyl)oxy]-2-methylimidazo[1,2-a]pyridine-3-carboxylic acid

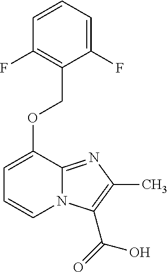 [SMILES: Cc1nc2c(OCc3c(F)cccc3F)cccn2c1C(=O)O]

107 g of ethyl 8-[(2,6-difluorobenzyl)oxy]-2-methylimidazo[1,2-a]pyridine-3-carboxylate (Example 2A; 300 mmol, 1 equivalent) were dissolved in 2.8 l of THF/methanol (1:1), 1.5 l of 1 N aqueous lithium hydroxide solution (1.5 mol, 5 equivalents) were added and the mixture was stirred at RT for 16 h. The organic solvents were removed under reduced pressure and the resulting aqueous solution was, in an ice bath, adjusted to pH 3-4 using 1 N hydrochloric acid. The resulting solid was filtered off with suction, washed with water and isopropanol and dried under reduced pressure. This gave 92 g (95% of theory) of the title compound.

LC-MS (Method 2): Rₜ=0.62 min

MS (ESpos): m/z=319.1 (M+H)⁺

¹H-NMR (400 MHz, DMSO-d₆): δ=2.55 (s, 3H; superposed by DMSO signal); 5.32 (s, 2H); 7.01 (t, 1H); 7.09 (d, 1H); 7.23 (t, 2H); 7.59 (quint, 1H); 9.01 (d, 1H).

### Example 4A

### 3-(Cyclohexylmethoxy)pyridine-2-amine

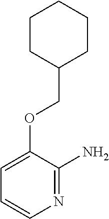 [SMILES: Nc1ncccc1OCC1CCCCC1]

96 g of aqueous sodium hydroxide solution (45% strength, 1081 mmol, 1 equivalent) were initially charged in 1170 ml of methanol at RT, 119 g of 2-amino-3-hydroxypyridine (1080 mmol, 1 equivalent) were added and the mixture was stirred at RT for another 10 min. The reaction mixture was substantially concentrated under reduced pressure, the residue was taken up in 2900 ml of DMSO, and 101 g of cyclohexylmethyl bromide (1135 mmol, 1.05 equivalents) were added. After 16 h at RT, the reaction mixture was stirred into 6 l of water, the aqueous solution was extracted twice with in each case 21 of ethyl acetate, and the combined organic phases were washed with in each case 1 l of saturated aqueous sodium bicarbonate solution and water, dried, filtered and concentrated. The residue was stirred with 500 ml of pentane, filtered off with suction and dried under reduced pressure. This gave 130 g (58.3% of theory).

LC-MS (Method 3): Rₜ=1.41 min

MS (ESpos): m/z=207.1 (M+H)⁺

### Example 5A

### Ethyl 8-(cyclohexylmethoxy)-2-methylimidazo[1,2-a]pyridine-3-carboxylate

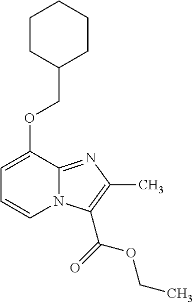 [SMILES: CCOC(=O)c1c(C)nc2c(OCC3CCCCC3)cccn12]

130 g of 3-(cyclohexylmethoxy)pyridine-2-amine (Example 4A; 630 mmol, 1 equivalent) were initially charged in 3950 ml of ethanol, and 436 ml of ethyl 2-chloroacetoacetate (3.2 mol, 5 equivalents) were added. The resulting reaction mixture was heated at reflux for 24 h and then concentrated under reduced pressure. The crude product thus obtained was chromatographed on silica gel using the mobile phase cyclohexane/diethyl ether, giving 66.2 g (33.2% of theory) of the title compound.

LC-MS (Method 2): Rₜ=1.17 min

MS (ESpos): m/z=317.1 (M+H)⁺

¹H-NMR (400 MHz, DMSO-d₆): δ=1.02-1.31 (m, 5H); 1.36 (t, 3H); 1.64-1.77 (m, 3H); 1.79-1.90 (m, 3H); 2.60 (s, 3H); 3.97 (d, 2H); 4.35 (q, 2H); 6.95 (d, 1H); 7.03 (t, 1H); 8.81 (d, 1H).

### Example 6A

### 8-(Cyclohexylmethoxy)-2-methylimidazo[1,2-a]pyridine-3-carboxylic acid

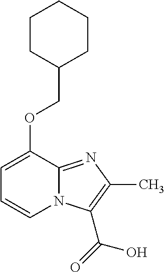 [SMILES: Cc1nc2c(OCC3CCCCC3)cccn2c1C(=O)O]

50 g of ethyl 8-(cyclohexylmethoxy)-2-methylimidazo[1,2-a]pyridine-3-carboxylate (Example 5A; 158 mmol, 1 equivalent) were dissolved in 600 ml of dioxane, 790 ml of 2 N aqueous sodium hydroxide solution (1.58 mol, 10 equivalents) were added and the mixture was stirred at RT for 16 h. 316 ml of 6 N aqueous hydrochloric acid were added and the mixture was concentrated to about ⅕ of the total volume. The resulting solid was filtered off with suction, washed with water and tert-butyl methyl ether and dried under reduced pressure. This gave 35 g (74% of theory) of the title compound.

LC-MS (Method 2): Rₜ=0.81 min

MS (ESpos): m/z=289.0 (M+H)⁺

¹H-NMR (400 MHz, DMSO-d₆): δ=1.03-1.44 (m, 5H); 1.64-1.78 (m, 3H); 1.81-1.92 (m, 3H); 2.69 (s, 3H); 4.07 (d, 2H); 7.30-7.36 (m, 2H); 9.01 (d, 1H).

### Example 7A

### 5-Fluoro-2-nitropyridin-3-ol

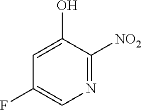 [SMILES: O=[N+]([O-])c1ncc(F)cc1O]

With ice cooling, 5 g of 5-fluoropyridin-3-ol (44 mmol, 1 equivalent) were dissolved in 43 ml of concentrated sulphuric acid, and 2.8 ml of concentrated nitric acid were added at 0° C. over 5 min. The reaction was warmed to RT and stirred overnight. The mixture was poured onto 100 g of ice and stirred for 30 min. The crystals were filtered off with suction and dried under reduced pressure. This gave 5.6 g (81% of theory) of the title compound which was used without further purification for the next reaction.

LC-MS (Method 2): Rₜ=0.45 min

MS (ESneg): m/z=156.9 (M−H)⁻

¹H-NMR (400 MHz, DMSO-d₆): δ=7.5 (dd, 1H); 8.08 (d, 1H); 12.2 (br. s, 1H).

### Example 8A

### 2-Amino-5-fluoropyridin-3-ol

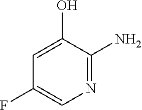

5.6 g of 5-fluoro-2-nitropyridin-3-ol (Example 7A; 36 mmol) were dissolved in 2 l of ethanol, a catalytic amount of palladium on activated carbon (10%) was added and the mixture was hydrogenated under 1 atmosphere of hydrogen for 16 h. The mixture was filtered off through kieselguhr, and the filtrate was concentrated. The filter cake was rinsed with methanol until the filtrate no longer had a yellow colour. The filtrate was concentrated, giving a second product batch. A total of 4.26 g (85% of theory) of the title compound were obtained.

LC-MS (Method 2): Rₜ=0.17 min

MS (ESpos): m/z=128.9 (M+H)⁺

¹H-NMR (400 MHz, DMSO-d₆): δ=5.4 (br. s, 2H); 6.8 (dd, 1H); 7.4 (d, 1H).

### Example 9A

### Ethyl 6-fluoro-8-hydroxy-2-methylimidazo[1,2-a]pyridine-3-carboxylate

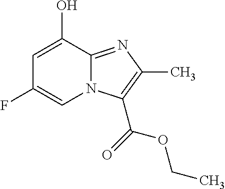 [SMILES: CCOC(=O)c1c(C)nc2c(O)cc(F)cn12]

3.2 g of 2-amino-5-fluoropyridin-3-ol (Example 8A; 25 mmol, 1 equivalent) were initially charged in 155 ml of ethanol, 1.5 g of powdered molecular sieve 3 Å and 20.6 g of ethyl 2-chloroacetoacetate (125 mmol, 5 equivalents) were added and the mixture was heated at reflux overnight. The reaction solution was concentrated and chromatographed (Biotage Isolera Four; SNAP Cartridge KP-Sil 50 g; cyclohexane/ethyl acetate gradient; then dichloromethane/methanol gradient). The crude product was subjected to incipient dissolution in a little methanol, tert-butyl methyl ether was added and the crystals were filtered off with suction and rinsed with tert-butyl methyl ether. 570 mg (10% of theory) of the title compound were obtained.

LC-MS (Method 2): Rₜ=0.77 min

MS (ESpos): m/z=239.2 (M+H)⁺

¹H-NMR (400 MHz, DMSO-d₆): δ=1.39 (t, 3H); 2.64 (s, 3H); 4.40 (q, 2H); 7.20 (br. d, 1H); 8.9 (dd, 1H); 12.5 (br., 1H).

### Example 10A

### Ethyl 8-[(2,6-difluorobenzyl)oxy]-6-fluoro-2-methylimidazo[1,2-a]pyridine-3-carboxylate

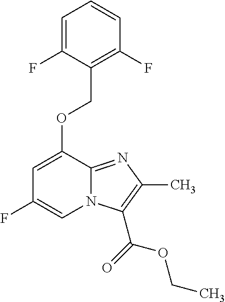 [SMILES: CCOC(=O)c1c(C)nc2c(OCc3c(F)cccc3F)cc(F)cn12]

560 mg of ethyl 6-fluoro-8-hydroxy-2-methylimidazo[1,2-a]pyridine-3-carboxylate (Example 9A; 2.4 mmol, 1.0 equivalent), 1.7 g of caesium carbonate (5.17 mmol, 2.2 equivalents) and 535 mg of 2,6-difluorobenzyl bromide (2.6 mmol, 1.1 equivalents) were initially charged in 34 ml of dry DMF, and the mixture was warmed to 50° C. for 15 min. Water was added, the mixture was stirred for 30 min and the crystals were filtered off with suction and washed with water. This gave 560 mg (65% of theory) of the title compound.

LC-MS (Method 2): Rₜ=1.18 min

MS (ESpos): m/z=365.1 (M+H)⁺

¹H-NMR (400 MHz, DMSO-d₆): δ=1.37 (t, 3H); 2.55 (s, 3H; superposed by DMSO signal); 4.38 (q, 2H); 5.89 (s, 2H); 7.23 (t, 2H); 7.44 (dd, 1H); 7.60 (quint., 1H); 8.90 (dd, 1H).

### Example 11A

### 8-[(2,6-Difluorobenzyl)oxy]-6-fluoro-2-methylimidazo[1,2-a]pyridine-3-carboxylic acid

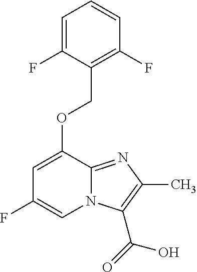 [SMILES: Cc1nc2c(OCc3c(F)cccc3F)cc(F)cn2c1C(=O)O]

550 mg of ethyl 8-[(2,6-difluorobenzyl)oxy]-6-fluoro-2-methylimidazo[1,2-a]pyridine-3-carboxylate (Example 10A; 1.5 mmol, 1 equivalent) were dissolved in 64 ml of THF and 12 ml of methanol, 7.5 ml of 1N aqueous lithium hydroxide solution were added and the mixture was stirred at RT overnight. 8 ml of 1N hydrochloric acid were added, and the mixture was concentrated. The crystals formed were filtered off with suction and washed with water. This gave 429 mg of the title compound (80% of theory).

LC-MS (Method 1): Rₜ=0.90 min

MS (ESpos): m/z=337.1 (M+H)⁺

¹H-NMR (400 MHz, DMSO-d₆): δ=2.54 (s, 3H; superposed by DMSO signal); 5.84 (s, 2H); 7.23 (t, 2H); 7.40 (dd, 1H); 7.51 (quint., 1H); 8.92 (dd, 1H); 13.28 (br. s, 1H).

### Example 12A

### 5-Chloro-2-nitropyridin-3-ol

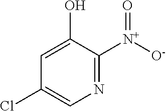 [SMILES: O=[N+]([O-])c1ncc(Cl)cc1O]

With ice cooling, 30 g of 5-chloropyridin-3-ol (232 mmol, 1 equivalent) were dissolved in 228 ml of concentrated sulphuric acid, and 24 ml of concentrated nitric acid were added slowly at 0° C. The reaction was warmed to RT and stirred overnight. The mixture was stirred into an ice/water mixture and stirred for 30 min. The crystals were filtered off, washed with cold water and air-dried. This gave 33 g (82% of theory) of the title compound which was used without further purification for the next reaction.

LC-MS (Method 2): Rₜ=0.60 min

MS (ESneg): m/z=172.9/174.9 (M−H)⁻

¹H-NMR (400 MHz, DMSO-d₆): δ=7.71 (d, 1H); 8.10 (d, 1H); 12.14 (br. 1H).

### Example 13A

### 5-Chloro-3-[(2,6-difluorobenzyl)oxy]-2-nitropyridine

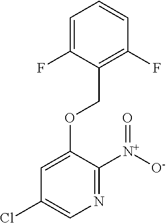 [SMILES: O=[N+]([O-])c1ncc(Cl)cc1OCc1c(F)cccc1F]

33 g of 5-chloro-2-nitropyridin-3-ol (Example 12A; 189 mmol, 1 equivalent) and 61.6 g of caesium carbonate (189 mmol, 1 equivalent) were initially charged in 528 ml of DMF, 40.4 g of 2,6-difluorobenzyl bromide (189 mmol, 1 equivalent) were added and the mixture was stirred at RT overnight. The reaction mixture was stirred into a mixture of water/1N hydrochloric acid, and the crystals were filtered off with suction, washed with water and air-dried. 54.9 g (97% of theory) of the title compound were obtained.

¹H-NMR (400 MHz, DMSO-d₆): δ=5.46 (s, 2H); 7.22 (t, 2H); 7.58 (quint., 1H); 8.28 (d, 1H); 8.47 (d, 1H).

### Example 14A

### 5-Chloro-3-[(2,6-difluorobenzyl)oxy]pyridine-2-amine

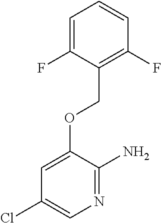 [SMILES: Nc1ncc(Cl)cc1OCc1c(F)cccc1F]

59.7 g of 5-chloro-3[(2,6-difluorobenzyl)oxy]-2-nitropyridine (Example 13A; 199 mmol, 1 equivalent) were initially charged in 600 ml of ethanol, 34.4 g of iron powder (616 mmol, 3.1 equivalents) were added and the mixture was heated to the boil. 152 ml of concentrated hydrochloric acid were slowly added dropwise, and the mixture was heated at reflux for a further 30 min. The reaction mixture was cooled and stirred into an ice/water mixture. The resulting mixture was adjusted to pH 5 using sodium acetate, the crystals were filtered off with suction and air-dried and then dried under reduced pressure at 50° C. 52.7 g (98% of theory) of the title compound were obtained.

LC-MS (Method 2): Rₜ=0.93 min

MS (ESpos): m/z=271.1/273.1 (M+H)⁺

¹H-NMR (400 MHz, DMSO-d₆): δ=5.14 (s, 2H); 5.82 (br. s, 2H); 7.20 (t, 2H); 7.35 (d, 1H); 7.55 (quint., 1H); 7.56 (d, 1H).

### Example 15A

### Ethyl 6-chloro-8-[(2,6-difluorobenzyl)oxy]-2-methylimidazo[1,2-a]pyridine-3-carboxylate

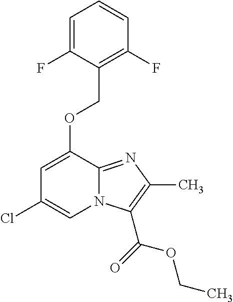 [SMILES: CCOC(=O)c1c(C)nc2c(OCc3c(F)cccc3F)cc(Cl)cn12]

40 g of 5-chloro-3[(2,6-difluorobenzyl)oxy]pyridine-2-amine (Example 14A; 147.8 mmol, 1 equivalent) were initially charged in 800 ml of ethanol, 30 g of powdered molecular sieve 3 Å and 128 g of ethyl 2-chloroacetoacetate (739 mmol, 5 equivalents) were added and the mixture was heated at reflux overnight. The reaction mixture was concentrated, and the residue was taken up in ethyl acetate and filtered. The ethyl acetate phase was washed with water, dried, filtered and concentrated. 44 g (78% of theory) of the title compound were obtained.

LC-MS (Method 2): Rₜ=1.27 min

MS (ESpos): m/z=381.2/383.2 (M+H)⁺

¹H-NMR (400 MHz, DMSO-d₆): δ=1.36 (t, 3H); 2.54 (s, 3H; obscured by DMSO signal); 4.37 (q, 2H); 5.36 (s, 2H); 7.26 (t, 2H); 7.38 (d, 1H); 7.62 (quint., 1H); 8.92 (d, 1H).

### Example 16A

### 6-Chloro-8-[(2,6-difluorobenzyl)oxy]-2-methylimidazo[1,2-a]pyridine-3-carboxylic acid

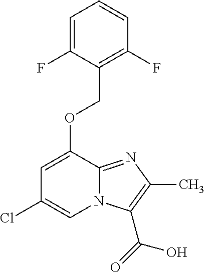 [SMILES: Cc1nc2c(OCc3c(F)cccc3F)cc(Cl)cn2c1C(=O)O]

44 g of ethyl 6-chloro-8-[(2,6-difluorobenzyl)oxy]-2-methylimidazo[1,2-a]pyridine-3-carboxylate (Example 15A; 115.5 mmol, 1 equivalent) were dissolved in 550 ml of THF and 700 ml of methanol, 13.8 g of lithium hydroxide (dissolved in 150 ml of water; 577 mmol, 5 equivalents) were added and the mixture was stirred at RT overnight. 1N hydrochloric acid was added, and the mixture was concentrated. The crystals formed were filtered off with suction and washed with water. This gave 34 g of the title compound (84% of theory).

LC-MS (Method 1): Rₜ=1.03 min

MS (ESpos): m/z=353.0/355.0 (M+H)⁺

¹H-NMR (400 MHz, DMSO-d₆): δ=2.54 (s, 3H; superposed by DMSO signal); 5.36 (s, 2H); 7.26 (t, 2H); 7.34 (d, 1H); 7.61 (quint., 1H); 8.99 (d, 1H); 13.36 (br. s, 1H).

### Example 17A

### 5-Bromo-3-[(2,6-difluorobenzyl)oxy]pyridine-2-amine

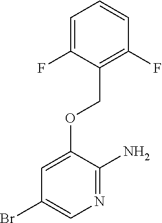 [SMILES: Nc1ncc(Br)cc1OCc1c(F)cccc1F]

32.6 g of 3[(2,6-difluorobenzyl)oxy]pyridine-2-amine (Example 1A; 138 mmol, 1 equivalent) were suspended in 552 ml of 10% strength sulphuric acid, and the mixture was cooled to 0° C. 8.5 ml of bromine (165 mmol, 1.2 equivalents) were dissolved in 85 ml of acetic acid and then, over 90 min, added dropwise to a solution, cooled with ice, of the aminopyridine in sulphuric acid. After the addition had ended, the mixture was stirred at 0° C. for 90 min and then diluted with 600 ml of ethyl acetate, and the aqueous phase was separated off. The aqueous phase was re-extracted with ethyl acetate, and the organic phases were combined, washed with saturated aqueous sodium bicarbonate solution, dried and concentrated. The residue was dissolved in dichloromethane and chromatographed on silica gel (petroleum ether/ethyl acetate gradient as mobile phase). This gave 24 g (55% of theory) of the title compound as bright crystals.

LC-MS (Method 2): Rₜ=0.96 min

MS (ESpos): m/z=315.1/317.1 (M+H)⁺

¹H-NMR (400 MHz, DMSO-d₆): δ=5.14 (s, 2H); 5.83 (br. s, 2H); 7.20 (t, 2H); 7.42 (d, 1H); 7.54 (quint., 1H); 7.62 (d, 1H).

### Example 18A

### Ethyl 6-bromo-8-[(2,6-difluorobenzyl)oxy]-2-methylimidazo[1,2-a]pyridine-3-carboxylate

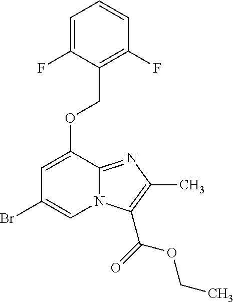 [SMILES: CCOC(=O)c1c(C)nc2c(OCc3c(F)cccc3F)cc(Br)cn12]

24 g of 5-bromo-3[(2,6-difluorobenzyl)oxy]pyridine-2-amine (Example 17A; 76.2 mmol, 1 equivalent) were initially charged in 400 ml of ethanol, 16 g of powdered molecular sieve 3 Å and 52.7 ml of ethyl 2-chloroacetoacetate (380.8 mmol, 5 equivalents) were added and the mixture was heated at reflux overnight. A further 8 g of molecular sieve were added, and the mixture was heated at reflux for a further 24 h. The reaction mixture was concentrated, and the residue was taken up in dichloromethane and chromatographed on silica gel (mobile phase: dichloromethane/methanol 20:1). The product-containing fractions were concentrated, and the residue was stirred in 100 ml of diethyl ether for 30 min, filtered off with suction, washed with a little diethyl ether and dried. 15 g (45% of theory) of the title compound were obtained.

LC-MS (Method 1): Rₜ=1.43 min

MS (ESpos): m/z=414.9/416.8 (M+H)⁺

¹H-NMR (400 MHz, DMSO-d₆): δ=1.36 (t, 3H); 2.54 (s, 3H; obscured by DMSO signal); 4.37 (q, 2H); 5.36 (s, 2H); 7.25 (t, 2H); 7.42 (d, 1H); 7.61 (quint., 1H); 9.00 (d, 1H).

### Example 19A

### 6-Bromo-8-[(2,6-difluorobenzyl)oxy]-2-methylimidazo[1,2-a]pyridine-3-carboxylic acid

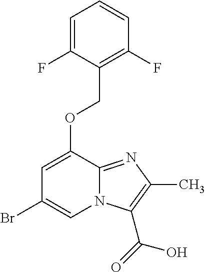 [SMILES: Cc1nc2c(OCc3c(F)cccc3F)cc(Br)cn2c1C(=O)O]

1.5 g of ethyl 6-bromo-8-[(2,6-difluorobenzyl)oxy]-2-methylimidazo[1,2-a]pyridine-3-carboxylate (Example 18A; 3.5 mmol, 1 equivalent) were dissolved in 72 ml of THF/methanol 5:1, 17.6 ml of 1N aqueous lithium hydroxide solution (17.6 mmol, 5 equivalents) were added and the mixture was warmed to 40° C. and stirred at this temperature for 6 h. The mixture was adjusted to pH 4 using 6N hydrochloric acid and concentrated. Water was added to the crystals formed, the mixture was stirred and the crystals were filtered off with suction, washed with water and dried under reduced pressure. This gave 1.24 g of the title compound (88% of theory).

LC-MS (Method 2): Rₜ=0.93 min

MS (ESpos): m/z=397.0/399.1 (M+H)⁺

¹H-NMR (400 MHz, DMSO-d₆): δ=2.54 (s, 3H; superposed by DMSO signal); 5.36 (s, 2H); 7.25 (t, 2H); 7.40 (d, 1H); 7.61 (quint., 1H); 9.06 (d, 1H); 13.35 (br. s, 1H).

### Example 20A

### Ethyl 8-[(2,6-difluorobenzyl)oxy]-2,6-dimethylimidazo[1,2-a]pyridine-3-carboxylate

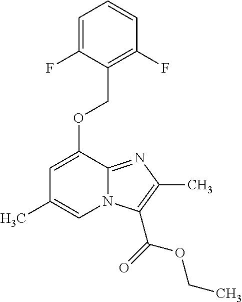 [SMILES: CCOC(=O)c1c(C)nc2c(OCc3c(F)cccc3F)cc(C)cn12]

600 mg of ethyl 6-bromo-8-[(2,6-difluorobenzyl)oxy]-2-methylimidazo[1,2-a]pyridine-3-carboxylate (1.4 mmol, 1 equivalent) and 230 mg of 1,1′-bis(diphenylphosphino)ferrocenepalladium(II) chloride/dichloromethane complex (0.282 mmol, 20 mol %) were dissolved in 25 ml of THF, and 0.88 ml (1.76 mmol, 1.2 equivalents) of a 2 M solution of methylzinc chloride in THF was added. In a microwave oven, the reaction mixture was heated at 100° C. for 40 min. The reaction mixture was filtered through Celite and concentrated on a rotary evaporator. The residue was chromatographed (Biotage Isolera Four). This gave 225 mg (38% of theory) of the title compound.

LC-MS (Method 2): Rₜ=1.05 min

MS (ESpos): m/z=361.1 (M+H)⁺

¹H-NMR (400 MHz, DMSO-d₆): δ=1.38 (t, 3H); 2.36 (s, 3H); 4.35 (q, 2H); 5.30 (s, 2H); 7.10 (s, 1H); 7.23 (t, 2H); 7.59 (quint., 1H); 8.70 (s, 1H).

### Example 21A

### 8-[(2,6-Difluorobenzyl)oxy]-2,6-dimethylimidazo[1,2-a]pyridine-3-carboxylic acid

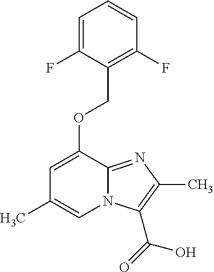

220 mg of ethyl 8-[(2,6-difluorobenzyl)oxy]-2,6-dimethylimidazo[1,2-a]pyridine-3-carboxylate (Example 20A; 0.524 mmol, 1 equivalent) were dissolved in 7 ml of THF/methanol (1:1), 2.6 ml of 1 N aqueous lithium hydroxide solution (2.6 mmol, 5 equivalents) were added and the mixture was stirred at RT for 16 h. The mixture was concentrated and the residue was acidified with 1 N hydrochloric acid. The crystals formed were stirred and filtered off with suction, washed with water and dried under reduced pressure. This gave 120 mg of the title compound (60% of theory).

LC-MS (Method 2): Rₜ=0.68 min

MS (ESpos): m/z=333.1 (M+H)⁺

¹H-NMR (400 MHz, DMSO-d₆): δ=2.34 (s, 3H); 5.28 (s, 2H); 7.09 (s, 1H); 7.23 (t, 2H); 7.58 (quint., 1H); 8.76 (s, 1H); 13.1 (br. s, 1H).

### Example 22A

### Ethyl 8-[(2,6-difluorobenzyl)oxy]-2-methyl-6-(pyrrolidin-1-yl)imidazo[1,2-a]pyridine-3-carboxylate

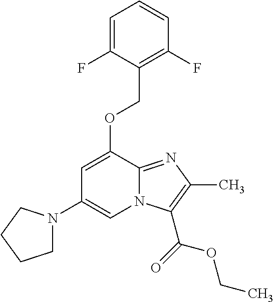 [SMILES: CCOC(=O)c1c(C)nc2c(OCc3c(F)cccc3F)cc(N3CCCC3)cn12]

500 mg of ethyl 6-bromo-8-[(2,6-difluorobenzyl)oxy]-2-methylimidazo[1,2-a]pyridine-3-carboxylate (1.18 mmol, 1 equivalent), 43 mg of tris(dibenzylideneacetone)dipalladium (0.047 mmol, 4 mol %), 158 mg of sodium tert-butoxide (1.65 mmol, 1.4 equivalents), 67 mg of XPHOS (0.141 mmol, 12 mol %) and 294 μl of pyrrolidine (3.5 mmol, 3 equivalents) were dissolved in 30 ml of dry toluene and reacted in an oil bath which had been pre-heated to 100° C. After 16 h at this temperature, the reaction mixture was cooled, filtered through kieselguhr, concentrated and chromatographed (Biotage Isolera Four; mobile phase: cyclohexane/ethyl acetate gradient). 100 mg (19% of theory) of the title compound were obtained.

LC-MS (Method 2): Rₜ=1.08 min

MS (ESpos): m/z=416.2 (M+H)⁺

¹H-NMR (400 MHz, DMSO-d₆): δ=1.34 (t, 3H); 1.95-2.04 (m, 4H); 2.55 (s, 3H; obscured by DMSO signal); 3.21-3.29 (m, 4H); 4.31 (q, 2H); 5.38 (s, 2H); 6.80 (s, 1H); 7.22 (t, 2H); 7.58 (quint., 1H); 8.13 (s, 1H).

### Example 23A

### 8-[(2,6-Difluorobenzyl)oxy]-2-methyl-6-(pyrrolidin-1-yl)imidazo[1,2-a]pyridine-3-carboxylic acid

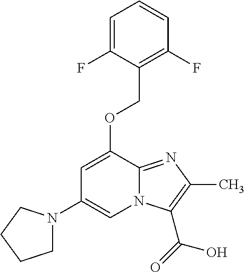 [SMILES: Cc1nc2c(OCc3c(F)cccc3F)cc(N3CCCC3)cn2c1C(=O)O]

90 mg of ethyl 8-[(2,6-difluorobenzyl)oxy]-2-methyl-6-(pyrrolidin-1-yl)imidazo[1,2-a]pyridine-3-carboxylate (Example 22A; 0.217 mmol, 1 equivalent) were dissolved in 6 ml of THF/methanol (5:1), 1.1 ml of 1 N aqueous lithium hydroxide solution (1 l mmol, 5 equivalents) were added and the mixture was warmed to 40° C. and stirred at this temperature for 20 h. The mixture was cooled, acidified to pH 4 using 6 N hydrochloric acid and concentrated. Water was added to the crystals formed, the mixture was stirred and the crystals were filtered off with suction, washed with water and dried under reduced pressure. This gave 87 mg of the title compound (93% of theory).

LC-MS (Method 2): Rₜ=0.83 min

MS (ESpos): m/z=388.2 (M+H)⁺

¹H-NMR (400 MHz, DMSO-d₆): δ=2.00-2.08 (m, 4H); 2.60 (s, 3H); 3.30-3.38 (m, 4H); 5.52 (s, 2H); 7.24 (s, 1H); 7.25 (t, 2H); 7.60 (quint., 1H); 8.30 (s, 1H).

### Example 24A

### Ethyl 8-[(2,6-difluorobenzyl)oxy]-2-methyl-6-(morpholin-4-yl)imidazo[1,2-a]pyridine-3-carboxylate

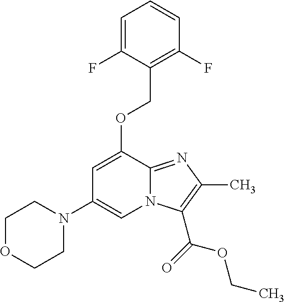 [SMILES: CCOC(=O)c1c(C)nc2c(OCc3c(F)cccc3F)cc(N3CCOCC3)cn12]

500 mg of ethyl 6-bromo-8-[(2,6-difluorobenzyl)oxy]-2-methylimidazo[1,2-a]pyridine-3-carboxylate (1.18 mmol, 1 equivalent), 43 mg of tris(dibenzylideneacetone)dipalladium (0.047 mmol, 4 mol %), 158 mg of sodium tert-butoxide (1.65 mmol, 1.4 equivalents), 67 mg of XPHOS (0.141 mmol, 12 mol %) and 307 μl of morpholine (3.5 mmol, 3 equivalents) were dissolved in 30 ml of dry toluene and reacted in an oil bath which had been pre-heated to 100° C. After 16 h at this temperature, the reaction mixture was cooled, filtered through kieselguhr, concentrated and chromatographed (Biotage Isolera Four; mobile phase: cyclohexane/ethyl acetate gradient). 352 mg (63% of theory) of the title compound were obtained.

LC-MS (Method 2): Rₜ=1.05 min

MS (ESpos): m/z=432.2 (M+H)⁺

¹H-NMR (400 MHz, DMSO-d₆): δ=1.35 (t, 3H); 2.55 (s, 3H; obscured by DMSO signal); 3.08-3.13 (m, 4H); 3.75-3.80 (m, 4H); 4.31 (q, 2H); 5.30 (s, 2H); 7.20 (s, 1H); 7.23 (t, 2H); 7.59 (quint., 1H); 8.40 (s, 1H).

### Example 25A

### 8-[(2,6-Difluorobenzyl)oxy]-2-methyl-6-(morpholin-4-yl)imidazo[1,2-a]pyridine-3-carboxylic acid

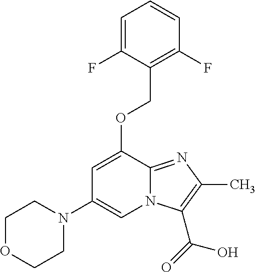 [SMILES: Cc1nc2c(OCc3c(F)cccc3F)cc(N3CCOCC3)cn2c1C(=O)O]

400 mg of ethyl 8-[(2,6-difluorobenzyl)oxy]-2-methyl-6-(pyrrolidin-1-yl)imidazo[1,2-a]pyridine-3-carboxylate (Example 24A; 0.927 mmol, 1 equivalent) were dissolved in 24 ml of THF/methanol (5:1), 4.6 ml of 1 N aqueous lithium hydroxide solution (4.6 mmol, 5 equivalents) were added and the mixture was warmed to 40° C. and stirred at this temperature for 4 h. The mixture was cooled, acidified to pH 4 using 6 N hydrochloric acid and concentrated. Water was added to the residue and the mixture was extracted repeatedly with dichloromethane. The combined organic phases were washed with saturated aqueous sodium chloride solution, dried, filtered and concentrated. This gave 145 mg of the title compound (35% of theory), which was converted further without further purification.

LC-MS (Method 2): Rₜ=0.72 min

MS (ESpos): m/z=404.2 (M+H)⁺

¹H-NMR (400 MHz, DMSO-d₆): δ=2.55 (s, 3H; superposed by DMSO signal); 3.10-3.20 (m, 4H); 3.75-3.82 (m, 4H); 5.38 (s, 2H); 7.23 (t, 2H); 7.25 (s, 1H); 7.58 (quint., 1H); 8.48 (s, 1H).

### Example 26A

### 6-Chloro-8-[(2,3-difluorobenzyl)oxy]-2-methylimidazo[1,2-a]pyridine-3-carboxylic acid

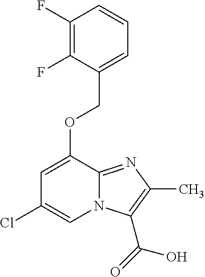 [SMILES: Cc1nc2c(OCc3cccc(F)c3F)cc(Cl)cn2c1C(=O)O]

### Step a): 2-Amino-5-chloropyridin-3-ol

Nitro reduction of 5-chloro-2-nitropyridin-3-ol (Example 12A) analogously to the preparation of Example 14A to give 2-amino-5-chloropyridin-3-ol; yield 84% (contained 33% of dichlorinated product).

LC-MS (Method 2): Rₜ=0.20 min

MS (ESpos): m/z=144.9/146.9 (M+H)⁺

### Step b): 5-Chloro-3-[(2,6-difluorobenzyl)oxy]pyridine-2-amine

Reaction of 2-amino-5-chloropyridin-3-ol with 1.1 equivalents of 2,3-difluorobenzyl bromide and 2.2 equivalents of caesium carbonate in DMF (15 min at 50° C.), aqueous work-up, extraction with ethyl acetate and subsequent chromatography of the organic residue (gradient: cyclohexane/ethyl acetate 8:1 to pure ethyl acetate) to give 5-chloro-3[(2,6-difluorobenzyl)oxy]pyridine-2-amine; yield 10%.

LC-MS (Method 2): Rₜ=0.94 min

MS (ESpos): m/z=271.0/273.0 (M+H)⁺

### Step c): Ethyl 6-chloro-8-[(2,3-difluorobenzyl)oxy]-2-methylimidazo[1,2-a]pyridine-3-carboxylate

Cyclization (analogously to the preparation of Example 15A) to ethyl 6-chloro-8-[(2,3-difluorobenzyl)oxy]-2-methylimidazo[1,2-a]pyridine-3-carboxylate; yield 48%.

LC-MS (Method 2): Rₜ=1.25 min

MS (ESpos): m/z=381.1/383.0 (M+H)⁺

### Step d): 6-Chloro-8-[(2,3-difluorobenzyl)oxy]-2-methylimidazo[1,2-a]pyridine-3-carboxylic acid

Ester hydrolysis (analogously to the preparation of Example 16A) to 6-chloro-8-[(2,3-difluorobenzyl)oxy]-2-methylimidazo[1,2-a]pyridine-3-carboxylic acid; yield 67%.

LC-MS (Method 2): Rₜ=0.87 min

MS (ESpos): m/z=353.1/355.1 (M+H)⁺

¹H-NMR (400 MHz, DMSO-d₆): δ=2.54 (s, 3H; superposed by DMSO signal); 5.41 (s, 2H); 7.27 (s, 1H); 7.25-7.31 (m, 1H); 7.43-7.55 (m, 2H); 8.99 (s, 1H); 13.39 (br. s, 1H).

### Example 27A

### 8-[(2,6-Difluorobenzyl)oxy]-2-methylimidazo[1,2-a]pyridine-3-carbonyl chloride hydrochloride

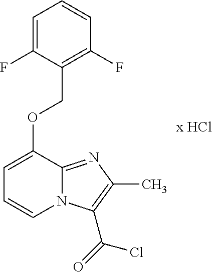 [SMILES: Cc1nc2c(OCc3c(F)cccc3F)cccn2c1C(=O)Cl]

2.0 g (6.28 mmol) of 8-[(2,6-difluorobenzyl)oxy]-2-methylimidazo[1,2-a]pyridine-3-carboxylic acid were initially charged in abs. THF, 4 drops of DMF were added and 3.19 g (25.14 mmol) of oxalyl chloride were added dropwise. The reaction mixture was stirred at RT for 3 h. Another 0.80 g (6.29 mmol) of oxalyl chloride was added and the reaction was stirred at RT for a further 4 h. The reaction mixture was concentrated and evaporated three times with toluene, and the residue was dried under high vacuum. 2.43 g of the target compound were obtained (103% of theory).

DCI-MS (Method 4): MS (ESpos): m/z=437 (M-HCl+H)⁺

### Example 28A

### tert-Butyl 3-amino-1H-indazole-1-carboxylate

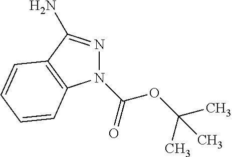 [SMILES: CC(C)(C)OC(=O)n1nc(N)c2ccccc21]

150 mg (1.13 mmol) of 1H-indazole-3-amine were initially charged in 3 ml of THF, 320 mg (1.46 mmol) of di-tert-butyl dicarbonate, 137 mg (1.35 mmol) of triethylamine and 48 mg (0.39 mmol) of dimethylaminopyridine were then added and the mixture was stirred at RT for 1.5 h. The reaction solution was diluted with ethyl acetate and washed in each case once with water, saturated aqueous ammonium chloride solution and saturated sodium chloride solution. The organic phase was dried over sodium sulphate and filtered and the filtrate was concentrated. The residue was purified by silica gel chromatography (mobile phase: cyclohexane/ethyl acetate 3/1->1/1). This gave 126 mg of the target compound (48% of theory).

LC-MS (Method 2): Rₜ=0.88 min

MS (ESpos): m/z=234 (M+H)⁺

¹H-NMR (400 MHz, DMSO-d₆): δ=1.58 (s, 9H), 6.30 (s, 2H), 7.25 (t, 1H), 7.50 (t, 1H), 7.82 (d, 1H), 7.94 (d, 1H).

### Example 29A

### tert-Butyl 3-[({8-[(2,6-difluorobenzyl)oxy]-2-methylimidazo[1,2-a]pyridin-3-yl}carbonyl)amino]-1H-indazole-1-carboxylate trifluoroacetate

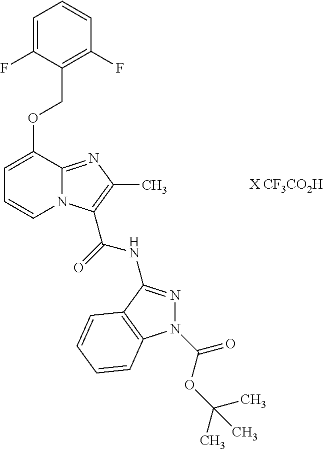 [SMILES: Cc1nc2c(OCc3c(F)cccc3F)cccn2c1C(=O)Nc1nn(C(=O)OC(C)(C)C)c2ccccc12 ; "X CF3CO2H"]

100 mg (0.27 mmol) of 8-[(2,6-difluorobenzyl)oxy]-2-methylimidazo[1,2-a]pyridine-3-carbonyl chloride hydrochloride were initially charged suspended in abs. THF, and 75 mg (0.32 mmol) of tert-butyl 3-amino-1H-indazole-1-carboxylate and 139 mg (1.07 mmol) of N,N-diisopropylethylamine were added and the mixture was stirred at 60° C. for 4 days. The reaction mixture was filtered and the filtrate was concentrated slightly and purified by preparative HPLC (RP18 column, mobile phase: acetonitrile/water gradient with addition of 0.1% trifluoroacetic acid). This gave 74 mg of the target compound (43% of theory, purity 93%).

LC-MS (Method 1): Rₜ=1.38 min

MS (ESpos): m/z=534 (M-TFA+H)⁺

¹H-NMR (400 MHz, DMSO-d₆): δ=1.65 (s, 9H), 2.69 (s, 3H), 5.40 (s, 2H), 7.19-7.29 (m, 3H), 7.32-7.40 (m, 2H), 7.58-7.68 (m, 2H), 7.88 (d, 1H), 8.15 (d, 1H), 8.70 (d, 1H), 11.28 (br s, 1H).

### Example 30A

### Methyl 4-{[(benzyloxy)carbonyl]amino}-1-methyl-1H-pyrazole-3-carboxylate

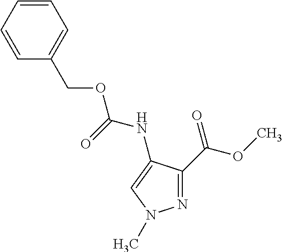 [SMILES: COC(=O)c1nn(C)cc1NC(=O)OCc1ccccc1]

300 mg of methyl 4-amino-1-methyl-1H-pyrazole-3-carboxylate (1.9 mmol, 1 equivalent) were dissolved in 8 ml of dry tetrahydrofuran, and 0.3 ml of benzyl chloroformate (2.12 mmol, 1.1 equivalents), 1.01 ml of diisopropylethylamine (5.8 mmol, 3 equivalents) and 47 mg of N,N-dimethylaminopyridine (0.387 mmol, 0.2 equivalents) were added in succession and the mixture was stirred at RT. To improve solubility, an additional 2 ml of dimethylformamide were added after 30 minutes. After a total of 3.5 h at RT, water was added, the reaction mixture was extracted three times with dichloromethane and the combined organic phases were dried with magnesium sulphate, filtered and concentrated to dryness by rotary evaporation. The residue (491 mg, purity 81%, 73% of theory) was used for the next reaction without further purification.

LC-MS (Method 1): Rₜ=1.22 min

MS (ESpos): m/z=290.1 (M+H)⁺

### Example 31A

### Benzyl [3-(hydroxymethyl)-1-methyl-1H-pyrazol-4-yl]carbamate

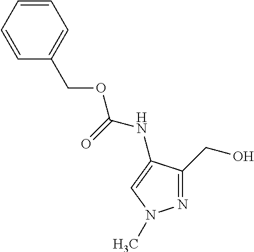 [SMILES: Cn1cc(NC(=O)OCc2ccccc2)c(CO)n1]

493 mg of methyl 4-{[(benzyloxy)carbonyl]amino}-1-methyl-1H-pyrazole-3-carboxylate (purity 81%, 1.39 mmol, 1 equivalent) were initially charged in 4 ml of dry tetrahydrofuran and cooled to −78° C., and 5.5 ml of a 1M solution of diisobutylaluminum hydride in toluene (5.5 mmol, 4 equivalents) were added. Over 30 minutes, the mixture was warmed to RT, and stirred at this temperature for a further 2 h. Water was then added, the reaction mixture was extracted three times with dichloromethane and the combined organic phases were dried with magnesium sulphate, filtered and concentrated to dryness by rotary evaporation. The residue was chromatographed (Biotage Isolera; mobile phase: cyclohexane/ethyl acetate gradient from 6:1 to pure ethyl acetate), giving 293 mg (78% of theory) of the title compound.

LC-MS (Method 2): Rₜ=0.69 min

MS (ESpos): m/z=262.1 (M+H)⁺

### Example 32A

### (4-Amino-1-methyl-1H-pyrazol-3-yl)methanol

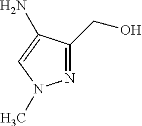

290 mg of benzyl [3-(hydroxymethyl)-1-methyl-1H-pyrazol-4-yl]carbamate (1.1 mmol, 1 equivalent) were initially charged in 50 ml of ethanol, a spatula tip of palladium (10% on activated carbon) was added and the mixture was stirred under one atmosphere of hydrogen at RT for 2 h. The reaction mixture was filtered through kieselguhr and the filtrate was concentrated under reduced pressure. This gave 164 mg (96% of theory) of the title compound.

LC-MS (Method 2): Rₜ=0.16 min

MS (ESpos): m/z=128.0 (M+H)⁺

¹H-NMR (400 MHz, DMSO-d₆): δ=3.70 (s, 3H); 4.40 (d, 2H); 4.81 (t, 1H); 6.80 (s, 1H).

### Example 33A

### 2-{4-[({8-[(2,6-Difluorobenzyl)oxy]-2-methylimidazo[1,2a]pyridin-3-yl}carbonyl)amino]-1H-pyrazol-1-yl}ethylmethanesulphonate

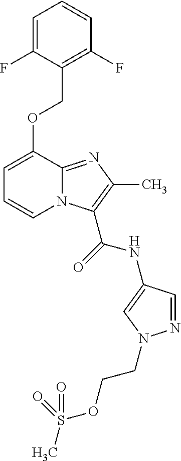 [SMILES: Cc1nc2c(OCc3c(F)cccc3F)cccn2c1C(=O)Nc1cnn(CCOS(C)(=O)=O)c1]

1.3 g (3.1 mmol) of 8-[(2,6-difluorobenzyl)oxy]-N-[1-(2-hydroxyethyl)-1H-pyrazol-4-yl]-2-methylimidazo[1,2-a]pyridine-3-carboxamide (Example 22) were dissolved in 7 ml of dichloromethane, and 0.86 ml of triethylamine (6.14 mmol) and 1.48 ml of methanesulphonyl chloride (3.7 mmol) were added with ice cooling. The resulting mixture was warmed to room temperature and stirred for 2 h. 0.74 ml of methanesulphonyl chloride (1.85 mmol) was added and the mixture was stirred for a further 30 min Saturated aqueous sodium chloride solution was added to the reaction mixture, and the organic phase was dried and concentrated. The crude product was reacted further without further purification.

LC-MS (Method 2): Rₜ=0.79 min

MS (ESpos): m/z=506.3 (M+H)⁺

¹H-NMR (400 MHz, DMSO-d₆): δ=2.30 (s, 3H); 2.62 (s, 3H), 3.05-3.15 (m, 2H), 4.45-4.55 (m, 2H), 5.43 (s, 2H); 7.23 (t, 2H); 7.40 (br. t, 1H), 7.55-7.65 (m, 2H); 7.65 (s, 1H); 8.14 (s, 1H); 8.70 (d, 1H); 10.55 (s, 1H).

### Example 34A

### 8-[(2,6-Difluorobenzyl)oxy]-N-{1-[2-(1,3-dioxo-1,3-dihydro-2H-isoindol-2-yl)ethyl]-1H-pyrazol-4-yl}-2-methylimidazo[1,2-a]pyridine-3-carboxamide

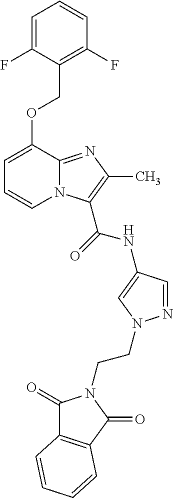 [SMILES: Cc1nc2c(OCc3c(F)cccc3F)cccn2c1C(=O)Nc1cnn(CCN2C(=O)c3ccccc3C2=O)c1]

312 mg of phthalimide (2.1 mmol) were dissolved in 18 ml of 1-methyl-2-pyrrolidone (NMP) and, with ice cooling, 7.4 ml of a 0.6 M sodium bis(trimethylsilyl)amide solution in toluene (4.4 mmol) were added. The mixture was stirred at room temperature for 5 minutes, and 894 mg (3.1 mmol) of 2-{4-[({8-[(2,6-difluorobenzyl)oxy]-2-methylimidazo[1,2-a]pyridin-3-yl}carbonyl)amino]-1H-pyrazol-1-yl}ethylmethanesulphonate (Example 33A) and 662 mg of sodium iodide (4.4 mmol) were added. The resulting mixture was stirred at 100° C. for 16 h. Water was then added, the mixture was extracted four times with ethyl acetate and the combined organic phases were washed with water and saturated aqueous sodium chloride solution, dried and concentrated. The crude product was purified chromatographically (Biotage Isolera, cyclohexane/ethyl acetate gradient). This gave 355 mg (35% of theory) of the title compound.

LC-MS (Method 2): Rₜ=0.83 min

MS (ESpos): m/z=557.3 (M+H)⁺

¹H-NMR (400 MHz, DMSO-d₆): δ=2.55 (s, 3H; superposed by DMSO signal), 3.95 (t, 2H), 4.38 (t, 2H), 5.31 (s, 2H); 6.95 (t, 1H), 7.05 (d, 1H), 7.23 (t, 2H), 7.48 (s, 1H), 7.60 (quint., 1H), 7.80-7.90 (m, 4H), 8.06 (s, 1H), 8.55 (d, 1H), 9.92 (s, 1H).

The examples shown in Table 1A were prepared analogously to Example 48 by reacting the appropriate carboxylic acids with the appropriate commercially available amines (1-3 equivalents), HATU (1-2.5 equivalents) and N,N-diisopropylethylamine (3-4 equivalents) at RT. The reaction times were 1-3 days. The purifications were optionally carried out by preparative HPLC (RP18 column, mobile phase: acetonitrile/water gradient using 0.1% trifluoroacetic acid) or by silica gel chromatography (mobile phase gradient: dichloromethane/methanol). The product-containing fractions were optionally concentrated, the residue was dissolved in ethyl acetate or dichloromethane/methanol and washed with a little saturated aqueous sodium bicarbonate solution, and the organic phase was then dried over sodium sulphate and filtered and the filtrate was concentrated.

TABLE 1A

| | IUPAC name/structure | |
|---|---|---|
| Example | (Yield) | Analytical data |
| 35A | 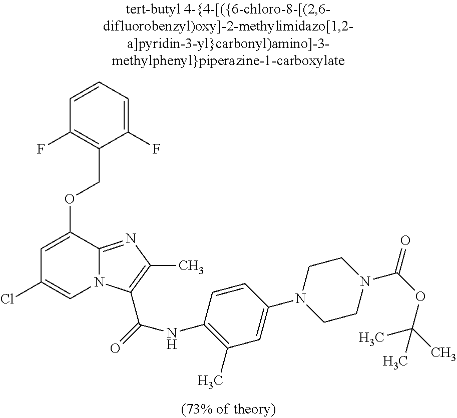 [SMILES: Cc1cc(N2CCN(C(=O)OC(C)(C)C)CC2)ccc1NC(=O)c1c(C)nc2c(OCc3c(F)cccc3F)cc(Cl)cn12] | LC-MS (Method 2): Rₜ = 1.34 min MS (ESpos): m/z = 626.5 (M + H)⁺ |
| 36A | 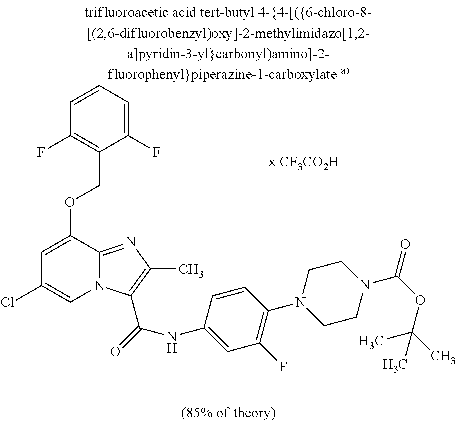 [SMILES: Cc1nc2c(OCc3c(F)cccc3F)cc(Cl)cn2c1C(=O)Nc1ccc(N2CCN(C(=O)OC(C)(C)C)CC2)c(F)c1] | LC-MS (Method 2): Rₜ = 1.41 min MS (ESpos): m/z = 630 (M − TFA + H)⁺ |

^{a)} The reaction was stirred at room temperature for one day and then at 60° C. for one day.

### Example 37A

tert-Butyl {2-[({8-[(2,6-difluorobenzyl)oxy]-2-methylimidazo[1,2-a]pyridin-3-yl}carbonyl)amino]-5-fluorobenzyl}carbamate trifluoroacetate

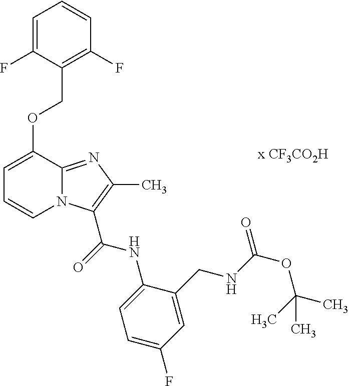 [SMILES: Cc1nc2c(OCc3c(F)cccc3F)cccn2c1C(=O)Nc1ccc(F)cc1CNC(=O)OC(C)(C)C]

65 mg (0.21 mmol) of 8-[(2,6-difluorobenzyl)oxy]-2-methylimidazo[1,2-a]pyridine-3-carboxylic acid, 82 mg (0.22 mmol) of O-(7-azabenzotriazol-1-yl)-N,N,N′N′-tetramethyluronium hexafluorophosphate (HATU) and 106 mg (0.82 mmol) of N,N-diisopropylethylamine were initially charged in 0.9 ml of DMF, and the mixture was stirred at RT for 15 min 80 mg (0.23 mmol) of tert-butyl (2-amino-5-fluorobenzyl)carbamate [M. Munson et al. US2004/180896] as trifluoroacetate salt were then added, and the reaction mixture was stirred at 60° C. overnight. 33 mg (0.11 mmol) of 8-[(2,6-difluorobenzyl)oxy]-2-methylimidazo[1,2-a]pyridine-3-carboxylic acid were dissolved in 0.47 ml of DMF and stirred with 41 mg (0.11 mmol) of O-(7-azabenzotriazol-1-yl)-N,N,N′N′-tetramethyluronium hexafluorophosphate (HATU) and 53 mg (0.41 mmol) of N,N-diisopropylethylamine in a separate reaction flask at RT for 15 min. This solution was then added to the reaction mixture, and the mixture was stirred at 60° C. for 11 h. The reaction solution was purified by preparative HPLC (RP18 column; mobile phase: acetonitrile/water gradient with addition of 0.1% trifluoroacetic acid). 44 mg of the target compound (30% of theory) were obtained.

LC-MS (Method 2): Rₜ=1.10 min

MS (ESpos): m/z=541 (M+H)⁺

### Example 38A

### tert-Butyl {2-[({8-[(2,6-difluorobenzyl)oxy]-2-methylimidazo[1,2-a]pyridin-3-yl}carbonyl)amino]benzyl}carbamate

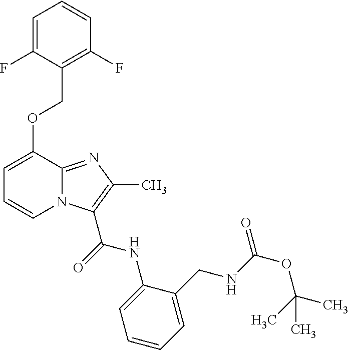 [SMILES: Cc1nc2c(OCc3c(F)cccc3F)cccn2c1C(=O)Nc1ccccc1CNC(=O)OC(C)(C)C]

150 mg (0.47 mmol) of 8-[(2,6-difluorobenzyl)oxy]-2-methylimidazo[1,2-a]pyridine-3-carboxylic acid, 358 mg (0.94 mmol) of O-(7-azabenzotriazol-1-yl)-N,N,N′N′-tetramethyluronium hexafluorophosphate (HATU) and 152 mg (1.18 mmol) of N,N-diisopropylethylamine were initially charged in 3 ml of DMF, and the mixture was stirred at RT for 10 min 157 mg (0.71 mmol) of tert-butyl (2-aminobenzyl)carbamate were then added, and the reaction mixture was stirred at RT overnight. The mixture was stirred first at 40° C. overnight and then at 60° C. overnight. About 24 ml of water were added and the resulting precipitate was stirred for another 30 min, filtered off with suction and washed thoroughly with water. 265 mg of the target compound were obtained (88% of theory, purity about 82%).

LC-MS (Method 2): Rₜ=1.04 min

MS (ESpos): m/z=523 (M+H)⁺

### Example 39A

### tert-Butyl {2-[({6-chloro-8-[(2,6-difluorobenzyl)oxy]-2-methylimidazo[1,2-a]pyridin-3-yl}carbonyl)amino]benzyl}carbamate trifluoroacetate

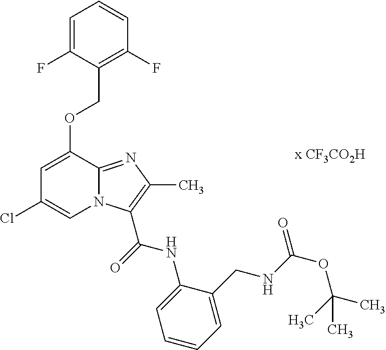 [SMILES: Cc1nc2c(OCc3c(F)cccc3F)cc(Cl)cn2c1C(=O)Nc1ccccc1CNC(=O)OC(C)(C)C]

150 mg (0.43 mmol) of 6-chloro-8-[(2,6-difluorobenzyl)oxy]-2-methylimidazo[1,2-a]pyridine-3-carboxylic acid, 323 mg (0.85 mmol) of O-(7-azabenzotriazol-1-yl)-N,N,N′N′-tetramethyluronium hexafluorophosphate (HATU) and 137 mg (1.06 mmol) of N,N-diisopropylethylamine were initially charged in 2.7 ml of DMF, and the mixture was stirred at RT for 10 min 142 mg (0.64 mmol) of tert-butyl (2-aminobenzyl)carbamate were then added, and the mixture was stirred at 60° C. overnight. About 22 ml of water were added to the reaction solution, and the resulting precipitate was stirred for 30 min, filtered off with suction and washed thoroughly with water. The residue was purified by silica gel chromatography (mobile phase: dichloromethane/methanol=100/1). The crude product was purified again by preparative HPLC (RP18 column; mobile phase: acetonitrile/water gradient with addition of 0.1% trifluoroacetic acid). 153 mg of the target compound (54% of theory) were obtained.

LC-MS (Method 2): Rₜ=1.26 min

MS (ESpos): m/z=557 (M+H)⁺

### Example 40A

### tert-Butyl {2-[({8-[(2,6-difluorobenzyl)oxy]-2,6-dimethylimidazo[1,2-a]pyridin-3-yl}carbonyl)amino]benzyl}carbamate trifluoroacetate

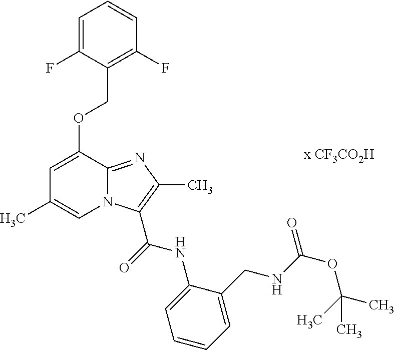 [SMILES: Cc1cc(OCc2c(F)cccc2F)c2nc(C)c(C(=O)Nc3ccccc3CNC(=O)OC(C)(C)C)n2c1]

150 mg (0.45 mmol) of 8-[(2,6-difluorobenzyl)oxy]-2,6-dimethylimidazo[1,2-a]pyridine-3-carboxylic acid, 429 mg (1.13 mmol) of O-(7-azabenzotriazol-1-yl)-N,N,N′N′-tetramethyluronium hexafluorophosphate (HATU) and 146 mg (1.13 mmol) of N,N-diisopropylethylamine were initially charged in 2.9 ml of DMF, and the mixture was stirred at RT for 10 min 151 mg (0.68 mmol) of tert-butyl (2-aminobenzyl)carbamate were then added, and the mixture was stirred at 60° C. overnight. Another 50 mg (0.23 mmol) of tert-butyl (2-aminobenzyl)carbamate were added, and the mixture was stirred at 60° C. overnight. About 40 ml of water were added to the reaction solution, and the resulting precipitate was stirred for 30 min, filtered off with suction and washed thoroughly with water. The residue was purified by preparative HPLC (RP18 column, mobile phase: acetonitrile/water gradient with addition of 0.1% trifluoroacetic acid). 112 mg of the target compound (38% of theory) were obtained.

LC-MS (Method 2): Rₜ=1.05 min

MS (ESpos): m/z=537 (M+H)⁺

The examples shown in Table 2A were prepared analogously to Example 40A by reacting the appropriate carboxylic acids with the appropriate commercially available amines (1-3 equivalents), HATU (1-2.5 equivalents) and N,N-diisopropylethylamine (4 equivalents). The reaction times were 1-3 days. The purifications were optionally carried out by preparative HPLC (RP18 column, mobile phase: acetonitrile/water gradient using 0.1% trifluoroacetic acid) and/or by silica gel chromatography (mobile phase gradient: dichloromethane/methanol or ethyl acetate/cyclohexane). The product-containing fractions were optionally concentrated, the residue was dissolved in ethyl acetate or dichloromethane/methanol and washed with a little saturated aqueous sodium bicarbonate solution, and the organic phase was then dried over sodium sulphate and filtered and the filtrate was concentrated.

TABLE 2A

| | IUPAC name/structure | |
|---|---|---|
| Example | (Yield) | Analytical data |
| 41A | 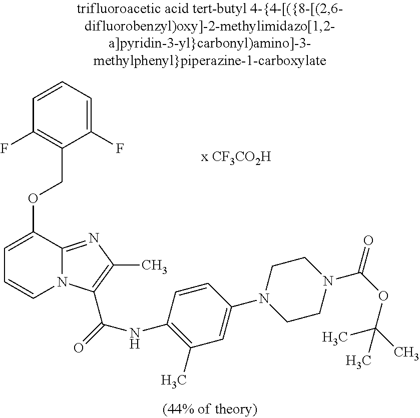 [SMILES: Cc1cc(N2CCN(C(=O)OC(C)(C)C)CC2)ccc1NC(=O)c1c(C)nc2c(OCc3c(F)cccc3F)cccn12] | LC-MS (Method 2): Rₜ = 1.10 min MS (ESpos): m/z = 592 (M + H)⁺ |
| 42A | 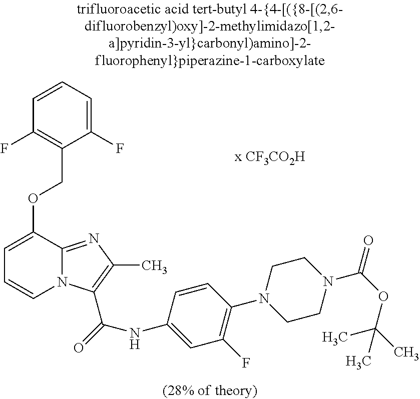 [SMILES: Cc1nc2c(OCc3c(F)cccc3F)cccn2c1C(=O)Nc1ccc(N2CCN(C(=O)OC(C)(C)C)CC2)c(F)c1] | LC-MS (Method 2): Rₜ = 1.16 min MS (ESpos): m/z = 596 (M + H)⁺ |
| 43A | 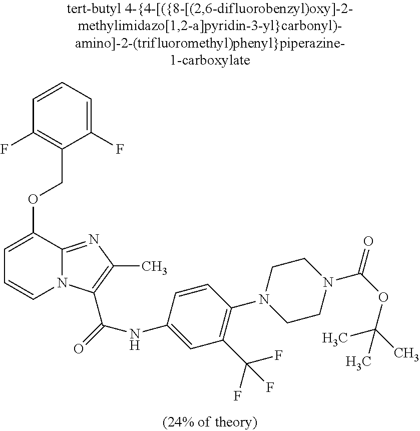 [SMILES: Cc1nc2c(OCc3c(F)cccc3F)cccn2c1C(=O)Nc1ccc(N2CCN(C(=O)OC(C)(C)C)CC2)c(C(F)(F)F)c1] | LC-MS (Method 2): Rₜ = 1.34 min MS (ESpos): m/z = 646 (M + H)⁺ |

### Example 44A

### tert-Butyl {2-[({8-[(2,6-difluorobenzyl)oxy]-2,6-dimethylimidazo[1,2-a]pyridin-3-yl}carbonyl)amino]-5-fluorobenzyl}carbamate trifluoroacetate

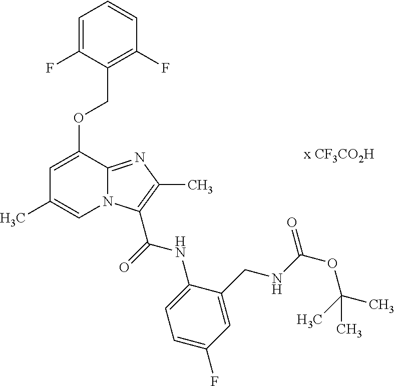 [SMILES: Cc1cc(OCc2c(F)cccc2F)c2nc(C)c(C(=O)Nc3ccc(F)cc3CNC(=O)OC(C)(C)C)n2c1]

100 mg (0.30 mmol) of 8-[(2,6-difluorobenzyl)oxy]-2,6-dimethylimidazo[1,2-a]pyridine-3-carboxylic acid, 120 mg (0.32 mmol) of O-(7-azabenzotriazol-1-yl)-N,N,N′N′-tetramethyluronium hexafluorophosphate (HATU) and 156 mg (1.20 mmol) of N,N-diisopropylethylamine were initially charged in 1.4 ml of DMF, and the mixture was stirred at RT for 15 min 117 mg (0.33 mmol) of tert-butyl (2-amino-5-fluorobenzyl)carbamate were then added, and the mixture was stirred at 60° C. overnight. Another 120 mg (0.32 mmol) of O-(7-azabenzotriazol-1-yl)-N,N,N′N′-tetramethyluronium hexafluorophosphate (HATU) and 78 mg (0.60 mmol) of N,N-diisopropylethylamine and, after 10 min, 117 mg (0.33 mmol) of tert-butyl (2-amino-5-fluorobenzyl)carbamate trifluoroacetate were added, and the mixture was stirred at 60° C. overnight. Acetonitrile and trifluoroacetic acid were added to the reaction solution and the mixture was quickly purified by preparative HPLC (RP18 column, mobile phase: acetonitrile/water gradient with addition of 0.1% trifluoroacetic acid). 103 mg of the target compound (50% of theory) were obtained.

LC-MS (Method 2): Rₜ=1.07 min

MS (ESpos): m/z=555 (M+H)⁺

The examples shown in Table 3A were prepared analogously to Example 28A by reacting the appropriate amines with di-tert-butyl dicarbonate (1.2-2.1 equivalents) and 4-dimethylaminopyridine (0.2 equivalents) at RT. The reaction times were 1-3 h. The purifications were carried out by silica gel chromatography (mobile phase gradient: ethyl acetate/cyclohexane).

TABLE 3A

| Exam- | IUPAC name/structure | |
|---|---|---|
| ple | (Yield) | Analytical data |
| 45A | 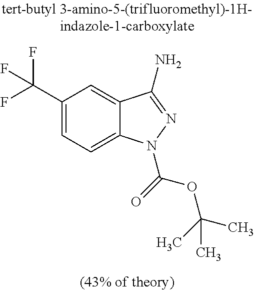 [SMILES: CC(C)(C)OC(=O)n1nc(N)c2cc(C(F)(F)F)ccc21] | LC-MS (Method 2): Rₜ = 1.08 min MS (ESpos): m/z = 302 (M + H)⁺ |
| 46A | 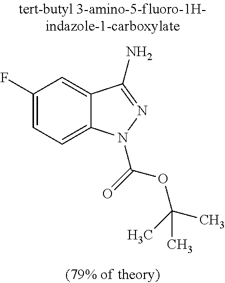 [SMILES: CC(C)(C)OC(=O)n1nc(N)c2cc(F)ccc21] | LC-MS (Method 2): Rₜ = 0.94 min MS (ESpos): m/z = 252 (M + H)⁺ |

The examples shown in Table 4A were prepared analogously to Example 29A by reacting the carbonyl chlorides with the appropriate amines (1 equivalent) and N,N-diisopropylethylamine (4 equivalents) in THF at 60° C. The reaction times were 4-6 days. The purifications were optionally carried out by preparative HPLC (RP18 column, mobile phase: acetonitrile/water gradient using 0.1% trifluoroacetic acid) and/or by silica gel chromatography (mobile phase gradient: dichloromethane/methanol). The product-containing fractions were optionally concentrated, the residue was dissolved in ethyl acetate or dichloromethane/methanol and washed with a little saturated aqueous sodium bicarbonate solution, and the organic phase was then dried over sodium sulphate and filtered and the filtrate was concentrated.

TABLE 4A

| | IUPAC name/structure | |
|---|---|---|
| Example | (Yield) | Analytical data |
| 47A | 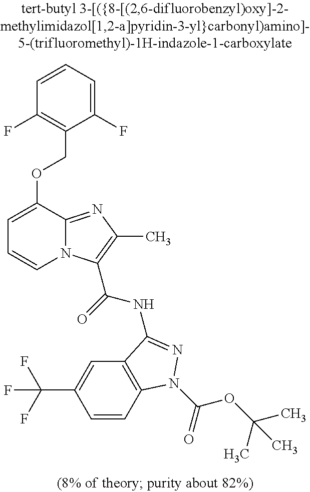 [SMILES: Cc1nc2c(OCc3c(F)cccc3F)cccn2c1C(=O)Nc1nn(C(=O)OC(C)(C)C)c2ccc(C(F)(F)F)cc12] | LC-MS (Method 10): Rₜ = 1.35 min MS (ESpos): m/z = 602 (M + H)⁺ |
| 48A | 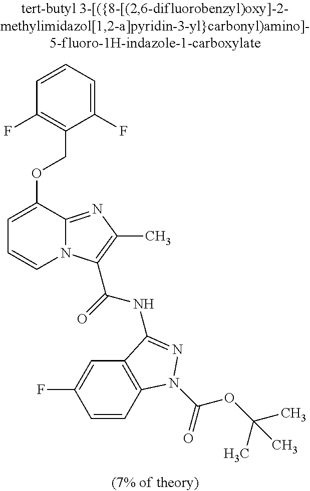 [SMILES: Cc1nc2c(OCc3c(F)cccc3F)cccn2c1C(=O)Nc1nn(C(=O)OC(C)(C)C)c2ccc(F)cc12] | LC-MS (Method 2): Rₜ = 1.24 min MS (ESpos): m/z = 552 (M + H)⁺ |

### Example 49A

### 2-(5-Methyl-4-nitro-1H-pyrazol-3-yl)propan-2-ol

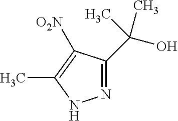

2.23 g (12.05 mmol) of methyl 5-methyl-4-nitro-1H-pyrazole-3-carboxylate [described in: DE 1945430, Minnesota Mining and Manufacturing Co.] were initially charged in 89 ml of dry tetrahydrofuran. At −50° C., 26.35 ml (42.16 mmol) of methyllithium (1.6 M in diethyl ether) were added dropwise, and the mixture was allowed to warm slowly to 0° C. The reaction mixture was once more cooled to −50° C., 7.52 ml (12.04 mmol) of methyllithium (1.6 M in diethyl ether) were added and the mixture was allowed to warm slowly to 0° C. The reaction solution was diluted with dichloromethane and washed with saturated aqueous ammonium chloride solution. The organic phase was dried over sodium sulphate, filtered and concentrated by rotary evaporation. The crude product was purified by silica gel chromatography (mobile phase: dichloromethane to dichloromethane/methanol=50/1). 640 mg of the target compound (29% of theory) were obtained.

LC-MS (Method 10): Rₜ=0.60 min

MS (ESpos): m/z=186 (M+H)⁺

### Example 50A

### 2-(4-Amino-5-methyl-1H-pyrazol-3-yl)propan-2-ol trifluoroacetate

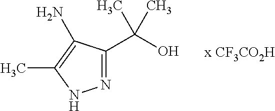

175 mg (0.95 mmol) of 2-(5-methyl-4-nitro-1H-pyrazol-3-yl)propan-2-ol from Example 49A were initially charged in 20 ml of ethanol/ethyl acetate, 596 mg (9.45 mmol) of ammonium formate and 75 mg of palladium (10% on activated carbon) were added and the mixture was stirred at 80° C. for 4 h. The reaction mixture was filtered through a Millipore filter and washed through with ethanol/ethyl acetate, and the filtrate was concentrated on a rotary evaporator. The crude product was purified by preparative HPLC (RP18 column; mobile phase: acetonitrile/water gradient with addition of 0.1% TFA). 179 mg of the target compound (68% of theory) were obtained.

LC-MS (Method 11): Rₜ=0.32 min

MS (ESpos): m/z=156 (M+H)⁺

### Example 51A

### Ethyl 2-chloro-3-cyclopropyl-3-oxopropanoate

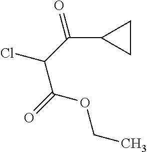

3.1 ml of sulphuryl chloride (38.2 mmol, 1.05 equivalents) were initially charged in 21 ml of dichloromethane, and 5.68 g of ethyl 3-cyclopropyl-3-oxopropanoate (36.4 mmol) were added dropwise on a water bath. The reaction mixture was stirred at RT for 2 h, and the mixture was then washed with water, 5% strength aqueous sodium bicarbonate solution and saturated aqueous sodium chloride solution, dried over magnesium sulphate and concentrated. The crude product (6.8 g) was used further without additional purification.

### Example 52A

### Ethyl 2-cyclopropyl-8-[(2,6-difluorobenzyl)oxy]imidazo[1,2-a]pyridine-3-carboxylate

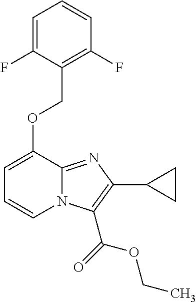 [SMILES: CCOC(=O)c1c(C2CC2)nc2c(OCc3c(F)cccc3F)cccn12]

1.69 g of 3[(2,6-difluorobenzyl)oxy]pyridine-2-amine (Example 1A; 7.13 mmol, 1 equivalent) were initially charged in 44.4 ml of ethanol, and 425 g of powdered molecular sieve (3A) and 6.8 g of ethyl 2-chloro-3-cyclopropyl-3-oxopropanoate (crude product from Example 51A) were added. The resulting reaction mixture was heated at reflux for 48 h and then concentrated, and the residue was chromatographed (mobile phase: cyclohexane/ethyl acetate). The product-containing fractions were combined and concentrated. The residue obtained in this manner was taken up in methanol, dimethyl sulphoxide and water, and the solid formed was filtered off and dried. This gave 410 mg (15.4% of theory) of the title compound.

LC-MS (Method 1): Rₜ=1.22 min

MS (ESpos): m/z=373.2 (M+H)⁺

¹H-NMR (400 MHz, DMSO-d₆): δ=0.95-1.05 (m, 4H); 1.39 (t, 3H); 2.36 (s, 3H); 2.70-2.80 (m, 1H); 4.39 (q, 2H); 5.30 (s, 2H); 7.08 (t, 1H); 7.15 (d, 1H); 7.20 (t, 2H); 7.59 (quint., 1H); 8.88 (d, 1H).

### Example 53A

### 2-Cyclopropyl-8-[(2,6-difluorobenzyl)oxy]imidazo[1,2-a]pyridine-3-carboxylic acid

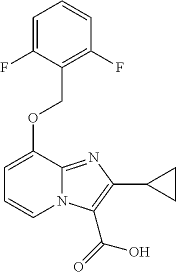 [SMILES: O=C(O)c1c(C2CC2)nc2c(OCc3c(F)cccc3F)cccn12]

410 mg of ethyl 2-cyclopropyl-8-[(2,6-difluorobenzyl)oxy]imidazo[1,2-a]pyridine-3-carboxylate (Example 52A, 1.1 mmol, 1 equivalent) were initially charged in 15 ml of methanol/tetrahydrofuran (1:1), and 5.5 ml of a 1 N aqueous lithium hydroxide solution (5.5 mmol, 5 equivalents) were added. The reaction mixture was stirred at RT overnight, after which no complete conversion had been achieved. Another 5.5 ml of 1 N aqueous lithium hydroxide solution were added, and the mixture was stirred at RT for another night. The mixture was concentrated and the residue was taken up in water and acidified with 1 N aqueous hydrochloric acid. The precipitated product was filtered off and dried under high vacuum. 293 mg (77% of theory) of the title compound were obtained.

LC-MS (Method 1): Rₜ=0.83 min

MS (ESpos): m/z=345.2 (M+H)⁺

¹H-NMR (400 MHz, DMSO-d₆): δ=0.95-1.02 (m, 4H); 2.80 (quint., 1H); 5.30 (s, 2H); 7.02 (t, 1H); 7.15 (d, 1H); 7.22 (t, 2H); 7.59 (quint., 1H); 8.92 (s, 1H); 13.3 (br. s, 1H).

### Example 54A

### 3-(Benzyloxy)-5-bromopyridine-2-amine

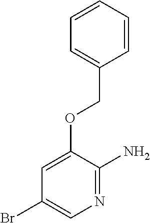 [SMILES: Nc1ncc(Br)cc1OCc1ccccc1]

200 g (1 mol) of 2-amino-3-benzyloxypyridine were initially charged in 4 l of dichloromethane, and at 0° C. a solution of 62 ml (1.2 mol) of bromine in 620 ml of dichloromethane was added over 30 min After the addition had ended, the reaction solution was stirred at 0° C. for 60 min. The mixture was then quenched with about 4 l of saturated sodium bicarbonate solution. The organic phase was removed and concentrated. The residue was purified by silica gel column chromatography (petroleum ether/ethyl acetate=6:4) and the product fractions were concentrated. This gave 214 g (77% of theory) of the title compound.

LC-MS (Method 2): Rₜ=0.92 min

MS (ESpos): m/z=279 (M+H)⁺

¹H-NMR (400 MHz, DMSO-d₆): δ=5.16 (s, 2H), 5.94-6.00 (m, 2H), 7.26-7.29 (m, 1H), 7.31-7.36 (m, 1H), 7.37-7.43 (m, 2H), 7.47-7.52 (m, 2H), 7.57-7.59 (m, 1H).

### Example 55A

### Ethyl 8-(benzyloxy)-6-bromo-2-methylimidazo[1,2-a]pyridine-3-carboxylate

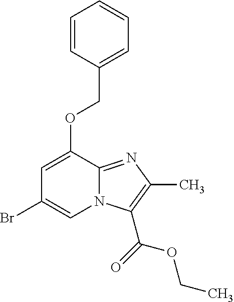 [SMILES: CCOC(=O)c1c(C)nc2c(OCc3ccccc3)cc(Br)cn12]

Under argon, 200 g (0.72 mol) of 3-(benzyloxy)-5-bromopyridine-2-amine, 590 g (3.58 mol) of ethyl 2-chloroacetoacetate and 436 g of 3 A molecular sieve were suspended in 6 l of ethanol, and the suspension was boiled at reflux for 72 h. The reaction mixture was filtered off with suction through silica gel and concentrated. The residue was purified by silica gel chromatography (petroleum ether:ethyl acetate=9:1, then 6:4) and the product fractions were concentrated. This gave 221 g (79% of theory) of the target compound.

LC-MS (Method 10): Rₜ=1.31 min

MS (ESpos): m/z=389 (M+H)⁺

¹H-NMR (400 MHz, DMSO-d₆): δ=1.36 (t, 3H), 2.58 (s, 3H), 4.32-4.41 (m, 2H), 5.33 (s, 2H), 7.28-7.32 (m, 1H), 7.36-7.47 (m, 3H), 7.49-7.54 (m, 2H), 8.98 (d, 1H).

### Example 56A

### Ethyl 8-(benzyloxy)-2,6-dimethylimidazo[1,2-a]pyridine-3-carboxylate

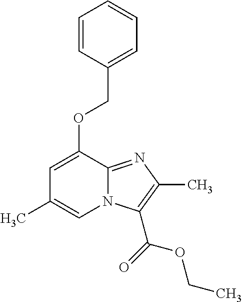 [SMILES: CCOC(=O)c1c(C)nc2c(OCc3ccccc3)cc(C)cn12]

Under argon, 105 g (270 mmol) of ethyl 8-(benzyloxy)-6-bromo-2-methylimidazo[1,2-a]pyridine-3-carboxylate from Example 55A were suspended in 4.2 μl of 1,4-dioxane, and 135.4 g (539 mmol, purity 50%) of trimethylboroxine, 31.2 g (27 mmol) of tetrakis(triphenylphosphine)palladium(0) and 78.3 g (566 mmol) of potassium carbonate were added in succession and the mixture was stirred under reflux for 8 h. The precipitate of the reaction mixture, cooled to RT, was removed by filtration with suction over silica gel, and the filtrate was concentrated. The residue was dissolved in dichloromethane and purified by silica gel chromatography (dichloromethane:ethyl acetate=9:1). This gave 74 g (84.6% of theory) of the target compound.

LC-MS (Method 10): Rₜ=1.06 min

MS (ESpos): m/z=325 (M+H)⁺

¹H-NMR (400 MHz, DMSO-d₆): δ=1.35 (t, 3H), 2.34 (br. s, 3H), 2.56 (s, 3H), 4.31-4.38 (m, 2H), 5.28 (br. s, 2H), 6.99-7.01 (m, 1H), 7.35-7.47 (m, 3H), 7.49-7.54 (m, 2H), 8.68-8.70 (m, 1H).

### Example 57A

### Ethyl 8-hydroxy-2,6-dimethylimidazo[1,2-a]pyridine-3-carboxylate

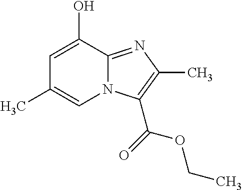 [SMILES: CCOC(=O)c1c(C)nc2c(O)cc(C)cn12]

74 g (228 mmol) of ethyl 8-(benzyloxy)-2,6-dimethylimidazo[1,2-a]pyridine-3-carboxylate from Example 56A were initially charged in 1254 ml of dichloromethane and 251 ml of ethanol, 20.1 g of 10% strength palladium on activated carbon (moist with water, 50%) were added under argon and the mixture was hydrogenated at RT and under standard pressure overnight. The reaction mixture was filtered off with suction through silica gel and concentrated. The crude product was purified by silica gel chromatography (dichloromethane:methanol=95:5). This gave 50.4 g (94% of theory) of the target compound.

DCI-MS: (Method 4) (ESpos): m/z=235.2 (M+H)⁺

¹H-NMR (400 MHz, DMSO-d₆): δ=1.35 (t, 3H), 2.27 (s, 3H), 2.58 (s, 3H), 4.30-4.38 (m, 2H), 6.65 (d, 1H), 8.59 (s, 1H), 10.57 (br. s, 1H).

### Example 58A

### Ethyl 2,6-dimethyl-8-[4,4,4-trifluoro-3-(trifluoromethyl)butoxy]imidazo[1,2-a]pyridine-3-carboxylate

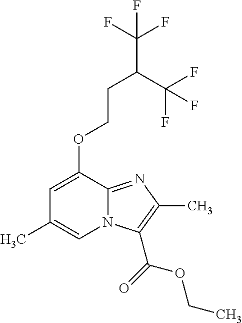 [SMILES: CCOC(=O)c1c(C)nc2c(OCCC(C(F)(F)F)C(F)(F)F)cc(C)cn12]

1.89 g (8.07 mmol) of ethyl 8-hydroxy-2,6-dimethylimidazo[1,2-a]pyridine-3-carboxylate from Example 57A were initially charged in 60 ml of DMF, 7.89 g (24.2 mol) of caesium carbonate and 2.30 g (8.88 mmol) of 4,4,4-trifluoro-3-(trifluoromethyl)butyl bromide were added and the reaction mixture was stirred at RT for 90 min 60 ml of water were added to the reaction mixture, the solid formed was filtered off and the filter residue was washed with 100 ml of water and twice with 20 ml of MTBE. The precipitate from the filtrate was filtered off and washed with mother liquor. The two filter residues were taken up in 50 ml of ethyl acetate, the solution was concentrated on a rotary evaporator and the residue was dried under reduced pressure overnight. This gave 2.25 g of the target compound (64% of theory).

LC-MS (Method 2): Rₜ=1.16 min

MS (ESpos): m/z=413 (M+H)⁺

¹H-NMR (400 MHz, DMSO-d₆): δ=1.36 (t, 3H), 2.34 (s, 3H), 2.32-2.38 (m, 2H), 2.58 (s, 3H), 4.18-4.30 (m, 1H), 4.31-4.38 (m, 4H), 6.93 (s, 1H), 8.71 (s, 1H).

### Example 59A

### 2,6-Dimethyl-8-[4,4,4-trifluoro-3-(trifluoromethyl)butoxy]imidazo[1,2-a]pyridine-3-carboxylic acid

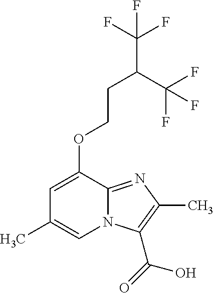 [SMILES: Cc1cc(OCCC(C(F)(F)F)C(F)(F)F)c2nc(C)c(C(=O)O)n2c1]

1.95 g (4.73 mmol) of ethyl 2,6-dimethyl-8-[4,4,4-trifluoro-3-(trifluoromethyl)butoxy]imidazo[1,2-a]pyridine-3-carboxylate from Example 58A were initially charged in 30 ml of methanol, 3.28 g (10.4 mmol) of barium hydroxide octahydrate were added and the mixture was stirred at RT for 3 days. The suspension was diluted with 30 ml of water and adjusted to pH 6 with 1 M aqueous hydrochloric acid. The solid was filtered off, washed with 50 ml of water and dried at 70° C. under reduced pressure for 2 h. 1.64 g of the target compound were obtained (81% of theory, 90% purity).

LC-MS (Method 2): Rₜ=0.78 min

MS (ESpos): m/z=385 (M+H)⁺

¹H-NMR (400 MHz, DMSO-d₆): δ=2.29 (s, 3H), 2.28-2.37 (m, 2H), 2.56 (s, 3H), 4.22-4.35 (m, 3H), 6.74 (s, 1H), 8.99 (s, 1H).

### Example 60A

### (3,3-Difluorocyclobutyl)methyl methanesulphonate

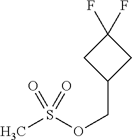

1.35 g (11.06 mmol) of (3,3-difluorocyclobutyl)methanol were initially charged in 41.8 ml of abs. dichloromethane, 3.08 ml (22.11 mmol) of triethylamine and 1.03 ml (13.27 mmol) of methanesulphonyl chloride were added and the mixture was stirred at room temperature overnight. Water was added to the reaction mixture, the aqueous phase was extracted twice with dichloromethane, the combined organic phases were washed once with saturated aqueous sodium chloride solution, dried over sodium sulphate and filtered, and the filtrate was concentrated. This gave 2.37 g (quantitative yield) of the target compound.

DCI-MS (Method 12): Rₜ=4.18 min m/z=218 (M+NH₄)⁺.

¹H-NMR (400 MHz, DMSO-d₆): δ=2.34-2.59 (m, 3H), 2.62-2.74 (m, 2H), 3.21 (s, 3H), 4.26 (d, 2H).

### Example 61A

### Ethyl 8-[(3,3-difluorocyclobutyl)methoxy]-2,6-dimethylimidazo[1,2-a]pyridine-3-carboxylate

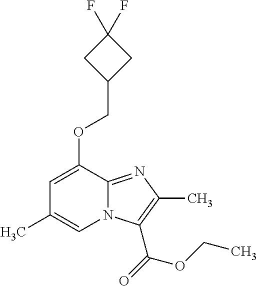 [SMILES: CCOC(=O)c1c(C)nc2c(OCC3CC(F)(F)C3)cc(C)cn12]

1.85 g (7.89 mmol) of ethyl 8-hydroxy-2,6-dimethylimidazo[1,2-a]pyridine-3-carboxylate from Example 57A and 2.37 g (9.47 mmol) of (3,3-difluorocyclobutyl)methyl methanesulphonate from Example 60A were initially charged in 104.4 ml of DMF, and 10.28 g (31.56 mmol) of caesium carbonate were added. The reaction mixture was stirred at 60° C. overnight. After cooling, the reaction mixture was filtered, the solid was washed with ethyl acetate, the filtrate was concentrated and about 150 ml of water were added to the residue. The solid formed was filtered off and dried under high vacuum. This gave 2.51 g (89% of theory) of the title compound.

LC-MS (Method 2): Rₜ=1.00 min

MS (ESpos): m/z=339 (M+H)⁺

¹H-NMR (400 MHz, DMSO-d₆): δ=1.35 (t, 3H), 2.32 (s, 3H), 2.42-2.60 (m, 5H), 2.62-2.84 (m, 3H), 4.22 (d, 2H), 4.33 (q, 2H), 6.90 (s, 1H), 8.68 (s, 1H).

### Example 62A

### 8-[(3,3-Difluorocyclobutyl)methoxy]-2,6-dimethylimidazo[1,2-a]pyridine-3-carboxylic acid

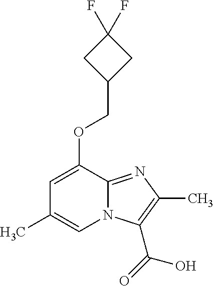

2.39 g (7.06 mmol) of ethyl 8[(3,3-difluorocyclobutyl)methoxy]-2,6-dimethylimidazo[1,2-a]pyridine-3-carboxylate from Example 61A were dissolved in 151 ml THF/methanol (5/1), 35.3 ml (35.3 mmol) of 1 N aqueous lithium hydroxide solution were added and the mixture was stirred at RT for 2 d. The reaction mixture was acidified to pH 4 using 1 N aqueous hydrochloric acid solution and then concentrated. The solid was filtered off with suction, washed with water and dried under high vacuum. This gave 1.63 g (71% of theory) of the title compound.

LC-MS (Method 2): Rₜ=0.63 min

MS (ESpos): m/z=311 (M+H)⁺

¹H-NMR (500 MHz, DMSO-d₆): δ=2.32 (s, 3H), 2.42-2.60 (m, 5H), 2.62-2.82 (m, 3H), 4.22 (d, 2H), 6.87 (s, 1H), 8.71 (s, 1H), 12.93 (br. s, 1H).

### WORKING EXAMPLES

### Example 1

### 8-[(2,6-Difluorobenzyl)oxy]-2-methyl-N-(pyrazolo[1,5-a]pyridin-3-yl)imidazo[1,2-a]pyridine-3-carboxamide

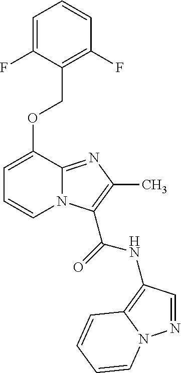 [SMILES: Cc1nc2c(OCc3c(F)cccc3F)cccn2c1C(=O)Nc1cnn2ccccc12]

75 mg (0.24 mmol) of 8-[(2,6-difluorobenzyl)oxy]-2-methylimidazo[1,2-a]pyridine-3-carboxylic acid, 91 mg (0.28 mmol) of (benzotriazol-1-yloxy)bisdimethylaminomethylium fluoroborate (TBTU) and 143 mg (1.41 mmol) of 4-methylmorpholine were initially charged in 1.5 ml of DMF, 63 mg (0.35 mmol) of pyrazolo[1,5-a]pyridine-3-amine hydrochloride were then added and the mixture was stirred at RT overnight. About 12 ml of water were added to the reaction solution, and the resulting precipitate was stirred for a further 30 min, filtered off with suction and washed thoroughly with water. The precipitate was stirred with 1.5 ml of acetonitrile, filtered with suction and dried under high vacuum overnight. This gave 71 mg of the target compound (70% of theory).

LC-MS (Method 2): Rₜ=0.83 min

MS (ESpos): m/z=434 (M+H)⁺

¹H-NMR (400 MHz, DMSO-d₆): δ=2.66 (s, 3H), 5.33 (s, 2H), 6.88 (t, 1H), 6.99 (t, 1H), 7.08 (d, 1H), 7.18-7.28 (m, 3H), 7.60 (quint, 1H), 7.78 (d, 1H), 8.32 (s, 1H), 8.62 (t, 2H), 9.98 (s, 1H).

Analogously to Example 1, the examples shown in Table 1 were prepared by reacting 8-[(2,6-difluorobenzyl)oxy]-2-methylimidazo[1,2-a]pyridine-3-carboxylic acid with the appropriate commercially available amines under the reaction conditions described in Representative Procedure 1:

TABLE 1

| Exam- | IUPAC name/structure | |
|---|---|---|
| ple | (Yield) | Analytical data |
| 2 | 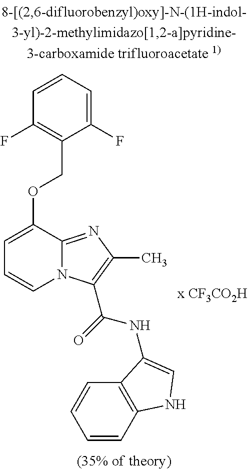 [SMILES: Cc1nc2c(OCc3c(F)cccc3F)cccn2c1C(=O)Nc1c[nH]c2ccccc12] | LC-MS (Method 2): Rₜ = 0.89 min MS (ESpos): m/z = 433 (M + H)⁺ ¹H-NMR (400 MHz, DMSO- d₆): δ = 2.70 (s, 3H), 5.42 (s, 2H), 7.05 (t, 1H), 7.14 (t, 1H), 7.25 (t, 2H), 7.39 (d, 2H), 7.62 (quint, 1H), 7.78 (d, 1H), 7.88 (s, 1H), 8.72 (d, 1H), 10.25 (s, 1H), 11.02 (s, 1H). |
| 3 | 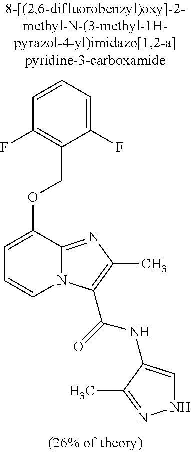 [SMILES: Cc1n[nH]cc1NC(=O)c1c(C)nc2c(OCc3c(F)cccc3F)cccn12] | LC-MS (Method 5): Rₜ = 0.82 min MS (ESpos): m/z = 398.14 (M + H)⁺ |
| 4 | 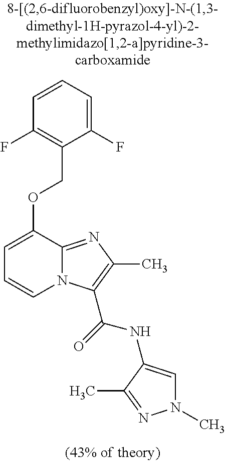 [SMILES: Cc1nn(C)cc1NC(=O)c1c(C)nc2c(OCc3c(F)cccc3F)cccn12] | LC-MS (Method 5): Rₜ = 0.87 min MS (ESpos): m/z = 412.12 (M + H)⁺ |
| 5 | 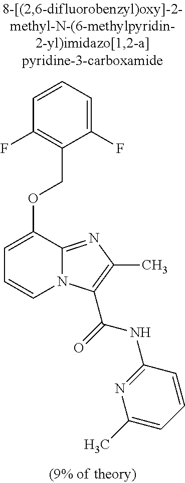 [SMILES: Cc1cccc(NC(=O)c2c(C)nc3c(OCc4c(F)cccc4F)cccn23)n1] | LC-MS (Method 5): Rₜ = 1.05 min MS (ESpos): m/z = 409.05 (M + H)⁺ |
| 6 | 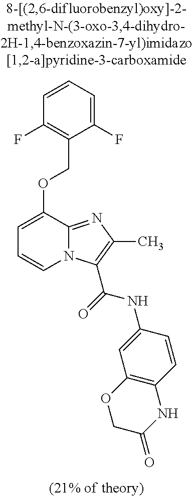 [SMILES: Cc1nc2c(OCc3c(F)cccc3F)cccn2c1C(=O)Nc1ccc2c(c1)OCC(=O)N2] | LC-MS (Method 5): Rₜ = 0.91 min MS (ESpos): m/z = 465.18 (M + H)⁺ |
| 7 | 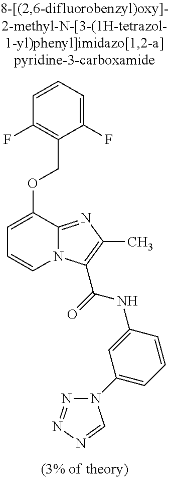 [SMILES: Cc1nc2c(OCc3c(F)cccc3F)cccn2c1C(=O)Nc1cccc(-n2cnnn2)c1] | LC-MS (Method 5): Rₜ = 1.01 min MS (ESpos): m/z = 464.20 (M + H)⁺ |
| 8 | 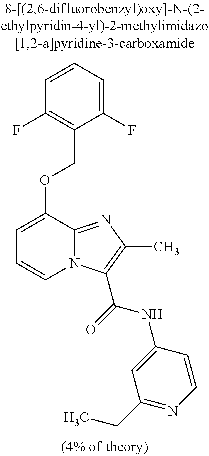 [SMILES: CCc1cc(NC(=O)c2c(C)nc3c(OCc4c(F)cccc4F)cccn23)ccn1] | LC-MS (Method 5): Rₜ = 0.80 min MS (ESpos): m/z = 423.11 (M + H)⁺ |
| 9 | 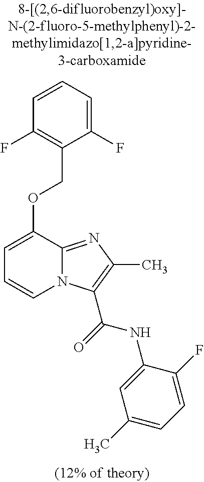 [SMILES: Cc1ccc(F)c(NC(=O)c2c(C)nc3c(OCc4c(F)cccc4F)cccn23)c1] | LC-MS (Method 5): Rₜ = 1.12 min MS (ESpos): m/z = 426.09 (M + H)⁺ |
| 10 | 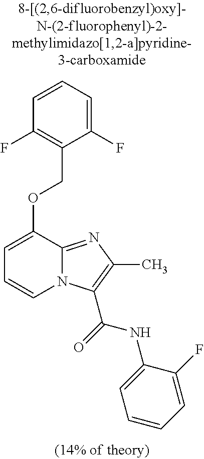 [SMILES: Cc1nc2c(OCc3c(F)cccc3F)cccn2c1C(=O)Nc1ccccc1F] | LC-MS (Method 5): Rₜ = 1.07 min MS (ESpos): m/z = 412.07 (M + H)⁺ |
| 11 | 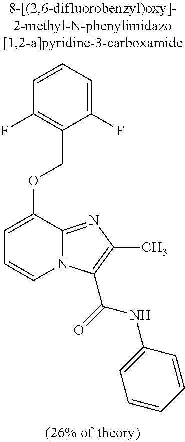 [SMILES: Cc1nc2c(OCc3c(F)cccc3F)cccn2c1C(=O)Nc1ccccc1] | LC-MS (Method 5): Rₜ = 1.02 min MS (ESpos): m/z = 394.06 (M + H)⁺ |
| 12 | 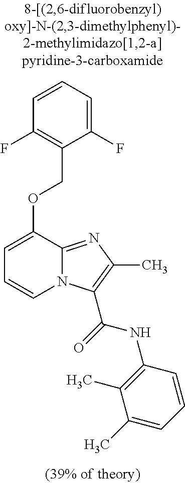 [SMILES: Cc1cccc(NC(=O)c2c(C)nc3c(OCc4c(F)cccc4F)cccn23)c1C] | LC-MS (Method 5): Rₜ = 1.06 min MS (ESpos): m/z = 422.15 (M + H)⁺ |
| 13 | 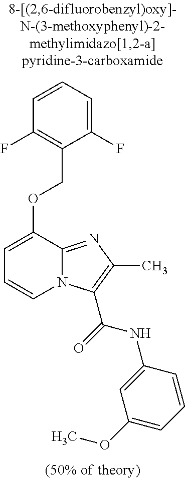 [SMILES: COc1cccc(NC(=O)c2c(C)nc3c(OCc4c(F)cccc4F)cccn23)c1] | LC-MS (Method 5): Rₜ = 1.04 min MS (ESpos): m/z = 424.14 (M + H)⁺ |
| 14 | 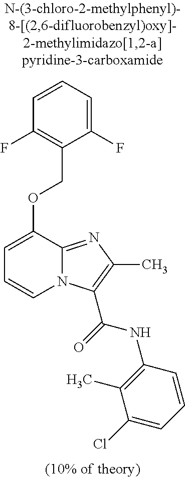 [SMILES: Cc1nc2c(OCc3c(F)cccc3F)cccn2c1C(=O)Nc1cccc(Cl)c1C] | LC-MS (Method 5): Rₜ = 1.11 min MS (ESpos): m/z = 442.09 (M + H)⁺ |
| 15 | 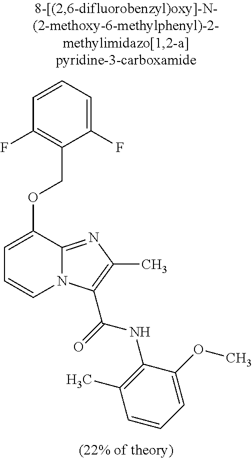 [SMILES: COc1cccc(C)c1NC(=O)c1c(C)nc2c(OCc3c(F)cccc3F)cccn12] | LC-MS (Method 5): Rₜ = 1.00 min MS (ESpos): m/z = 438.15 (M + H)⁺ |
| 16 | 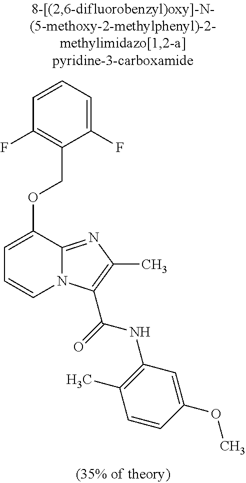 [SMILES: COc1ccc(C)c(NC(=O)c2c(C)nc3c(OCc4c(F)cccc4F)cccn23)c1] | LC-MS (Method 5): Rₜ = 1.06 min MS (ESpos): m/z = 438.15 (M + H)⁺ |
| 17 | 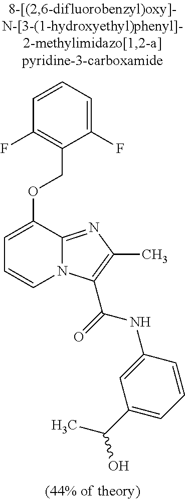 [SMILES: Cc1nc2c(OCc3c(F)cccc3F)cccn2c1C(=O)Nc1cccc(C(C)O)c1] | LC-MS (Method 5): Rₜ = 0.92 min MS (ESpos): m/z = 438.15 (M + H)⁺ |
| 18 | 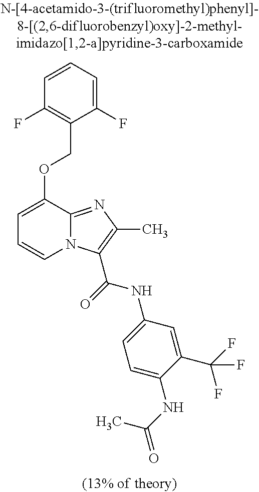 [SMILES: CC(=O)Nc1ccc(NC(=O)c2c(C)nc3c(OCc4c(F)cccc4F)cccn23)cc1C(F)(F)F] | LC-MS (Method 5): Rₜ = 0.99 min MS (ESpos): m/z = 519.17 (M + H)⁺ |
| 19 | 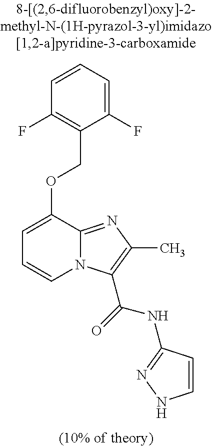 [SMILES: Cc1nc2c(OCc3c(F)cccc3F)cccn2c1C(=O)Nc1cc[nH]n1] | LC-MS (Method 5): Rₜ = 0.92 min MS (ESpos): m/z = 384.12 (M + H)⁺ |
| 20 | 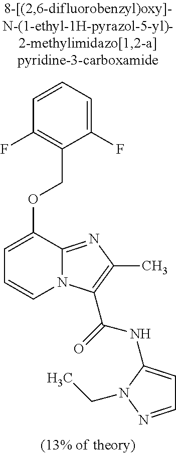 [SMILES: CCn1nccc1NC(=O)c1c(C)nc2c(OCc3c(F)cccc3F)cccn12] | LC-MS (Method 5): Rₜ = 0.95 min MS (ESpos): m/z = 412.16 (M + H)⁺ |
| 21 | 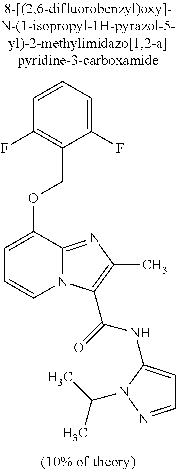 [SMILES: Cc1nc2c(OCc3c(F)cccc3F)cccn2c1C(=O)Nc1ccnn1C(C)C] | LC-MS (Method 5): Rₜ = 0.98 min MS (ESpos): m/z = 426.14 (M + H)⁺ |

¹⁾ After stirring with acetonitrile, the precipitate was purifed once more by preparative TLC (dichloromethane:methanol = 10:1) and then by preparative HPLC [Sunfire C 18, 5 μm, 250 × 20 mm, mobile phase: 50% methanol, 40% water, 10% of a 1% strength aqueous trifluoroacetic acid solution]

### Example 22

### 8-[(2,6-Difluorobenzyl)oxy]-N-[1-(2-hydroxyethyl)-1H-pyrazol-4-yl]-2-methylimidazo[1,2-a]pyridine-3-carboxamide

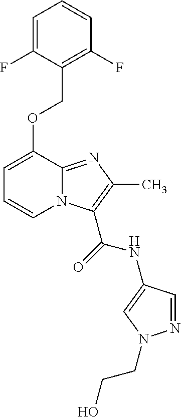 [SMILES: Cc1nc2c(OCc3c(F)cccc3F)cccn2c1C(=O)Nc1cnn(CCO)c1]

70 mg of 8-[(2,6-difluorobenzyl)oxy]-2-methylimidazo[1,2-a]pyridine-3-carboxylic acid (Example 3A; 0.22 mmol, 1 equivalent), 109 mg of O-(7-azabenzotriazol-1-yl)-N,N,N′N′-tetramethyluronium hexafluorophosphate (HATU; 0.286 mmol, 1.3 equivalents) and 153 μl of N,N-diisopropylethylamine (DIPEA; 0.880 mmol, 4 equivalents) were initially charged in 1 ml of DMF, and 54 mg of 2-(4-amino-1H-pyrazol-1-yl)ethan-1-ol hydrochloride (0.33 mmol, 1.5 equivalents) were added and the mixture was stirred at RT overnight. Water was added to the reaction solution, the resulting precipitate was stirred for another 5 min, filtered off with suction and washed thoroughly with water and dried under high vacuum overnight. 70 mg (75% of theory) of the title compound were obtained.

LC-MS (Method 2): Rₜ=0.64 min

MS (ESpos): m/z=428.1 (M+H)⁺

¹H-NMR (400 MHz, DMSO-d₆): δ=2.56 (s, 3H; superposed by DMSO signal); 3.72 (q, 2H); 4.13 (t, 2H); 4.90 (t, 1H); 5.32 (s, 2H); 6.96 (t, 1H); 7.04 (d, 1H); 7.24 (t, 2H); 7.59 (quint., 1H); 7.60 (s, 1H); 8.04 (s, 1H); 8.57 (d, 1H); 9.97 (s, 1H).

Analogously to Example 22, the Examples shown in Table 2 were prepared by reacting 8-[(2,6-difluorobenzyl)oxy]-2-methylimidazo[1,2-a]pyridine-3-carboxylic acid (Example 3A) with the appropriate commercially available amines under the reaction conditions described in Representative Procedure 2:

TABLE 2

| | IUPAC name/structure | |
|---|---|---|
| Example | (Yield) | Analytical data |
| 23 | 8-[(2,6-difluorobenzyl)oxy]-N-(3,5-dimethyl-1,2- oxazol-4-yl)-2-methylimidazo[1,2-a]pyridine-3- carboxamide 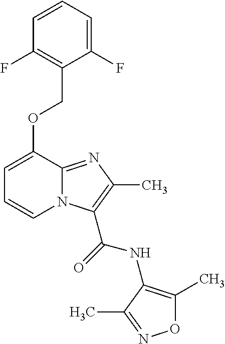 [SMILES: Cc1noc(C)c1NC(=O)c1c(C)nc2c(OCc3c(F)cccc3F)cccn12] (43% of theory) | LC-MS (Method 2): Rₜ = 0.81 min MS (ESpos): m/z = 413.1 (M + H)⁺ ¹H-NMR (400 MHz, DMSO-d₆): δ = 2.16 (s, 3 H); 2.34 (s, 3 H); 2.63 (s, 3 H); 5.31 (s, 2 H); 6.99 (t, 1 H); 7.05 (d, 1 H); 7.24 (t, 2 H); 7.59 (quint., 1 H); 8.60 (d, 1 H); 9.20 (s, 1 H). |
| 24 | 8-[(2,6-difluorobenzyl)oxy]-N-(1-ethyl-1H- pyrazol-4-yl)-2-methylimidazo[1,2-a]pyridine-3- carboxamide 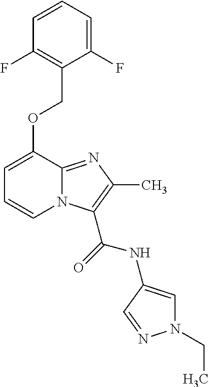 [SMILES: CCn1cc(NC(=O)c2c(C)nc3c(OCc4c(F)cccc4F)cccn23)cn1] (59% of theory) | LC-MS (Method 2): Rₜ = 0.76 min MS (ESpos): m/z = 413.1 (M + H)⁺ ¹H-NMR (400 MHz, DMSO-d₆): δ = 1.35 (t, 3 H); 2.57 (s, 3 H; superposed by DMSO signal); 4.11 (q, 2 H); 5.30 (s, 2 H); 6.95 (t, 1 H); 7.02 (d, 1 H); 7.21 (t, 2 H); 7.58 (s, 1 H); 7.59 (quint., 1 H); 8.03 (s, 1 H); 8.58 (d, 1 H); 9.95 (s, 1 H). |
| 25 | N-[1-(2-amino-2-oxoethyl)-1H-pyrazol-4-yl]-8- [(2,6-difluorobenzyl)oxy]-2-methylimidazo[1,2- a]pyridine-3-carboxamide 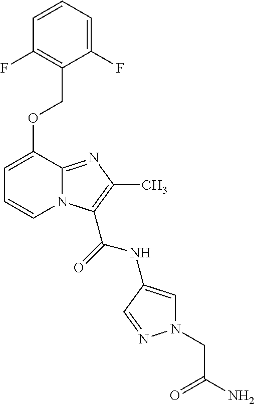 [SMILES: Cc1nc2c(OCc3c(F)cccc3F)cccn2c1C(=O)Nc1cnn(CC(N)=O)c1] (56% of theory) | LC-MS (Method 2): Rₜ = 0.62 min MS (ESpos): m/z = 441.1 (M + H)⁺ ¹H-NMR (400 MHz, DMSO-d₆): δ = 2.57 (s, 3 H; superposed by DMSO signal); 4.72 (s, 2 H); 5.30 (s, 2 H); 6.95 (t, 1 H); 7.02 (d, 1 H); 7.21 (t, 2 H); 7.21 (s, 1 H); 7.49 (s, 1 H); 7.58 (quint., 1 H); 7.59 (s, 1 H); 8.05 (s, 1 H); 8.59 (d, 1 H); 10.00 (s, 1 H). |
| 26 | 8-[(2,6-difluorobenzyl)oxy]-N-[1-(2- methoxyethyl)-1H-pyrazol-4-yl]-2- methylimidazo[1,2-a]pyridine-3-carboxamide 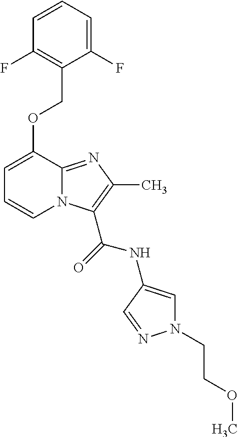 [SMILES: COCCn1cc(NC(=O)c2c(C)nc3c(OCc4c(F)cccc4F)cccn23)cn1] (64% of theory) | LC-MS (Method 2): Rₜ = 0.73 min MS (ESpos): m/z = 442.2 (M + H)⁺ ¹H-NMR (400 MHz, DMSO-d₆): δ = 2.57 (s, 3 H; superposed by DMSO signal); 3.22 (s, 3 H); 3.55 (t, 2 H); 4.25 (t, 2 H); 5.30 (s, 2 H); 6.95 (t, 1 H); 7.02 (d, 1H); 7.21 (t, 2 H); 7.58 (quint., 1 H); 7.59 (s, 1 H); 8.02 (s, 1 H); 8.58 (d, 1 H); 9.95 (s, 1 H). |
| 27 | 8-[(2,6-difluorobenzyl)oxy]-N-(1-isopropyl-1H- pyrazol-4-yl)-2-methylimidazo[1,2-a]pyridine-3- carboxamide 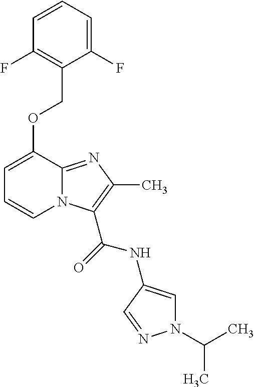 [SMILES: Cc1nc2c(OCc3c(F)cccc3F)cccn2c1C(=O)Nc1cnn(C(C)C)c1] (80% of theory) | LC-MS (Method 2): Rₜ = 0.85 min MS (ESpos): m/z = 426.2 (M + H)⁺ ¹H-NMR (400 MHz, DMSO-d₆): δ = 1.40 (d, 6 H); 2.57 (s, 3 H; superposed by DMSO signal); 4.50 (septett, 1 H); 5.30 (s, 2 H); 6.95 (t, 1 H); 7.02 (d, 1 H); 7.22 (t, 2 H); 7.58 (s, 1 H); 7.59 (quint., 1 H); 8.04 (s, 1 H); 8.56 (d, 1 H); 9.95 (s, 1 H). |
| 28 | 8-[(2,6-difluorobenzyl)oxy]-N-(1,5-dimethyl-1H- pyrazol-4-yl)-2-methylimidazo[1,2-a]pyridine-3- carboxamide 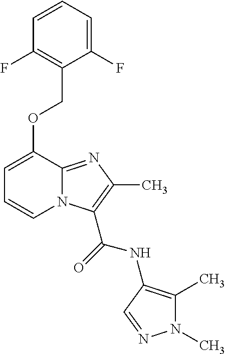 (54% of theory) | LC-MS (Method 2): Rₜ = 0.70 min MS (ESpos): m/z = 426.2 (M + H)⁺ ¹H-NMR (400 MHz, DMSO-d₆): δ = 2.20 (s, 3 H); 2.61 (s, 3 H); 3.71 (s, 3 H); 5.30 (s, 2 H); 6.95 (t, 1 H); 7.03 (d, 1 H); 7.22 (t, 2 H); 7.57 (s, 1 H); 7.59 (quint., 1 H); 8.58 (d, 1 H); 9.30 (s, 1 H). |
| 29 | 8-[(2,6-difluorobenzyl)oxy]-N-(3,5-dimethyl-1H- pyrazol-4-yl)-2-methylimidazo[1,2-a]pyridine-3- carboxamide 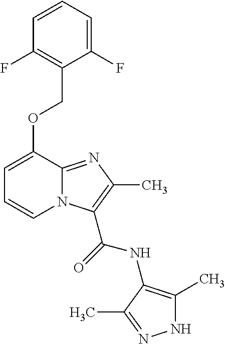 [SMILES: Cc1n[nH]c(C)c1NC(=O)c1c(C)nc2c(OCc3c(F)cccc3F)cccn12] (92% of theory) | LC-MS (Method 2): Rₜ = 0.71 min MS (ESpos): m/z = 412.2 (M + H)⁺ 1H-NMR (400 MHz, DMSO-d₆): δ = 2.04 (s, 3 H); 2.12 (s, 3 H); 2.62 (s, 3 H); 5.31 (s, 2 H); 6.94 (t, 1 H); 6.99 (d, 1 H); 7.21 (t, 2 H); 7.58 (quint., 1 H); 8.56 (d, 1 H); 8.95 (s, 1 H); 12.15 (s, 1 H). |
| 30 | 8-[(2,6-difluorobenzyl)oxy]-2-methyl-N-[1-(2,2,2- trifluoroethyl)-1H-pyrazol-4-yl]imidazo[1,2- a]pyridine-3-carboxamide 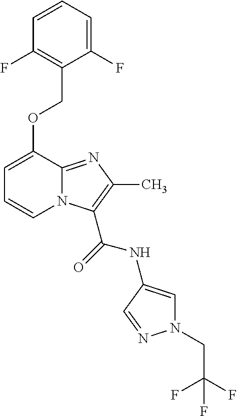 [SMILES: Cc1nc2c(OCc3c(F)cccc3F)cccn2c1C(=O)Nc1cnn(CC(F)(F)F)c1] (63% of theory) | LC-MS (Method 2): Rₜ = 0.88 min MS (ESpos): m/z = 466.1 (M + H)⁺ ¹H-NMR (400 MHz, DMSO-d₆): δ = 2.58 (s, 3 H; superposed by DMSO signal); 5.13 (q, 2 H); 5.30 (s, 2 H); 6.95 (t, 1 H); 7.04 (d, 1 H); 7.23 (t, 2 H); 7.59 (quint., 1 H); 7.70 (s, 1 H); 8.20 (s, 1 H); 8.58 (d, 1 H); 10.06 (s, 1 H). |
| 31 | 8-[(2,6-difluorobenzyl)oxy]-2-methyl-N-(1H- pyrazol-4-yl)imidazo[1,2-a]pyridine-3- carboxamide 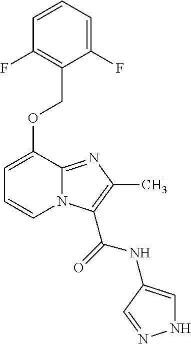 [SMILES: Cc1nc2c(OCc3c(F)cccc3F)cccn2c1C(=O)Nc1cn[nH]c1] (41% of theory) | LC-MS (Method 1): Rₜ = 0.80 min MS (ESpos): m/z = 384.0 (M + H)⁺ ¹H-NMR (400 MHz, DMSO-d₆): δ = 2.57 (s, 3 H; superposed by DMSO signal); 5.30 (s, 2 H); 6.93 (t, 1 H); 7.03 (d, 1 H); 7.23 (t, 2 H); 7.59 (quint., 1 H); 7.65 (s, 1 H); 8.00 (s, 1 H); 8.58 (d, 1 H); 9.92 (s, 1 H); 12.63 (br. s, 1 H). |
| 32 | 8-[(2,6-difluorobenzyl)oxy]-N-[3,5-dimethyl-1- (2,2,2-trifluoroethyl)-1H-pyrazol-4-yl]-2- methylimidazo[1,2-a]pyridine-3-carboxamide 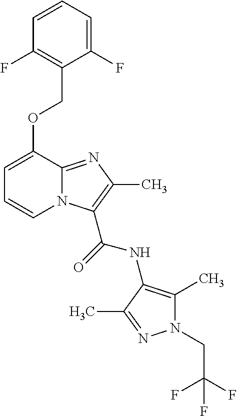 [SMILES: Cc1nn(CC(F)(F)F)c(C)c1NC(=O)c1c(C)nc2c(OCc3c(F)cccc3F)cccn12] (70% of theory) isolated as HCl salt | LC-MS (Method 2): Rₜ = 0.84 min MS (ESpos): m/z = 494.1 (M + H)⁺ ¹H-NMR (400 MHz, DMSO-d₆): δ = 2.09 (s, 3 H); 2.20 (s, 3 H); 2.62 (s, 3 H); 5.01 (q, 2 H); 5.38 (s, 2 H); 7.1 (br. s, 1 H); 7.20 (br.s, 1 H); 7.21 (t, 2 H); 7.59 (quint., 1 H); 8.59 (d, 1 H); 9.2 (br. s, 1 H). |
| 33 | 8-[(2,6-difluorobenzyl)oxy]-2-methyl-N-[3- methyl-1-(2,2,2-trifluoroethyl)-1H-pyrazol-4- yl]imidazo[1,2-a]pyridine-3-carboxamide 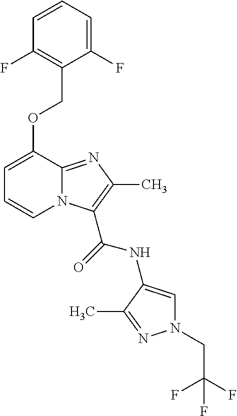 [SMILES: Cc1nn(CC(F)(F)F)cc1NC(=O)c1c(C)nc2c(OCc3c(F)cccc3F)cccn12] (68% of theory) | LC-MS (Method 2): Rₜ = 0.86 min MS (ESpos): m/z = 480.1 (M + H)⁺ ¹H-NMR (400 MHz, DMSO-d₆): δ = 2.20 (s, 3 H); 2.62 (s, 3 H); 5.06 (q, 2 H); 5.31 (s, 2 H); 6.96 (t, 1 H); 7.06 (d, 1 H); 7.22 (t, 2 H); 7.60 (quint., 1 H); 8.20 (s, 1 H); 8.59 (d, 1 H); 9.98 (s, 1 H). |
| 34 | 8-[(2,6-difluorobenzyl)oxy]-N-[1,3-dimethyl-5- (morpholin-4-yl)-1H-pyrazol-4-yl]-2- methylimidazo[1,2-a]pyridine-3-carboxamide 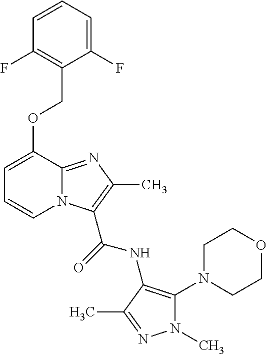 [SMILES: Cc1nn(C)c(N2CCOCC2)c1NC(=O)c1c(C)nc2c(OCc3c(F)cccc3F)cccn12] (65% of theory) | LC-MS (Method 1): Rₜ = 0.91 min MS (ESpos): m/z = 497.1 (M + H)⁺ ¹H-NMR (400 MHz, DMSO-d₆): δ = 1.99 (s, 3 H); 2.62 (s, 3 H); 3.02-3.07 (m, 4 H); 3.59 (s, 3 H); 3.63-3.69 (m, 4 H); 5.31 (s, 2 H); 6.97 (t, 1 H); 7.06 (d, 1 H); 7.23 (t, 2 H); 7.59 (quint., 1 H); 8.59 (d, 1 H); 9.95 (s, 1 H). |
| 35 | 8-[(2,6-difluorobenzyl)oxy]-N-[1-(3- fluorobenzyl)-1H-pyrazol-4-yl]-2- methylimidazo[1,2-a]pyridine-3-carboxamide 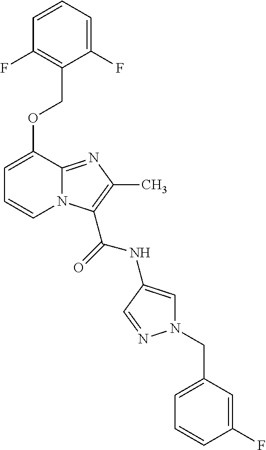 [SMILES: Cc1nc2c(OCc3c(F)cccc3F)cccn2c1C(=O)Nc1cnn(Cc2cccc(F)c2)c1] (63% of theory) | LC-MS (Method 2): Rₜ = 0.92 min MS (ESpos): m/z = 492.1 (M + H)⁺ ¹H-NMR (400 MHz, DMSO-d₆): δ = 2.57 (s, 3 H; superposed by DMSO signal); 5.30 (s, 2 H); 5.32 (s, 2 H); 6.97 (t, 1 H); 7.05 (d, 1 H); 7.08-7.18 (m, 3 H); 7.23 (t, 2 H); 7.40 (q, 1 H); 7.59 (quint., 1 H); 7.62 (s, 1 H); 8.18 (s, 1 H); 8.59 (d, 1 H); 10.00 (s, 1 H). |
| 36 | 8-[(2,6-difluorobenzyl)oxy]-2-methyl-N-[1- methyl-5-(methylcarbamoyl)-1H-pyrazol-4- yl]imidazo[1,2-a]pyridine-3-carboxamide 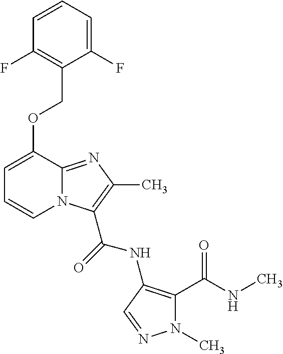 [SMILES: CNC(=O)c1c(NC(=O)c2c(C)nc3c(OCc4c(F)cccc4F)cccn23)cnn1C] (10% of theory) | LC-MS (Method 2): Rₜ = 0.80 min MS (ESpos): m/z = 455.2 (M + H)⁺ ¹H-NMR (400 MHz, DMSO-d₆): δ = 2.62 (s, 3 H); 2.80 (d, 3 H); 3.95 (s, 3 H); 5.30 (s, 2 H); 7.00 (t, 1 H); 7.09 (d, 1 H); 7.23 (t, 2 H); 7.59 (quint., 1 H); 7.92 (s, 1 H); 8.22 (br. q, 1 H); 8.85 (d, 1 H); 9.50 (s, 1 H). |
| 37 | 8-[(2,6-difluorobenzyl)oxy]-N-[1-(2- hydroxyethyl)-3,5-dimethyl-1H-pyrazol-4-yl]-2- methylimidazo[1,2-a]pyridine-3-carboxamide 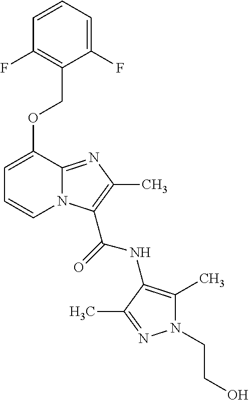 [SMILES: Cc1nn(CCO)c(C)c1NC(=O)c1c(C)nc2c(OCc3c(F)cccc3F)cccn12] | LC-MS (Method 2): Rₜ = 0.73 min MS (ESpos): m/z = 456.3 (M + H)⁺ |

### Example 38

### 8-[(2,6-Difluorobenzyl)oxy]-N-(1-ethyl-3,5-dimethyl-1H-pyrazol-4-yl)-2-methylimidazo[1,2-a]pyridine-3-carboxamide

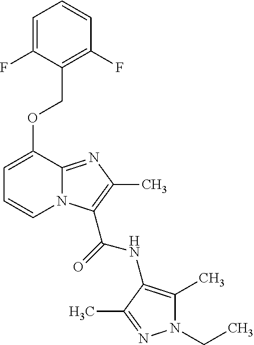 [SMILES: CCn1nc(C)c(NC(=O)c2c(C)nc3c(OCc4c(F)cccc4F)cccn23)c1C]

50 mg (0.16 mmol) of 8-[(2,6-difluorobenzyl)oxy]-2-methylimidazo[1,2-a]pyridine-3-carboxylic acid, 149 mg (0.39 mmol) of O-(7-azabenzotriazol-1-yl)-N,N,N′N′-tetramethyluronium hexafluorophosphate (HATU) and 51 mg (0.39 mmol) of N,N-diisopropylethylamine were initially charged in 1 ml of DMF, the mixture was stirred for 20 min, 44 mg (0.31 mmol) of 1-ethyl-3,5-dimethyl-1H-pyrazole-4-amine were then added and the mixture was stirred at RT overnight. Another 11 mg (0.08 mmol) of 1-ethyl-3,5-dimethyl-1H-pyrazole-4-amine were then added and the mixture was stirred at RT for 2 h. About 8 ml of water were added and the reaction mixture was extracted three times with ethyl acetate. The combined organic phases were concentrated and the residue was purified by preparative HPLC (RP18 column, mobile phase: acetonitrile/water gradient with addition of 0.1% trifluoroacetic acid). The product-containing fractions were then concentrated and purified by preparative thin layer chromatography (dichloromethane:methanol=15:1). 25 mg of the target compound (36% of theory) were obtained.

LC-MS (Method 2): Rₜ=0.80 min

MS (ESpos): m/z=440 (M+H)⁺

¹H-NMR (400 MHz, DMSO-d₆): δ=1.29 (t, 3H), 2.05 (s, 3H), 2.16 (s, 3H), 2.62 (s, 3H), 3.99 (q, 2H), 5.32 (s, 2H), 6.96 (t, 1H), 7.04 (d, 1H), 7.24 (t, 2H), 7.59 (quint, 1H), 8.56 (d, 1H), 8.97 (s, 1H).

### Example 39

### 6-Chloro-8-[(2,6-difluorobenzyl)oxy]-N-(3,5-dimethyl-1,2-oxazol-4-yl)-2-methylimidazo[1,2-a]pyridine-3-carboxamide

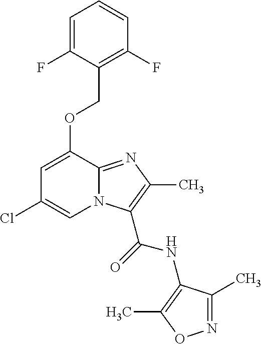 [SMILES: Cc1noc(C)c1NC(=O)c1c(C)nc2c(OCc3c(F)cccc3F)cc(Cl)cn12]

60 mg of 6-chloro-8-[(2,6-difluorobenzyl)oxy]-2-methylimidazo[1,2-a]pyridine-3-carboxylic acid (Example 16A; 0.17 mmol, 1 equivalent), 84 mg of O-(7-azabenzotriazol-1-yl)-N,N,N′N′-tetramethyluronium hexafluorophosphate (HATU; 0.221 mmol, 1.3 equivalents) and 84 μl of N,N-diisopropylethylamine (DIPEA; 0.51 mmol, 3 equivalents) were initially charged in 0.5 ml of DMF, and 27 mg of 4-amino-3,5-dimethylisoxazole (0.24 mmol, 1.4 equivalents) were added and the mixture was stirred at RT for 4 h. Water was added to the reaction solution, the resulting precipitate was stirred for another 5 min, filtered off with suction and washed thoroughly with water and dried under high vacuum overnight. This gave 63 mg (79% of theory) of the title compound as beige crystals.

LC-MS (Method 1): Rₜ=1.29 min

MS (ESpos): m/z=447.0/449.0 (M+H)⁺

¹H-NMR (400 MHz, DMSO-d₆): δ=2.16 (s, 3H); 2.33 (s, 3H); 2.62 (s, 3H); 5.32 (s, 2H); 7.22 (t, 2H); 7.23 (s, 1H); 7.59 (quint., 1H); 8.70 (s, 1H); 9.25 (s, 1H).

Analogously to Example 39, the Examples shown in Table 3 were prepared by reacting 6-chloro-8-[(2,6-difluorobenzyl)oxy]-2-methylimidazo[1,2-a]pyridine-3-carboxylic acid (Example 16A) with the appropriate commercially available amines under the reaction conditions described in Representative Procedure 2:

TABLE 3

| | IUPAC name/structure | |
|---|---|---|
| Example | (Yield) | Analytical data |
| 40 | 6-chloro-8-[(2,6-difluorobenzyl)oxy]-2-methyl-N- [3-methyl-5-(trifluoromethyl)-1H-pyrazol-4- yl]imidazo[1,2-a]pyridine-3-carboxamide 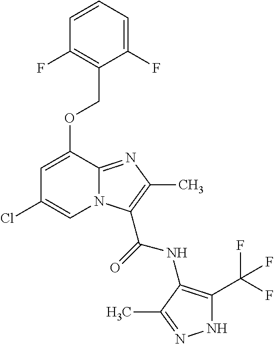 [SMILES: Cc1n[nH]c(C(F)(F)F)c1NC(=O)c1c(C)nc2c(OCc3c(F)cccc3F)cc(Cl)cn12] (12% of theory) | LC-MS (Method 1): Rₜ = 1.26 min MS (ESpos): m/z = 500.0/501.9 (M + H)⁺ ¹H-NMR (400 MHz, DMSO- d₆): δ = 2.20 (s, 3 H); 2.60 (s, 3 H); 5.38 (s, 2 H); 7.25 (t, 2 H); 7.25 (s, 1 H); 7.60 (quint., 1 H); 8.61 (s, 1 H); 9.30 (s, 1 H); 13.5 (br. s, 1 H). |
| 41 | 6-chloro-8-[(2,6-difluorobenzyl)oxy]-N-(3,5- dimethyl-1H-pyrazol-4-yl)-2-methylimidazo[1,2- a]pyridine-3-carboxamide 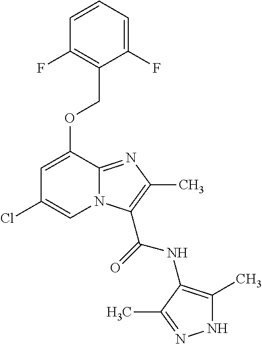 [SMILES: Cc1n[nH]c(C)c1NC(=O)c1c(C)nc2c(OCc3c(F)cccc3F)cc(Cl)cn12] (62% of theory) | LC-MS (Method 2): Rₜ = 0.87 min MS (ESpos): m/z = 446.1/448.1 (M + H)⁺ ¹H-NMR (400 MHz, DMSO- d₆): δ = 2.08 (br. s, 3 H); 2.15 (br. s, 3 H); 2.63 (s, 3 H); 5.35 (s, 2 H); 7.22 (s, 1 H); 7.25 (t, 2 H); 7.60 (quint., 1 H); 8.65 (s, 1 H); 9.02 (s, 1 H); 12.2 (br. s, 1 H). |
| 42 | 6-chloro-8-[(2,6-difluorobenzyl)oxy]-N-[1-(2- hydroxyethyl)-3,5-dimethyl-1H-pyrazol-4-yl]-2- methylimidazo[1,2-a]pyridine-3-carboxamide 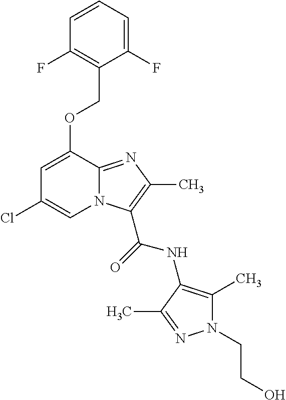 [SMILES: Cc1nn(CCO)c(C)c1NC(=O)c1c(C)nc2c(OCc3c(F)cccc3F)cc(Cl)cn12] | LC-MS (Method 2): Rₜ = 0.91 min MS (ESpos): m/z = 490.3/492.3 (M + H)⁺ |
| 43 | 6-chloro-8-[(2,6-difluorobenzyl)oxy]-N-[3,5- dimethyl-1-(2,2,2-trifluoroethyl)-1H-pyrazol-4- yl]-2-methylimidazo[1,2-a]pyridine-3- carboxamide 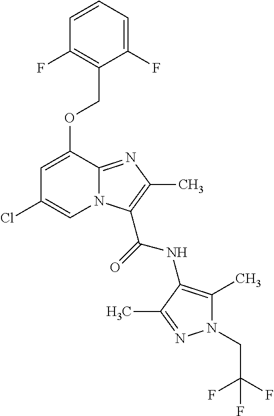 [SMILES: Cc1nn(CC(F)(F)F)c(C)c1NC(=O)c1c(C)nc2c(OCc3c(F)cccc3F)cc(Cl)cn12] (76% of theory) | LC-MS (Method 2): Rₜ = 1.05 min MS (ESpos): m/z = 528.3/530.2 (M + H)⁺ ¹H-NMR (400 MHz, DMSO- d₆): δ = 2.09 (s, 3 H); 2.20 (br. s, 3 H); 2.61 (s, 3 H); 5.02 (q, 2 H); 5.37 (s, 2 H); 7.21-7.29 (m, 3 H); 7.60 (quint., 1 H); 8.68 (s, 1 H); 9.11 (s, 1 H). |
| 44 | 6-chloro-8-[(2,6-difluorobenzyl)oxy]-2-methyl-N- [1-(2,2,2-trifluoroethyl)-1H-pyrazol-4- yl]imidazo[1,2-a]pyridine-3-carboxamide 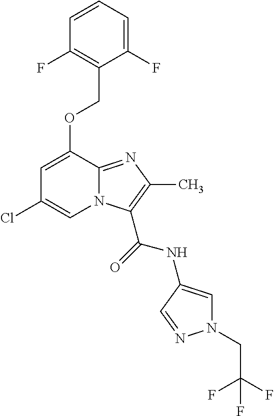 [SMILES: Cc1nc2c(OCc3c(F)cccc3F)cc(Cl)cn2c1C(=O)Nc1cnn(CC(F)(F)F)c1] (64% of theory) | LC-MS (Method 1): Rₜ = 1.29 min MS (ESpos): m/z = 500.0/501.9 (M + H)⁺ ¹H-NMR (400 MHz, DMSO- d₆): δ = 2.56 (s, 3 H; superposed by DMSO signal); 5.11 (q, 2 H); 5.37 (s, 2 H); 7.21-7.29 (m, 3 H); 7.60 (quint., 1 H); 7.72 (s, 1 H); 8.21 (s, 1 H); 8.69 (s, 1 H); 10.11 (s, 1 H). |
| 45 | 6-chloro-8-[(2,6-difluorobenzyl)oxy]-N-(1- isopropyl-1H-pyrazol-4-yl)-2-methylimidazo[1,2- a]pyridine-3-carboxamide 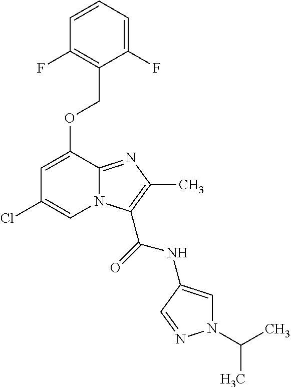 [SMILES: Cc1nc2c(OCc3c(F)cccc3F)cc(Cl)cn2c1C(=O)Nc1cnn(C(C)C)c1] (61% of theory) | LC-MS (Method 2): Rₜ = 1.09 min MS (ESpos): m/z = 460.3/462.3 (M + H)⁺ ¹H-NMR (400 MHz, DMSO- d₆): δ = 1.41 (d, 6 H); 2.57 (s, 3 H; superposed by DMSO signal); 4.50 (sept., 1 H); 5.37 (s, 2 H); 7.21 (d, 1 H); 7.25 (t, 2 H); 7.58 (s, 1 H); 7.60 (quint., 1 H); 8.06 (s, 1 H); 8.71 (d, 1 H); 10.01 (s, 1 H). |
| 46 | 6-chloro-8-[(2,6-difluorobenzyl)oxy]-N-[1-(2- hydroxyethyl)-1H-pyrazol-4-yl]-2- methylimidazo[1,2-a]pyridine-3-carboxamide 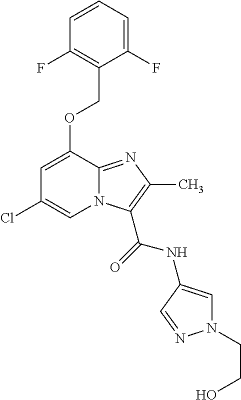 [SMILES: Cc1nc2c(OCc3c(F)cccc3F)cc(Cl)cn2c1C(=O)Nc1cnn(CCO)c1] (85% of theory) | LC-MS (Method 2): Rₜ = 0.84 min MS (ESpos): m/z = 462.2/464.2 (M + H)⁺ ¹H-NMR (400 MHz, DMSO- d₆): δ = 2.57 (s, 3 H; superposed by DMSO signal); 3.70 (q, 2 H); 4.13 (t, 2 H); 4.90 (t, 1 H); 5.36 (s, 2 H); 7.21 (d, 1 H); 7.25 (t, 2 H); 7.59 (s, 1 H); 7.60 (quint., 1 H); 8.05 (s, 1 H); 8.69 (d, 1 H); 10.03 (s, 1 H). |
| 47 | 6-chloro-8-[(2,6-difluorobenzyl)oxy]-2-methyl-N- (5-methyl-1H-pyrazol-4-yl)imidazo[1,2- a]pyridine-3-carboxamide + 6-chloro-8-[(2,6- difluorobenzyl)oxy]-2-methyl-N-(3-methyl-1H- pyrazol-4-yl)imidazo[1,2-a]pyridine-3- carboxamide (tautomer mixture) 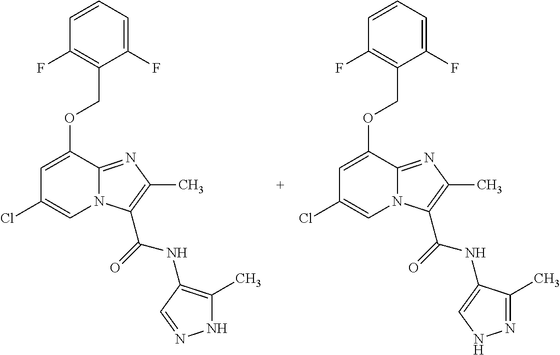 [SMILES: Cc1nc2c(OCc3c(F)cccc3F)cc(Cl)cn2c1C(=O)Nc1cn[nH]c1C ; Cc1n[nH]cc1NC(=O)c1c(C)nc2c(OCc3c(F)cccc3F)cc(Cl)cn12] (8% of theory) | LC-MS (Method 2): Rₜ = 0.92 min MS (ESpos): m/z = 432.3/434.2 (M + H)⁺ ¹H-NMR (400 MHz, DMSO- d₆): δ = 2.18 (s, 3 H); 2.59 (s, 3 H); 5.35 (s, 2 H); 7.21 (s, 1 H); 7.23 (t, 2 H); 7.61 (quint., 1 H); 7.60 + 7.95 (br. s, 1 H, tautomers); 8.71 (s, 1 H); 9.40 (s, 1 H); 12.40 + 12.50 (br.s, 1 H, tautomers). |

### Example 48

### 8-[(2,6-Difluorobenzyl)oxy]-N-(3,5-dimethyl-1H-pyrazol-4-yl)-6-fluoro-2-methylimidazo[1,2-a]pyridine-3-carboxamide

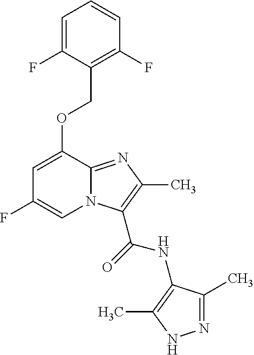 [SMILES: Cc1n[nH]c(C)c1NC(=O)c1c(C)nc2c(OCc3c(F)cccc3F)cc(F)cn12]

80 mg of 8-[(2,6-difluorobenzyl)oxy]-6-fluoro-2-methylimidazo[1,2-a]pyridine-3-carboxylic acid (Example 11A; 0.24 mmol, 1 equivalent), 118 mg of O-(7-azabenzotriazol-1-yl)-N,N,N′N′-tetramethyluronium hexafluorophosphate (HATU; 0.31 mmol, 1.3 equivalents) and 118 μl of N,N-diisopropylethylamine (DIPEA; 0.714 mmol, 3 equivalents) were initially charged in 0.75 ml of DMF, and 37 mg of 3,5-dimethyl-1H-pyrazole-4-amine (0.33 mmol, 1.4 equivalents) were added and the mixture was stirred at RT overnight. Water was added, the reaction solution was filtered off with suction and the product was washed with water and dried under high vacuum overnight. This gave 97 g (93% of theory) of the title compound as slightly beige crystals.

LC-MS (Method 2): Rₜ=0.80 min

MS (ESpos): m/z=430.1 (M+H)⁺

¹H-NMR (400 MHz, DMSO-d₆): δ=2.05 (br. s, 3H); 2.12 (br. s, 3H); 2.60 (s, 3H); 5.32 (s, 2H); 7.23 (t, 2H); 7.30 (d, 1H); 7.61 (quint., 1H); 8.62 (d, 1H); 8.96 (s, 1H); 12.20 (s, 1H).

The examples shown in Table 4 were prepared analogously to Example 48 by reacting the respective carboxylic acid (e.g. Example 6A, 21A, 23A, 25A or 26A) in each case with 3,5-dimethyl-1H-pyrazole-4-amine under the reaction conditions described in Representative Procedure 2:

TABLE 4

| | IUPAC name/structure | |
|---|---|---|
| Example | (Yield) | Analytical data |
| 49 | 8-(cyclohexylmethoxy)-N-(3,5-dimethyl-1H- pyrazol-4-yl)-2-methylimidazo[1,2-a]pyridine-3- carboxamide 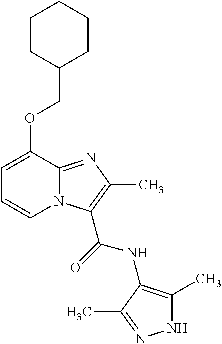 [SMILES: Cc1n[nH]c(C)c1NC(=O)c1c(C)nc2c(OCC3CCCCC3)cccn12] (48% of theory) | LC-MS (Method 2): Rₜ = 0.81 min MS (ESpos): m/z = 382.3 (M + H)⁺ ¹H-NMR (400 MHz, DMSO-d₆): δ = 1.03-1.12 (m, 2 H); 1.20-1.35 (m, 3 H); 1.65-1.80 (m, 3 H); 1.81-1.90 (m, 3 H); 2.06 (s, 3 H); 2.14 (s, 3 H); 2.65 (s, 3 H); 3.95 (d, 2 H); 6.79 (d, 1 H); 6.89 (t, 1 H); 8.49 (d, 1 H); 8.90 (s, 1 H); 12.20 (s, 1 H). |
| 50 | 8-[(2,6-difluorobenzyl)oxy]-N-(3,5-dimethyl-1H- pyrazol-4-yl)-2-methyl-6-(pyrrolidin-1- yl)imidazo[1,2-a]pyridine-3-carboxamide 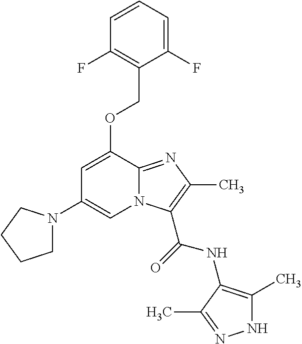 [SMILES: Cc1n[nH]c(C)c1NC(=O)c1c(C)nc2c(OCc3c(F)cccc3F)cc(N3CCCC3)cn12] (49% of theory) | LC-MS (Method 2): Rₜ = 0.82 min MS (ESpos): m/z = 481.3 (M + H)⁺ ¹H-NMR (400 MHz, DMSO-d₆): δ = 1.92-1.98 (m, 4 H); 2.05 (s, 3 H); 2.12 (s, 3 H); 2.58 (s, 3 H; superposed by DMSO signal); 5.33 (s, 2 H); 6.72 (s, 1 H); 7.21 (t, 2 H); 7.58 (quint., 1 H); 7.82 (s, 1 H); 8.71 (s, 1 H); 12.15 (s, 1 H). |
| 51 | 8-[(2,6-difluorobenzyl)oxy]-N-(3,5-dimethyl-1H- pyrazol-4-yl)-2-methyl-6-(morpholin-4- yl)imidazo[1,2-a]pyridine-3-carboxamide 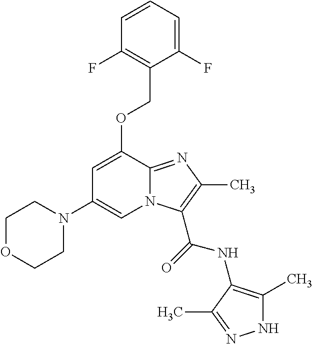 [SMILES: Cc1n[nH]c(C)c1NC(=O)c1c(C)nc2c(OCc3c(F)cccc3F)cc(N3CCOCC3)cn12] (54% of theory) | LC-MS (Method 2): Rₜ = 0.75 min MS (ESpos): m/z = 497.3 (M + H)⁺ ¹H-NMR (400 MHz, DMSO-d₆): δ = 2.02 (s, 3 H); 2.12 (s, 3 H); 2.59 (s, 3 H; superposed by DMSO signal); 3.02- 3.09 (m, 4 H); 3.72-3.80 (m, 4 H); 5.31 (s, 2 H); 7.04 (s, 1 H); 7.21 (t, 2 H); 7.58 (quint., 1 H); 8.07 (s, 1 H); 8.82 (s, 1 H); 12.15 (s, 1 H). |
| 52 | 8-[(2,6-difluorobenzyl)oxy]-N-(3,5-dimethyl-1H- pyrazol-4-yl)-2,6-dimethylimidazo[1,2-a]pyridine- 3-carboxamide 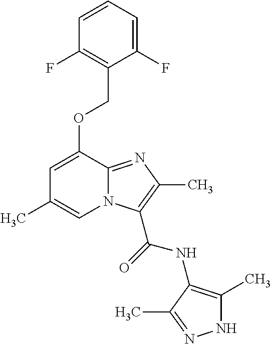 (10% of theory) | LC-MS (Method 1): Rₜ = 0.87 min MS (ESpos): m/z = 426.1 (M + H)⁺ ¹H-NMR (400 MHz, DMSO-d₆): δ = 2.04 (s, 3 H); 2.12 (s, 3 H); 2.30 (s, 3 H); 2.62 (s, 3 H); 5.30 (s, 2 H); 6.91 (s, 1 H); 7.22 (t, 2 H); 7.58 (quint., 1 H); 8.36 (s, 1 H); 8.90 (s, 1 H); 12.15 (s, 1 H). |
| 53 | 6-chloro-8-[(2,3-difluorobenzyl)oxy]-N-(3,5- dimethyl-1H-pyrazol-4-yl)-2-methylimidazo[1,2- a]pyridine-3-carboxamide 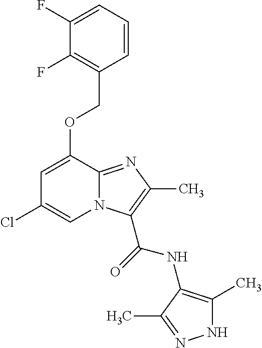 [SMILES: Cc1n[nH]c(C)c1NC(=O)c1c(C)nc2c(OCc3cccc(F)c3F)cc(Cl)cn12] (50% of theory) | LC-MS (Method 2): Rₜ = 0.92 min MS (ESpos): m/z = 446.2 (M + H)⁺ ¹H-NMR (400 MHz, DMSO-d₆): δ = 2.04 (s, 3 H); 2.12 (s, 3 H); 2.67 (s, 3 H); 5.41 (s, 2 H); 7.19 (s, 1 H); 7.30 (q, 1 H); 7.46-7.58 (m, 2 H); 8.62 (s, 1 H); 9.02 (s, 1 H); 12.20 (s, 1 H). |

### Example 54

### 8-[(2,6-Difluorobenzyl)oxy]-N-(6-fluoroquinolin-4-yl)-2-methylimidazo[1,2-a]pyridine-3-carboxamide

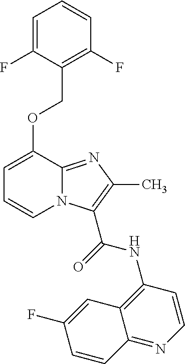 [SMILES: Cc1nc2c(OCc3c(F)cccc3F)cccn2c1C(=O)Nc1ccnc2ccc(F)cc12]

150 mg (0.47 mmol) of 8-[(2,6-difluorobenzyl)oxy]-2-methylimidazo[1,2-a]pyridine-3-carboxylic acid, 448 mg (1.18 mmol) of O-(7-azabenzotriazol-1-yl)-N,N,N′N′-tetramethyluronium hexafluorophosphate (HATU) and 152 mg (1.18 mmol) of N,N-diisopropylethylamine were initially charged in 3 ml of DMF, and the mixture was stirred at RT for 20 min 153 mg (0.94 mmol) of 6-fluoroquinoline-4-amine were then added, and the reaction mixture was stirred at RT overnight. Another 38 mg (0.24 mmol) of 6-fluoroquinoline-4-amine were added, and the reaction mixture was stirred at 60° C. overnight. About 100 ml of water were added to the reaction solution, and the resulting precipitate was stirred for a further 30 min, filtered off with suction and washed thoroughly with water. The residue obtained was purified by silica gel chromatography (mobile phase: dichloromethane:methanol=50:1). The crude product obtained was stirred with acetonitrile and filtered off. This gave 80 mg of the target compound (37% of theory).

LC-MS (Method 2): Rₜ=0.91 min

MS (ESpos): m/z=463 (M+H)⁺

¹H-NMR (400 MHz, DMSO-d₆): δ=2.72 (s, 3H), 5.36 (s, 2H), 7.05 (t, 1H), 7.12 (d, 1H), 7.25 (t, 2H), 7.60 (quint, 1H), 7.69-7.76 (m, 1H), 8.06-8.20 (m, 3H), 8.68 (d, 1H), 8.88 (d, 1H), 10.22 (s, 1H).

### Example 55

### 8-[(2,6-Difluorobenzyl)oxy]-2-methyl-N-(5-methyl-3-phenyl-1,2-oxazol-4-yl)imidazo[1,2-a]pyridine-3-carboxamide

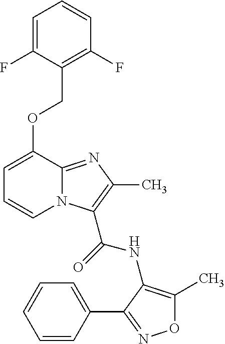 [SMILES: Cc1nc2c(OCc3c(F)cccc3F)cccn2c1C(=O)Nc1c(-c2ccccc2)noc1C]

75 mg (0.24 mmol) of 8-[(2,6-difluorobenzyl)oxy]-2-methylimidazo[1,2-a]pyridine-3-carboxylic acid, 179 mg (0.47 mmol) of O-(7-azabenzotriazol-1-yl)-N,N,N′N′-tetramethyluronium hexafluorophosphate (HATU) and 76 mg (0.59 mmol) of N,N-diisopropylethylamine were initially charged in 1.5 ml of DMF, the mixture was stirred for 10 min, 63 mg (0.35 mmol) of 5-methyl-3-phenyl-1,2-oxazole-4-amine were added and the mixture was stirred at RT overnight. The reaction mixture was then stirred at 40° C. overnight and subsequently at 60° C. overnight. About 24 ml of water were added to the reaction solution, the precipitate formed was stirred for another 30 min, filtered off with suction, washed thoroughly with water and purified by preparative HPLC (RP18 column, mobile phase: acetonitrile/water gradient with addition of 0.1% trifluoroacetic acid). The product-containing fractions were concentrated, and the residue was dissolved in ethyl acetate and washed twice with a little saturated aqueous sodium bicarbonate solution. The organic phase was concentrated and the residue was dissolved in acetonitrile/water and lyophilized. This gave 26 mg of the target compound (22% of theory, purity 95%).

LC-MS (Method 2): Rₜ=0.97 min

MS (ESpos): m/z=475 (M+H)⁺

¹H-NMR (400 MHz, DMSO-d₆): δ=2.43 (s, 3H), 2.60 (s, 3H), 5.32 (s, 2H), 6.95 (t, 1H), 7.08 (d, 1H), 7.23 (t, 2H), 7.45-7.52 (m, 3H), 7.59 (quint, 1H), 7.70-7.80 (m, 2H), 8.59 (br s, 1H), 9.42 (s, 1H).

Analogously to Example 55, the examples shown in Table 5 were prepared by reacting 8-[(2,6-difluorobenzyl)oxy]-2-methylimidazo[1,2-a]pyridine-3-carboxylic acid (Example 3A) with the appropriate commercially available amine under the conditions described in General Procedure 1:

TABLE 5

| | IUPAC name/structure | |
|---|---|---|
| Example | (Yield) | Analytical data |
| 56 | 8-[(2,6-difluorobenzyl)oxy]-2-methyl-N- (naphthalen-1-yl)imidazo[1,2-a]pyridine-3- carboxamide 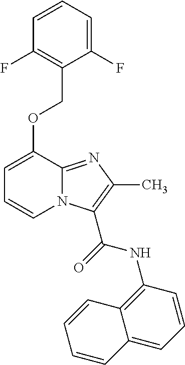 [SMILES: Cc1nc2c(OCc3c(F)cccc3F)cccn2c1C(=O)Nc1cccc2ccccc12] (36% of theory) | LC-MS (Method 2): Rₜ = 1.01 min MS (ESpos): m/z = 444 (M + H)⁺ ¹H-NMR (400 MHz, DMSO-d₆): δ = 2.76 (s, 3H), 5.35 (s, 2H), 6.98 (t, 1H), 7.08 (d, 1H), 7.25 (t, 2H), 7.52-7.65 (m, 4H), 7.73 (d, 1H), 7.83 (d, 1H), 7.94-8.08 (m, 2H), 8.63 (d, 1H), 9.97 (s, 1H). |

### Example 57

### 8-[(2,6-Difluorobenzyl)oxy]-2-methyl-N-(1,3,5-trimethyl-1H-pyrazol-4-yl)imidazo[1,2-a]pyridine-3-carboxamide

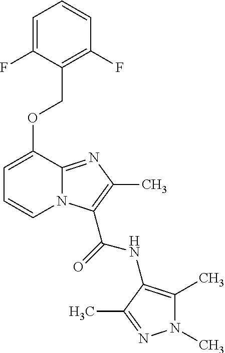 [SMILES: Cc1nn(C)c(C)c1NC(=O)c1c(C)nc2c(OCc3c(F)cccc3F)cccn12]

50 mg (0.16 mmol) of 8-[(2,6-difluorobenzyl)oxy]-2-methylimidazo[1,2-a]pyridine-3-carboxylic acid, 149 mg (0.39 mmol) of O-(7-azabenzotriazol-1-yl)-N,N,N′N′-tetramethyluronium hexafluorophosphate (HATU) and 51 mg (0.39 mmol) of N,N-diisopropylethylamine were initially charged in 1 ml of DMF, the mixture was stirred for 20 min, 39 mg (0.31 mmol) of 1,3,5-trimethyl-1H-pyrazole-4-amine were added and the mixture was stirred at 60° C. overnight. About 20 ml of water were added to the reaction solution, and the resulting precipitate was stirred for a further 30 min, filtered off with suction and washed thoroughly with water. This gave 46 mg of the target compound (65% of theory, purity 95%).

LC-MS (Method 1): Rₜ=0.87 min

MS (ESpos): m/z=426 (M+H)⁺

¹H-NMR (400 MHz, DMSO-d₆): δ=2.06 (s, 3H), 2.14 (s, 3H), 2.66 (s, 3H), 3.68 (s, 3H), 5.39 (s, 2H), 7.09-7.39 (m, 4H), 7.60 (quint, 1H), 8.61 (d, 1H), 9.23 (br s, 1H).

Analogously to Example 57, the examples shown in Table 6 were prepared by reacting 8-[(2,6-difluorobenzyl)oxy]-2-methylimidazo[1,2-a]pyridine-3-carboxylic acid (Example 3A) with the appropriate commercially available amine under the conditions described in the general procedure. Optionally, the product was purified by preparative HPLC (RP18 column; mobile phase: acetonitrile/water gradient with addition of 0.1% trifluoroacetic acid). The product-containing fractions were optionally concentrated, the residue was dissolved in ethyl acetate and washed with a little saturated aqueous sodium bicarbonate solution, and the organic phase was then dried over sodium sulphate and filtered and the filtrate was concentrated.

TABLE 6

| | IUPAC name/structure | |
|---|---|---|
| Example | (Yield) | Analytical data |
| 58 | 8-[(2,6-difluorobenzyl)oxy]-N-(2,6- dimethylphenyl)-2-methylimidazo[1,2-a]pyridine- 3-carboxamide 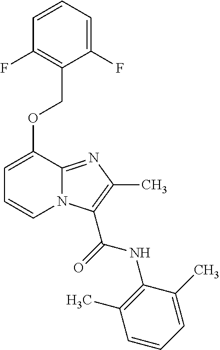 [SMILES: Cc1cccc(C)c1NC(=O)c1c(C)nc2c(OCc3c(F)cccc3F)cccn12] (45% of theory) | LC-MS (Method 2): Rₜ = 0.98 min MS (ESpos): m/z = 422 (M + H)⁺ ¹H-NMR (400 MHz, DMSO-d₆): δ = 2.24 (s, 6H), 2.51 (s, 3H), 5.33 (s, 2H), 6.97 (t, 1H), 7.05 (d, 1H), 7.18 (s, 3H), 7.24 (t, 2H), 7.60 (quint, 1H), 8.58 (d, 1H), 9.23 (s, 1H). |
| 59 | 8-[(2,6-difluorobenzyl)oxy]-N-(isoquinolin-1-yl)- 2-methylimidazo[1,2-a]pyridin-3-carboxamide trifluoroacetate 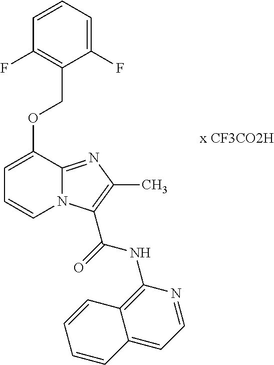 [SMILES: Cc1nc2c(OCc3c(F)cccc3F)cccn2c1C(=O)Nc1nccc2ccccc12] (72% of theory) | LC-MS (Method 2): Rₜ = 0.99 min MS (ESpos): m/z = 445 (M − TFA + H)⁺ ¹H-NMR (400 MHz, DMSO-d₆): δ = 2.97 (s, 3H), 5.48 (s, 2H), 7.28 (t, 2H), 7.30-7.51 (m, 2H), 7.53-7.68 (m, 2H), 7.71-7.79 (m, 1H), 7.82 (d, 1H), 7.93 (d, 2H), 8.75 (d, 1H), 9.61 (br s, 1H), 14.60 (br s, 1H). |

### Example 60

### N-(8-Chloronaphthalen-1-yl)-8-[(2,6-difluorobenzyl)oxy]-2-methylimidazo[1,2-a]pyridine-3-carboxamide trifluoroacetate

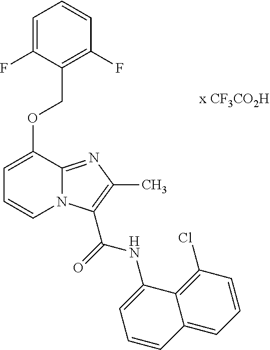 [SMILES: Cc1nc2c(OCc3c(F)cccc3F)cccn2c1C(=O)Nc1cccc2cccc(Cl)c12]

70 mg (0.22 mmol) of 8-[(2,6-difluorobenzyl)oxy]-2-methylimidazo[1,2-a]pyridine-3-carboxylic acid were initially charged in 3 ml of THF, 44 mg (0.33 mmol) of 1-chloro-N,N,2-trimethylprop-1-ene-1-amine were added and the mixture was stirred at RT for 30 min 47 mg (0.26 mmol) of 8-chloronaphthalene-1-amine were then added, and the suspension was stirred at RT overnight. Another 44 mg (0.33 mmol) of 1-chloro-N,N,2-trimethylprop-1-ene-1-amine were added, and the suspension was stirred at RT overnight. The reaction mixture was concentrated and the crude product was purified by preparative HPLC (RP18 column, mobile phase: methanol/water gradient with addition of 0.1% TFA). 81 mg of the target compound (62% of theory) were obtained.

LC-MS (Method 2): Rt=1.03 min

MS (ESpos): m/z=478 (M+H)+

1H-NMR (400 MHz, DMSO-d6): δ=2.79 (s, 3H), 5.43 (s, 2H), 7.19-7.28 (m, 3H), 7.37-7.45 (m, 1H), 7.52 (t, 1H), 7.56-7.69 (m, 4H), 8.03-8.07 (m, 2H), 8.76 (d, 1H), 10.21 (br s, 1H).

Analogously to Example 60, the examples shown in Table 7 were prepared by reacting 8-[(2,6-difluorobenzyl)oxy]-2-methylimidazo[1,2-a]pyridine-3-carboxylic acid (Example 3A) with the appropriate commercially available amines under the conditions described in General Procedure 3.

TABLE 7

| | IUPAC name/structure | |
|---|---|---|
| Example | (Yield) | Analytical data |
| 61 | 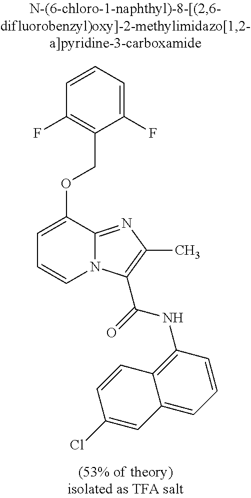 [SMILES: Cc1nc2c(OCc3c(F)cccc3F)cccn2c1C(=O)Nc1cccc2cc(Cl)ccc12] | LC-MS (Method 2): Rₜ = 1.10 min MS (ESpos): m/z = 478 (M + H)⁺ ¹H-NMR (400 MHz, DMSO-d₆): δ = 2.78 (s, 3H), 5.40 (s, 2H), 7.10-7.18 (m, 1H), 7.20-7.30 (m, 3H), 7.55-7.67 (m, 3H), 7.73 (d, 1H), 7.88 (d, 1H), 8.08 (d, 1H), 8.12 (d, 1H), 8.69 (d, 1H), 10.18 (br s, 1H). |
| 62 | 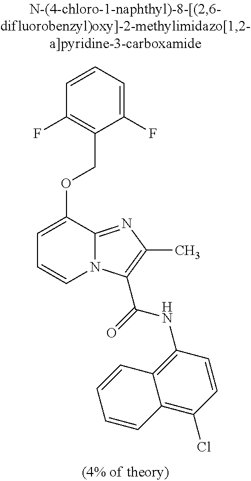 [SMILES: Cc1nc2c(OCc3c(F)cccc3F)cccn2c1C(=O)Nc1ccc(Cl)c2ccccc12] | LC-MS (Method 5): Rₜ = 1.17 min MS (ESpos): m/z = 478.1/480.1 (M + H)⁺ |
| 63 | 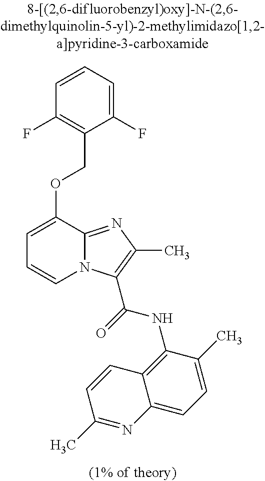 [SMILES: Cc1ccc2c(NC(=O)c3c(C)nc4c(OCc5c(F)cccc5F)cccn34)c(C)ccc2n1] | LC-MS (Method 5): Rₜ = 0.83 min MS (ESpos): m/z = 473.16 (M + H)⁺ |
| 64 | 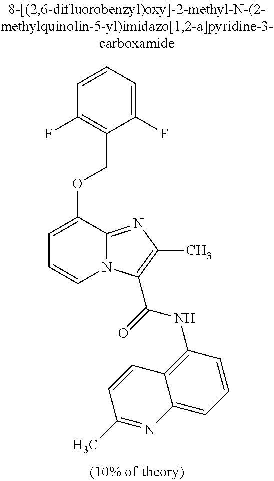 [SMILES: Cc1ccc2c(NC(=O)c3c(C)nc4c(OCc5c(F)cccc5F)cccn34)cccc2n1] | LC-MS (Method 5): Rₜ = 0.83 min MS (ESpos): m/z = 459.13 (M + H)⁺ |
| 65 | 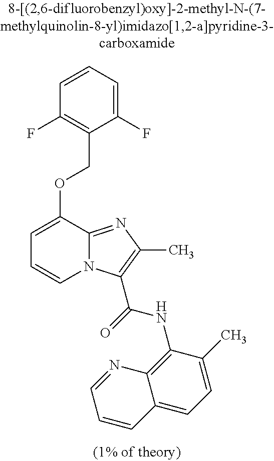 [SMILES: Cc1ccc2cccnc2c1NC(=O)c1c(C)nc2c(OCc3c(F)cccc3F)cccn12] | LC-MS (Method 5): Rₜ = 0.97 min MS (ESpos): m/z = 459.09 (M + H)⁺ |
| 66 | 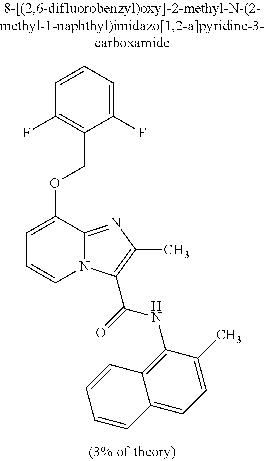 [SMILES: Cc1ccc2ccccc2c1NC(=O)c1c(C)nc2c(OCc3c(F)cccc3F)cccn12] | LC-MS (Method 5): Rₜ = 1.10 min MS (ESpos): m/z = 458.12 (M + H)⁺ |
| 67 | 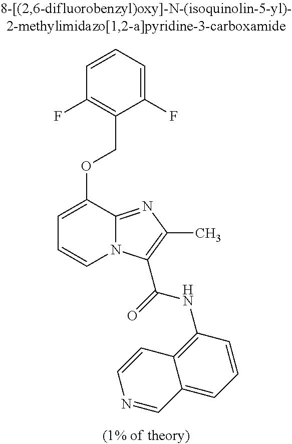 [SMILES: Cc1nc2c(OCc3c(F)cccc3F)cccn2c1C(=O)Nc1cccc2cnccc12] | LC-MS (Method 5): Rₜ = 0.90 min MS (ESpos): m/z = 445.11 (M + H)⁺ |
| 68 | 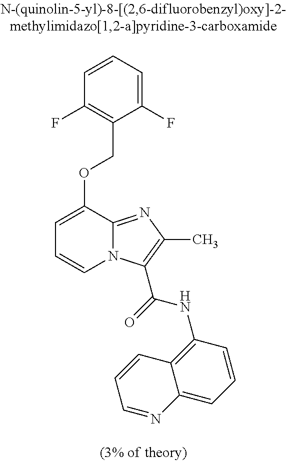 [SMILES: Cc1nc2c(OCc3c(F)cccc3F)cccn2c1C(=O)Nc1cccc2ncccc12] | LC-MS (Method 5): Rₜ = 0.94 min MS (ESpos): m/z = 445.16 (M + H)⁺ |
| 69 | 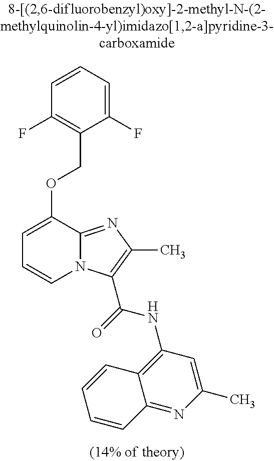 [SMILES: Cc1cc(NC(=O)c2c(C)nc3c(OCc4c(F)cccc4F)cccn23)c2ccccc2n1] | LC-MS (Method 5): Rₜ = 0.87 min MS (ESpos): m/z = 459.13 (M + H)⁺ |
| 70 | 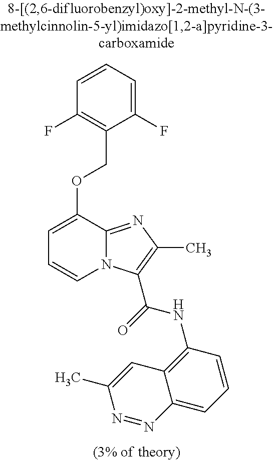 [SMILES: Cc1cc2c(NC(=O)c3c(C)nc4c(OCc5c(F)cccc5F)cccn34)cccc2nn1] | LC-MS (Method 5): Rₜ = 1.02 min MS (ESpos): m/z = 460.19 (M + H)⁺ |
| 71 | 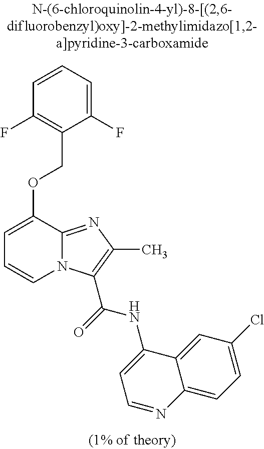 [SMILES: Cc1nc2c(OCc3c(F)cccc3F)cccn2c1C(=O)Nc1ccnc2ccc(Cl)cc12] | LC-MS (Method 5): Rₜ = 1.13 min MS (ESpos): m/z = 479.02 (M + H)⁺ |
| 72 | 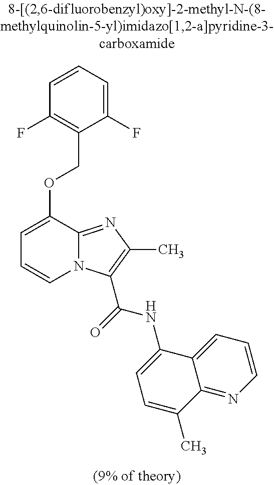 [SMILES: Cc1nc2c(OCc3c(F)cccc3F)cccn2c1C(=O)Nc1ccc(C)c2ncccc12] | LC-MS (Method 5): Rₜ = 0.98 min MS (ESpos): m/z = 459.18 (M + H)⁺ |
| 73 | 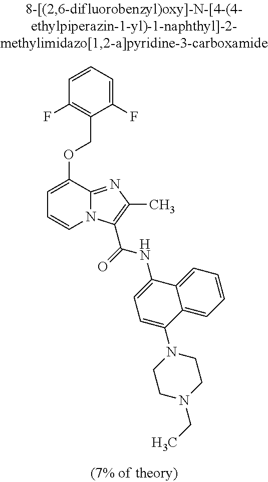 [SMILES: CCN1CCN(c2ccc(NC(=O)c3c(C)nc4c(OCc5c(F)cccc5F)cccn34)c3ccccc23)CC1] | LC-MS (Method 5): Rₜ = 0.81 min MS (ESneg): m/z = 555.23 (M − H)⁻ |
| 74 | 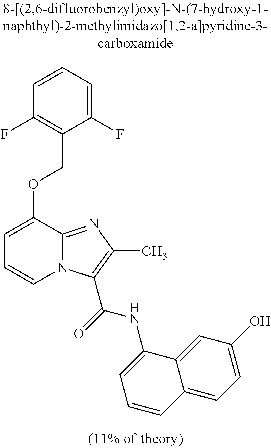 [SMILES: Cc1nc2c(OCc3c(F)cccc3F)cccn2c1C(=O)Nc1cccc2ccc(O)cc12] | LC-MS (Method 5): Rₜ = 0.99 min MS (ESpos): m/z = 460.14 (M + H)⁺ |
| 75 | 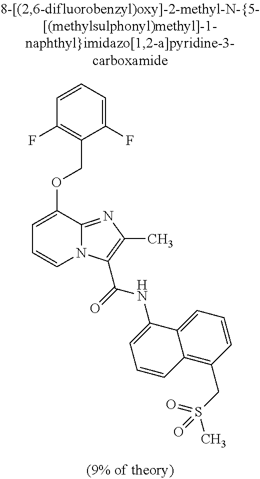 | LC-MS (Method 5): Rₜ = 1.00 min MS (ESpos): m/z = 550.10 (M + H)⁺ |

### Example 76

### 8-[(2,6-Difluorobenzyl)oxy]-2-methyl-N-[5-methyl-3-(trifluoromethyl)-1H-pyrazol-4-yl]imidazo[1,2-a]pyridine-3-carboxamide

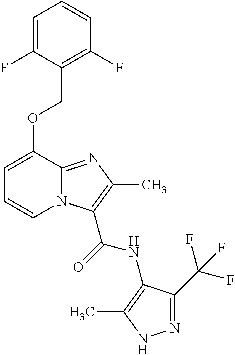 [SMILES: Cc1nc2c(OCc3c(F)cccc3F)cccn2c1C(=O)Nc1c(C(F)(F)F)n[nH]c1C]

384 mg (1.03 mmol) of 8-[(2,6-difluorobenzyl)oxy]-2-methylimidazo[1,2-a]pyridine-3-carbonyl chloride hydrochloride were initially charged in 37 ml of abs. THF, a solution of 204 mg (1.24 mmol) of 5-methyl-3-(trifluoromethyl)-1H-pyrazole-4-amine and 532 mg (4.12 mmol) of N,N-diisopropylethylamine in 5.4 ml of abs. THF was added and the mixture was stirred at RT overnight. The reaction solution was concentrated and re-dissolved in a little acetonitrile, and water was added. The precipitated solid was stirred for about 30 min, filtered off and washed thoroughly with water. 428 mg of the target compound (87% of theory) were obtained.

LC-MS (Method 2): Rₜ=0.81 min

MS (ESpos): m/z=466 (M+H)⁺

¹H-NMR (400 MHz, DMSO-d₆): δ=2.22 (s, 3H), 2.62 (s, 3H), 5.32 (s, 2H), 6.98 (t, 1H), 7.08 (d, 1H), 7.23 (t, 2H), 7.59 (quint, 1H), 8.54 (d, 1H), 9.22 (s, 1H), 13.48 (s, 1H).

### Example 77

### N-(7-Chloroquinolin-4-yl)-8-[(2,6-difluorobenzyl)oxy]-2-methylimidazo[1,2-a]pyridine-3-carboxamide

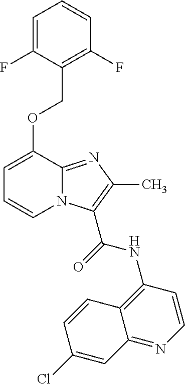 [SMILES: Cc1nc2c(OCc3c(F)cccc3F)cccn2c1C(=O)Nc1ccnc2cc(Cl)ccc12]

70 mg (0.19 mmol) of 8-[(2,6-difluorobenzyl)oxy]-2-methylimidazo[1,2-a]pyridine-3-carbonyl chloride hydrochloride were initially charged in 7 ml of abs. THF, 40 mg (0.23 mmol) of 7-chloroquinoline-4-amine and 97 mg (0.75 mmol) of N,N-diisopropylethylamine were then added and the mixture was stirred at RT overnight. The reaction solution was then purified by preparative HPLC (RP18 column; mobile phase: acetonitrile/water gradient with addition of 0.1% trifluoroacetic acid). The product-containing fractions were concentrated, and the residue was dissolved in ethyl acetate and washed with a little saturated aqueous sodium bicarbonate solution. The organic phase was dried over sodium sulphate and filtered and the filtrate was concentrated. The crude product was purified by silica gel chromatography (mobile phase: dichloromethane:methanol=40:1 isocratic). 34 mg of the target compound (37% of theory) were obtained.

LC-MS (Method 2): Rₜ=0.99 min

MS (ESpos): m/z=479 (M+H)⁺

¹H-NMR (400 MHz, DMSO-d₆): δ=2.72 (s, 3H), 5.38 (s, 2H), 7.06 (t, 1H), 7.12 (d, 1H), 7.25 (t, 2H), 7.60 (quint, 1H), 7.69 (dd, 1H), 8.10 (d, 1H), 8.14-8.20 (m, 1H), 8.31 (d, 1H), 8.68 (d, 1H), 8.90 (d, 1H), 10.35 (s, 1H).

Analogously to Examples 76 and 77, the Examples shown in Table 8 were prepared by reacting 8-[(2,6-difluorobenzyl)oxy]-2-methylimidazo[1,2-a]pyridine-3-carbonyl chloride hydrochloride (Example 27A) with the appropriate commercially available amines under the conditions described in General Procedure 4.

TABLE 8

| | IUPAC name/structure | |
|---|---|---|
| Example | (Yield) | Analytical data |
| 78 | 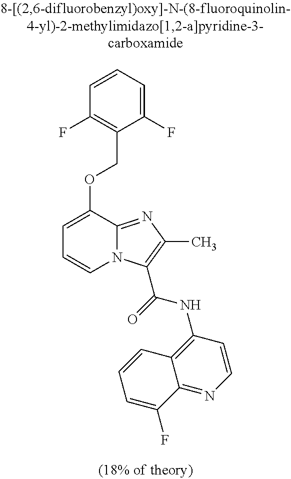 [SMILES: Cc1nc2c(OCc3c(F)cccc3F)cccn2c1C(=O)Nc1ccnc2c(F)cccc12] | LC-MS (Method 2): Rₜ = 0.91 min MS (ESpos): m/z = 463 (M + H)⁺ ¹H-NMR (400 MHz, DMSO-d₆): δ = 2.73 (s, 3H), 5.37 (s, 2H), 7.08 (t, 1H), 7.14 (d, 1H), 7.26 (t, 2H), 7.56-7.65 (m, 3H), 8.09-8.14 (m, 1H), 8.28 (d, 1H), 8.70 (d, 1H), 8.90 (d, 1H), 10.32 (s, 1H). |
| 79 | 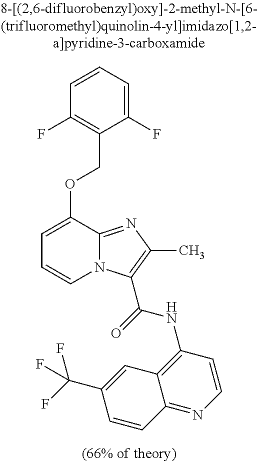 [SMILES: Cc1nc2c(OCc3c(F)cccc3F)cccn2c1C(=O)Nc1ccnc2ccc(C(F)(F)F)cc12] | LC-MS (Method 2): Rₜ = 1.03 min MS (ESpos): m/z = 513 (M + H)⁺ ¹H-NMR (400 MHz, DMSO-d₆): δ = 2.72 (s, 3H), 5.38 (s, 2H), 7.08 (t, 1H), 7.13 (d, 1H), 7.23 (t, 2H), 7.60 (quint, 1H), 8.05 (d, 1H), 8.22 (d, 1H), 8.28 (d, 1H), 8.68 (br s, 1H), 8.82 (s, 1H), 8.99 (br s, 1H), 10.55 (s, 1H). |

### Example 80

### 8-[(2,6-Difluorobenzyl)oxy]-N-(1H-indazol-3-yl)-2-methylimidazo[1,2-a]pyridine-3-carboxamide

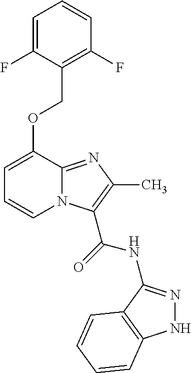 [SMILES: Cc1nc2c(OCc3c(F)cccc3F)cccn2c1C(=O)Nc1n[nH]c2ccccc12]

1.7 ml of a 1M solution of hydrogen chloride in diethyl ether were added to 110 mg (0.17 mmol) of tert-butyl 3-[({8[(2,6-difluorobenzyl)oxy]-2-methylimidazo[1,2-a]pyridin-3-yl}carbonyl)amino]-1H-indazole-1-carboxylate trifluoroacetate, and the mixture was stirred at RT overnight. The same amount of a 1M solution of hydrogen chloride in dioxane was added, and the mixture was stirred at 40° C. overnight. The same amount of a 1M solution of hydrogen chloride in dioxane was added again, and the mixture was stirred at 60° C. for 12 h. The reaction mixture was concentrated and the residue was purified by preparative HPLC (RP18 column, mobile phase: acetonitrile/water gradient with addition of 0.1% trifluoroacetic acid). The resulting product was dissolved in ethyl acetate and washed with saturated aqueous sodium hydrogencarbonate solution. The organic phase was dried over sodium sulphate and filtered and the filtrate was concentrated. The product was dissolved in acetonitrile/water and lyophilized. 60 mg of the target compound (82% of theory) were obtained.

LC-MS (Method 2): Rₜ=0.84 min

MS (ESpos): m/z=434 (M+H)⁺

¹H-NMR (400 MHz, DMSO-d₆): δ=2.63 (s, 3H), 5.33 (s, 2H), 6.99 (t, 1H), 7.15-7.11 (m, 2H), 7.25 (t, 2H), 7.38 (t, 1H), 7.49 (d, 1H), 7.60 (quint, 1H), 7.78 (d, 1H), 8.62 (d, 1H), 10.38 (s, 1H), 12.80 (1H).

### Example 81

### 8-[(2,6-Difluorobenzyl)oxy]-N-(1H-indazol-3-yl)-2-methylimidazo[1,2-a]pyridine-3-carboxamide

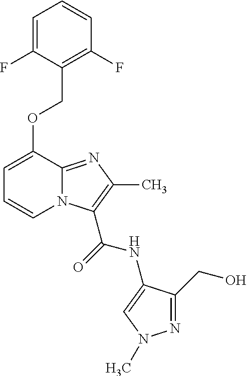 [SMILES: Cc1nc2c(OCc3c(F)cccc3F)cccn2c1C(=O)Nc1cn(C)nc1CO]

70 mg of 8-[(2,6-difluorobenzyl)oxy]-2-methylimidazo[1,2-a]pyridine-3-carboxylic acid (Example 3A; 0.22 mmol, 1 equivalent), 109 mg of O-(7-azabenzotriazol-1-yl)-N,N,N′N′-tetramethyluronium hexafluorophosphate (HATU; 0.286 mmol, 1.3 equivalents) and 109 μl of N,N-diisopropylethylamine (DIPEA; 0.66 mmol, 3 equivalents) were initially charged in 0.7 ml of DMF, 39 mg of (4-amino-1-methyl-1H-pyrazol-3-yl)methanol (Example 30A; 0.31 mmol, 1.4 equivalents) were added and the mixture was stirred at RT overnight. Water was added to the reaction solution, the resulting precipitate was stirred for another 5 min, filtered off with suction and washed thoroughly with water and dried under high vacuum overnight. The crude product obtained was purified further by HPLC (column: Macherey-Nagel VP 50/21 Nucleodur C18 Gravity, 5 μm Cat. No.: 762103.210, Ser. No.: E9051009, Batch 37508074, 50×21 mm, gradient: A=water+0.1% conc. aq ammonia, B=methanol, 0 min=30% B, 2 min=30% B, 6 min=100% B, 7 min=100% B, 7.1 min=30% B, 8 min=30% B, flow rate 25 ml/min, wavelength 220 nm), giving 21.5 mg (22% of theory) of the title compound.

LC-MS (Method 2): Rₜ=0.66 min

MS (ESpos): m/z=428.2 (M+H)⁺

¹H-NMR (400 MHz, DMSO-d₆): δ=2.60 (s, 3H); 3.82 (s, 3H); 4.60 (d, 2H); 5.30 (t, 1H); 5.32 (s, 2H); 6.96 (t, 1H); 7.04 (d, 1H); 7.24 (t, 2H); 7.59 (quint., 1H); 7.74 (s, 1H); 8.69 (d, 1H); 9.38 (s, 1H).

The examples shown in Table 9 were prepared analogously to Example 1 by reacting the appropriate carboxylic acids with the appropriate commercially available amines (1-3 equivalents), TBTU (1-2.5 equivalents) and 4-methylmorpholine (4-5 equivalents). The reaction times were 1-3 days. Optionally, the purifications were carried out by preparative HPLC (RP18 column; mobile phase: acetonitrile/water gradient with addition of 0.1% trifluoroacetic acid) and/or by silica gel chromatography (mobile phase gradient: dichloromethane/methanol). The product-containing fractions were optionally concentrated, the residue was dissolved in ethyl acetate or dichloromethane/methanol and washed with a little saturated aqueous sodium bicarbonate solution, and the organic phase was then dried over sodium sulphate and filtered and the filtrate was concentrated.

TABLE 9

| | IUPAC name/structure | |
|---|---|---|
| Example | (Yield) | Analytical data |
| 82 | 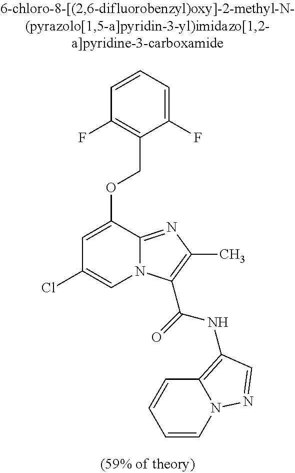 [SMILES: Cc1nc2c(OCc3c(F)cccc3F)cc(Cl)cn2c1C(=O)Nc1cnn2ccccc12] | LC-MS (Method 2): Rₜ = 1.01 min MS (ESpos): m/z = 468 (M + H)⁺ ¹H-NMR (400 MHz, DMSO-d₆): δ = 2.68 (s, 3H), 5.38 (s, 2H), 6.89 (t, 1H), 7.19-7.29 (m, 4H), 7.61 (quint, 1H), 7.68 (d, 1H), 8.32 (s, 1H), 8.62 (d, 1H), 8.74 (s, 1H), 10.03 (s, 1H). |
| 83 | 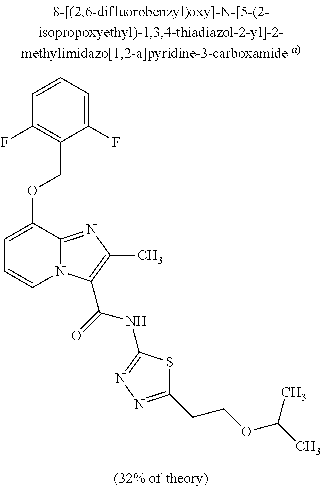 [SMILES: Cc1nc2c(OCc3c(F)cccc3F)cccn2c1C(=O)Nc1nnc(CCOC(C)C)s1] | LC-MS (Method 1): Rₜ = 1.16 min MS (ESpos): m/z = 488 (M + H)⁺ ¹H-NMR (400 MHz, DMSO-d₆): δ = 1.13 (d, 6H), 2.69 (s, 3H), 3.10-3.20 (m, 2H), 3.59-3.68 (m, 1H), 3.70 (t, 2H), 5.32 (s, 2H), 7.03 (t, 1H), 7.13 (d, 1H), 7.25 (t, 2H), 7.61 (quint, 1H), 8.90 (br s, 1H). |
| 84 | 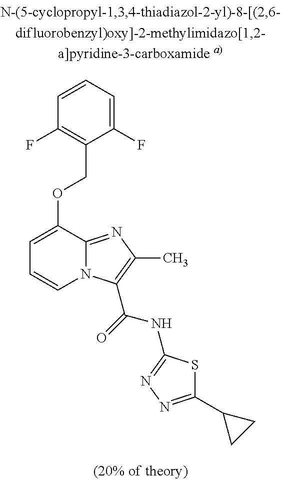 [SMILES: Cc1nc2c(OCc3c(F)cccc3F)cccn2c1C(=O)Nc1nnc(C2CC2)s1] | LC-MS (Method 1): Rₜ = 1.10 min MS (ESpos): m/z = 442 (M + H)⁺ ¹H-NMR (400 MHz, DMSO-d₆): δ = 0.98-1.03 (m, 2H), 1.13-1.19 (m, 2H), 2.30-2.42 (m, 1H), 2.69 (s, 3H), 5.32 (s, 2H), 7.03 (t, 1H), 7.13 (d, 1H), 7.25 (t, 2H), 7.60 (quint, 1H), 8.90 (br s, 1H). |

^{a)} The reaction temperature was 50° C.

The examples shown in Table 10 were prepared analogously to Example 48 by reacting the appropriate carboxylic acids with the appropriate commercially available amines (1-3 equivalents), HATU (1-2.5 equivalents) and N,N-diisopropylethylamine (4-6 equivalents) at RT. The reaction times were 1-3 days. Optionally, the purifications were carried out by preparative HPLC (RP18 column; mobile phase: acetonitrile/water gradient with addition of 0.1% trifluoroacetic acid) and/or by silica gel chromatography (mobile phase gradient: dichloromethane/methanol). The product-containing fractions were optionally concentrated, the residue was dissolved in ethyl acetate or dichloromethane/methanol and washed with a little saturated aqueous sodium bicarbonate solution, and the organic phase was then dried over sodium sulphate and filtered and the filtrate was concentrated.

TABLE 10

| | IUPAC name/structure | |
|---|---|---|
| Example | (Yield) | Analytical data |
| 85 | 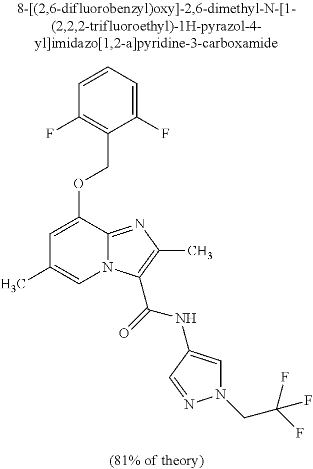 [SMILES: Cc1cc(OCc2c(F)cccc2F)c2nc(C)c(C(=O)Nc3cnn(CC(F)(F)F)c3)n2c1] | LC-MS (Method 2): Rₜ = 0.93 min MS (ESpos): m/z = 480 (M + H)⁺ ¹H-NMR (400 MHz, DMSO-d₆): δ = 2.30 (s, 3H), 2.50 (s, 3H), 5.18 (q, 2H), 5.30 (s, 2H), 6.95 (s, 1H), 7.24 (t, 2H), 7.60 (quint, 1H), 7.72 (s, 1H), 8.22 (s, 1H), 8.42 (s, 1H), 10.04 (s, 1H). |
| 86 | 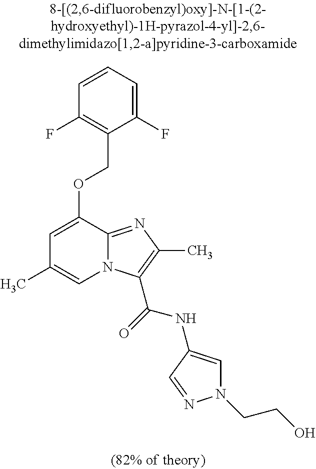 [SMILES: Cc1cc(OCc2c(F)cccc2F)c2nc(C)c(C(=O)Nc3cnn(CCO)c3)n2c1] | LC-MS (Method 2): Rₜ = 0.75 min MS (ESpos): m/z = 442 (M + H)⁺ ¹H-NMR (400 MHz, DMSO-d₆): δ = 2.32 (s, 3H), 2.50 (s, 3H), 3.72 (q, 2H), 4.12 (t, 2H), 4.90 (t, 1H), 5.30 (s, 2H), 6.95 (s, 1H), 7.24 (t, 2H), 7.56-7.63 (m, 2H), 8.06 (s, 1H), 8.39 (s, 1H), 9.94 (s, 1H). |
| 87 | 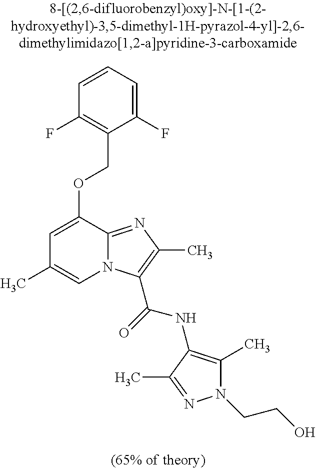 [SMILES: Cc1cc(OCc2c(F)cccc2F)c2nc(C)c(C(=O)Nc3c(C)nn(CCO)c3C)n2c1] | LC-MS (Method 2): Rₜ = 0.76 min MS (ESpos): m/z = 470 (M + H)⁺ ¹H-NMR (400 MHz, DMSO-d₆): δ = 2.07 (s, 3H), 2.18 (s, 3H), 2.32 (s, 3H), 2.61 (s, 3H), 3.66-3.72 (m, 2H), 4.02 (t, 2H), 4.87 (br s, 1H), 5.30 (s, 2H), 6.96 (br s, 1H), 7.24 (t, 2H), 7.60 (quint., 1H), 8.38 (s, 1H), 8.96 (br s, 1H). |
| 88 | 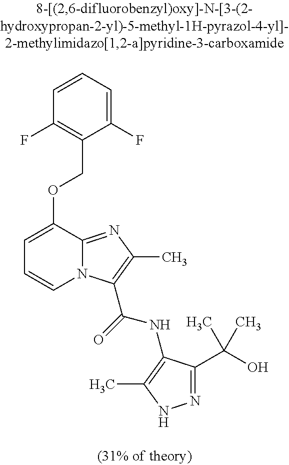 [SMILES: Cc1nc2c(OCc3c(F)cccc3F)cccn2c1C(=O)Nc1c(C(C)(C)O)n[nH]c1C] | LC-MS (Method 2): Rₜ = 0.74 min MS (ESpos): m/z = 456 (M + H)⁺ ¹H-NMR (400 MHz, DMSO-d₆): δ = 1.44 (s, 6H), 2.12 (s, 3H), 2.64 (s, 3H), 5.24 (br s, 1H), 5.32 (s, 2H), 6.99 (t, 1H), 7.07 (d, 1H), 7.24 (t, 2H), 7.59 (quint., 1H), 8.75 (d, 1H), 8.80 (s, 1H), 12.14 (br s, 1H). |
| 89 | 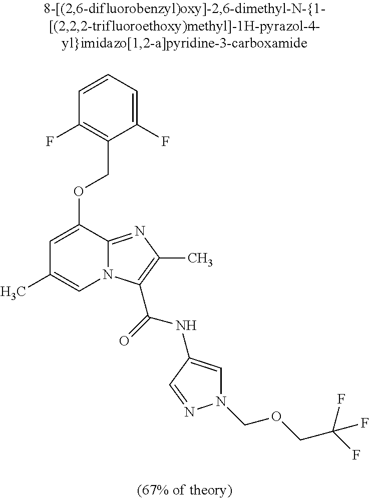 [SMILES: Cc1cc(OCc2c(F)cccc2F)c2nc(C)c(C(=O)Nc3cnn(COCC(F)(F)F)c3)n2c1] | LC-MS (Method 2): Rₜ = 0.96 min MS (ESpos): m/z = 510 (M + H)⁺ ¹H-NMR (400 MHz, DMSO-d₆): δ = 2.32 (s, 3H), 2.50 (s, 3H), 4.17 (q, 2H), 5.30 (s, 2H), 5.60 (s, 2H), 6.95 (s, 1H), 7.24 (t, 2H), 7.56-7.64 (m, 1H), 7.74 (s, 1H), 8.28 (s, 1H), 8.42 (s, 1H), 10.02 (s, 1H). |
| 90 | 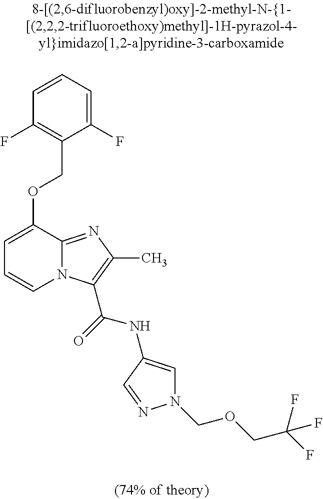 [SMILES: Cc1nc2c(OCc3c(F)cccc3F)cccn2c1C(=O)Nc1cnn(COCC(F)(F)F)c1] | LC-MS (Method 2): Rₜ = 0.94 min MS (ESpos): m/z = 496 (M + H)⁺ ¹H-NMR (400 MHz, DMSO-d₆): δ = 2.58 (s, 3H), 4.16 (q, 2H), 5.31 (s, 2H), 5.60 (s, 2H), 6.97 (t, 1H), 7.04 (d, 1H), 7.23 (t, 2H), 7.56-7.64 (m, 1H), 7.74 (s, 1H), 8.29 (s, 1H), 8.59 (d, 1H), 10.04 (s, 1H). |

### Example 91

### 8-[(2,6-Difluorobenzyl)oxy]-N-(6-fluoroquinolin-4-yl)-2,6-dimethylimidazo[1,2-a]pyridine-3-carboxamide

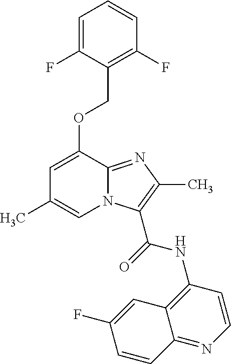 [SMILES: Cc1cc(OCc2c(F)cccc2F)c2nc(C)c(C(=O)Nc3ccnc4ccc(F)cc34)n2c1]

100 mg (0.30 mmol) of 8-[(2,6-difluorobenzyl)oxy]-2,6-dimethylimidazo[1,2-a]pyridine-3-carboxylic acid (Example 21A), 73 mg (0.45 mmol) of O-(7-azabenzotriazol-1-yl)-N,N,N′N′-tetramethyluronium hexafluorophosphate (HATU) and 97 mg (0.75 mmol) of N,N-diisopropylethylamine were initially charged in 1.9 ml of DMF, the mixture was stirred for 20 min, 73 mg (0.45 mmol) of 6-fluoroquinoline-4-amine were then added and the mixture was stirred at 60° C. for two days. About 40 ml of water were added to the reaction solution, and the resulting precipitate was stirred for a further 30 min, filtered off with suction and washed thoroughly with water. The residue was dissolved in acetonitrile and purified by preparative HPLC (RP18 column, mobile phase: acetonitrile/water gradient with addition of 0.1% TFA). The product fractions were taken up in dichloromethane, washed once with saturated aqueous sodium bicarbonate solution and concentrated. The crude product was stirred with acetonitrile and the solid was filtered off. 16 mg of the target compound (11% of theory) were obtained.

LC-MS (Method 2): Rₜ=0.91 min

MS (ESpos): m/z=477 (M+H)⁺

¹H-NMR (400 MHz, DMSO-d₆): δ=2.36 (s, 3H), 2.70 (s, 3H), 5.32 (s, 2H), 7.04 (s, 1H), 7.24 (t, 2H), 7.60 (quint, 1H), 7.68-7.75 (m, 1H), 8.05-8.15 (m, 2H), 8.19 (d, 1H), 8.48 (s, 1H), 8.87 (d, 1H), 10.21 (s, 1H).

The examples shown in Table 11 were prepared analogously to Example 91 by reacting the appropriate carboxylic acids with the appropriate commercially available amines (1-3 equivalents), HATU (1-2.5 equivalents) and N,N-diisopropylethylamine (4 equivalents). The reaction times were 1-3 days. Optionally, the purifications were carried out by preparative HPLC (RP18 column; mobile phase: acetonitrile/water gradient with addition of 0.1% trifluoroacetic acid) and/or by silica gel chromatography (mobile phase gradient: dichloromethane/methanol). The product-containing fractions were optionally concentrated, the residue was dissolved in ethyl acetate or dichloromethane/methanol and washed with a little saturated aqueous sodium bicarbonate solution, and the organic phase was then dried over sodium sulphate and filtered and the filtrate was concentrated.

TABLE 11

| | IUPAC name/structure | |
|---|---|---|
| Example | (Yield) | Analytical data |
| 92 | 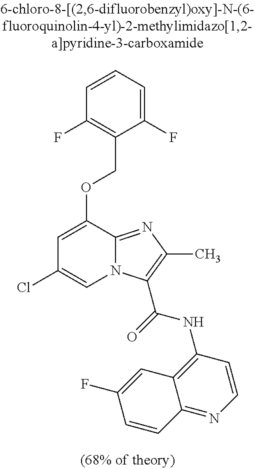 [SMILES: Cc1nc2c(OCc3c(F)cccc3F)cc(Cl)cn2c1C(=O)Nc1ccnc2ccc(F)cc12] | LC-MS (Method 2): Rₜ = 1.04 min MS (ESpos): m/z = 497 (M + H)⁺ ¹H-NMR (400 MHz, DMSO-d₆): δ = 2.71 (s, 3H), 5.39 (s, 2H), 7.25 (t, 2H), 7.32 (s, 1H), 7.61 (quint, 1H), 7.68- 7.75 (m, 1H), 8.04-8.18 (m, 3H), 8.75 (s, 1H), 8.88 (d, 1H), 10.32 (s, 1H). |
| 93 | 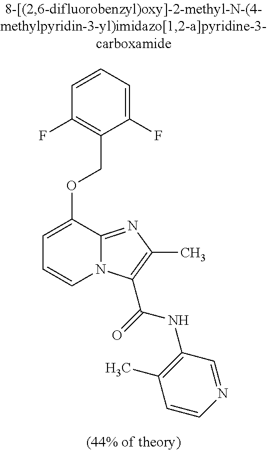 [SMILES: Cc1ccncc1NC(=O)c1c(C)nc2c(OCc3c(F)cccc3F)cccn12] | LC-MS (Method 2): Rₜ = 0.76 min MS (ESpos): m/z = 409 (M + H)⁺ ¹H-NMR (400 MHz, DMSO-d₆): δ = 2.28 (s, 3H), 2.68 (s, 3H), 5.32 (s, 2H), 6.99 (t, 1H), 7.08 (d, 1H), 7.24 (t, 2H), 7.34 (d, 1H), 7.60 (quint, 1H), 8.33 (d, 1H), 8.68 (d, 2H), 9.59 (s, 1H). |
| 94 | 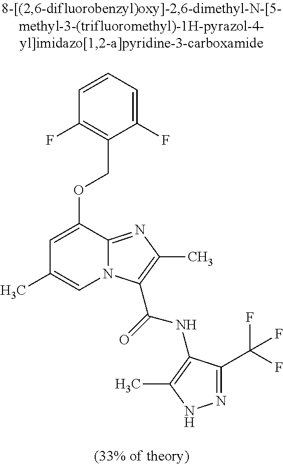 [SMILES: Cc1cc(OCc2c(F)cccc2F)c2nc(C)c(C(=O)Nc3c(C(F)(F)F)n[nH]c3C)n2c1] | LC-MS (Method 2): Rₜ = 0.89 min MS (ESpos): m/z = 480 (M + H)⁺ ¹H-NMR (400 MHz, DMSO-d₆): δ = 2.22 (s, 3H), 2.30 (s, 3H), 2.58 (s, 3H), 5.31 (s, 2H), 6.98 (s, 1H), 7.24 (t, 2H), 7.60 (quint, 1H), 8.38 (s, 1H), 9.19 (s, 1H), 13.48 (s, 1H). |
| 95 | 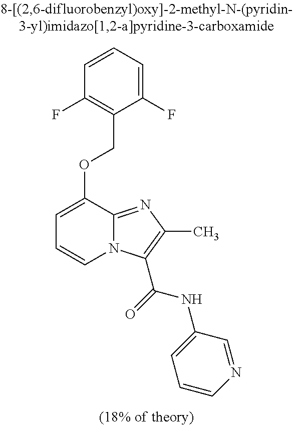 [SMILES: Cc1nc2c(OCc3c(F)cccc3F)cccn2c1C(=O)Nc1cccnc1] | LC-MS (Method 1): Rₜ = 0.87 min MS (ESpos): m/z = 395 (M + H)⁺ ¹H-NMR (400 MHz, DMSO-d₆): δ = 2.61 (s, 3H), 5.32 (s, 2H), 6.99 (t, 1H), 7.09 (d, 1H), 7.24 (t, 2H), 7.40 (dd, 1H), 7.60 (quint, 1H), 8.09-8.13 (m, 1H), 8.32 (d, 1H), 8.58 (d, 1H), 8.88 (d, 1H), 10.10 (s, 1H). |
| 96 | 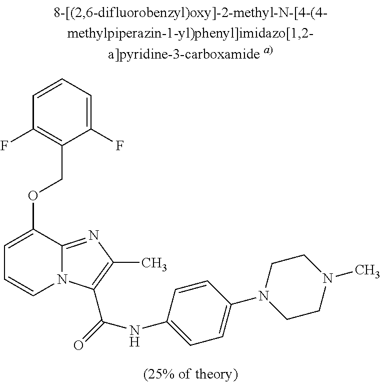 [SMILES: Cc1nc2c(OCc3c(F)cccc3F)cccn2c1C(=O)Nc1ccc(N2CCN(C)CC2)cc1] | LC-MS (Method 2): Rₜ = 0.70 min MS (ESpos): m/z = 492 (M + H)⁺ ¹H-NMR (400 MHz, DMSO-d₆): δ = 2.23 (s, 3H), 2.45-2.59 (m, 7H), 3.11 (t, 4H), 5.32 (s, 2H), 6.90-6.98 (m, 3H), 7.04 (d, 1H), 7.24 (t, 2H), 7.53- 7.64 (m, 3H), 8.53 (d, 1H), 9.73 (s, 1H). |
| 97 | 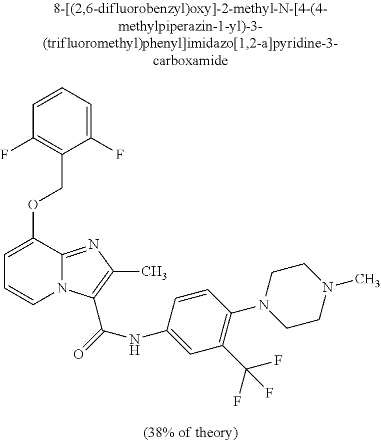 [SMILES: Cc1nc2c(OCc3c(F)cccc3F)cccn2c1C(=O)Nc1ccc(N2CCN(C)CC2)c(C(F)(F)F)c1] | LC-MS (Method 2): Rₜ = 0.76 min MS (ESpos): m/z = 560 (M + H)⁺ ¹H-NMR (400 MHz, DMSO-d₆): δ = 2.23 (s, 3H), 2.48 (br s, 4H), 2.59 (s, 3H), 2.85 (t, 4H), 5.34 (s, 2H), 6.98 (t, 1H), 7.09 (d, 1H), 7.24 (t, 2H), 7.55- 7.65 (m, 2H), 7.91 (d, 1H), 8.10 (d, 1H), 8.58 (d, 1H), 10.12 (s, 1H). |
| 98 | 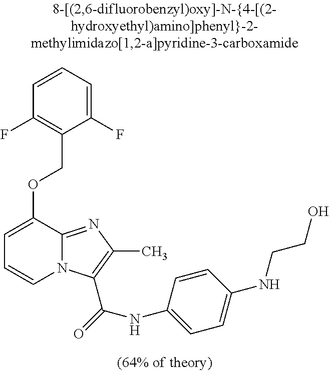 [SMILES: Cc1nc2c(OCc3c(F)cccc3F)cccn2c1C(=O)Nc1ccc(NCCO)cc1] | LC-MS (Method 2): Rₜ = 0.68 min MS (ESpos): m/z = 453 (M + H)⁺ ¹H-NMR (400 MHz, DMSO-d₆): δ = 2.50 (s, 3H), 3.09 (t, 2H), 3.58 (q, 2H), 4.68 (t, 1H), 5.32 (s, 2H), 5.39 (s, 1H), 6.58 (d, 2H), 6.94 (t, 1H), 7.03 (d, 1H), 7.25 (t, 2H), 7.39 (d, 2H), 7.59 (quint., 1H), 8.53 (d, 1H), 9.58 (s, 1H). |
| 99 | 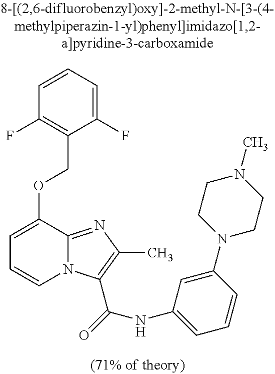 [SMILES: Cc1nc2c(OCc3c(F)cccc3F)cccn2c1C(=O)Nc1cccc(N2CCN(C)CC2)c1] | LC-MS (Method 2): Rₜ = 0.65 min MS (ESpos): m/z = 492 (M + H)⁺ ¹H-NMR (400 MHz, DMSO-d₆): δ = 2.22 (s, 3H), 2.46 (t, 4H), 2.57 (s, 3H), 3.12 (t, 4H), 5.32 (s, 2H), 6.70 (d, 1H), 6.98 (t, 1H), 7.07 (d, 1H), 7.10-7.28 (m, 4H), 7.33 (s, 1H), 7.59 (quint., 1H), 8.56 (d, 1H), 9.77 (s, 1H). |

^{a)} A further chromatographic separation was carried out: Sunfire C18, 5 μm, 250 × 20 mm, methanol: 1% strength TFA solution (30:70), flow rate: 25 ml/min, wavelength: 210 nm, temperature: 40° C.

The examples shown in Table 12 were prepared analogously to Examples 76 and 77 by reacting the appropriate carbonyl chlorides with the appropriate commercially available amines (0.3-2 equivalents) and N,N-diisopropylethylamine (2-4 equivalents). The reaction times were 1-5 days. Optionally, the purifications were carried out by preparative HPLC (RP18 column; mobile phase: acetonitrile/water gradient with addition of 0.1% trifluoroacetic acid) or by silica gel chromatography (mobile phase gradient: dichloromethane/methanol). The product-containing fractions were optionally concentrated, the residue was dissolved in ethyl acetate or dichloromethane/methanol and washed with a little saturated aqueous sodium bicarbonate solution, and the organic phase was then dried over sodium sulphate and filtered and the filtrate was concentrated.

TABLE 12

| | IUPAC name/structure | |
|---|---|---|
| Example | (Yield) | Analytical data |
| 100 | 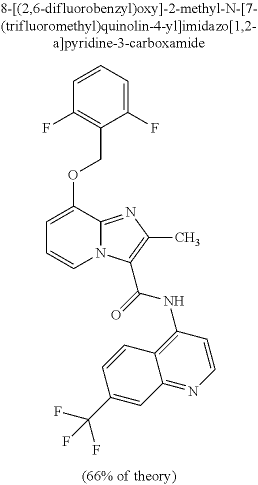 [SMILES: Cc1nc2c(OCc3c(F)cccc3F)cccn2c1C(=O)Nc1ccnc2cc(C(F)(F)F)ccc12] | LC-MS (Method 2): Rₜ = 1.07 min MS (ESpos): m/z = 513 (M + H)⁺ ¹H-NMR (400 MHz, DMSO-d₆): δ = 2.76 (s, 3H), 5.37 (s, 2H), 7.08 (t, 1H), 7.15 (d, 1H), 7.25 (t, 2H), 7.60 (quint, 1H), 7.93 (d, 1H), 8.28 (d, 1H), 8.88 (s, 1H), 8.53 (d, 1H), 8.69 (d, 1H), 9.03 (d, 1H), 10.45 (s, 1H). |
| 101 | 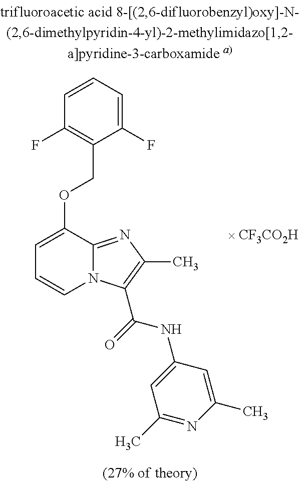 | LC-MS (Method 10): Rₜ = 0.66 min MS (ESpos): m/z = 423 (M − TFA + H)⁺ ¹H-NMR (400 MHz, DMSO-d₆): δ = 2.62 (s, 3H), 2.64 (s, 6H), 5.34 (s, 2H), 7.11 (t, 1H), 7.19-7.29 (m, 3H), 7.60 (quint., 1H), 7.82 (s, 2H), 8.63 (d, 1H), 11.03 (s, 1H), 14.48 (br s, 1H). |
| 102 | 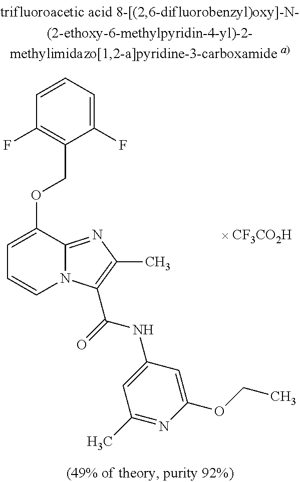 | LC-MS (Method 2): Rₜ = 0.97 min MS (ESpos): m/z = 453 (M − TFA + H)⁺ ¹H-NMR (400 MHz, DMSO-d₆): δ = 1.32 (t, 3H), 2.40 (s, 3H), 2.61 (s, 3H), 4.30 (q, 2H), 5.39 (s, 2H), 7.10 (s, 1H), 7.18 (s, 1H), 7.19-7.29 (m, 3H), 7.34 (d, 1H), 7.60 (quint., 1H), 8.63 (d, 1H), 10.49 (br s, 1H). |

^{a)} The reaction temperature was 40° C.

### Example 103

### 8-[(2,6-Difluorobenzyl)oxy]-2-methyl-N-[1-methyl-5-(trifluoromethyl)-1H-indazol-3-yl]imidazo[1,2-a]pyridine-3-carboxamide

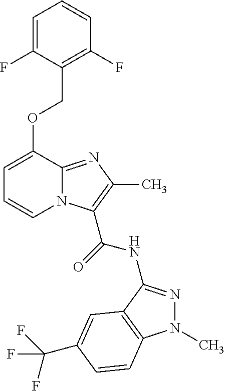 [SMILES: Cc1nc2c(OCc3c(F)cccc3F)cccn2c1C(=O)Nc1nn(C)c2ccc(C(F)(F)F)cc12]

82 mg (0.22 mmol) of 1-methyl-5-(trifluoromethyl)-1H-indazole-3-amine were suspended in 7.1 ml of THF, a solution of 47 mg (0.22 mmol) of 8-[(2,6-difluorobenzyl)oxy]-2-methylimidazo[1,2-a]pyridine-3-carbonyl chloride hydrochloride (Example 27A) and 0.15 ml of N,N-diisopropylethylamine in 1.1 ml of THF was added and the mixture was stirred at RT overnight. The reaction solution was then stirred at 40° C. overnight. 82 mg (0.22 mmol) of 1-methyl-5-(trifluoromethyl)-1H-indazole-3-amine were added to the reaction solution, and the mixture was stirred at 40° C. overnight. 82 mg (0.22 mmol) of 1-methyl-5-(trifluoromethyl)-1H-indazole-3-amine and 0.038 ml of N,N-diisopropylethylamine were then added to the reaction solution, and the mixture was stirred at 40° C. overnight. The reaction solution was concentrated slightly, TFA was added and the product was purified by preparative HPLC (RP18 column, mobile phase: acetonitrile/water gradient with addition of 0.1% TFA). This was followed by another chromatographic separation [XBridge C 18, 5 μm, 100×30 mm; mobile phase: water/acetonitrile/1% strength TFA in water=65/30/5); flow rate: 25 ml/min, wavelength: 210 nm]. The product fraction was dissolved in dichloromethane and washed once with saturated aqueous sodium bicarbonate solution. The organic phase was dried over sodium sulphate, filtered and concentrated by rotary evaporation. 45 mg of the target compound (40% of theory) were obtained.

LC-MS (Method 1): Rₜ=1.29 min

MS (ESpos): m/z=516 (M+H)⁺

¹H-NMR (400 MHz, DMSO-d₆): δ=2.64 (s, 3H), 4.07 (s, 3H), 5.35 (s, 2H), 7.00 (t, 1H), 7.10 (d, 1H), 7.25 (t, 2H), 7.59 (quint, 1H), 7.70 (d, 1H), 7.86 (d, 1H), 8.32 (s, 1H), 8.63 (d, 1H), 10.67 (s, 1H).

### Example 104

### N-[2-(Aminomethyl)phenyl]-6-chloro-8-[(2,6-difluorobenzyl)oxy]-2-methylimidazo[1,2-a]pyridine-3-carboxamide

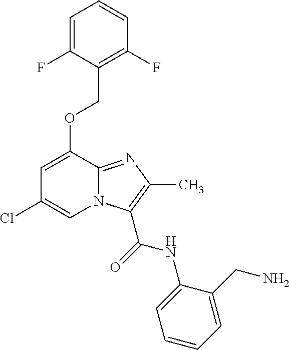 [SMILES: Cc1nc2c(OCc3c(F)cccc3F)cc(Cl)cn2c1C(=O)Nc1ccccc1CN]

151 mg (0.27 mmol) of tert-butyl {2-[({6-chloro-8-[(2,6-difluorobenzyl)oxy]-2-methylimidazo pyridin-3-yl}carbonyl)amino]benzyl}carbamate trifluoroacetate (Example 39A) were suspended in 1.4 ml of diethyl ether, 1.35 ml (2.7 mmol) of a 2 M solution of hydrogen chloride in diethyl ether were added and the mixture was stirred at RT overnight. The precipitate was filtered off with suction, washed with diethyl ether and purified by preparative HPLC (RP18 column, mobile phase: acetonitrile/water gradient with addition of 0.1% trifluoroacetic acid). The resulting product was dissolved in ethyl acetate and washed with saturated aqueous sodium hydrogencarbonate solution. The organic phase was dried over sodium sulphate and filtered and the filtrate was concentrated. 92 mg of the target compound (74% of theory) were obtained.

LC-MS (Method 2): Rₜ=0.76 min

MS (ESpos): m/z=457 (M+H)⁺

¹H-NMR (400 MHz, DMSO-d₆): δ=2.69 (s, 3H), 3.89 (s, 2H), 5.37 (s, 2H), 5.80-5.25 (br s, 2H), 7.09 (t, 1H), 7.21-7.32 (m, 5H), 7.61 (quint, 1H), 8.10 (d, 1H), 8.89 (s, 1H).

The examples shown in Table 13 were prepared analogously to Example 104 by reacting the appropriate protected amines with hydrochloric acid (10-15 equivalents; 1 M or 2 M in diethyl ether). The reaction times were 5 h-3 days. Optionally, the purifications were carried out by preparative HPLC (RP18 column; mobile phase: acetonitrile/water gradient with addition of 0.1% trifluoroacetic acid) and/or by silica gel chromatography (mobile phase gradient: dichloromethane/methanol). The product-containing fractions were optionally concentrated, the residue was dissolved in ethyl acetate or dichloromethane/methanol and washed with a little saturated aqueous sodium bicarbonate solution, and the organic phase was then dried over sodium sulphate and filtered and the filtrate was concentrated.

Optionally, the reaction mixture was diluted with diethyl ether, the precipitate was filtered off and partitioned between ethyl acetate or dichloromethane and saturated aqueous sodium bicarbonate solution. The organic phase was washed once with saturated sodium chloride solution, dried over sodium sulphate and filtered, and the filtrate was concentrated and dried under high vacuum.

TABLE 13

| | IUPAC name/structure | |
|---|---|---|
| Example | (Yield) | Analytical data |
| 105 | 8-[(2,6-difluorobenzyl)oxy]-2-methyl-N-[2- methyl-4-(piperazin-1-yl)phenyl]imidazo[1,2- a]pyridine-3-carboxamide 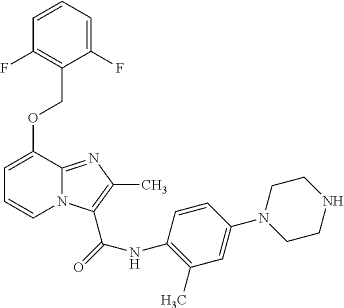 (86% of theory) | LC-MS (Method 2): Rₜ = 0.68 min MS (ESpos): m/z = 592 (M + H)⁺ ¹H-NMR (400 MHz, DMSO-d₆): δ = 2.22 (s, 3H), 2.64 (s, 3H), 2.83 (t, 4H), 3.04 (t, 4H), 5.33 (s, 2H), 6.78 (dd, 1H), 6.83 (d, 1H), 6.98 (t, 1H), 7.06 (d, 1H), 7.20-7.31 (m, 3H), 7.59 (quint., 1H), 8.62 (d, 1H), 9.22 (s, 1H). |
| 106 | 8-[(2,6-difluorobenzyl)oxy]-N-[3-fluoro-4- (piperazin-1-yl)phenyl]-2-methylimidazo[1,2- a]pyridine-3-carboxamide 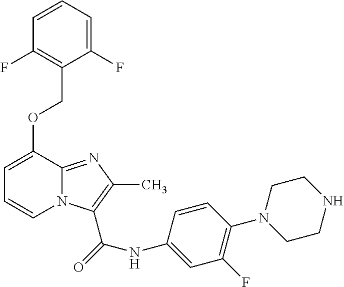 [SMILES: Cc1nc2c(OCc3c(F)cccc3F)cccn2c1C(=O)Nc1ccc(N2CCNCC2)c(F)c1] (60% of theory) | LC-MS (Method 2): Rₜ = 0.69 min MS (ESpos): m/z = 496 (M + H)⁺ ¹H-NMR (400 MHz, DMSO-d₆): δ = 2.58 (s, 3H), 2.83-2.92 (m, 8H), 5.32 (s, 2H), 6.95-7.09 (m, 3H), 7.24 (t, 2H), 7.38 (d, 1H), 7.56-7.63 (m, 2H), 8.53 (d, 1H), 9.94 (s, 1H). |
| 107 | 8-[(2,6-difluorobenzyl)oxy]-2-methyl-N-[4- (piperazin-1-yl)-3- (trifluoromethyl)phenyl]imidazo[1,2-a]pyridine-3- carboxamide 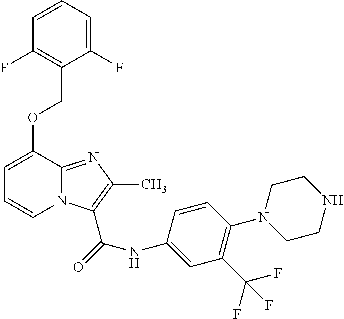 [SMILES: Cc1nc2c(OCc3c(F)cccc3F)cccn2c1C(=O)Nc1ccc(N2CCNCC2)c(C(F)(F)F)c1] (75% of theory) | LC-MS (Method 10): Rₜ = 0.72 min MS (ESpos): m/z = 546 (M + H)⁺ ¹H-NMR (400 MHz, DMSO-d₆): δ = 2.50-59 (m, 5H), 2.68 (s, 2H), 2.73- 2.83 (m, 4H), 5.33 (s, 2H), 6.98 (t, 1H), 7.08 (d, 1H), 7.24 (t, 2H), 7.49- 7.64 (m, 2H), 7.87-7.93 (m, 1H), 8.08 (s, 1H), 8.58 (d, 1H), 10.13 (s, 1H). |
| 108 | 6-chloro-8-[(2,6-difluorobenzyl)oxy]-2-methyl-N- [2-methyl-4-(piperazin-1-yl)phenyl]imidazo[1,2- a]pyridine-3-carboxamide 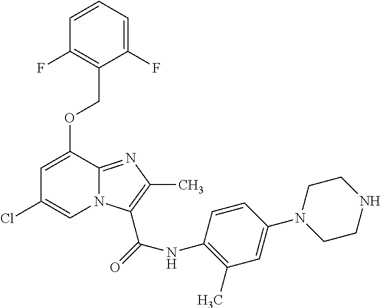 [SMILES: Cc1cc(N2CCNCC2)ccc1NC(=O)c1c(C)nc2c(OCc3c(F)cccc3F)cc(Cl)cn12] (82% of theory) | LC-MS (Method 2): Rₜ = 0.87 min MS (ESpos): m/z = 526 (M + H)⁺ ¹H-NMR (400 MHz, DMSO-d₆): δ = 2.22 (s, 3H), 2.63 (s, 3H), 2.83 (t, 4H), 3.04 (t, 4H), 5.38 (s, 2H), 6.78 (dd, 1H), 6.84 (d, 1H), 7.20-7.32 (m, 4H), 7.62 (quint., 1H), 8.72 (d, 1H), 9.28 (s, 1H). |
| 109 | 6-chloro-8-[(2,6-difluorobenzyl)oxy]-N-[3-fluoro- 4-(piperazin-1-yl)phenyl]-2-methylimidazo[1,2- a]pyridine-3-carboxamide 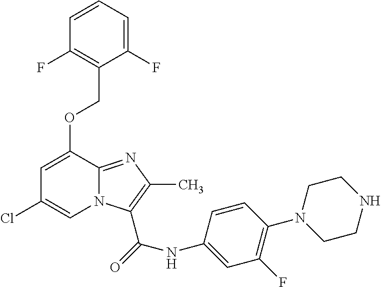 [SMILES: Cc1nc2c(OCc3c(F)cccc3F)cc(Cl)cn2c1C(=O)Nc1ccc(N2CCNCC2)c(F)c1] (79% of theory) | LC-MS (Method 2): Rₜ = 0.80 min MS (ESpos): m/z = 530 (M + H)⁺ ¹H-NMR (400 MHz, DMSO-d₆): δ = 2.29 (br s, 1H), 2.50 (s, 3H), 2.81-2.92 (m, 8H), 5.38 (s, 2H), 7.02 (t, 1H), 7.20-7.29 (m, 3H), 7.38 (d, 1H), 7.57- 7.68 (m, 2H), 8.63 (s, 1H), 10.01 (s, 1H). |

### Example 110

### N-[2-(Aminomethyl)-4-fluorophenyl]-8-[(2,6-difluorobenzyl)oxy]-2-methylimidazo[1,2-a]pyridine-3-carboxamide

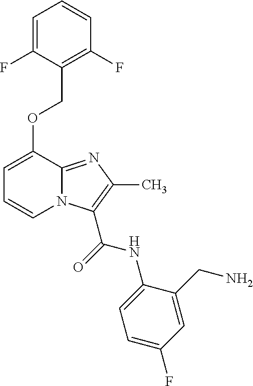 [SMILES: Cc1nc2c(OCc3c(F)cccc3F)cccn2c1C(=O)Nc1ccc(F)cc1CN]

58 mg (0.09 mmol) of tert-butyl {2-[({8-[(2,6-difluorobenzyl)oxy]-2-methylimidazo[1,2-a]pyridin-3-yl}carbonyl)amino]-5-fluorobenzyl}carbamate trifluoroacetate (Example 37A) were suspended in 2.5 ml of diethyl ether, 0.44 ml (0.89 mmol) of a 2 M solution of hydrogen chloride in diethyl ether were added and the mixture was stirred at RT overnight. Another 0.88 ml of the 2 M solution of hydrogen chloride in diethyl ether was added, and the reaction mixture was stirred at room temperature overnight. The reaction mixture was concentrated on a rotary evaporator, 2 ml of a 4 N solution of hydrogen chloride in dioxane were added and the mixture was stirred at 40° C. for 6 h. The reaction mixture was filtered off and washed thoroughly with diethyl ether. The residue was dissolved in dichloromethane with a little methanol, and washed once with saturated aqueous sodium bicarbonate solution. The organic phase was dried over sodium sulphate, filtered and concentrated by rotary evaporation. This gave 25 mg of the target compound (59% of theory, purity 92%).

LC-MS (Method 2): Rₜ=0.60 min

MS (ESpos): m/z=442 (M+H)⁺

¹H-NMR (400 MHz, DMSO-d₆): δ=2.69 (s, 3H), 3.86 (s, 2H), 5.31 (s, 2H), 4.80-5.70 (br s, 2H), 7.00 (t, 1H), 7.06-7.29 (m, 5H), 7.60 (quint, 1H), 8.02 (dd, 1H), 8.78 (s, 1H).

### Example 111

### N-[2-(Aminomethyl)phenyl]-8-[(2,6-difluorobenzyl)oxy]-2-methylimidazo[1,2-a]pyridine-3-carboxamide

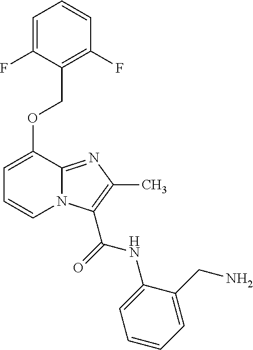 [SMILES: Cc1nc2c(OCc3c(F)cccc3F)cccn2c1C(=O)Nc1ccccc1CN]

2.54 ml (5.07 mmol) of a 2 M solution of hydrogen chloride in diethyl ether were added to 265 mg (0.51 mmol) of tert-butyl {2-[({8-[(2,6-difluorobenzyl)oxy]-2-methylimidazo]1,2-a]pyridin-3-yl}carbonyl)amino]benzyl}carbamate (Example 38A), and the mixture was stirred at RT for 5.5 h. The precipitate was filtered off with suction and washed with diethyl ether, and the crude product was purified by silica gel chromatography (mobile phase: dichloromethane:methanol 40:1->20:1). The product was then re-purified by preparative HPLC (RP18 column, mobile phase: acetonitrile/water gradient with addition of 0.1% TFA). The product-containing fractions were concentrated, and the residue was dissolved in ethyl acetate and washed twice with a little saturated aqueous sodium bicarbonate solution. The organic phase was concentrated and the residue was dissolved in acetonitrile/water and lyophilized. This gave 89 mg of the target compound (39% of theory, purity 95%).

LC-MS (Method 2): Rₜ=0.64 min

MS (ESpos): m/z=423 (M+H)⁺

¹H-NMR (400 MHz, DMSO-d₆): δ=2.70 (s, 3H), 3.88 (s, 2H), 5.32 (s, 2H), 5.85-4.80 (br s, 2H), 7.00 (t, 1H), 7.06-7.10 (m, 2H), 7.20-7.31 (m, 4H), 7.59 (quint, 1H), 8.12 (d, 1H), 8.79 (d, 1H).

### Example 112

### N-[2-(Aminomethyl)phenyl]-8-[(2,6-difluorobenzyl)oxy]-2,6-dimethylimidazo[1,2-a]pyridine-3-carboxamide

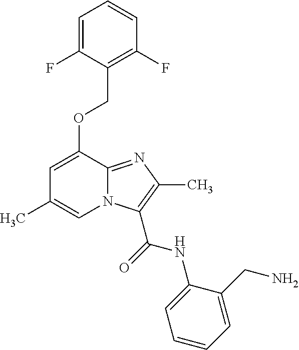 [SMILES: Cc1cc(OCc2c(F)cccc2F)c2nc(C)c(C(=O)Nc3ccccc3CN)n2c1]

110 mg (0.17 mmol) of tert-butyl {2-[({8-[(2,6-difluorobenzyl)oxy]-2,6-dimethylimidazo[1,2-a]pyridin-3-yl}carbonyl)amino]benzyl}carbamate trifluoroacetate (Example 40A) were suspended in 4 ml of diethyl ether, 3.38 ml (3.38 mmol) of a 1 M solution of hydrogen chloride in diethyl ether were added and the mixture was stirred at RT overnight. 3 ml of a 2 M solution of hydrogen chloride in diethyl ether were then added, and the reaction mixture was stirred further at room temperature overnight. The precipitate was filtered off with suction and washed with diethyl ether. The solid was suspended in dichloromethane and a little methanol, and washed with saturated aqueous sodium bicarbonate solution. The organic phase was dried over sodium sulphate, filtered, concentrated on a rotary evaporator and dried under high vacuum. 61 mg of the target compound (83% of theory) were obtained.

LC-MS (Method 2): Rₜ=0.65 min

MS (ESpos): m/z=437 (M+H)⁺

¹H-NMR (400 MHz, DMSO-d₆): δ=2.32 (s, 3H), 2.68 (s, 3H), 3.88 (s, 2H), 5.32 (s, 2H), 6.00-4.90 (br s, 2H), 6.99 (s, 1H), 7.07 (t, 1H), 7.20-7.31 (m, 4H), 7.60 (quint, 1H), 8.12 (d, 1H), 8.63 (s, 1H).

### Example 113

### N-[2-(Aminomethyl)-4-fluorophenyl]-8-[(2,6-difluorobenzyl)oxy]-2,6-dimethylimidazo[1,2-a]pyridine-3-carboxamide

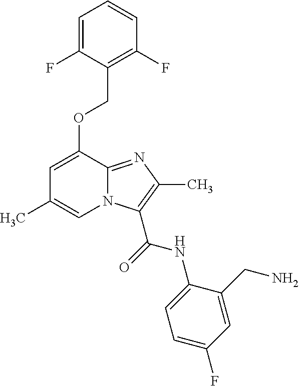 [SMILES: Cc1cc(OCc2c(F)cccc2F)c2nc(C)c(C(=O)Nc3ccc(F)cc3CN)n2c1]

100 mg (0.15 mmol) of tert-butyl {2-[({8-[(2,6-difluorobenzyl)oxy]-2,6-dimethylimidazo[1,2-a]pyridin-3-yl}carbonyl)amino]-5-fluorobenzyl}carbamate trifluoroacetate (Example 44A) were suspended in 0.7 ml of diethyl ether, 0.75 ml (1.50 mmol) of a 2 M solution of hydrogen chloride in diethyl ether were added and the mixture was stirred at RT overnight. The reaction mixture was then concentrated on a rotary evaporator and taken up in 2 ml of dioxane, 0.19 ml of a 4 N solution of hydrogen chloride in dioxane was added and the mixture was stirred at 40° C. for 4 h. Another 1 ml of the 4 N solution of hydrogen chloride in dioxane was added, and the reaction mixture was stirred at 40° C. overnight. The precipitate was filtered off and washed thoroughly with diethyl ether. The residue was taken up in dichloromethane and a little methanol, and washed with saturated aqueous sodium bicarbonate solution. The organic phase was dried over sodium sulphate, filtered and concentrated by rotary evaporation. 55 mg of the target compound (80% of theory) were obtained.

LC-MS (Method 10): Rₜ=0.62 min

MS (ESpos): m/z=455 (M+H)⁺

¹H-NMR (400 MHz, DMSO-d₆): δ=2.32 (s, 3H), 2.66 (s, 3H), 3.84 (s, 2H), 5.30 (s, 2H), 6.20-4.80 (br s, 2H), 6.99 (s, 1H), 7.09-7.30 (m, 4H), 7.60 (quint, 1H), 8.02 (dd, 1H), 8.60 (s, 1H).

### Example 114

### 8-[(2,6-Difluorobenzyl)oxy]-2-methyl-N-[5-(trifluoromethyl)-1H-indazol-3-yl]imidazo[1,2-a]pyridine-3-carboxamide

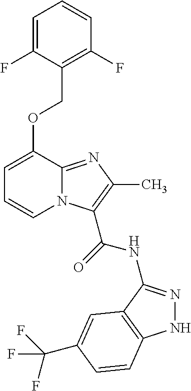 [SMILES: Cc1nc2c(OCc3c(F)cccc3F)cccn2c1C(=O)Nc1n[nH]c2ccc(C(F)(F)F)cc12]

47 mg (0.07 mmol) of tert-butyl 3-[({8-[(2,6-difluorobenzyl)oxy]-2-methylimidazo[1,2-a]pyridin-3-yl}carbonyl)amino]-5-(trifluoromethyl)-1H-indazole-1-carboxylate (Example 47A) were suspended in 0.3 ml of diethyl ether, 0.33 ml (0.66 mmol) of a 2 M solution of hydrogen chloride in diethyl ether were added and the mixture was stirred at RT overnight. The reaction mixture was then concentrated, the residue was taken up in 2 ml of dioxane, 0.08 ml of a 4 N solution of hydrogen chloride in dioxane was added and the mixture was stirred at 40° C. for 4 h. 1 ml of a 4 N solution of hydrogen chloride in dioxane was added, and the reaction mixture was stirred at 40° C. overnight. Another 1 ml of the 4 N solution of hydrogen chloride in dioxane was added, and the reaction mixture was stirred at 40° C. for 5 h. The precipitate was filtered off and washed thoroughly with diethyl ether. The residue was taken up in dichloromethane and a little methanol, and washed with saturated aqueous sodium bicarbonate solution. The organic phase was dried over sodium sulphate, filtered and concentrated. 25 mg of the target compound (74% of theory) were obtained.

LC-MS (Method 2): Rₜ=0.98 min

MS (ESpos): m/z=502 (M+H)⁺

¹H-NMR (400 MHz, DMSO-d₆): δ=2.66 (s, 3H), 5.34 (s, 2H), 6.99 (t, 1H), 7.09 (d, 1H), 7.24 (t, 2H), 7.60 (quint, 1H), 7.63 (d, 1H), 7.70 (d, 1H), 8.31 (s, 1H), 8.64 (d, 1H), 10.63 (s, 1H), 13.23 (s, 1H).

### Example 115

### 8-[(2,6-Difluorobenzyl)oxy]-N-(5-fluoro-1H-indazol-3-yl)-2-methylimidazo[1,2-a]pyridine-3-carboxamide

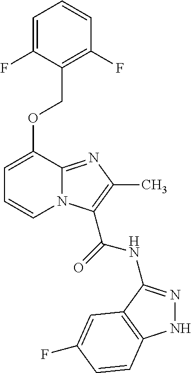 [SMILES: Cc1nc2c(OCc3c(F)cccc3F)cccn2c1C(=O)Nc1n[nH]c2ccc(F)cc12]

26 mg (0.05 mmol) of tert-butyl 3-[({8-[(2,6-difluorobenzyl)oxy]-2-methylimidazo[1,2-a]pyridin-3-yl}carbonyl)amino]-5-fluoro-1H-indazole-1-carboxylate (Example 48A) were suspended in 0.2 ml of diethyl ether, 0.24 ml (0.47 mmol) of a 2 M solution of hydrogen chloride in diethyl ether were added and the mixture was stirred at RT overnight. The precipitate was filtered off, washed thoroughly with diethyl ether and partitioned between ethyl acetate and saturated aqueous sodium bicarbonate solution. The aqueous phase was extracted twice with ethyl acetate, the combined organic phases were dried over sodium sulphate and filtered and the filtrate was concentrated and lyophilized. The crude product was once more dissolved in ethyl acetate and washed twice with saturated aqueous sodium bicarbonate solution, the organic phase was dried over sodium sulphate and filtered and the filtrate was concentrated and lyophilized. The crude product was dissolved in ethyl acetate and washed twice with water, the organic phase was dried over sodium sulphate and filtered and the filtrate was concentrated and lyophilized. This gave 16 mg of the target compound (71% of theory, purity 95%).

LC-MS (Method 2): Rₜ=0.87 min

MS (ESpos): m/z=452 (M+H)⁺

¹H-NMR (400 MHz, DMSO-d₆): δ=2.64 (s, 3H), 5.33 (s, 2H), 6.98 (t, 1H), 7.08 (d, 1H), 7.22-7.29 (m, 3H), 7.50-7.65 (m, 3H), 8.63 (d, 1H), 10.42 (s, 1H), 12.96 (s, 1H).

### Example 116

### 8-[(2,6-Difluorobenzyl)oxy]-N-[1-(2-hydroxyethyl)-5-methyl-3-(trifluoromethyl)-1H-pyrazol-4-yl]-2-methylimidazo[1,2-a]pyridine-3-carboxamide

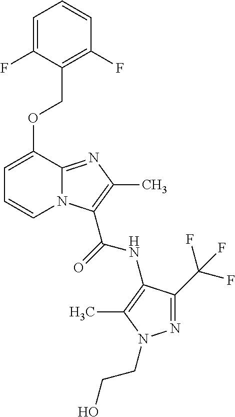 [SMILES: Cc1nc2c(OCc3c(F)cccc3F)cccn2c1C(=O)Nc1c(C(F)(F)F)nn(CCO)c1C]

20 mg (0.043 mmol) of 8-[(2,6-difluorobenzyl)oxy]-2-methyl-N-[5-methyl-3-(trifluoromethyl)-1H-pyrazol-4-yl]imidazo[1,2-a]pyridine-3-carboxamide (Example 76) were initially charged in 0.24 ml of DMF, 36.4 mg (0.112 mmol) of caesium carbonate, 0.7 mg (0.004 mmol) of potassium iodide and 7 mg (0.056 mmol) of bromoethanol were then added and the mixture was stirred at 50° C. overnight. The mixture was then stirred at 70° C. overnight. About 20 ml of water were added to the reaction solution. The precipitated solid was stirred for about 30 min, filtered off and washed thoroughly with water. 10.5 mg of the target compound (48% of theory) were obtained.

LC-MS (Method 2): Rₜ=0.79 min

MS (ESpos): m/z=510 (M+H)⁺

¹H-NMR (400 MHz, DMSO-d₆): δ=2.18 (s, 3H), 2.66 (s, 3H), 3.76 (t, 2H), 4.21 (t, 2H), 5.40 (s, 2H), 7.20-7.34 (m, 3H), 7.49 (br s, 1H), 7.60 (quint, 1H), 8.61 (d, 1H), 9.69 (br s, 1H).

The examples shown in Table 14 were prepared analogously to Example 116:

TABLE 14

| | IUPAC name/structure | |
|---|---|---|
| Example | (Yield) | Analytical data |
| 117 | 6-chloro-8-[(2,6-difluorobenzyl)oxy]-N-[1-(2- hydroxyethyl)-5-methyl-3-(trifluoromethyl)-1H- pyrazol-4-yl]-2-methylimidazo[1,2-a]pyridine-3- carboxamide ^{a)} 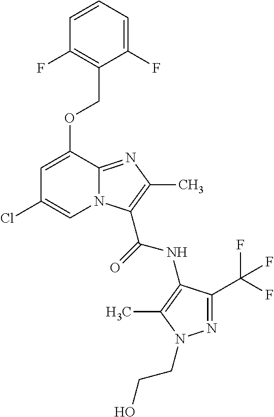 [SMILES: Cc1nc2c(OCc3c(F)cccc3F)cc(Cl)cn2c1C(=O)Nc1c(C(F)(F)F)nn(CCO)c1C] (24% of theory) | LC-MS (Method 2): Rₜ = 1.03 min MS (ESpos): m/z = 544 (M + H)⁺ ¹H-NMR (400 MHz, DMSO-d₆): δ = 2.14/2.28 (s, 3H), 2.60 (s, 3H), 3.72- 3.80 (m, 2H), 4.18-4.23 (m, 2H), 4.99 (t, 1H), 5.37 (s, 2H), 7.20-7.29 (m, 3H), 7.60 (quint., 1H), 8.62 (s, 1H), 9.34/9.42 (s, 1H). |
| 118 | 8-[(2,6-difluorobenzyl)oxy]-N-[1-(2- hydroxyethyl)-5-methyl-3-(trifluoromethyl)-1H- pyrazol-4-yl]-2,6-dimethylimidazo[1,2-a]pyridine- 3-carboxamide (tautomer mixture) ^{b)} 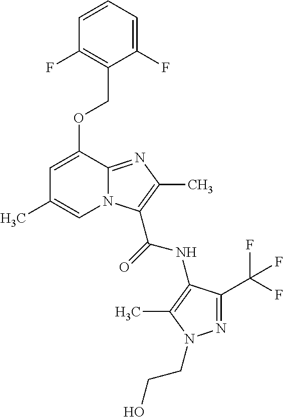 [SMILES: Cc1cc(OCc2c(F)cccc2F)c2nc(C)c(C(=O)Nc3c(C(F)(F)F)nn(CCO)c3C)n2c1] | LC-MS (Method 2): Rₜ = 0.88 min MS (ESpos): m/z = 524 (M + H)⁺ ¹H-NMR (400 MHz, DMSO-d₆): δ = 2.13/2.26 (s, 3H), 2.32 (s, 3H), 2.58 (s, 3H), 3.73-3.81 (m, 2H), 4.18-4.23 (m, 2H), 4.99 (t, 1H), 5.31 (s, 2H), 6.97 (m, 1H), 7.23 (t, 2H), 7.59 (quint., 1H), 8.37 (s, 1H), 9.22/9.30 (s, 1H). |
| | + | |
| | 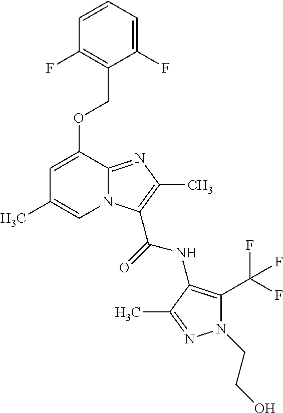 [SMILES: Cc1cc(OCc2c(F)cccc2F)c2nc(C)c(C(=O)Nc3c(C)nn(CCO)c3C(F)(F)F)n2c1] (40% of theory) | |

^{a)} Work-up: The precipitated solid was subsequently purified by silica gel chromatography (mobile phase gradient: dichloromethane/methanol 100/1 to 60/1). The product fractions were concentrated and the residue was purified by preparative HPLC (RP18 column, mobile phase: acetonitrile/water gradient with addition of 0.1% TFA). The product-containing fractions were concentrated, taken up in ethyl acetate, washed once with saturated aqueous sodium bicarbonate solution, dried over sodium sulphate and filtered and the filtrate was concentrated and lyophilized.
^{b)} Work-up: The solid which had been filtered off was stirred with acetonitrile and the solid that remained was filtered off. The filtrate was purified by thick-layer chromatography (mobile phase: dichloromethane/methanol =10/1). The product fractions of the thick-layer chromatography were combined with the solid.

Analogously to Example 1, the Examples shown in Table 15 were prepared by reacting 8-[(2,6-difluorobenzyl)oxy]-2-methylimidazo[1,2-a]pyridine-3-carboxylic acid with the appropriate commercially available amines under the reaction conditions described in Representative Procedure 1:

TABLE 15

| | IUPAC name/structure | |
|---|---|---|
| Example | (Yield) | Analytical data |
| 119 | 8-[(2,6-difluorobenzyl)oxy]-2- methyl-N-(1-methyl-1H-pyrazol- 4-yl)imidazo[1,2-a]pyridine-3- carboxamide 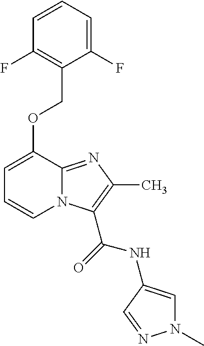 [SMILES: Cc1nc2c(OCc3c(F)cccc3F)cccn2c1C(=O)Nc1cnn(C)c1] Instead of DMF, the solvent used was dichloromethane. (22% of theory) | LC-MS (Method 1): Rₜ = 0.83 min MS (ESpos): m/z = 398.0 (M + H)⁺ ¹H-NMR (400 MHz, DMSO-d₆): δ = 2.55 (s, 3 H; superposed by DMSO signal), 3.81 (s, 3 H), 5.31 (s, 2 H), 6.95 (t, 1 H), 7.03 (d, 1 H), 7.21 (t, 1 H), 7.55 (s, 1 H), 7.57 (quint., 1 H), 8.00 (s, 1 H), 8.55 (d, 1 H), 9.92 (s, 1 H). |

Analogously to Example 22, the Examples shown in Table 16 were prepared by reacting 8-[(2,6-difluorobenzyl)oxy]-2-methylimidazo[1,2-a]pyridine-3-carboxylic acid (Example 3A) with the appropriate commercially available amines under the reaction conditions described in Representative Procedure 2:

TABLE 16

| | IUPAC name/structure | |
|---|---|---|
| Example | (Yield) | Analytical data |
| 120 | 8-[(2,6-difluorobenzyl)oxy]-N-(3- fluoropyridin-4-yl)-2- methylimidazo[1,2-a]pyridine-3- carboxamide 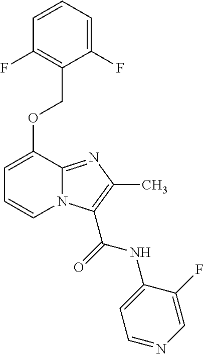 [SMILES: Cc1nc2c(OCc3c(F)cccc3F)cccn2c1C(=O)Nc1ccncc1F] (8% of theory) | LC-MS (Method 2): Rₜ = 0.84 min MS (ESpos): m/z = 413.2 (M + H)⁺ ¹H-NMR (400 MHz, DMSO-d₆): δ = 2.62 (s, 3 H), 5.31 (s, 3 H), 7.03 (t, 1 H), 7.10 (d, 1 H), 7.23 (t, 2 H), 7.58 (quint., 1 H), 8.10 (t, 1 H), 8.38 (d, 1 H), 8.58 (d, 1 H), 8.68 (d, 1 H), 10.0 (s, 1 H). |
| 121 | 8-[(2,6-difluorobenzyl)oxy]- N-(1-ethyl-1H-pyrazol-3-yl)- 2-methylimidazo[1,2-a] pyridine-3-carboxamide 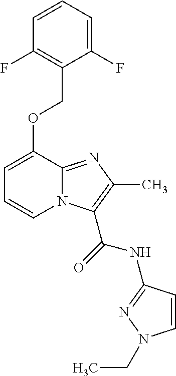 [SMILES: CCn1ccc(NC(=O)c2c(C)nc3c(OCc4c(F)cccc4F)cccn23)n1] (9% of theory) | LC-MS (Method 2): Rₜ = 0.81 min MS (ESpos): m/z = 412.1 (M + H)⁺ ¹H-NMR (400 MHz, DMSO-d₆): δ = 1.37 (t, 3 H), 2.55 (s, 3 H; superposed by DMSO signal), 5.05 (q, 2 H), 5.29 (s, 2 H), 6.55 (s, 1 H), 6.92 (t, 1 H), 7.03 (d, 1 H), 7.20 (t, 2 H), 7.55 (quint., 1 H), 7.61 (s, 1 H), 8.55 (d, 1 H), 10.40 (s, 1 H). |
| 122 | 8-[(2,6-difluorobenzyl)oxy]-N-(1,5- dimethyl-1H-pyrazol-3-yl)-2- methylimidazo[1,2-a] pyridine-3-carboxamide 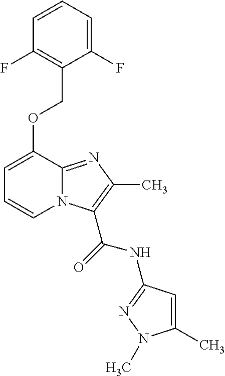 [SMILES: Cc1nc2c(OCc3c(F)cccc3F)cccn2c1C(=O)Nc1cc(C)n(C)n1] (39% of theory) | LC-MS (Method 2): Rₜ = 0.84 min MS (ESpos): m/z = 412.2 (M + H)⁺ ¹H-NMR (400 MHz, DMSO-d₆): δ = 2.20 (s, 3 H), 2.55 (s, 3 H; superposed by DMSO signal), 3.65 (s, 3 H), 5.29 (s, 2 H), 6.40 (s, 1 H), 6.92 (t, 1 H), 7.02 (d, 1 H), 7.23 (t, 2 H), 7.58 (quint., 1 H), 8.52 (d, 1 H), 10.21 (s, 1 H). |

Analogously to Example 22, the Examples shown in Table 17 were prepared by reacting 8-[(2,6-difluorobenzyl)oxy]-2-methylimidazo[1,2-a]pyridine-3-carboxylic acid (Example 3A) with the appropriate commercially available amines under the reaction conditions described in Representative Procedure 2, where owing to the low conversion a larger excess of the amine component was used and the reaction temperature was increased to 66° C.

TABLE 17

| | IUPAC name/structure | |
|---|---|---|
| Example | (Yield) | Analytical data |
| 123 | 8-[(2,6-difluorobenzyl)oxy]-2-methyl-N-[3- (trifluoromethyl)pyridin-4-yl)imidazo[1,2- a]pyridine-3-carboxamide trifluoroacetate 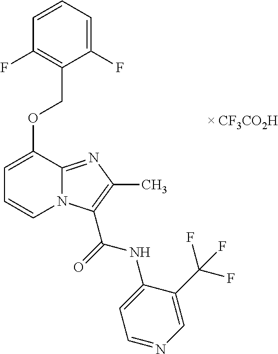 [SMILES: Cc1nc2c(OCc3c(F)cccc3F)cccn2c1C(=O)Nc1ccncc1C(F)(F)F] (7% of theory) | LC-MS (Method 1): Rₜ = 1.23 min MS (ESpos): m/z = 463.0 (M − TFA + H)⁺ ¹H-NMR (400 MHz, DMSO-d₆): δ = 2.68 (s, 3 H), 5.40 (s, 2 H), 7.08-7.30 (m, 4 H), 7.60 (quint., 1 H), 8.03 (m, 1 H), 8.71 (m, 1 H), 8.82 (m, 1 H), 8.92 (m, 1 H), 9.60 (s, 1 H). |

Analogously to Example 48, the Examples shown in Table 18 were prepared by reacting the respective carboxylic acids in each case with commercially available amines under the reaction conditions described in Representative Procedure 2:

TABLE 18

| | IUPAC name/structure | |
|---|---|---|
| Example | (Yield) | Analytical data |
| 124 | 8-(cyclohexylmethoxy)-N-[1-(2- hydroxyethyl)-1H-pyrazol-4-yl]- 2-methylimidazo[1,2-a]pyridine- 3-carboxamide 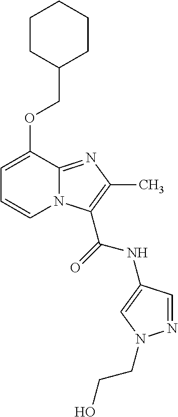 [SMILES: Cc1nc2c(OCC3CCCCC3)cccn2c1C(=O)Nc1cnn(CCO)c1] (56% of theory) | LC-MS (Method 2): Rₜ = 0.73 min MS (ESpos): m/z = 398.3 (M + H)⁺ ¹H-NMR (400 MHz, DMSO-d₆): δ = 1.00-1.40 (m, 5H), 1.60-1.75 (m, 3 H), 1.80-1.90 (m, 3 H), 2.60 (s, 3 H), 3.70 (q, 2 H), 3.95 (d, 2 H), 4.10 (t, 2 H), 4.90 (t, 1 H), 6.80 (d, 1 H), 6.90 (t, 1 H), 7.58 (s, 1 H), 8.08 (s, 1 H), 8.50 (d, 1 H), 9.80 (s, 1 H). |
| 125 | 6-bromo-8-[(2,6-difluorobenzyl)oxy]-N-(3,5- dimethyl-1H-pyrazol-4-yl)-2-methylimidazo [1,2-a]pyridine-3-carboxamide 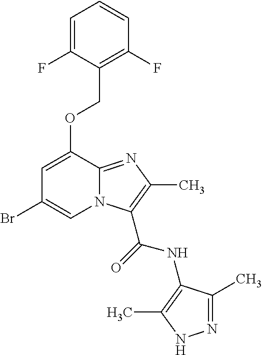 [SMILES: Cc1n[nH]c(C)c1NC(=O)c1c(C)nc2c(OCc3c(F)cccc3F)cc(Br)cn12] (98% of theory) | LC-MS (Method 2): Rₜ = 0.87 min MS (ESpos): m/z = 390.2 (M + H)⁺ 1H-NMR (400 MHz, DMSO-d₆): δ = 2.04 (s, 6 H), 2.60 (s, 3 H), 5.35 (s, 2 H), 7.23 (t, 1 H), 7.26 (s, 1 H), 7.58 (s, 1 H), 8.70 (s, 1 H), 9.02 (s, 1 H), 12.20 (s, 1 H). |

### Example 126

### 8-[(2,6-Difluorobenzyl)oxy]-2-methyl-N-(4-methylpyrimidin-2-yl)imidazo[1,2-a]pyridine-3-carboxamide

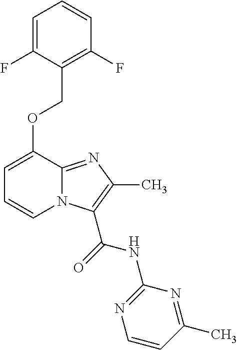 [SMILES: Cc1ccnc(NC(=O)c2c(C)nc3c(OCc4c(F)cccc4F)cccn23)n1]

At room temperature and under argon protective gas, 50 mg (0.157 mmol) of 8-[(2,6-difluorobenzyl)oxy]-2-methylimidazo[1,2-a]pyridine-3-carboxylic acid (Example 3A) were initially charged, 200 μl of thionyl chloride were added and the mixture was stirred overnight. The mixture was then concentrated to dryness under high vacuum and the residue was taken up in 300 μl of dry THF. In a second flask, 21 mg of 2-amino-4-methylpyrimidine (0.189 mmol) were initially charged in 200 μl of dry THF, 79 μl of 2.6 M n-butyllithium solution (in toluene; 0.2 mmol) were added with ice cooling and the mixture was stirred for another 15 minutes. The resulting solution was added dropwise to the ice-cooled solution of the acid chloride, and the resulting mixture was warmed to room temperature and stirred for a further 16 h. Following aqueous work-up, the product was purified by HPLC (Method 9). This gave 16.6 mg (24% of theory).

LC-MS (Method 3): Rₜ=1.49 min

MS (ESpos): m/z=410.2 (M+H)⁺

¹H-NMR (400 MHz, DMSO-d₆): δ=2.40 (s, 3H), 2.55 (s, 3H; superposed by DMSO signal), 5.31 (s, 2H), 6.98 (t, 1H), 7.07-7.12 (m, 2H), 7.23 (t, 2H), 7.59 (quint., 1H), 8.50 (d, 1H), 8.62 (d, 1H), 10.52 (s, 1H).

### Example 127

### 8-[(2,6-Difluorobenzyl)oxy]-N-{1-[2-(4,4-difluoropiperidin-1-yl)ethyl]-1H-pyrazol-4-yl}-2-methylimidazo[1,2-a]pyridine-3-carboxamide

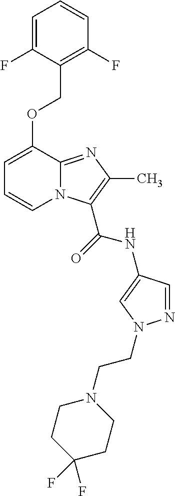 [SMILES: Cc1nc2c(OCc3c(F)cccc3F)cccn2c1C(=O)Nc1cnn(CCN2CCC(F)(F)CC2)c1]

100 mg (0.198 mmol) of 2-{4-[({8-[(2,6-difluorobenzyl)oxy]-2-methylimidazo[1,2-a]pyridin-3-yl}carbonyl)amino]-1H-pyrazol-1-yl}ethyl methanesulphonate (Example 33A) were dissolved in 2 ml of tetrahydrofuran, 119 mg of 4,4-difluoropiperidine (1 mmol), 69 μl of diisopropylethylamine (0.396 mmol), 59 mg of sodium iodide (0.396 mmol), 83 μl of triethylamine (0.593 mmol) and a catalytic amount of 4-dimethylaminopyridine were added in succession and the mixture was heated at reflux for 16 h. The mixture was then diluted with ethyl acetate and washed with water and saturated sodium chloride solution. The aqueous phase was extracted twice with ethyl acetate. The combined organic phases were dried, concentrated and purified by HPLC (Method 7). This gave 28 mg (26% of theory) of the desired product.

LC-MS (Method 2): Rₜ=0.73 min

MS (ESpos): m/z=531.3 (M+H)⁺

¹H-NMR (400 MHz, DMSO-d₆): δ=1.88-1.95 (m, 4H), 2.57 (s, 3H; partially superposed by DMSO signal), 2.78 (t, 2H), 4.20 (t, 2H), 5.31 (s, 2H), 6.92 (t, 1H), 7.02 (d, 1H), 7.23 (t, 2H), 7.53 (s, 1H), 7.55 (quint., 1H), 8.02 (s, 1H), 8.55 (d, 1H), 9.92 (s, 1H). [a further signal is hidden under the DMSO and water solvent signals]

Analogously to the above example, the example compounds listed in Table 19 below were prepared by reacting 2-{4-[({8-[(2,6-difluorobenzyl)oxy]-2-methylimidazo[1,2-a]pyridin-3-yl}carbonyl)amino]-1H-pyrazol-1-yl}ethyl methanesulphonate (Example 33A) with the appropriate commercially available amines

TABLE 19

| | IUPAC name/structure | |
|---|---|---|
| Example | (Yield) | Analytical data |
| 128 | 8-[(2,6-difluorobenzyl)oxy]-2- methyl-N-{1-[2-(pyrrolidin-1-yl) ethyl]-1H-pyrazol-4-yl}imidazo [1,2-a]pyridine-3-carboxamide 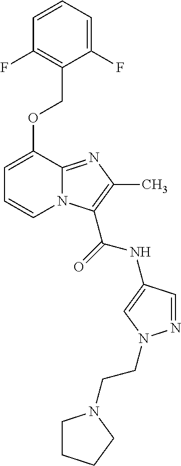 [SMILES: Cc1nc2c(OCc3c(F)cccc3F)cccn2c1C(=O)Nc1cnn(CCN2CCCC2)c1] (37% of theory) | LC-MS (Method 2): Rₜ = 0.66 min MS (ESpos): m/z = 481.4 (M + H)⁺ ¹H-NMR (400 MHz, DMSO-d₆): δ = 1.60-1.70 (m, 4 H), 2.57 (s, 3 H; partially superimposed by the DMSO signal), 2.80 (br. s, 2 H), 4.20 (t, 2 H), 5.31 (s, 2 H), 6.92 (t, 1 H), 7.02 (d, 1 H), 7.23 (t, 2 H), 7.53 (s, 1 H), 7.55 (quint., 1 H), 8.02 (s, 1 H), 8.55 (d, 1 H), 9.96 (s, 1 H), [a further signal is hidden under the DMSO and water signal] |
| 129 | 8-[(2,6-difluorobenzyl)oxy]- N-{1-[2-(1,1- dioxidothiomorpholin- 4-yl)ethyl]-1H-pyrazol-4-yl}- 2-methylimidazo[1,2-a] pyridine-3-carboxamide 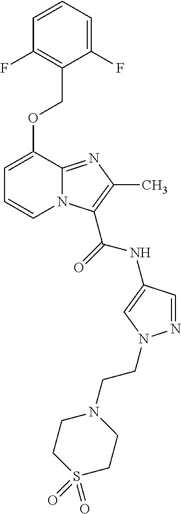 [SMILES: Cc1nc2c(OCc3c(F)cccc3F)cccn2c1C(=O)Nc1cnn(CCN2CCS(=O)(=O)CC2)c1] (22% of theory) | LC-MS (Method 2): Rₜ = 0.76 min MS (ESpos): m/z = 545.3 (M+H)⁺ ¹H-NMR (400 MHz, DMSO-d₆): δ = 2.57 (s, 3 H; partially superposed by DMSO signal), 2.85 (t, 2 H), 2.88- 2.93 (m, 4 H), 3.00-3.10 (m, 4 H), 4.20 (t, 2 H), 5.31 (s, 2 H), 6.92 (t, 1 H), 7.02 (d, 1 H), 7.23 (t, 2 H), 7.55 (quint., 1 H), 7.56 (s, 1 H), 8.10 (s, 1 H), 8.55 (d, 1 H), 9.96 (s, 1 H). |
| 130 | 8-[(2,6-difluorobenzyl)oxy]-N- {1-[2-(4-hydroxypiperidin-1-yl) ethyl]-1H-pyrazol-4-yl}-2- methylimidazol[1,2-a] pyridine-3-carboxamide 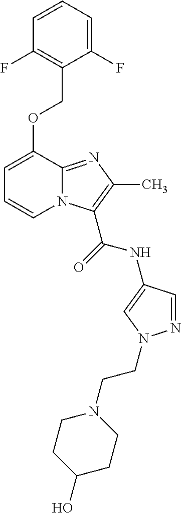 [SMILES: Cc1nc2c(OCc3c(F)cccc3F)cccn2c1C(=O)Nc1cnn(CCN2CCC(O)CC2)c1] (35% of theory) | LC-MS (Method 2): Rₜ = 0.63 min MS (ESpos): m/z = 511.4 (M + H)⁺ ¹H-NMR (400 MHz, DMSO-d₆): δ = 1.25-1.38 (m, 2 H), 1.66-1.74 (m, 2 H), 2.05-2.12 (m, 2 H), 2.57 (s, 3 H; partially superposed by DMSO signal). 2.65 (t, 2 H), 2.68-2.75 (m, 2 H), 3.38-3.45 (m, 1 H), 4.12 (t, 2 H), 4.52 (d, 1 H), 5.31 (s, 2 H), 6.92 (t, 1 H), 7.02 (d, 1 H), 7.23 (t, 2 H), 7.54 (s, 1 H), 7.55 (quint., 1 H), 8.03 (s, 1 H), 8.55 (d, 1 H), 9.93 (s, 1 H). |
| 131 | rac-8-[(2,6-difluorobenzyl)oxy]- N-{1-[2-(3-hydroxypyrrolidin- 1-yl)ethyl]-1H-pyrazol-4-yl}-2- methylimidazol[1,2-a] pyridine-3-carboxamide (racemate) 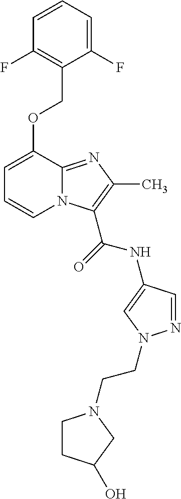 [SMILES: Cc1nc2c(OCc3c(F)cccc3F)cccn2c1C(=O)Nc1cnn(CCN2CCC(O)C2)c1] (25% of theory) | LC-MS (Method 2): Rₜ = 0.63 min MS (ESpos): m/z = 497.4 (M + H)⁺ ¹H-NMR (400 MHz, DMSO-d₆): δ = 1.45-1.55 (m, 1 H), 1.90-2.00 (m, 1 H), 2.30 (dd, 1 H), 2.45-2.60 (m, 2 + 3 H; partially superposed by DMSO signal), 2.70-2.76 (m, 1 H), 2.80 (t, 2 H), 4.13-4.22 (m, 3 H), 4.70 (d, 1 H), 5.31 (s, 2 H), 6.92 (t, 1 H), 7.02 (d, 1 H), 7.23 (t, 2 H), 7.53 (s, 1 H), 7.55 (quint., 1 H), 8.06 (s, 1 H), 8.60 (d, 1 H), 9.95 (s, 1 H). |
| 132 | 8-[(2,6-difluorobenzyl)oxy]-2- methyl-N-{1-[2-(morpholin- 4-yl)ethyl]-1H-pyrazol-4-yl} imidazo[1,2-a]pyridine-3- carboxamide 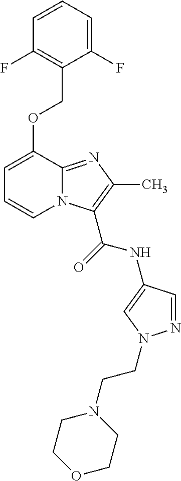 [SMILES: Cc1nc2c(OCc3c(F)cccc3F)cccn2c1C(=O)Nc1cnn(CCN2CCOCC2)c1] (24% of theory) | LC-MS (Method 2): Rₜ = 0.54 min MS (ESpos): m/z = 497.3 (M + H)⁺ ¹H-NMR (400 MHz, DMSO-d₆): δ = 2.40-2.42 (m, 4 H), 2.57 (s, 3 H; partially superposed by DMSO signal). 2.68 (t, 2 H), 3.56 (t, 4 H), 4.20 (t, 2 H), 5.31 (s, 2 H), 6.92 (t, 1 H), 7.02 (d, 1 H), 7.23 (t, 2 H), 7.54 (s, 1 H), 7.55 (quint., 1 H), 8.08 (s, 1 H), 8.55 (d, 1 H), 9.95 (s, 1 H). |
| 133 | 8-[(2,6-difluorobenzyl)oxy]-2- methyl-N-{1-[2-(4- methylpiperazin-1-yl)ethyl]- 1H-pyrazol-4-yl)imidazo [1,2-a]pyridine-3-carboxamide 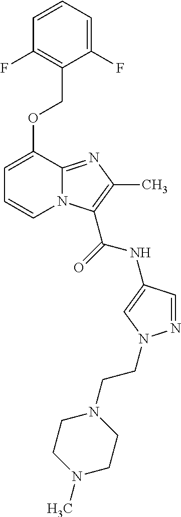 [SMILES: Cc1nc2c(OCc3c(F)cccc3F)cccn2c1C(=O)Nc1cnn(CCN2CCN(C)CC2)c1] (35% of theory) | LC-MS (Method 2): Rₜ = 0.64 min MS (ESpos): m/z = 510.4 (M + H)⁺ ¹H-NMR (400 MHz, DMSO-d₆): δ = 2.10 (s, 3 H), 2.20-2.50 (m, 8 H), 2.57 (s, 3 H; partially superposed by DMSO signal), 2.70 (t, 2 H), 4.20 (t, 2 H), 5.31 (s, 2 H), 6.92 (t, 1 H), 7.02 (d, 1 H), 7.23 (t, 2 H), 7.54 (s, 1 H), 7.55 (quint., 1 H), 8.05 (s, 1 H), 8.55 (d, 1 H), 9.95 (s, 1 H). |
| 134 | 8-[(2,6-difluorobenzyl)oxy]-N- {1-[2-(dimethylamino)ethyl]- 1H-pyrazol-4-yl}-2- methylimidazol[1,2-a] pyridine-3-carboxamide 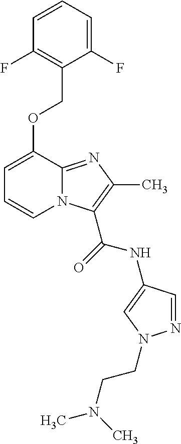 [SMILES: Cc1nc2c(OCc3c(F)cccc3F)cccn2c1C(=O)Nc1cnn(CCN(C)C)c1] (41% of theory) | LC-MS (Method 2): Rₜ = 0.63 min MS (ESpos): m/z = 455.3 (M + H)⁺ ¹H-NMR (400 MHz, DMSO-d₆): δ = 2.14 (s, 6 H), 2.57 (s, 3 H; partially superposed by DMSO signal), 2.63 (t, 2 H), 4.18 (t, 2 H), 5.31 (s, 2 H), 6.92 (t, 1 H), 7.02 (d, 1 H), 7.23 (t, 2 H), 7.54 (s, 1 H), 7.55 (quint., 1 H), 8.04 (s, 1 H), 8.58 (d, 1 H), 9.96 (s, 1 H). |

### Example 135

### 8-[(2,6-Difluorobenzyl)oxy]-2-methyl-N-{1-[2-(methylsulphonyl)ethyl]-1H-pyrazol-4-yl}imidazo[1,2-a]pyridine-3-carboxamide

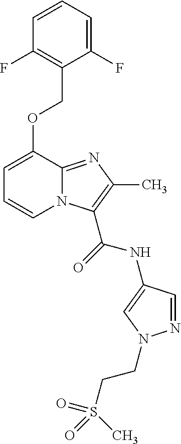 [SMILES: Cc1nc2c(OCc3c(F)cccc3F)cccn2c1C(=O)Nc1cnn(CCS(C)(=O)=O)c1]

100 mg (0.198 mmol) of 2-{4-[({8-[(2,6-difluorobenzyl)oxy]-2-methylimidazo pyridin-3-yl}carbonyl)amino]-1H-pyrazol-1-yl}ethyl methanesulphonate (Example 33A) were dissolved in 2 ml of dimethylformamide, 201 mg of sodium methylsulphinate (2 mmol) and 297 mg of sodium iodide (2 mmol) were added in succession and the mixture was heated at 100° C. for 4 h. The mixture was then diluted with ethyl acetate and washed with water. The aqueous phase was extracted with ethyl acetate. The combined organic phases were washed with sodium thiosulphate solution, dried and concentrated and the residue was chromatographed (Biotage Isolera SP4, ethyl acetate/cyclohexane gradient). This gave 45 mg (47% of theory) of the target compound.

LC-MS (Method 2): Rₜ=0.68 min

MS (ESpos): m/z=490.3 (M+H)⁺

¹H-NMR (400 MHz, DMSO-d₆): δ=2.57 (s, 3H; partially superposed by DMSO signal), 2.90 (s, 3H), 3.70 (t, 2H), 4.55 (t, 2H), 5.31 (s, 2H), 6.92 (t, 1H), 7.04 (d, 1H), 7.23 (t, 2H), 7.60 (quint., 1H), 7.65 (s, 1H), 8.15 (s, 1H), 8.55 (d, 1H), 10.01 (s, 1H).

### Example 136

### N-[1-(2-Aminoethyl)-1H-pyrazol-4-yl]-8-[(2,6-difluorobenzyl)oxy]-2-methylimidazo[1,2-a]pyridine-3-carboxamide

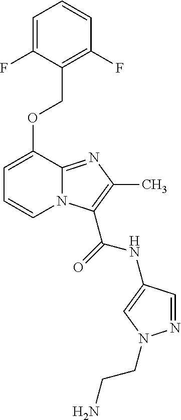 [SMILES: Cc1nc2c(OCc3c(F)cccc3F)cccn2c1C(=O)Nc1cnn(CCN)c1]

1 ml of 40% aqueous methylamine solution was added to 335 mg (0.6 mmol) of 8-[(2,6-difluorobenzyl)oxy]-N-{1-[2-(1,3-dioxo-1,3-dihydro-2H-isoindol-2-yl)ethyl]-1H-pyrazol-4-yl}-2-methylimidazo[1,2-a]pyridine-3-carboxamide (Example 34A), and the mixture was stirred in a pressure vessel at 60° C. for 16 h. The solid was filtered off with suction, washed with a little water and dried. 195 mg (76% of theory) of the title compound were obtained.

LC-MS (Method 2): Rₜ=0.54 min

MS (ESpos): m/z=427.3 (M+H)⁺

¹H-NMR (400 MHz, DMSO-d₆): δ=1.60 (br. s, 2H), 2.55 (s, 3H; superposed by DMSO signal), 2.90 (t, 2H), 4.03 (t, 2H), 5.30 (s, 2H), 6.95 (t, 1H), 7.03 (d, 1H), 7.23 (t, 2H), 7.51-7.61 (m, 2H), 8.04 (s, 1H), 8.55 (d, 1H), 9.95 (s, 1H).

### Example 137

### 8-[(2,6-Difluorobenzyl)oxy]-N-(1-{2-[(ethylsulphonyl)amino]ethyl}-1H-pyrazol-4-yl)-2-methylimidazo[1,2-a]pyridine-3-carboxamide

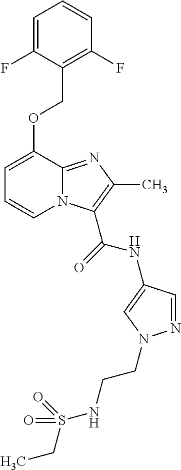 [SMILES: CCS(=O)(=O)NCCn1cc(NC(=O)c2c(C)nc3c(OCc4c(F)cccc4F)cccn23)cn1]

32 mg (0.075 mmol) of N41-(2-aminoethyl)-1H-pyrazol-4-yl]-8-[(2,6-difluorobenzyl)oxy]-2-methylimidazo[1,2-a]pyridine-3-carboxamide were initially charged in 150 μl of THF and 150 μl of pyridine, and 7.8 μl of ethylsulphonyl chloride (0.08 mmol) were added with ice cooling. The reaction was stirred at room temperature. A further 17.8 μl of ethylsulphonyl chloride (0.187 mmol) were added a little at a time, and the mixture was stirred at room temperature for a total of 48 h. A small amount of N-methylpiperazine was then added, the mixture was concentrated and the residue was taken up in methanol and purified by HPLC (Method 7). This gave 8.4 mg (22% of theory) of the target compound as colourless crystals.

LC-MS (Method 2): Rₜ=0.69 min

MS (ESpos): m/z=519.2 (M+H)⁺

¹H-NMR (400 MHz, DMSO-d₆): δ=1.12 (t, 3H), 2.55 (s, 3H; superposed by DMSO signal), 2.92 (q, 2H), 3.30 (q, 2H; partially superposed by water signal), 4.15 (t, 2H), 5.30 (s, 2H), 6.95 (t, 1H), 7.03 (d, 1H), 7.23 (t, 2H), 7.57 (quint., 1H), 7.61 (s, 1H), 8.09 (s, 1H), 8.55 (d, 1H), 9.95 (s, 1H).

### Example 138

### 2-{4-[({8-[(2,6-Difluorobenzyl)oxy]-2-methylimidazo[1,2-a]pyridin-3-yl}carbonyl)amino]-1H-pyrazol-1-yl}ethyl carbamate

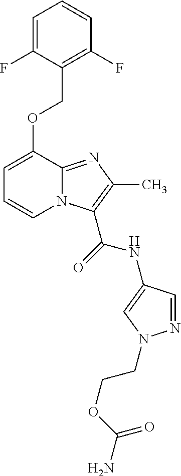 [SMILES: Cc1nc2c(OCc3c(F)cccc3F)cccn2c1C(=O)Nc1cnn(CCOC(N)=O)c1]

100 mg (0.234 mmol) of 8-[(2,6-difluorobenzyl)oxy]-N-[1-(2-hydroxyethyl)-1H-pyrazol-4-yl]-2-methylimidazo[1,2-a]pyridine-3-carboxamide (Example 22) were dissolved in 24 ml of dichloromethane, and 41 μl of chlorosulphonyl isocyanate (0.468 mmol) were added with ice cooling. The resulting mixture was warmed to room temperature and stirred for another 1 h and then purified by HPLC (Method 7). The crude product thus obtained was separated by a second HPLC purification (Sunfire C 18.5 μM, 250×20 mm, 25 ml/min, isocratic 65% water, 30% acetonitrile, 1% TFA in water) into the target compound and the minor components Example 39 shown below. This gave 12 mg (11% of theory) of the target compound.

LC-MS (Method 2): Rₜ=0.71 min

MS (ESpos): m/z=471.3 (M+H)⁺

¹H-NMR (400 MHz, DMSO-d₆): δ=2.60 (s, 3H), 4.25-4.35 (m, 4H), 5.40 (s, 2H), 6.40-6.70 (br. m, 2H), 7.20-7.25 (m, 3H), 7.40 (br. s, 1H), 7.58 (quint., 1H), 7.61 (s, 1H), 8.09 (s, 1H), 8.61 (d, 1H), 10.32 (br.s, 1H).

### Example 139

### 2-{4-[({8-[(2,6-Difluorobenzyl)oxy]-2-methylimidazo[1,2-a]pyridin-3-yl}carbonyl)amino]-1H-pyrazol-1-yl}ethyl sulphamate trifluoroacetate

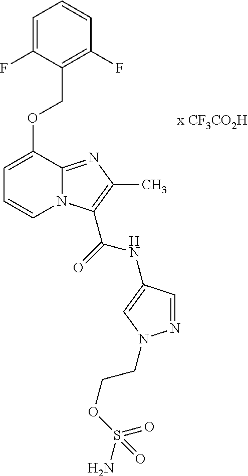 [SMILES: Cc1nc2c(OCc3c(F)cccc3F)cccn2c1C(=O)Nc1cnn(CCOS(N)(=O)=O)c1]

2-{4-[({8-[(2,6-Difluorobenzyl)oxy]-2-methylimidazo[1,2-a]pyridin-3-yl}carbonyl)amino]-1H-pyrazol-1-yl}ethyl sulphamate was isolated as by-product from the above-described preparation of 2-{4-[({8-[(2,6-difluorobenzyl)oxy]-2-methylimidazo[1,2-a]pyridin-3-yl}carbonyl)amino]-1H-pyrazol-1-yl}ethyl carbamate. 25 mg (21% of theory) were obtained.

LC-MS (Method 2): Rₜ=0.68 min

MS (ESpos): m/z=507.3 (M+H)⁺

¹H-NMR (400 MHz, DMSO-d₆): δ=2.60 (s, 3H), 4.33 (t, 2H), 4.43 (t, 2H), 5.40 (s, 2H), 7.14 (br. s, 1H), 7.23 (t, 2H), 7.25 (br. s, 1H), 7.60 (s, 2H), 7.61 (quint., 1H), 7.64 (s, 1H), 8.12 (s, 1H), 8.61 (d, 1H), 10.26 (br. s, 1H).

### Example 140

### 8-[(2,6-Difluorobenzyl)oxy]-N-[1-(2-fluoroethyl)-1H-pyrazol-4-yl]-2-methylimidazo[1,2-a]pyridine-3-carboxamide

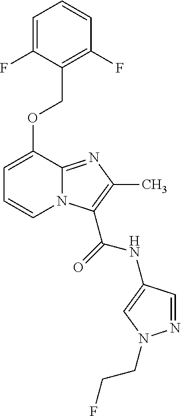 [SMILES: Cc1nc2c(OCc3c(F)cccc3F)cccn2c1C(=O)Nc1cnn(CCF)c1]

100 mg (0.234 mmol) of 8-[(2,6-difluorobenzyl)oxy]-N-[1-(2-hydroxyethyl)-1H-pyrazol-4-yl]-2-methylimidazo[1,2-a]pyridine-3-carboxamide (Example 22) were dissolved in 1 ml of dichloromethane, and 83 μl of diethylaminosulphur trifluoride (DAST) (0.632 mmol) were added at −78° C. The mixture was gradually warmed to room temperature. After 3 h, a further 46 μl of DAST (0.35 mmol) were added dropwise and the reaction mixture was stirred at room temperature for 16 h. The reaction mixture was applied to Isolute and chromatographed (Biotage Isolera, gradient cyclohexane/ethyl acetate). 11.5 mg (12% of theory) of the title compound were obtained.

LC-MS (Method 2): Rₜ=0.73 min

MS (ESpos): m/z=430.3 (M+H)⁺

¹H-NMR (400 MHz, DMSO-d₆): δ=2.55 (s, 3H; superposed by DMSO signal), 4.41 (dt, 2H), 4.75 (dt, 2H), 5.30 (s, 2H), 6.95 (t, 1H), 7.03 (d, 1H), 7.21 (t, 2H), 7.57 (quint., 1H), 7.62 (s, 1H), 8.10 (s, 1H), 8.58 (d, 1H), 10.01 (s, 1H).

Analogously to the example above, the exemplary compound listed in Table 20 below was prepared by reaction of 6-chloro-8-[(2,6-difluorobenzyl)oxy]-N-[1-(2-hydroxyethyl)-1H-pyrazol-4-yl]-2-methylimidazo[1,2-a]pyridine-3-carboxamide (Example 46).

TABLE 20

| | IUPAC name/structure | |
|---|---|---|
| Example | (Yield) | Analytical data |
| 141 | 6-chloro-8-[(2,6-difluorobenzyl)oxy]- N-[1-(2-fluoroethyl)-1H-pyrazol-4-yl]- 2-methylimidazo[1,2-a]pyridine-3- carboxamide 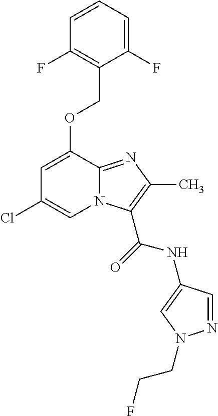 [SMILES: Cc1nc2c(OCc3c(F)cccc3F)cc(Cl)cn2c1C(=O)Nc1cnn(CCF)c1] (21% of theory) | LC-MS (Method 2): Rₜ = 1.01 min MS (ESpos): m/z = 464.2 (M + H)⁺ ¹H-NMR (400 MHz, DMSO-d₆): δ = 2.55 (s, 3 H; superposed by DMSO signal), 4.42 (dt, 2 H), 4.75 (dt, 2 H), 5.36 (s, 2 H), 7.20-7.28 (m, 3 H), 7.59 (quint., 1 H), 7.63 (s, 1 H), 8.13 (s, 1 H), 8.68 (s, 1 H), 10.07 (s, 1 H). |

### Example 142

### 2-Cyclopropyl-8-[(2,6-difluorobenzyl)oxy]-N-[1-(2-hydroxyethyl)-1H-pyrazol-4-yl]imidazo[1,2-a]pyridine-3-carboxamide

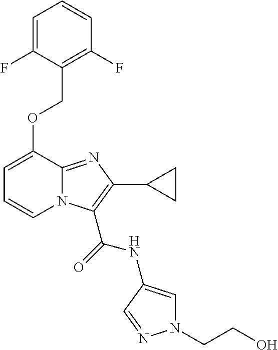 [SMILES: O=C(Nc1cnn(CCO)c1)c1c(C2CC2)nc2c(OCc3c(F)cccc3F)cccn12]

70 mg (0.20 mmol) of 2-cyclopropyl-8-[(2,6-difluorobenzyl)oxy]imidazo[1,2-a]pyridine-3-carboxylic acid Example 53A were initially charged in 0.93 ml of DMF, 39 mg (0.31 mmol) of 2-(4-amino-1H-pyrazol-1-yl)ethanol, 100 mg (0.26 mmol) of O-(7-azabenzotriazol-1-yl)-N,N,N′N′-tetramethyluronium hexafluorophosphate (HATU) and 79 mg (0.61 mmol) of N,N-diisopropylethylamine were added and the mixture was stirred at RT overnight. Water was added to the reaction solution and the resulting precipitate was filtered off with suction, washed with water and dried under high vacuum. 30 mg of the target compound (33% of theory) were obtained.

LC-MS (Method 2): Rₜ=0.77 min

MS (ESpos): m/z=454 (M+H)⁺

¹H-NMR (400 MHz, DMSO-d₆): δ=0.91-1.04 (m, 4H), 2.30-2.41 (m, 1H), 3.72 (q, 2H), 4.13 (t, 2H), 4.90 (t, 1H), 5.31 (s, 2H), 6.93 (t, 1H), 7.02 (d, 1H), 7.24 (t, 2H), 7.53-5.64 (m, 2H), 8.06 (s, 1H), 8.52 (d, 1H), 10.25 (s, 1H).

### Example 143

### N-(2-Amino-3,5-difluorophenyl)-8-[(2,6-difluorobenzyl)oxy]-2-methylimidazo[1,2-a]pyridine-3-carboxamide

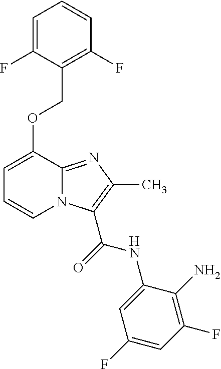 [SMILES: Cc1nc2c(OCc3c(F)cccc3F)cccn2c1C(=O)Nc1cc(F)cc(F)c1N]

17 mg (0.12 mmol) of 3,5-difluorobenzene-1,2-diamine were initially charged, a solution of 32 mg (0.10 mmol) of 8-[(2,6-difluorobenzyl)oxy]-2-methylimidazo[1,2-a]pyridine-3-carboxylic acid from Example 3A in 0.4 ml of DMF, a solution of 42 mg (0.13 mmol) of TBTU in 0.2 ml of DMF and then 20 mg (0.20 mmol) of 4-methylmorpholine were added and the mixture was shaken at RT overnight. The target compound was isolated by preparative HPLC (Method 6). 3 mg (5% of theory) were obtained.

LC-MS (Method 5): Rₜ=0.99 min

MS (ESpos): m/z=445 (M+H)⁺

Analogously to Example 143, the example compounds shown in Table 21 were prepared by reacting 8-[(2,6-difluorobenzyl)oxy]-2-methylimidazo[1,2-a]pyridine-3-carboxylic acid from Example 3A with the appropriate commercially available amines, under the conditions described:

TABLE 21

| | IUPAC name/structure | |
|---|---|---|
| Example | (Yield) | Analytical data |
| 144 | 8-[(2,6-difluorobenzyl)oxy]-N-(4,5- dimethyl-1,3-oxazol-2-yl)-2- methylimidazo[1,2-a]pyridine-3- carboxamide 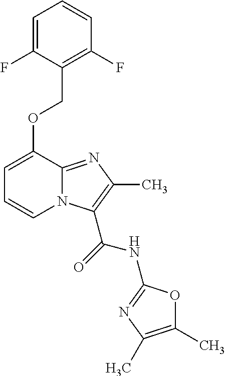 [SMILES: Cc1nc(NC(=O)c2c(C)nc3c(OCc4c(F)cccc4F)cccn23)oc1C] (20% of theory) | LC-MS (Method 5): Rₜ = 0.90 min MS (ESpos): m/z = 413 (M + H)⁺ |
| 145 | 8-[(2,6-difluorobenzyl)oxy]-2- methyl-N-(1,3-thiazol-2-yl) imidazo[1,2-a]pyridine-3- carboxamide 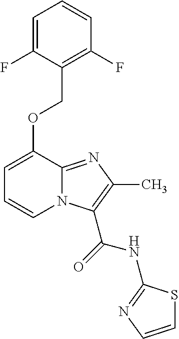 [SMILES: Cc1nc2c(OCc3c(F)cccc3F)cccn2c1C(=O)Nc1nccs1] (58% of theory; purity 90%) | LC-MS (Method 5): Rₜ = 0.90 min MS (ESpos): m/z = 401 (M + H)⁺ |
| 146 | 8-[(2,6-difluorobenzyl)oxy]-2- methyl-N-(5-methyl-1,3- thiazol-2-yl)imidazo[1,2-a]pyridine-3- carboxamide 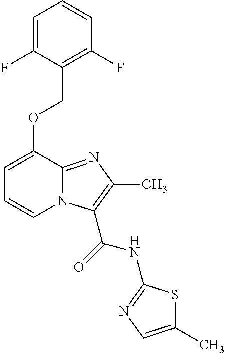 [SMILES: Cc1cnc(NC(=O)c2c(C)nc3c(OCc4c(F)cccc4F)cccn23)s1] (38% of theory; purity 89%) | LC-MS (Method 5): Rₜ = 0.92 min MS (ESpos): m/z = 415 (M + H)⁺ |

### Example 147

### N-(1-Ethyl-3,5-dimethyl-1H-pyrazol-4-yl)-2,6-dimethyl-8-[4,4,4-trifluoro-3-(trifluoromethyl)butoxy]imidazo[1,2-a]pyridine-3-carboxamide

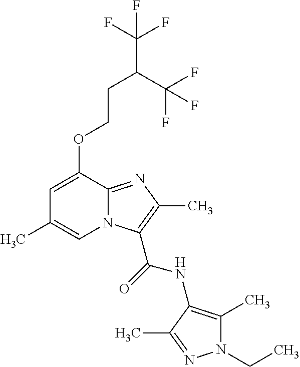 [SMILES: CCn1nc(C)c(NC(=O)c2c(C)nc3c(OCCC(C(F)(F)F)C(F)(F)F)cc(C)cn23)c1C]

11 mg (0.08 mmol) of 1-ethyl-3,5-dimethyl-1H-pyrazole-4-amine were initially charged, a solution of 31 mg (0.08 mmol) of 2,6-dimethyl-8-[4,4,4-trifluoro-3-(trifluoromethyl)butoxy]imidazo[1,2-a]pyridine-3-carboxylic acid from Example 59A in 0.3 ml of DMF, a solution of 40 mg (0.104 mmol) of HATU in 0.3 ml of DMF and then 16 mg (0.16 mmol) of 4-methylmorpholine were added and the mixture was shaken at RT overnight. The target compound was isolated by preparative HPLC (Method 6). 2.3 mg (6% of theory) were obtained.

LC-MS (Method 5): Rₜ=0.93 min

MS (ESpos): m/z=506 (M+H)⁺

Analogously to Example 147, the example compounds shown in Table 22 were prepared by reacting 2,6-dimethyl-8-[4,4,4-trifluoro-3-(trifluoromethyl)butoxy]imidazo[1,2-a]pyridine-3-carboxylic acid with the appropriate commercially available amines, under the conditions described:

TABLE 22

| | IUPAC name/structure | |
|---|---|---|
| Example | (Yield) | Analytical data |
| 148 | N-[1-(2-fluorobenzyl)-3,5-(dimethyl-1H-pyrazol-4- yl]-2,6-dimethyl-8-[4,4,4-trifluoro-3- (trifluoromethyl)butoxy]imidazo[1,2-a]pyridine-3- carboxamide 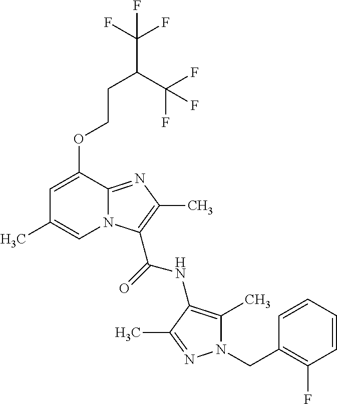 [SMILES: Cc1cc(OCCC(C(F)(F)F)C(F)(F)F)c2nc(C)c(C(=O)Nc3c(C)nn(Cc4ccccc4F)c3C)n2c1] (3% of theory) | LC-MS (Method 5): Rₜ = 1.04 min MS (ESpos): m/z = 586 (M + H)⁺ |
| 149 | 2,6-dimethyl-N-[3-methyl-5-(trifluoromethyl)- 1H-pyrazol-4-yl]-8-[4,4,4-trifluoro-3- (trifluoromethyl)butoxy]imidazo[1,2-a]pyridine- 3-carboxamide^{a)} 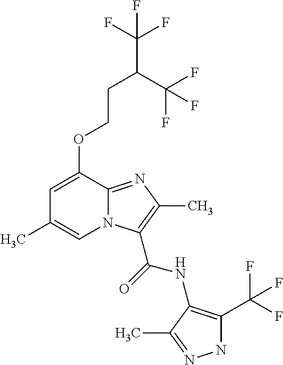 [SMILES: Cc1cc(OCCC(C(F)(F)F)C(F)(F)F)c2nc(C)c(C(=O)Nc3c(C)n[nH]c3C(F)(F)F)n2c1] (7% of theory) | LC-MS (Method 2): Rₜ = 0.95 min MS (ESpos): m/z = 532 (M + H)⁺ |

^{a}Instead of 4-methylmorpholine, the base used was N,N-diisopropylethylamine (3 equivalents). The reaction temperature was 60° C.

### Example 150

### 3-[3-({[8-(Cyclohexylmethoxy)-2-methylimidazo[1,2-a]pyridin-3-yl]carbonyl}amino)phenyl]propanoic acid

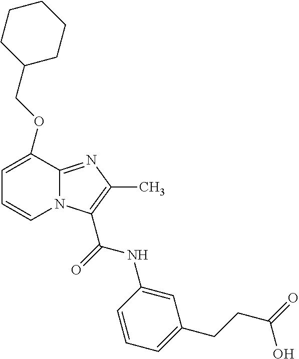 [SMILES: Cc1nc2c(OCC3CCCCC3)cccn2c1C(=O)Nc1cccc(CCC(=O)O)c1]

19 mg of ethyl 3-(3-aminophenyl)propanoate (0.1 mmol, 1.0 equivalents, possible preparation according to Strawn, Laurie M.; Martell, Robert E.; Simpson, Robert U.; Leach, Karen L.; Counsell, Raymond E.; Journal of Medicinal Chemistry, 1989, vol. 32, p. 2104-2110) were initially charged, and 29 mg of 8-(cyclohexylmethoxy)-2-methylimidazo[1,2-a]pyridine-3-carboxylic acid (Example 6A; 0.1 mmol, 1 equivalent) in 0.3 ml of DMSO, 41.7 mg of (benzotriazol-1-yloxy)bisdimethylaminomethylium fluoroborate (TBTU, 0.13 mmol, 1.3 equivalents) in 0.3 ml of DMSO and 26 mg of N,N-diisopropylethylamine (0.2 mmol, 2 equivalents) were added in succession. The mixture was shaken at RT overnight, 0.4 ml of 2 N aqueous sodium hydroxide solution was then added and the mixture was once more shaken at RT overnight. The solvent was removed and the residue was purified by preparative HPLC (Method 6). 31 mg (72% of theory) of the title compound were obtained.

LC-MS (Method 5): Rₜ=1.00 min

MS (ESpos): m/z=436.0 (M+H)⁺

### Example 151

### 8-[(3,3-Difluorocyclobutyl)methoxy]-2,6-dimethyl-N-[3-methyl-5-(trifluoromethyl)-1H-pyrazol-4-yl]imidazo[1,2-a]pyridine-3-carboxamide

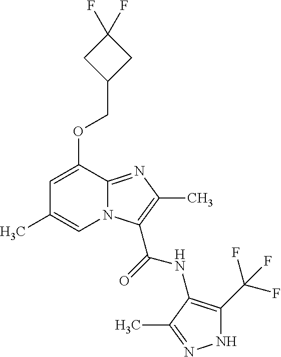 [SMILES: Cc1cc(OCC2CC(F)(F)C2)c2nc(C)c(C(=O)Nc3c(C)n[nH]c3C(F)(F)F)n2c1]

100 mg (0.322 mmol) of 8-[(3,3-difluorocyclobutyl)methoxy]-2,6-dimethylimidazo[1,2-a]pyridine-3-carboxylic acid (Example 62A) and 135 mg (0.354 mmol) of HATU were initially charged in 2 ml of DMF, 0.17 ml (0.97 mmol) of N,N-diisopropylethylamine and 64 mg (0.387 mmol) of 5-methyl-3-trifluoromethyl-1H-pyrazol-4-ylamine were added and the mixture was stirred at 60° C. overnight. Another 135 mg (0.354 mmol) of HATU and 0.17 ml (0.97 mmol) of N,N-diisopropylethylamine were added, and the mixture was stirred at 60° C. overnight. The pH was adjusted to pH 6 using 1 N aqueous hydrochloric acid and the reaction mixture was purified by preparative RP-HPLC (acetonitrile/water gradient with addition of 0.1% formic acid). The product fractions were combined and concentrated completely. This gave 55 mg of the target compound (36% of theory, purity 95%).

LC-MS (Method 2): Rₜ=0.81 min

MS (ESpos): m/z=458.2 (M+H)⁺

¹H-NMR (400 MHz, DMSO-d₆): δ=2.22 (s, 3H), 2.30 (s, 3H), 2.31-2.35 (m, 1H), 2.62 (s, 3H), 2.65-2.83 (m, 4H), 4.26 (d, 2H), 6.86 (br. s, 1H), 8.35 (s, 1H), 9.23 (br. s, 1H), 13.46 (s, 1H).

### Example 152

### 8-[(2,6-Difluorobenzyl)oxy]-2,6-dimethyl-N-[3-(methylsulphonyl)phenyl]imidazo[1,2-a]pyridine-3-carboxamide

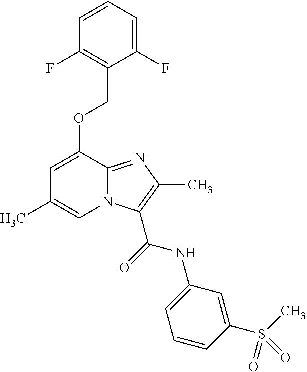 [SMILES: Cc1cc(OCc2c(F)cccc2F)c2nc(C)c(C(=O)Nc3cccc(S(C)(=O)=O)c3)n2c1]

75 mg (0.23 mmol) of 8-[(2,6-difluorobenzyl)oxy]-2,6-dimethylimidazo[1,2-a]pyridine-3-carboxylic acid (Example 21A), 112 mg (0.29 mmol) of O-(7-azabenzotriazol-1-yl)-N,N,N′N′-tetramethyluronium hexafluorophosphate (HATU) and 248 μl (1.42 mmol) of N,N-diisopropylethylamine (DIPEA) were initially charged in 0.8 ml of DMF. The mixture was stirred at RT for 20 min, and 61 mg (0.29 mmol) of 3-(methylsulphonyl)aniline hydrochloride were then added and the mixture was stirred at 60° C. for 1 h. The reaction mixture was diluted with acetonitrile, water/TFA was added and the mixture was purified by preparative HPLC (RP18 column, mobile phase: acetonitrile/water gradient with addition of 0.1% TFA). The product-containing fractions were concentrated and purified once more by preparative thick-layer chromatography (mobile phase: dichloromethane/methanol=50/1). This gave 2.5 mg (2% of theory; purity 90%) of the title compound.

LC-MS (Method 2): Rₜ=0.85 min

MS (ESpos): m/z=486 (M+H)⁺

### Example 153

### 8-[(2,6-Difluorobenzyl)oxy]-2,6-dimethyl-N-{3-[(methylsulphonyl)amino]phenyl}imidazo[1,2-a]pyridine-3-carboxamide

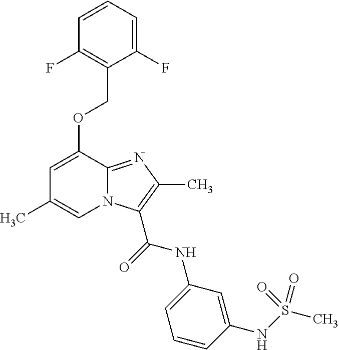

mg (0.23 mmol) of 8-[(2,6-difluorobenzyl)oxy]-2,6-dimethylimidazo[1,2-a]pyridine-3-carboxylic acid (Example 21A), 112 mg (0.29 mmol) of O-(7-azabenzotriazol-1-yl)-N,N,N′N′-tetramethyluronium hexafluorophosphate (HATU) and 197 μl (1.13 mmol) of N,N-diisopropylethylamine (DIPEA) were initially charged in 0.8 ml of DMF. The mixture was stirred at RT for 20 min, and 55 mg (0.29 mmol) of N-(3-aminophenyl)methanesulphonamide were then added and the mixture was stirred at 60° C. for 1 h. The reaction mixture was diluted with acetonitrile, water/TFA was added and the mixture was purified by preparative HPLC (RP18 column, mobile phase: acetonitrile/water gradient with addition of 0.1% TFA). The product-containing fractions were concentrated and purified once more by preparative thick-layer chromatography (mobile phase: dichloromethane/methanol=70/1). This gave 3.4 mg (3% of theory; purity 98%) of the title compound.

LC-MS (Method 2): Rₜ=0.80 min

MS (ESpos): m/z=501 (M+H)⁺

### Example 154

### 8-[(2,6-Difluorobenzyl)oxy]-N-[3,5-dimethyl-1-(methylsulphonyl)-1H-pyrazol-4-yl]-2-methylimidazo[1,2-a]pyridine-3-carboxamide

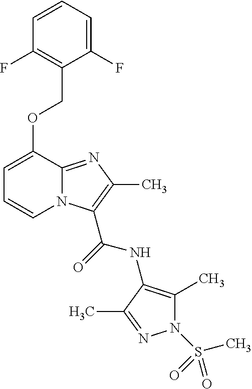 [SMILES: Cc1nn(S(C)(=O)=O)c(C)c1NC(=O)c1c(C)nc2c(OCc3c(F)cccc3F)cccn12]

40 mg (0.10 mmol) of 8-[(2,6-difluorobenzyl)oxy]-N-(3,5-dimethyl-1H-pyrazol-4-yl)-2-methylimidazo[1,2-a]pyridine-3-carboxamide from Example 29 were initially charged in 0.8 ml of dichloromethane, and 43 μl (0.31 mmol) of triethylamine and then 11 μl (0.15 mmol) of methanesulphonyl chloride were added. The reaction mixture was stirred at RT for 2 h. Another 2 μl (0.02 mmol) of methanesulphonyl chloride were added and the mixture was stirred at RT for 30 min. The reaction mixture was diluted with dichloromethane and washed twice with water, and the organic phase was dried over sodium sulphate, filtered, concentrated and dried under high vacuum. This gave 46 mg of the target compound (94% of theory, purity 98%).

LC-MS (Method 2): Rₜ=0.80 min

MS (ESpos): m/z=490 (M+H)⁺

### B. ASSESSMENT OF PHARMACOLOGICAL EFFICACY

The following abbreviations are used:

ATP adenosine triphosphate
Brij35 polyoxyethylene(23) lauryl ether
BSA bovine serum albumin
DTT dithiothreitol
TEA triethanolamine

The pharmacological action of the compounds of the invention can be demonstrated in the following assays:

### B-1. Measurement of sGC Enzyme Activity by Means of PPi Detection

Soluble guanylyl cyclase (sGC) converts GTP to cGMP and pyrophosphate (PPi) when stimulated. PPi is detected with the aid of the method described in WO 2008/061626. The signal that arises in the assay increases as the reaction progresses and serves as a measure of the sGC enzyme activity. With the aid of a PPi reference curve, the enzyme can be characterized in a known manner, for example in terms of conversion rate, stimulability or Michaelis constant.

### Test Procedure

To conduct the test, 29 μl of enzyme solution (0-10 nM soluble guanylyl cyclase (prepared according to Honicka et al., Journal of Molecular Medicine 77(1999)14-23), in 50 mM TEA, 2 mM magnesium chloride, 0.1% BSA (fraction V), 0.005% Brij 35, pH 7.5) were initially charged in the microplate, and 1 μl of the stimulator solution (0-10 μM 3-morpholinosydnonimine, SIN-1, Merck in DMSO) was added. The microplate was incubated at RT for 10 min Subsequently, 20 μl of detection mix (1.2 nM firefly luciferase (Photinus pyralis Luziferase, Promega), 29 μM dehydroluciferin (prepared according to Bitler & McElroy, Arch. Biochem. Biophys. 72 (1957) 358), 122 μM luciferin (Promega), 153 μM ATP (Sigma) and 0.4 mM DTT (Sigma) in 50 mM TEA, 2 mM magnesium chloride, 0.1% BSA (fraction V), 0.005% Brij 35, pH 7.5) were added. The enzyme reaction was started by adding 20 μl of substrate solution (1.25 mM guanosine 5′-triphosphate (Sigma) in 50 mM TEA, 2 mM magnesium chloride, 0.1% BSA (fraction V), 0.005% Brij 35, pH 7.5) and analysed continuously in a luminometer.

### B-2. Effect on a Recombinant Guanylate Cyclase Reporter Cell Line

The cellular action of the compounds of the invention is determined using a recombinant guanylate cyclase reporter cell line, as described in F. Wunder et al., Anal. Biochem. 339, 104-112 (2005).

Representative MEC values (MEC=minimum effective concentration) for the compounds of the invention are shown in the table below (in some cases as mean values for individual determinations):

TABLE A

| Example | MEC [μM] |
|---|---|
| 1 | 0.1 |
| 2 | 0.1 |
| 3 | 0.3 |
| 4 | 0.3 |
| 5 | 0.3 |
| 6 | 0.1 |
| 7 | 0.1 |
| 8 | 0.1 |
| 9 | 0.3 |
| 10 | 0.3 |
| 11 | 0.3 |
| 12 | 0.3 |
| 13 | 0.3 |
| 14 | 0.3 |
| 15 | 0.3 |
| 16 | 0.3 |
| 17 | 0.3 |
| 18 | 0.3 |
| 19 | 3.0 |
| 20 | 1.0 |
| 21 | 1.0 |
| 22 | 1.43 |
| 23 | 0.65 |
| 24 | 0.3 |
| 25 | 3.0 |
| 26 | 1.0 |
| 27 | 0.3 |
| 28 | 3.0 |
| 29 | 0.53 |
| 30 | 0.3 |
| 31 | 1.0 |
| 32 | 1.0 |
| 33 | 1.0 |
| 34 | 1.0 |
| 35 | 3.0 |
| 36 | 1.0 |
| 37 | 0.3 |
| 38 | 1.0 |
| 39 | 1.0 |
| 40 | 0.03 |
| 41 | 0.1 |
| 42 | 0.1 |
| 43 | 0.03 |
| 44 | 1.0 |
| 45 | 0.3 |
| 46 | 0.1 |
| 47 | 0.3 |
| 48 | 0.3 |
| 49 | 0.1 |
| 50 | 1.0 |
| 51 | 1.0 |
| 52 | 0.65 |
| 53 | 1.0 |
| 54 | 0.03 |
| 55 | 1.0 |
| 56 | 0.1 |
| 57 | 3.0 |
| 58 | 0.3 |
| 59 | 1.0 |
| 60 | 0.3 |
| 61 | 0.3 |
| 62 | 1.0 |
| 63 | 0.3 |
| 64 | 0.2 |
| 65 | 1.0 |
| 66 | 0.1 |
| 67 | 0.3 |
| 68 | 0.3 |
| 69 | 0.55 |
| 70 | 0.3 |
| 71 | 0.1 |
| 72 | 0.3 |
| 73 | 0.3 |
| 74 | 1.0 |
| 75 | 0.3 |
| 76 | 0.1 |
| 77 | 0.1 |
| 78 | 1.0 |
| 79 | 0.1 |
| 80 | 0.3 |
| 81 | 1.0 |
| 82 | 0.3 |
| 83 | 3.0 |
| 84 | 3.0 |
| 85 | 0.1 |
| 86 | 0.3 |
| 87 | 0.3 |
| 88 | 0.65 |
| 89 | 0.1 |
| 90 | 0.3 |
| 91 | 0.01 |
| 92 | 0.1 |
| 93 | 1.0 |
| 94 | 0.1 |
| 95 | 0.3 |
| 96 | 3.0 |
| 97 | 1.0 |
| 98 | 1.0 |
| 99 | 3.0 |
| 100 | 1.65 |
| 101 | 1.0 |
| 102 | 0.3 |
| 103 | 1.0 |
| 104 | 3.0 |
| 105 | 1.0 |
| 106 | 1.0 |
| 107 | 1.0 |
| 108 | 1.0 |
| 109 | 3.0 |
| 110 | 1.0 |
| 111 | 1.0 |
| 112 | 0.3 |
| 113 | 0.3 |
| 114 | 0.3 |
| 115 | 1.0 |
| 116 | 0.5 |
| 117 | 0.1 |
| 118 | 0.1 |
| 119 | 1.0 |
| 121 | 1.0 |
| 122 | 3.0 |
| 123 | 1.0 |
| 124 | 1.0 |
| 125 | 0.3 |
| 126 | 1.0 |
| 127 | 1.0 |
| 128 | 3.0 |
| 129 | 10 |
| 130 | 10 |
| 131 | 10 |
| 132 | 1.0 |
| 133 | 10 |
| 134 | 3.0 |
| 135 | 3.0 |
| 138 | 10 |
| 139 | 3.0 |
| 140 | 1.0 |
| 141 | 1.0 |
| 142 | 10 |
| 143 | 0.3 |
| 144 | 3.0 |
| 145 | 3.0 |
| 146 | 3.0 |
| 147 | 3.0 |
| 148 | 1.0 |
| 149 | 2.0 |
| 150 | 1.0 |
| 151 | 3.0 |
| 152 | 1.0 |
| 153 | 3.0 |
| 154 | 1.0 |

### B-3. Vasorelaxant Effect In Vitro

Rabbits are stunned by a blow to the neck and exsanguinated. The aorta is removed, freed from adhering tissue and divided into rings of width 1.5 mm, which are placed individually under prestress into 5 ml organ baths with carbogen-sparged Krebs-Henseleit solution at 37° C. having the following composition (each mM): sodium chloride: 119; potassium chloride: 4.8; calcium chloride dihydrate: 1; magnesium sulphate heptahydrate: 1.4; potassium dihydrogenphosphate: 1.2; sodium bicarbonate: 25; glucose: 10. The contractile force is determined with Statham UC2 cells, amplified and digitalized using A/D transducers (DAS-1802 HC, Keithley Instruments Munich), and recorded in parallel on linear recorders. To obtain a contraction, phenylephrine is added to the bath cumulatively in increasing concentration. After several control cycles, the substance to be studied is added in increasing dosage each time in every further run, and the magnitude of the contraction is compared with the magnitude of the contraction attained in the last preceding run. This is used to calculate the concentration needed to reduce the magnitude of the control value by 50% (IC₅₀ value). The standard administration volume is 5 μl; the DMSO content in the bath solution corresponds to 0.1%.

### B-4. Blood Pressure Measurement on Anaesthetized Rats

Male Wistar rats having a body weight of 300-350 g are anaesthetized with thiopental (100 mg/kg i.p.). After tracheotomy, a catheter is introduced into the femoral artery to measure the blood pressure. The substances to be tested are administered as solutions, either orally by means of a gavage or intravenously via the femoral vein (Stasch et al. Br. J. Pharmacol. 2002; 135: 344-355).

### B-5. Radiotelemetry Measurement of Blood Pressure in Conscious, Spontaneously Hypertensive Rats

A commercially available telemetry system from DATA SCIENCES INTERNATIONAL DSI, USA, is employed for the blood pressure measurement on conscious rats described below.

The system consists of 3 main components:

implantable transmitters (Physiotel® telemetry transmitter)
receivers (Physiotel® receiver) which are linked via a multiplexer (DSI Data Exchange Matrix) to a
data acquisition computer.

The telemetry system makes it possible to continuously record blood pressure, heart rate and body motion of conscious animals in their usual habitat.

### Animal Material

The studies are conducted on adult female spontaneously hypertensive rats (SHR Okamoto) with a body weight of >200 g. SHR/NCrl from the Okamoto Kyoto School of Medicine, 1963, were a cross of male Wistar Kyoto rats having greatly elevated blood pressure and female rats having slightly elevated blood pressure, and were handed over at F13 to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6.00 am and at 7.00 μm.

### Transmitter Implantation

The TA11 PA-C40 telemetry transmitters used are surgically implanted under aseptic conditions in the experimental animals at least 14 days before the first experimental use. The animals instrumented in this way can be used repeatedly after the wound has healed and the implant has settled.

For the implantation, the fasted animals are anaesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and the wound is closed layer by layer.

An antibiotic (Tardomyocel COMP, Bayer, 1 ml/kg s.c.) is administered postoperatively for prophylaxis of infection.

### Substances and Solutions

Unless stated otherwise, the substances to be studied are administered orally by gavage to a group of animals in each case (n=6). In accordance with an administration volume of 5 ml/kg of body weight, the test substances are dissolved in suitable solvent mixtures or suspended in 0.5% tylose.

A solvent-treated group of animals is used as control.

### Experimental Outline

The telemetry measuring unit present is configured for 24 animals Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch. They are switched to transmission in the run-up to the experiment. The signals emitted can be detected online by a data acquisition system (Dataquest™ A.R.T. for WINDOWS, DSI) and processed accordingly. The data are stored in each case in a file created for this purpose and bearing the experiment number.

In the standard procedure, the following are measured for 10-second periods in each case:
systolic blood pressure (SBP)
diastolic blood pressure (DBP)
mean arterial pressure (MAP)
heart rate (HR)
activity (ACT).

The acquisition of measurements is repeated under computer control at 5-minute intervals. The source data obtained as absolute values are corrected in the diagram with the currently measured barometric pressure (Ambient Pressure Reference Monitor; APR-1) and stored as individual data. Further technical details are given in the extensive documentation from the manufacturer company (DSI).

Unless indicated otherwise, the test substances are administered at 9:00 am on the day of the experiment. Following the administration, the parameters described above are measured over 24 hours.

### Evaluation

After the end of the experiment, the acquired individual data are sorted using the analysis software (DATAQUEST™ A.R.T.™ ANALYSIS). The blank value is assumed to be the time 2 hours before administration, and so the selected data set encompasses the period from 7:00 am on the day of the experiment to 9:00 am on the following day.

The data are smoothed over a predefinable period by determination of the average (15-minute average) and transferred as a text file to a storage medium. The measured values presorted and compressed in this way are transferred to Excel templates and tabulated. For each day of the experiment, the data obtained are stored in a dedicated file bearing the number of the experiment. Results and test protocols are stored in files in paper form sorted by numbers.

### LITERATURE

Klaus Witte, Kai Hu, Johanna Swiatek, Claudia Müssig, Georg Ertl and Björn Lemmer: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994.

### B-6. Determination of Pharmacokinetic Parameters Following Intravenous and Oral Administration

The pharmacokinetic parameters of the compounds according to the invention are determined in male CD-1 mice, male Wistar rats and female beagles. Intravenous administration in the case of mice and rats is effected by means of a species-specific plasma/DMSO formulation, and in the case of dogs by means of a water/PEG400/ethanol formulation. In all species, oral administration of the dissolved substance is performed via gavage, based on a water/PEG400/ethanol formulation. The removal of blood from rats is simplified by inserting a silicone catheter into the right Vena jugularis externa prior to substance administration. The operation is effected at least one day prior to the experiment with isofluran anaesthesia and administration of an analgesic (atropine/rimadyl (3/1) 0.1 ml s.c.). The blood is taken (generally more than 10 time points) within a time window including terminal time points of at least 24 to a maximum of 72 hours after substance administration. The blood is removed into heparinized tubes. The blood plasma is then obtained by centrifugation; if required, it can be stored at −20° C. until further processing.

An internal standard (which may also be a chemically unrelated substance) is added to the samples of the compounds of the invention, calibration samples and qualifiers, and there follows protein precipitation by means of acetonitrile in excess. Addition of a buffer solution matched to the LC conditions, and subsequent vortexing, is followed by centrifugation at 1000 g. The supernatant is analysed by LC-MS/MS using C18 reversed-phase columns and variable eluent mixtures. The substances are quantified via the peak heights or areas from extracted ion chromatograms of specific selected ion monitoring experiments.

The plasma concentration/time plots determined are used to calculate the pharmacokinetic parameters such as AUC, Cₘₐₓ, t_{1/2} (terminal half-life), F (bioavailability), MRT (mean residence time) and CL (clearance), by means of a validated pharmacokinetic calculation program.

Since the substance quantification is performed in plasma, it is necessary to determine the blood/plasma distribution of the substance in order to be able to adjust the pharmacokinetic parameters correspondingly. For this purpose, a defined amount of substance is incubated in heparinized whole blood of the species in question in a rocking roller mixer for 20 min After centrifugation at 1000 g, the plasma concentration is measured (by means of LC-MS/MS; see above) and determined by calculating the ratio of the C_{blood}/Cₚₗₐₛₘₐ value.

### B-7. Metabolic Study

To determine the metabolic profile of the compounds of the invention, they are incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds of the invention were incubated with a concentration of about 0.1-10 μM. To this end, stock solutions of the compounds of the invention having a concentration of 0.01-1 mM in acetonitrile were prepared, and then pipetted with 1:100 dilution into the incubation mixture. Liver microsomes and recombinant enzymes were incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation mixtures were stopped with acetonitrile (final concentration about 30%) and the protein was centrifuged off at about 15 000×g. The samples thus stopped were either analysed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable eluent mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic reduction of the compound of the invention in the incubation mixtures.

### B-8. Caco-2 Permeability Test

The permeability of a test substance was determined with the aid of the Caco-2 cell line, an established in vitro model for permeability prediction at the gastrointestinal barrier (Artursson, P. and Karlsson, J. (1991). Correlation between oral drug absorption in humans and apparent drug permeability coefficients in human intestinal epithelial (Caco-2) cells. Biochem. Biophys. 175 (3), 880-885). The Caco-2 cells (ACC No. 169, DSMZ, Deutsche Sammlung von Mikroorganismen and Zellkulturen, Braunschweig, Germany) were sown in 24-well plates having an insert and cultivated for 14 to 16 days. For the permeability studies, the test substance was dissolved in DMSO and diluted to the final test concentration with transport buffer (Hanks Buffered Salt Solution, Gibco/Invitrogen, with 19.9 mM glucose and 9.8 mM HEPES). In order to determine the apical to basolateral permeability (PₐₚₚA-B) of the test substance, the solution comprising the test substance was applied to the apical side of the Caco-2 cell monolayer, and transport buffer to the basolateral side. In order to determine the basolateral to apical permeability (PₐₚₚB-A) of the test substance, the solution comprising the test substance was applied to the basolateral side of the Caco-2 cell monolayer, and transport buffer to the apical side. At the start of the experiment, samples were taken from the respective donor compartment in order to ensure the mass balance. After an incubation time of two hours at 37° C., samples were taken from the two compartments. The samples were analysed by means of LC-MS/MS and the apparent permeability coefficients (Pₐₚₚ) were calculated. For each cell monolayer, the permeability of Lucifer Yellow was determined to ensure cell layer integrity. In each test run, the permeability of atenolol (marker for low permeability) and sulfasalazine (marker for active excretion) was also determined as quality control.

B-9. hERG Potassium Current Assay

The hERG (human ether-a-go-go related gene) potassium current makes a significant contribution to the repolarization of the human cardiac action potential (Scheel et al., 2011). Inhibition of this current by pharmaceuticals can in rare cases cause potentially lethal cardiac arrhythmia, and is therefore studied at an early stage during drug development.

The functional hERG assay used here is based on a recombinant HEK293 cell line which stably expresses the KCNH2(HERG) gene (Zhou et al., 1998). These cells are studied by means of the “whole-cell voltage-clamp” technique (Hamill et al., 1981) in an automated system (Patchliner™; Nanion, Munich, Germany), which controls the membrane voltage and measures the hERG potassium current at room temperature. The PatchControlHT™ software (Nanion) controls the Patchliner system, data capture and data analysis. The voltage is controlled by 2 EPC-10 quadro amplifiers controlled by the PatchMasterPro™ software (both: HEKA Elektronik, Lambrecht, Germany) NPC-16 chips with moderate resistance (˜2 MΩ; Nanion) serve as the planar substrate for the voltage clamp experiments.

NPC-16 chips are filled with intra- and extracellular solution (cf. Himmel, 2007) and with cell suspension. After forming a gigaohm seal and establishing whole-cell mode (including several automated quality control steps), the cell membrane is clamped at the −80 mV holding potential. The subsequent voltage clamp protocol changes the command voltage to +20 mV (for 1000 ms), −120 mV (for 500 ms), and back to the −80 mV holding potential; this is repeated every 12 s. After an initial stabilization phase (about 5-6 minutes), test substance solution is introduced by pipette in rising concentrations (e.g. 0.1, 1, and 10 μmol/l) (exposure about 5-6 minutes per concentration), followed by several washing steps.

The amplitude of the upward “tail” current which is generated by a change in potential from +20 mV to −120 mV serves to quantify the hERG potassium current, and is described as a function of time (IgorPro™ Software). The current amplitude at the end of various time intervals (for example stabilization phase before test substance, first/second/third concentration of test substance) serves to establish a concentration/effect curve, from which the half-maximum inhibiting concentration IC₅₀ of the test substance is calculated.
Hamill O P, Marty A, Neher E, Sakmann B, Sigworth F J Improved patch-clamp techniques for high-resolution current recording from cells and cell-free membrane patches. Pfluegers Arch 1981; 391:85-100.
Himmel H M. Suitability of commonly used excipients for electrophysiological in-vitro safety pharmacology assessment of effects on hERG potassium current and on rabbit Purkinje fiber action potential. J Pharmacol Toxicol Methods 2007; 56:145-158.
Scheel O, Himmel H, Rascher-Eggstein G, Knott T. Introduction of a modular automated voltage-clamp platform and its correlation with manual human ether-a-go-go related gene voltage-clamp data. Assay Drug Dev Technol 2011; 9:600-607.
Zhou Z F, Gong Q, Ye B, Fan Z, Makielski J C, Robertson G A, January C T. Properties of hERG channels stably expressed in HEK293 cells studied at physiological temperature. Biophys J 1998; 74:230-241.

### C. WORKING EXAMPLES FOR PHARMACEUTICAL COMPOSITIONS

The compounds of the invention can be converted to pharmaceutical formulations as follows:

### Tablet:

### Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

### Production:

The mixture of compound of the invention, lactose and starch is granulated with a 5% solution (w/w) of the PVP in water. The granules are dried and then mixed with the magnesium stearate for 5 minutes. This mixture is compressed in a conventional tabletting press (see above for format of the tablet). The guide value used for the pressing is a pressing force of 15 kN.

### Suspension for Oral Administration:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol; the compound of the invention is added to the suspension. The water is added while stirring. The mixture is stirred for about 6 h before swelling of the Rhodigel is complete.

### Solution for Oral Administration:

### Composition:

500 mg of the compound of the invention, 2.5 g of polysorbate and 97 g of polyethylene glycol 400. 20 g of oral solution correspond to a single dose of 100 mg of the compound of the invention.

### Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

1. Compound of the formula (I) 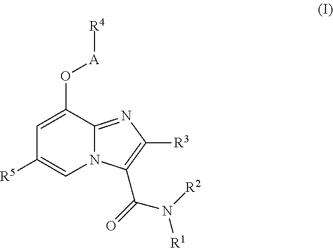
in which
A represents CH₂, CD₂ or CH(CH₃),
R¹ represents phenyl, naphthyl or 5- to 10-membered heteroaryl,
where phenyl, naphthyl and 5- to 10-membered heteroaryl may be substituted by 1 to 4 substituents independently of one another selected from the group consisting of halogen, cyano, difluoromethyl, trifluoromethyl, (C₁-C₆)-alkyl, (C₃-C₇)-cycloalkyl, (C₁-C₄)-alkylsulphonyl, (C₃-C₆)-cycloalkylsulphonyl, (C₁-C₄)-alkylsulphonylamino, (C₃-C₆)-cycloalkylsulphonylamino, hydroxy, difluoromethoxy, trifluoromethoxy, (C₁-C₄)-alkoxy, (C₁-C₄)-alkylcarbonylamino, amino, mono-(C₁-C₄)-alkylamino, di-(C₁-C₄)-alkylamino, mono-(C₁-C₄)-alkylaminocarbonyl, di-(C₁-C₄)-alkylaminocarbonyl, phenyl, benzyl, 4- to 7-membered heterocyclyl and 5-membered heteroaryl,
in which (C₁-C₆)-alkyl, mono-(C₁-C₄)-alkylamino and di-(C₁-C₄)-alkylamino may be substituted by 1 to 3 substituents independently of one another selected from the group consisting of fluorine, trifluoromethyl, (C₃-C₇)-cycloalkyl, hydroxy, (C₁-C₄)-alkoxy, trifluoromethoxy, 2,2,2-trifluoroethoxy, hydroxycarbonyl, (C₁-C₄)-alkoxycarbonyl, (C₁-C₄)-alkylcarbonylamino, aminocarbonyl, mono-(C₁-C₄)-alkylaminocarbonyl, di-(C₁-C₄)-alkylaminocarbonyl, (C₁-C₄)-alkylsulphonyl, (C₁-C₄)-alkylsulphonylamino, aminocarbonyloxy, phenyl, 4- to 7-membered heterocyclyl, 5-membered heteroaryl and a —NR⁶R⁷ group,
in which
R⁶ represents hydrogen, (C₁-C₄)-alkyl or (C₃-C₇)-cycloalkyl,
in which (C₁-C₄)-alkyl for its part may be substituted by 1 or 2 substituents independently of one another selected from the group consisting of fluorine, trifluoromethyl, (C₃-C₇)-cycloalkyl, hydroxy, (C₁-C₄)-alkoxy, amino, mono-(C₁-C₄)-alkylamino and di-(C₁-C₄)-alkylamino,
R⁷ represents hydrogen or (C₁-C₄)-alkyl,
or
in which R⁶ and R⁷ together with the nitrogen atom to which they are attached form a 4- to 7-membered heterocycle,
in which the 4- to 7-membered heterocycle for its part may be substituted by 1 to 3 substituents independently of one another selected from the group consisting of fluorine, (C₁-C₄)-alkyl, (C₃-C₇)-cycloalkyl, hydroxy, hydroxymethyl, oxo, (C₁-C₄)-alkoxy, amino, mono-(C₁-C₄)-alkylamino and di-(C₁-C₄)-alkylamino,
and
in which phenyl, benzyl, 4- to 7-membered heterocyclyl and 5-membered heteroaryl may be substituted by 1 to 3 substituents independently of one another selected from the group consisting of halogen, difluoromethyl, trifluoromethyl, (C₁-C₄)-alkyl, hydroxy, difluoromethoxy, trifluoromethoxy and (C₁-C₄)-alkoxy,
or
where two adjacent radicals at the phenyl together with the carbon atoms to
which they are attached form a 5- or 6-membered heterocycle,
in which the 5- or 6-membered heterocycle for its part may be substituted by 1 to 3 substituents independently of one another selected from the group consisting of fluorine, trifluoromethyl, (C₁-C₄)-alkyl, hydroxy, hydroxymethyl, oxo and (C₁-C₄)-alkoxy,
R² represents hydrogen,
R³ represents hydrogen, (C₁-C₄)-alkyl, cyclopropyl, monofluoromethyl, difluoromethyl or trifluoromethyl,
R⁴ represents (C₄-C₆)-alkyl, (C₃-C₇)-cycloalkyl or phenyl,
where (C₄-C₆)-alkyl may be substituted by 1 or 2 substituents independently of one another selected from the group consisting of fluorine and trifluoromethyl, where (C₃-C₇)-cycloalkyl may be substituted by 1 to 4 substituents selected independently from the group of fluorine, trifluoromethyl and (C₁-C₄)-alkyl, and
where phenyl may be substituted by 1 to 4 substituents independently of one another selected from the group consisting of halogen, cyano, monofluoromethyl, difluoromethyl, trifluoromethyl, (C₁-C₄)-alkyl, (C₁-C₄)-alkoxy, difluoromethoxy and trifluoromethoxy,
R⁵ represents hydrogen, halogen, cyano, difluoromethyl, trifluoromethyl, (C₁-C₄)-alkyl, ethynyl, (C₃-C₇)-cycloalkyl, (C₁-C₄)-alkoxy or 4- to 7-membered heterocyclyl,
and the N-oxides, salts, solvates, salts of the N-oxides and solvates of the N-oxides or salts thereof.

2. The compound of claim 1 in which
A represents CH₂,
R¹ represents phenyl, naphthyl, pyrazolyl, imidazolyl, isoxazolyl, 1,3,4-thiadiazol-2-yl, 1,3-thiazol-2-yl, 1,3-oxazol-2-yl, pyridyl, pyrimidin-2-yl, indolyl, pyrrolo[2,3-b]pyridine, indazolyl, pyrazolo[1,5-a]pyridine, quinolinyl, isoquinolinyl or cinnolinyl, where phenyl, naphthyl, pyrazolyl, isoxazolyl, 1,3,4-thiadiazol-2-yl, 1,3-thiazol-2-yl, 1,3-oxazol-2-yl, pyridyl, pyrimidin-2-yl, indolyl, pyrrolo[2,3-b]pyridine, indazolyl, pyrazolo[1,5-a]pyridine, quinolinyl, isoquinolinyl and cinnolinyl may be substituted by 1 to 4 substituents independently of one another selected from the group consisting of fluorine, chlorine, trifluoromethyl, (C₁-C₆)-alkyl, cyclopropyl, cyclobutyl, cyclopentyl, (C₁-C₄)-alkylsulphonyl, (C₁-C₄)-alkylsulphonylamino, trifluoromethoxy, (C₁-C₄)-alkoxy, methylcarbonylamino, ethylcarbonylamino, methylamino, ethylamino, dimethylamino, diethylamino, methylaminocarbonyl, ethylaminocarbonyl, dimethylaminocarbonyl, diethylaminocarbonyl, phenyl, benzyl, azetidinyl, pyrrolidinyl, piperidinyl, tetrahydrofuranyl, tetrahydropyranyl, piperazinyl, morpholinyl and tetrazolyl,
in which (C₁-C₆)-alkyl, ethylamino and diethylamino may be substituted by 1 to 3 substituents independently of one another selected from the group consisting of fluorine, trifluoromethyl, cyclopropyl, cyclobutyl, hydroxy, methoxy, ethoxy, 2,2,2-trifluoroethoxy, methylcarbonylamino, ethylcarbonylamino, methylaminocarbonyl, ethylaminocarbonyl, dimethylaminocarbonyl, diethylaminocarbonyl, methylsulphonyl, ethylsulphonyl, aminocarbonyloxy, azetidin-3-yl, pyrrolidin-2-yl, pyrrolidin-3-yl, piperidin-2-yl, piperidin-3-yl, piperidin-4-yl, tetrahydrofuranyl, tetrahydropyranyl, piperazin-2-yl, piperazin-3-yl, morpholin-2-yl, morpholin-3-yl and tetrazolyl and a —NR⁶R⁷ group,
in which
R⁶ represents hydrogen, (C₁-C₄)-alkyl, cyclopropyl or cyclobutyl,
in which (C₁-C₄)-alkyl may itself be substituted by 1 or 2 substituents each independently selected from the group of fluorine, trifluoromethyl, cyclopropyl, cyclobutyl, hydroxy, methoxy and ethoxy,
R⁷ represents hydrogen or (C₁-C₄)-alkyl,
or
in which R⁶ and R⁷ together with the nitrogen atom to which they are attached form an azetidinyl, pyrrolidinyl, piperidinyl, piperazinyl or morpholinyl ring,
in which the azetidinyl, pyrrolidinyl, piperidinyl, piperazinyl and morpholinyl ring for its part may be substituted by 1 to 3 substituents independently of one another selected from the group consisting of fluorine, methyl, ethyl, cyclopropyl, cyclobutyl, hydroxy, hydroxymethyl, oxo, methoxy and ethoxy,
or
where two adjacent radicals at the phenyl together with the carbon atoms to which they are attached form a dihydropyrrolyl, tetrahydropyridinyl, dihydrooxazinyl or dihydropyrazinyl ring,
in which the dihydropyrrolyl, tetrahydropyridinyl, dihydrooxazinyl and dihydropyrazinyl ring for its part may be substituted by 1 to 3 substituents independently of one another selected from the group consisting of fluorine, methyl, ethyl, hydroxy, hydroxymethyl and oxo,
R² represents hydrogen,
R³ is methyl,
R⁴ represents phenyl,
where phenyl is substituted by 1 to 4 substituents independently of one another selected from the group consisting of fluorine and chlorine,
R⁵ is hydrogen, fluorine, chlorine or methyl,
and the salts, solvates and solvates of the salts thereof.

3. The compound of claim 1, wherein
A represents CH₂,
R¹ represents indolyl, pyrrolo[2,3-b]pyridine, indazolyl, pyrazolo[1,5-a]pyridine, quinolinyl or isoquinolinyl,
where pyrrolo[2,3-b]pyridine, indolyl, indazolyl, pyrazolo[1,5-a]pyridine, quinolinyl and isoquinolinyl may be substituted by 1 to 3 substituents independently of one another selected from the group consisting of fluorine, chlorine, trifluoromethyl, (C₁-C₄)-alkyl, methoxy and ethoxy,
in which (C₁-C₄)-alkyl may be substituted by 1 to 3 substituents independently of one another selected from the group consisting of fluorine, trifluoromethyl, cyclopropyl, hydroxy, methoxy, ethoxy and methylsulphonyl,
R² represents hydrogen,
R³ represents methyl,
R⁴ represents phenyl,
where phenyl is substituted by 1 to 3 substituents independently of one another selected from the group consisting of fluorine and chlorine,
R⁵ represents hydrogen, fluorine, chlorine or methyl,
and the salts, solvates and solvates of the salts thereof.

4. The compound of claim 1, wherein
A represents CH₂,
R¹ represents pyrazol-4-yl,
where pyrazol-4-yl may be substituted by 1 to 3 substituents independently of one another selected from the group consisting of trifluoromethyl, (C₁-C₄)-alkyl and cyclopropyl,
in which (C₁-C₄)-alkyl may be substituted by 1 to 3 substituents independently of one another selected from the group consisting of fluorine, trifluoromethyl, cyclopropyl, hydroxy, methoxy, ethoxy, 2,2,2-trifluoroethoxy, methylsulphonyl and a —NR⁶R⁷ group,
in which
R⁶ represents hydrogen or (C₁-C₄)-alkyl,
in which (C₁-C₄)-alkyl may itself be substituted by 1 or 2 substituents each independently selected from the group consisting of fluorine, trifluoromethyl, cyclopropyl, hydroxy, methoxy and ethoxy,
R⁷ represents hydrogen or (C₁-C₄)-alkyl,
or
in which R⁶ and R⁷ together with the nitrogen atom to which they are attached form an azetidinyl, pyrrolidinyl, piperidinyl, piperazinyl or morpholinyl ring,
in which the azetidinyl, pyrrolidinyl, piperidinyl, piperazinyl and morpholinyl ring for its part may be substituted by 1 to 3 substituents independently of one another selected from the group consisting of fluorine, methyl, ethyl, hydroxy, oxo, methoxy and ethoxy,
R² represents hydrogen,
R³ represents methyl,
R⁴ represents phenyl,
where phenyl is substituted by 1 to 3 substituents independently of one another selected from the group consisting of fluorine and chlorine,
R⁵ represents hydrogen, fluorine, chlorine or methyl,
and the salts, solvates and solvates of the salts thereof.

5. Process for preparing the compound of claim 1, comprising
[A] converting a compound of the formula (II) 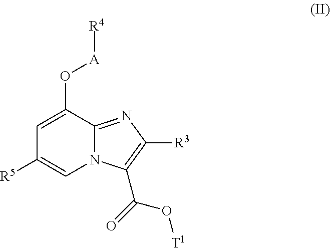
in which A, R³, R⁴, R⁵ each have the meanings given in claim 1 and
T¹ represents (C₁-C₄)-alkyl or benzyl,
in an inert solvent in the presence of a suitable base or acid into a carboxylic acid of the formula (III) 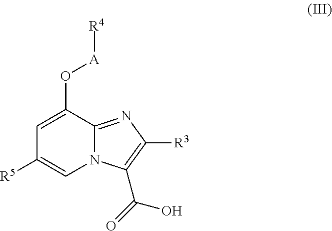
in which A, R³, R⁴ and R⁵ each have the meanings given in claim 1,
and reacting the carboxylic acid of the formula (III) in an inert solvent under amide coupling conditions with an amine of the formula (IV) 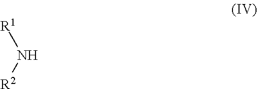
in which R¹ and R² each have the meanings given in claim 1,
or
[B] converting a compound of the formula (III-B) 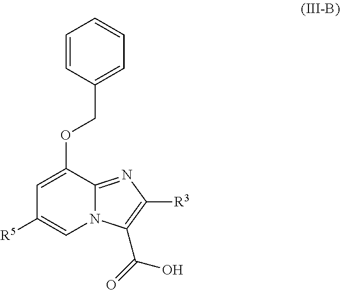
in which R³ and R⁵ each have the meanings given in claim 1, in an inert solvent under amide coupling conditions with an amine of the formula (IV) into a compound of the formula (I-B) 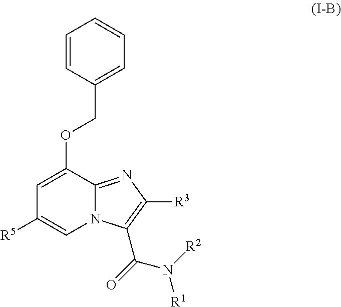
in which R¹, R², R³ and R⁵ each have the meanings given in claim 1,
and subsequently detaching the benzyl group therefrom to form a compound of the formula (V) 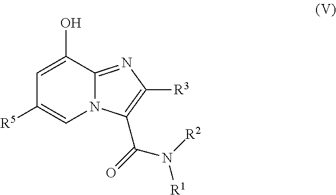
in which R¹, R², R³ and R⁵ each have the meanings given in claim 1,
reacting the compound of the formula (V) in an inert solvent in the presence of a suitable base with a compound of the formula (VI) 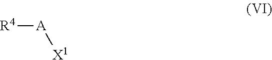
in which A and R⁴ have the meanings given in claim 1 and
X¹ represents a suitable leaving group,
and converting the resulting compounds of the formula (I), where appropriate, with the appropriate (i) solvents and/or (ii) bases or acids into solvates, salts and/or solvates of the salts thereof.

6. (canceled)

7. (canceled)

8. Medicament comprising the compound of claim 1 in combination with an inert, non-toxic, pharmaceutically suitable excipient.

9. Medicament comprising the compound of claim 1 in combination with a further active ingredient selected from the group consisting of organic nitrates, NO donors, cGMP-PDE inhibitors, antithrombotic agents, hypotensive agents and lipid metabolism modifiers.

10. (canceled)

11. Method for treatment and/or prophylaxis of heart failure, angina pectoris, hypertension, pulmonary hypertension, ischaemias, vascular disorders, thromboembolic disorders and arteriosclerosis in humans and animals comprising administering a therapeutically effective amount of the compound of claim 1 to a human or animal in need thereof.

12. A method for treatment and/or prophylaxis of heart failure, angina pectoris, hypertension, pulmonary hypertension, ischaemias, vascular disorders, thromboembolic disorders and arteriosclerosis in humans and animals comprising administering a therapeutically effective amount of the medicament of claim 8 to a human or animal in need thereof.

13. A method for treatment and/or prophylaxis of heart failure, angina pectoris, hypertension, pulmonary hypertension, ischaemias, vascular disorders, thromboembolic disorders and arteriosclerosis in humans and animals comprising administering a therapeutically effective amount of the medicament of claim 9 to a human or animal in need thereof.
